SEQUENCE LISTING (130 sequences)

SEQ ID NO: 1 - protein, 6 aa, Artificial Sequence
  C-terminal PP2Ac
TPDYFL

SEQ ID NO: 2 - protein, 4 aa, Artificial Sequence
  CDR-H3
RFAY

SEQ ID NO: 3 - protein, 16 aa, Artificial Sequence
  CDR-H2
YISYDGSNNYNPSLKN

SEQ ID NO: 4 - protein, 6 aa, Artificial Sequence
  CDR-H1
SGYYWN

SEQ ID NO: 5 - protein, 9 aa, Artificial Sequence
  CDR-L3
FQGSHVPWT

SEQ ID NO: 6 - protein, 7 aa, Artificial Sequence
  CDR-L2
KVSNRFS

SEQ ID NO: 7 - protein, 16 aa, Artificial Sequence
  CDR-L1
RSSQSIVHSNGNTYLE

SEQ ID NO: 8 - protein, 30 aa, Artificial Sequence
  H-FR1
DVQLQESGPGLVKPSQSLSLTCSVTGYSIT

SEQ ID NO: 9 - protein, 14 aa, Artificial Sequence
  H-FR2
WIRQFPGNKLEWMG

SEQ ID NO: 10 - protein, 32 aa, Artificial Sequence
  H-FR3
RISITRDTSKNQFFLKLNSVTTEDTATYYCAG

SEQ ID NO: 11 - protein, 11 aa, Artificial Sequence
  H-FR4
WGQGTLVTVSA

SEQ ID NO: 12 - protein, 23 aa, Artificial Sequence
  L-FR1
DVLMTQTPLSLPVSLGDQASISC

SEQ ID NO: 13 - protein, 15 aa, Artificial Sequence
  L-FR2
WYLQKPGQSPKLLIY

SEQ ID NO: 14 - protein, 32 aa, Artificial Sequence
  L-FR3
GVPDRFSGSGSGTDFTLKINRVEAEDLGVYYC

SEQ ID NO: 15 - protein, 10 aa, Artificial Sequence
  L-FR4
FGGGTKLEIK

SEQ ID NO: 16 - protein, 113 aa, Artificial Sequence
  7C10-C5 heavy chain variable region
DVQLQESGPGLVKPSQSLSLTCSVTGYSITSGYYWNWIRQFPGNKLEWMGYISYDGSNNYNPSLKNRISITRDTSKNQFFLKLNSVTTEDTATYYCAGRFAYWGQGTLVTVSA

SEQ ID NO: 17 - protein, 112 aa, Artificial Sequence
  7C10-C5 light chain variable region
DVLMTQTPLSLPVSLGDQASISCRSSQSIVHSNGNTYLEWYLQKPGQSPKLLIYKVSNRFSGVPDRFSGSGSGTDFTLKINRVEAEDLGVYYCFQGSHVPWTFGGGTKLEIK

SEQ ID NO: 18 - protein, 11 aa, Artificial Sequence
  C-terminal region of PP2Ac
HVTRRTPDYFL

SEQ ID NO: 19 - protein, 6 aa, Artificial Sequence
  C-terminal region of PP4c
VADYFL

SEQ ID NO: 20 - protein, 11 aa, Artificial Sequence
  C-terminal region of PP4c comprised in the PP4c peptide
PSKKPVADYFL

SEQ ID NO: 21 - protein, 6 aa, Artificial Sequence
  C-terminal region of PP6c
TTPYFL

SEQ ID NO: 22 - protein, 11 aa, Artificial Sequence
  C-terminal region of PP6c comprised in the PP6c peptide
IPPRTTTPYFL

SEQ ID NO: 23 - protein, 5 aa, Artificial Sequence
  immunization peptide/immunogen
PDYFL

SEQ ID NO: 24 - protein, 7 aa, Artificial Sequence
  immunization peptide/immunogen
RTPDYFL

SEQ ID NO: 25 - protein, 4 aa, Artificial Sequence
  CDR-H3 (Chothia numbering)
RFAY

SEQ ID NO: 26 - protein, 5 aa, Artificial Sequence
  CDR-H2 (Chothia numbering)
SYDGS

SEQ ID NO: 27 - protein, 8 aa, Artificial Sequence
  CDR-H1 (Chothia numbering)
GYSITSGY

SEQ ID NO: 28 - protein, 9 aa, Artificial Sequence
  CDR-L3 (Chothia numbering)
FQGSHVPWT

SEQ ID NO: 29 - protein, 7 aa, Artificial Sequence
  CDR-L2 (Chothia numbering)
KVSNRFS

SEQ ID NO: 30 - protein, 16 aa, Artificial Sequence
  CDR-L1 (Chothia numbering)
RSSQSIVHSNGNTYLE

SEQ ID NO: 31 - protein, 25 aa, Artificial Sequence
  H-FR1 (Chothia numbering)
DVQLQESGPGLVKPSQSLSLTCSVT

SEQ ID NO: 32 - protein, 19 aa, Artificial Sequence
  H-FR2 (Chothia numbering)
YWNWIRQFPGNKLEWMGYI

SEQ ID NO: 33 - protein, 41 aa, Artificial Sequence
  H-FR3 (Chothia numbering)
NNYNPSLKNRISITRDTSKNQFFLKLNSVTTEDTATYYCAG

SEQ ID NO: 34 - protein, 11 aa, Artificial Sequence
  H-FR4 (Chothia numbering)
WGQGTLVTVSA

SEQ ID NO: 35 - protein, 23 aa, Artificial Sequence
  L-FR1 (Chothia numbering)
DVLMTQTPLSLPVSLGDQASISC

SEQ ID NO: 36 - protein, 15 aa, Artificial Sequence
  L-FR2 (Chothia numbering)
WYLQKPGQSPKLLIY

SEQ ID NO: 37 - protein, 32 aa, Artificial Sequence; identical to SEQ ID NO: 14
  L-FR3 (Chothia numbering)

SEQ ID NO: 38 - protein, 10 aa, Artificial Sequence
  L-FR4 (Chothia numbering)
FGGGTKLEIK

SEQ ID NO: 39 - DNA, 339 bp, Artificial Sequence
  7C10-C5 heavy chain variable region
gatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagctacgacggtagcaataactacaacccatctctcaaaaatcgaatctccatcactcgtgacacatctaagaaccagtttttcctgaagttgaattctgtgactactgaggacacagctacatattactgtgctggacggtttgcttactggggccaagggactctggtcactgtctctgca

SEQ ID NO: 40 - DNA, 336 bp, Artificial Sequence
  7C10-C5 light chain variable region
gatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcaacagagtggaggctgaggatctgggagtttattactgctttcaaggttcacatgttccgtggacgttcggtggaggcaccaagctggaaatcaaa

SEQ ID NO: 41 - DNA, 900 bp, Artificial Sequence
  recombinant monovalent scFv antibody
atggacatgagggtccctgctcagctcctggggctcctgctgctctggctctcaggtgccagatgtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcaacagagtggaggctgaggatctgggagtttattactgctttcaaggttcacatgttccgtggacgttcggtggaggcaccaagctggaaatcaaatcctctggtggcggtggctcgggcggtggtgggggtggttcctctagatcttccctcgatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagctacgacggtagcaataactacaacccatctctcaaaaatcgaatctccatcactcgtgacacatctaagaaccagtttttcctgaagttgaattctgtgactactgaggacacagctacatattactgtgctggacggtttgcttactggggccaagggactctggtcactgtctctgcagccaaaacgacacccccatctgtcactagtggccaggccggccagcaccatcaccatcaccatggcgcatacccgtacgacgttccggactacgcttcttag

SEQ ID NO: 42 - protein, 299 aa, Artificial Sequence
  recombinant monovalent scFv antibody
MDMRVPAQLLGLLLLWLSGARCDVLMTQTPLSLPVSLGDQASISCRSSQSIVHSNGNTYLEWYLQKPGQSPKLLIYKVSNRFSGVPDRFSGSGSGTDFTLKINRVEAEDLGVYYCFQGSHVPWTFGGGTKLEIKSSGGGGSGGGGGGSSRSSLDVQLQESGPGLVKPSQSLSLTCSVTGYSITSGYYWNWIRQFPGNKLEWMGYISYDGSNNYNPSLKNRISITRDTSKNQFFLKLNSVTTEDTATYYCAGRFAYWGQGTLVTVSAAKTTPPSVTSGQAGQHHHHHHGAYPYDVPDYAS

SEQ ID NO: 43 - DNA, 1746 bp, Artificial Sequence
  recombinant bivalent scFv antibody
atggacatgagggtccctgctcagctcctggggctcctgctgctctggctctcaggtgccagatgtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcaacagagtggaggctgaggatctgggagtttattactgctttcaaggttcacatgttccgtggacgttcggtggaggcaccaagctggaaatcaaatcctctggtggcggtggctcgggcggtggtgggggtggttcctctagatcttccctcgatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagctacgacggtagcaataactacaacccatctctcaaaaatcgaatctccatcactcgtgacacatctaagaaccagtttttcctgaagttgaattctgtgactactgaggacacagctacatattactgtgctggacggtttgcttactggggccaagggactctggtcactgtctctgcagccaaaacgacacccccatctgtcggttctggctccggatcagggtctggtagtggcagcggatctgggtccggttcaggctcaggaagtgggagcggatcagggtccgggtcagatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcaacagagtggaggctgaggatctgggagtttattactgctttcaaggttcacatgttccgtggacgttcggtggaggcaccaagctggaaatcaaatcctctggtggcggtggctcgggcggtggtgggggtggttcctctagatcttccctcgatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagctacgacggtagcaataactacaacccatctctcaaaaatcgaatctccatcactcgtgacacatctaagaaccagtttttcctgaagttgaattctgtgactactgaggacacagctacatattactgtgctggacggtttgcttactggggccaagggactctggtcactgtctctgcagccaaaacgacacccccatctgtcactagtggccaggccggccagcaccatcaccatcaccatggcgcatacccgtacgacgttccggactacgcttcttag

SEQ ID NO: 44 - protein, 581 aa, Artificial Sequence
  recombinant bivalent scFv antibody
MDMRVPAQLLGLLLLWLSGARCDVLMTQTPLSLPVSLGDQASISCRSSQSIVHSNGNTYLEWYLQKPGQSPKLLIYKVSNRFSGVPDRFSGSGSGTDFTLKINRVEAEDLGVYYCFQGSHVPWTFGGGTKLEIKSSGGGGSGGGGGGSSRSSLDVQLQESGPGLVKPSQSLSLTCSVTGYSITSGYYWNWIRQFPGNKLEWMGYISYDGSNNYNPSLKNRISITRDTSKNQFFLKLNSVTTEDTATYYCAGRFAYWGQGTLVTVSAAKTTPPSVGSGSGSGSGSGSGSGSGSGSGSGSGSGSGSDVLMTQTPLSLPVSLGDQASISCRSSQSIVHSNGNTYLEWYLQKPGQSPKLLIYKVSNRFSGVPDRFSGSGSGTDFTLKINRVEAEDLGVYYCFQGSHVPWTFGGGTKLEIKSSGGGGSGGGGGGSSRSSLDVQLQESGPGLVKPSQSLSLTCSVTGYSITSGYYWNWIRQFPGNKLEWMGYISYDGSNNYNPSLKNRISITRDTSKNQFFLKLNSVTTEDTATYYCAGRFAYWGQGTLVTVSAAKTTPPSVTSGQAGQHHHHHHGAYPYDVPDYAS

SEQ ID NO: 45 - protein, 30 aa, Artificial Sequence
  alternative H-FR1 sequence
DVQLQESGPSLVKPSQSLSLTCSVTGYSIT

SEQ ID NO: 46 - protein, 25 aa, Artificial Sequence
  alternative H-FR1 (Chothia numbering)
DVQLQESGPSLVKPSQSLSLTCSVT

SEQ ID NO: 47 - protein, 4 aa, Artificial Sequence
  identical C-terminal amino acids of PP2Ac and PP4c
DYFL

SEQ ID NO: 48 - protein, 8 aa, Artificial Sequence
  immunization peptide/immunogen
  VARIANT 2: beta Ala
CATPDYFL

SEQ ID NO: 49 - protein, 15 aa, Artificial Sequence
  Peptide L309
CGEPHVTRRTPDYFL

SEQ ID NO: 50 - protein, 15 aa, Artificial Sequence
  Peptide Y307F
CGEPHVTRRTPDFFL

SEQ ID NO: 51 - protein, 15 aa, Artificial Sequence
  Peptide pY307
  MOD_RES 13: PHOSPHORYLATION
CGEPHVTRRTPDYFL

SEQ ID NO: 52 - protein, 15 aa, Artificial Sequence
  Abcam Cat#ab106262 (polyclonal antibody with epitope in the N terminus of PP2Ac)
FTKELDQWIEQLNEC

SEQ ID NO: 53 - DNA, 17 bp, Artificial Sequence
  1MFH_I forward heavy primer
aggaactgcaggtgtcc

SEQ ID NO: 54 - DNA, 20 bp, Artificial Sequence
  1MFH_II forward heavy primer
cagctacaggtgtccactcc

SEQ ID NO: 55 - DNA, 19 bp, Artificial Sequence
  1MFH_III forward heavy primer
tggcagcarcagctacagg

SEQ ID NO: 56 - DNA, 19 bp, Artificial Sequence
  1MFH_IV forward heavy primer
ctgcctggtgacattccca

SEQ ID NO: 57 - DNA, 17 bp, Artificial Sequence
  1MFH_V forward heavy primer
ccaagctgtgtcctgtc

SEQ ID NO: 58 - DNA, 20 bp, Artificial Sequence
  1MFH_VI forward heavy primer
ttttaaaaggtgtccagkgt

SEQ ID NO: 59 - DNA, 23 bp, Artificial Sequence
  1MFH_VII forward heavy primer
cctgtcagtaactrcaggtgtcc

SEQ ID NO: 60 - DNA, 20 bp, Artificial Sequence
  1MFH_VIII forward heavy primer
ttttaaaaggggtccagtgt

SEQ ID NO: 61 - DNA, 19 bp, Artificial Sequence
  1MFH_IX forward heavy primer
cgttcctggtatcctgtct

SEQ ID NO: 62 - DNA, 21 bp, Artificial Sequence
  1MFH_X forward heavy primer
atgaagttgtggytraactgg

SEQ ID NO: 63 - DNA, 18 bp, Artificial Sequence
  1MFH_XI forward heavy primer
tgttggggctkaagtggg

SEQ ID NO: 64 - DNA, 23 bp, Artificial Sequence
  1MRG reverse heavy primer
agaaggtgtgcacaccgctggac

SEQ ID NO: 65 - DNA, 24 bp, Artificial Sequence
  1mFK_I forward light primer
rgtgcagattttcagcttcctgct

SEQ ID NO: 66 - DNA, 24 bp, Artificial Sequence
  1mFK_II forward light primer
tggacatgagggcycctgctcagt

SEQ ID NO: 67 - DNA, 24 bp, Artificial Sequence
  1mFK_III forward light primer
ctstggttgtctggtgttgaygga

SEQ ID NO: 68 - DNA, 22 bp, Artificial Sequence
  1mFK_IV forward light primer
gttgctgctgctgtggcttaca

SEQ ID NO: 69 - DNA, 17 bp, Artificial Sequence
  1mFK_V forward light primer
gtatctggtacctgtgg

SEQ ID NO: 70 - DNA, 22 bp, Artificial Sequence
  1mFK_VI forward light primer
tgcctgttaggctgttggtgct

SEQ ID NO: 71 - DNA, 25 bp, Artificial Sequence
  1mFK_VII forward light primer
gctcagttccttggtctcctgttgc

SEQ ID NO: 72 - DNA, 20 bp, Artificial Sequence
  1mFK_VIII forward light primer
tgggtgctgctgctctgggt

SEQ ID NO: 73 - DNA, 27 bp, Artificial Sequence
  1mFK_IX forward light primer
cagttcctgtttctgttartgctctgg

SEQ ID NO: 74 - DNA, 26 bp, Artificial Sequence
  1mFK_X forward light primer
tgctctggttatatggtgctgatggg

SEQ ID NO: 75 - DNA, 21 bp, Artificial Sequence
  1mRK reverse light primer
actgaggcacctccagatgtt

SEQ ID NO: 76 - DNA, 23 bp, Artificial Sequence
  pJET1.2 forward sequencing primer
cgactcactatagggagagcggc

SEQ ID NO: 77 - DNA, 24 bp, Artificial Sequence
  pJET1.2 reverse sequencing primer
aagaacatcgattttccatggcag

SEQ ID NO: 78 - protein, 29 aa, Artificial Sequence
  HFR1 (Contact)
DVQLQESGPGLVKPSQSLSLTCSVTGYSI

SEQ ID NO: 79 - protein, 11 aa, Artificial Sequence
  CDR-H1 (AbM)
GYSITSGYYWN

SEQ ID NO: 80 - protein, 7 aa, Artificial Sequence
  CDR-H1 (Contact)
TSGYYWN

SEQ ID NO: 81 - protein, 9 aa, Artificial Sequence
  CDR-H1 (IMGT)
GYSITSGYY

SEQ ID NO: 82 - protein, 11 aa, Artificial Sequence
  HFR2 (Contact)
WIRQFPGNKLE

SEQ ID NO: 83 - protein, 17 aa, Artificial Sequence
  HFR2 (IMGT)
WNWIRQFPGNKLEWMGY

SEQ ID NO: 84 - protein, 9 aa, Artificial Sequence
  CDR-H2 (AbM)
YISYDGSNN

SEQ ID NO: 85 - protein, 12 aa, Artificial Sequence
  CDR-H2 (Contact)
WMGYISYDGSNN

SEQ ID NO: 86 - protein, 7 aa, Artificial Sequence
  CDR-H2 (IMGT)
ISYDGSN

SEQ ID NO: 87 - protein, 39 aa, Artificial Sequence
  HFR3 (AbM)
YNPSLKNRISITRDTSKNQFFLKLNSVTTEDTATYYCAG

SEQ ID NO: 88 - protein, 37 aa, Artificial Sequence
  HFR3 (Contact)
YNPSLKNRISITRDTSKNQFFLKLNSVTTEDTATYYC

SEQ ID NO: 89 - protein, 38 aa, Artificial Sequence
  HFR3 (IMGT)
NYNPSLKNRISITRDTSKNQFFLKLNSVTTEDTATYYC

SEQ ID NO: 90 - protein, 5 aa, Artificial Sequence
  CDR-H3 (Contact)
AGRFA

SEQ ID NO: 91 - protein, 6 aa, Artificial Sequence
  CDR-H3 (IMGT)
AGRFAY

SEQ ID NO: 92 - protein, 12 aa, Artificial Sequence
  HFR4 (Contact)
YWGQGTLVTVSA

SEQ ID NO: 93 - protein, 29 aa, Artificial Sequence
  alternative HFR1 (Contact)
DVQLQESGPSLVKPSQSLSLTCSVTGYSI

SEQ ID NO: 94 - protein, 29 aa, Artificial Sequence
  LFR1 (Contact)
DVLMTQTPLSLPVSLGDQASISCRSSQSI

SEQ ID NO: 95 - protein, 26 aa, Artificial Sequence
  LFR1 (IMGT)
DVLMTQTPLSLPVSLGDQASISCRSS

SEQ ID NO: 96 - protein, 12 aa, Artificial Sequence
  CDR-L1 (Contact)
VHSNGNTYLEWY

SEQ ID NO: 97 - protein, 11 aa, Artificial Sequence
  CDR-L1 (IMGT)
QSIVHSNGNTY

SEQ ID NO: 98 - protein, 9 aa, Artificial Sequence
  LFR2 (Contact)
LQKPGQSPK

SEQ ID NO: 99 - protein, 17 aa, Artificial Sequence
  LFR2 (IMGT)
LEWYLQKPGQSPKLLIY

SEQ ID NO: 100 - protein, 10 aa, Artificial Sequence
  CDR-L2 (Contact)
LLIYKVSNRF

SEQ ID NO: 101 - protein, 33 aa, Artificial Sequence
  LFR3 (Contact)
SGVPDRFSGSGSGTDFTLKINRVEAEDLGVYYC

SEQ ID NO: 102 - protein, 36 aa, Artificial Sequence
  LFR3 (IMGT)
NRFSGVPDRFSGSGSGTDFTLKINRVEAEDLGVYYC

SEQ ID NO: 103 - protein, 8 aa, Artificial Sequence
  CDR-L3 (Contact)
FQGSHVPW

SEQ ID NO: 104 - protein, 11 aa, Artificial Sequence
  LFR4 (Contact)
TFGGGTKLEIK

SEQ ID NO: 105 - DNA, 60 bp, Artificial Sequence
  light chain forward primer
cctggggctcctgctgctctggctctcaggtgccagatgtgatgttttgatgacccaaac

SEQ ID NO: 106 - DNA, 75 bp, Artificial Sequence
  light chain reverse primer
ggaagatctagaggaaccacccccaccaccgcccgagccaccgccaccagaggatttgatttccagcttggtgcc

SEQ ID NO: 107 - DNA, 44 bp, Artificial Sequence
  heavy chain forward primer
ggtggttcctctagatcttccctcgatgtacagcttcaggagtc

SEQ ID NO: 108 - DNA, 45 bp, Artificial Sequence
  heavy chain reverse primer
ctggccggcctggccactagtgacagatgggggtgtcgttttggc

SEQ ID NO: 109 - DNA, 51 bp, Artificial Sequence
  signal peptide forward primer
atggacatgagggtccctgctcagctcctggggctcctgctgctctggctc

SEQ ID NO: 110 - DNA, 41 bp, Artificial Sequence
  heavy chain reverse primer
gaggaggaggaggaggagcctggccggcctggccactagtg

SEQ ID NO: 111 - DNA, 20 bp, Artificial Sequence
  pcDNA3 forward sequencing primer
taatacgactcactataggg

SEQ ID NO: 112 - DNA, 18 bp, Artificial Sequence
  pcDNA3 reverse sequencing primer
tagaaggcacagtcgagg

SEQ ID NO: 113 - DNA, 73 bp, Artificial Sequence
  3′-terminal primer at the havy chain constant region
cctgaaccggacccagatccgctgccactaccagaccctgatccggagccagaaccgacagatgggggtgtcg

SEQ ID NO: 114 - DNA, 74 bp, Artificial Sequence
  5′-terminal primer of the second scFv
ggatctgggtccggttcaggctcaggaagtgggagcggatcagggtccgggtcagatgttttgatgacccaaac

SEQ ID NO: 115 - protein, 20 aa, Artificial Sequence
  Peptide
DPAPRRGEPHVTRRTPDYFL

SEQ ID NO: 116 - protein, 11 aa, Artificial Sequence
  C-terminal region of PP2Ac
  MOD_RES 6: PHOSPHORYLATION
HVTRRTPDYFL

SEQ ID NO: 117 - protein, 11 aa, Artificial Sequence
  C-terminal region of PP2Ac
  MOD_RES 9: PHOSPHORYLATION
HVTRRTPDYFL

SEQ ID NO: 118 - protein, 309 aa, Homo sapiens
  Serine/threonine-protein phosphatase 2A catalytic subunit alpha isoform
MDEKVFTKELDQWIEQLNECKQLSESQVKSLCEKAKEILTKESNVQEVRCPVTVCGDVHGQFHDLMELFRIGGKSPDTNYLFMGDYVDRGYYSVETVTLLVALKVRYRERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL

SEQ ID NO: 119 - protein, 309 aa, Homo sapiens
  Serine/threonine-protein phosphatase 2A catalytic subunit beta isoform
MDDKAFTKELDQWVEQLNECKQLNENQVRTLCEKAKEILTKESNVQEVRCPVTVCGDVHGQFHDLMELFRIGGKSPDTNYLFMGDYVDRGYYSVETVTLLVALKVRYPERITILRGNHESRQITQVYGFYDECLRKYGNANVWKYFTDLFDYLPLTALVDGQIFCLHGGLSPSIDTLDHIRALDRLQEVPHEGPMCDLLWSDPDDRGGWGISPRGAGYTFGQDISETFNHANGLTLVSRAHQLVMEGYNWCHDRNVVTIFSAPNYCYRCGNQAAIMELDDTLKYSFLQFDPAPRRGEPHVTRRTPDYFL

SEQ ID NO: 120 - protein, 307 aa, Homo sapiens
  Serine/threonine-protein phosphatase 4 catalytic subunit
MAEISDLDRQIEQLRRCELIKESEVKALCAKAREILVEESNVQRVDSPVTVCGDIHGQFYDLKELFRVGGDVPETNYLFMGDFVDRGFYSVETFLLLLALKVRYPDRITLIRGNHESRQITQVYGFYDECLRKYGSVTVWRYCTEIFDYLSLSAIIDGKIFCVHGGLSPSIQTLDQIRTIDRKQEVPHDGPMCDLLWSDPEDTTGWGVSPRGAGYLFGSDVVAQFNAANDIDMICRAHQLVMEGYKWHFNETVLTVWSAPNYCYRCGNVAAILELDEHLQKDFIIFEAAPQETRGIPSKKPVADYFL

SEQ ID NO: 121 - protein, 305 aa, Homo sapiens
  Serine/threonine-protein phosphatase 6 catalytic subunit
MAPLDLDKYVEIARLCKYLPENDLKRLCDYVCDLLLEESNVQPVSTPVTVCGDIHGQFYDLCELFRTGGQVPDTNYIFMGDFVDRGYYSLETFTYLLALKAKWPDRITLLRGNHESRQITQVYGFYDECQTKYGNANAWRYCTKVFDMLTVAALIDEQILCVHGGLSPDIKTLDQIRTIERNQEIPHKGAFCDLVWSDPEDVDTWAISPRGAGWLFGAKVTNEFVHINNLKLICRAHQLVHEGYKFMFDEKLVTVWSAPNYCYRCGNIASIMVFKDVNTREPKLFRAVPDSERVIPPRTTTPYFL

SEQ ID NO: 122 - DNA, 723 bp, Artificial Sequence
  DNA sequence of the recombinant 7C10-C5 light chain
atggacatgagggtccctgctcagctcctggggctcctgctgctctggctctcaggtgccagatgtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcaacagagtggaggctgaggatctgggagtttattactgctttcaaggttcacatgttccgtggacgttcggtggaggcaccaagctggaaatcaaagcagatgctgcaccaactgtatccatcttcccaccatccagtgagcagttaacatctggaggtgcctcagtcgtgtgcttcttgaacaacttctaccccaaagacatcaatgtcaagtggaagattgatggcagtgaacgacaaaatggcgtcctgaacagttggactgatcaggacagcaaagacagcacctacagcatgagcagcaccctcacgttgaccaaggacgagtatgaacgacataacagctatacctgtgaggccactcacaagacatcaacttcacccattgtcaagagcttcaacaggaatgagtgttag

SEQ ID NO: 123 - protein, 240 aa, Artificial Sequence
  Amino acid sequence of the recombinant 7C10-C5 light chain
MDMRVPAQLLGLLLLWLSGARCDVLMTQTPLSLPVSLGDQASISCRSSQSIVHSNGNTYLEWYLQKPGQSPKLLIYKVSNRFSGVPDRFSGSGSGTDFTLKINRVEAEDLGVYYCFQGSHVPWTFGGGTKLEIKADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 124 - DNA, 1392 bp, Artificial Sequence
  DNA sequence of the recombinant 7C10-C5 heavy chain coding sequence
atgtacaggatgcaactcctgtcttgcattgcactaagtcttgcacttgtcacgaattcggatgtacagcttcaggagtcaggacctggcctcgtgaaaccttctcagtctctgtctctcacctgctctgtcactggctactccatcaccagtggttattactggaactggatccggcagtttccaggaaacaaactggaatggatgggctacataagctacgacggtagcaataactacaacccatctctcaaaaatcgaatctccatcactcgtgacacatctaagaaccagtttttcctgaagttgaattctgtgactactgaggacacagctacatattactgtgctggacggtttgcttactggggccaagggactctggtcactgtctctgcagctaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacatcgagcacctggcccagccagtccatcacctgcaatgtggcccacccggcaagcagcaccaaggtggacaagaaaattgagcccagagggcccacaatcaagccctgtcctccatgcaaatgcccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaataa

SEQ ID NO: 125 - protein, 463 aa, Artificial Sequence
  Amino acid sequence of the recombinant 7C10-C5 heavy chain
MYRMQLLSCIALSLALVTNSDVQLQESGPGLVKPSQSLSLTCSVTGYSITSGYYWNWIRQFPGNKLEWMGYISYDGSNNYNPSLKNRISITRDTSKNQFFLKLNSVTTEDTATYYCAGRFAYWGQGTLVTVSAAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 126 - DNA, 18 bp, Artificial Sequence
  pTrioz H forward sequencing primer
tttgagcggagctaattc

SEQ ID NO: 127 - DNA, 40 bp, Artificial Sequence
  light chain forward primer
ggctctcaggtgccagatgtgatgttttgatgacccaaac

SEQ ID NO: 128 - DNA, 39 bp, Artificial Sequence
  light chain reverse primer
acagttggtgcagcatctgctttgatttccagcttggtg

SEQ ID NO: 129 - DNA, 41 bp, Artificial Sequence
  heavy chain forward primer
cttgcacttgtcacgaattcggatgtacagcttcaggagtc

SEQ ID NO: 130 - DNA, 43 bp, Artificial Sequence
  heavy chain reverse primer
agaccgatggggctgttgttttagctgcagagacagtgaccag

(19) United States
(12) Patent Application Publication
(10) Pub. No.: US 2023/0121197 A1
(43) Pub. Date: Apr. 20, 2023

(54) ANTIBODIES SPECIFICALLY BINDING THE CARBOXYMETHYLATED CATALYTIC SUBUNIT OF PROTEIN PHOSPHATASE 2A

(71) Applicants: Medizinische Universität Wien, Wien (AT); Regents of The University of Michigan, Ann Arbor, MI (US)

(72) Inventors: Egon OGRIS, Wien (AT); Goutham NARLA, Ann Arbor, MI (US)

(73) Assignees: Medizinische Universität Wien, Wien (AT); Regents of The University of Michigan, Ann Arbor, MI (US)

(21) Appl. No.: 17/759,320

(22) PCT Filed: Jan. 25, 2021
(86) PCT No.: PCT/EP2021/051645

§ 371 Date: Jul. 22, 2022

Related U.S. Application Data
(60) Provisional application No. 62/965,443, filed on Jan. 24, 2020.

(30) Foreign Application Priority Data
Jan. 28, 2020 (EP) 20154216.4

(51) Int. Cl. G01N 33/573; G01N 33/574

(52) U.S. Cl. CPC G01N 33/573; G01N 33/57484; G01N 2333/916

(57) ABSTRACT

The present invention relates to an antibody specifically binding the carboxymethylated catalytic subunit of protein phosphatase 2A (PP2Ac). Also provided are diagnostic uses of said antibody and screening methods employing the inventive antibody.

The present invention relates to an antibody specifically binding the carboxymethylated catalytic subunit of protein phosphatase 2A (PP2Ac). Also provided are diagnostic uses of said antibody and screening methods employing the inventive antibody.

Many signaling pathways regulating cellular behavior such as proliferation, cell death and differentiation comprise protein kinases and phosphatases. Aberrant activation of kinases leads to hyperphosphorylation of proteins and occurs in many diseases such as cancer, neurodegenerative disorders, diabetes or heart diseases. To date, most therapies aiming to normalize the aberrant signaling/hyperphosphorylation employ kinase inhibitors. However, the activation of phosphatases is a further possibility to reduce the hyperphosphorylation (see e.g. O'Connor (2018), Int J Biochem Cell Biol., 96:182-193; Allen-Petersen (2019), Cancer Res 79, 209-219; Rincon (2015), Oncotarget 6, 4299-4314); Sangodkar (2017), The Journal of clinical investigation 127, 2081-2090; Grossman (2017), Cell Chem Biol 24, 1368-1376 e1364; Gutierrez (2014), The Journal of clinical investigation 124, 644-655; Kauko (2018), Science translational medicine 10; McClinch (2018), Cancer Res 78, 2065-2080; O'Connor (2018), Int J Biochem Cell Biol 96, 182-193; Tohme (2019), JCI insight 4; Wiredja (2017), Proteomics 17).

The serine/threonine protein phosphatase 2A (PP2A) family of phosphatases plays an important role in maintaining cellular homeostasis and is often dysregulated in human diseases. PP2A family members are trimeric holoenzymes consisting of a catalytic C subunit (isoforms PPP2CA and PPP2CB), a structural A subunit (isoforms PPP2R1A and PPP2R1B) and one of many different B-type regulatory subunits which determine the substrate specificity of the individual holoenzyme (Virshup (2009), Mol Cell 33, 537-545). The B-subunits are grouped into four groups, namely B (B55/PPP2R2), B′ (B56/PPP2R5), B″ (PR72/PR130/PR59/PPP2R3) and B′″ (striatin family), each of which consists of several isoforms, raising the number of possible PP2A complexes to >80. PP2A family members are only active and substrate-targeted in the form of the trimeric holoenzyme. PP2A holoenzyme biogenesis is a multistep process that involves chaperones, regulatory and inhibitory proteins and post-translational modifications of the A, B and C subunits. The carboxy terminus of the catalytic PP2A subunit (PP2Ac) contains several highly conserved residues that are known or have been predicted to be post-translationally modified. It is further known that post-translational modifications of PP2Ac regulate the binding of B subunits to the core AC dimer, and thus control the PP2A substrate specificity.

Foremost, the methylesterification (Favre, (1994) J Biol Chem 269, 16311-7; Lee J A (2007), J Biol Chem. 282(42):30974-8; Hwang (2016), J Biol Chem. 291(40):21008-21019) of the α-carboxyl group at the C-terminal leucine 309 of PP2Ac (according to UniProtKB identifier P67775-1; Stone, (1988), Nucleic Acids Res. 16 (23), 11365; Arino, Proc. Natl. Acad. Sci. U.S.A. 1988 85 (12), 4252-4256) is an essential modification required for the assembly of many heterotrimers (holoenzymes) comprising B, certain B′ and also B″ subunits. PP2A family members with such B, B′ or B″ subunits, in particular B55 and B56, can be regarded as carboxymethylation-dependent trimers and said family members account for ˜30% of all PP2A complexes in a cell (Jackson (2012), Neoplasia 14, 585-599; Longin (2007), J Biol Chem 282, 26971-26980; Nunbhakdi-Craig (2007), J Neurochem 101, 959-971; Tolstykh (2000), Embo J 19, 5682-5691; Xing (2006), Cell 127, 341-353; Yu (2001), Mol Biol Cell 12, 185-199). Most of these carboxymethylation-dependent PP2A trimers have tumor-suppressive functions by negatively regulating inter alia Akt mediated cell survival, p70/p85 S6K mediated cell growth and proliferation and ERK/MAP kinase mitogenic pathways (Jackson (2012), Neoplasia 14, 585-599; Sontag (1993), Cell 75, 887-897).

Although carboxymethylation does not seem to be necessary for complex formation with B′ (Striatin) subunits, 70% to 90% of the PP2Ac molecules are α-carboxymethylated in a cell culture suggesting additional roles for carboxymethylation apart from promoting the assembly of the carboxymethylation-dependent PP2A trimers.

The carboxymethylation of PP2Ac is catalyzed by the essential Leucine Carboxyl Methyltransferase 1 (LCMT-1) and the respective demethylation by the Phosphatase Methylesterase (PME-1). Inhibition of LCMT-1 or overexpression of PME-1 cause the loss/reduction of tumor-suppressive PP2A holoenzymes and therefore promote tumorigenesis (Pusey (2016), Tumour Biol 37, 11835-11842). Thus, PP2A carboxymethylation is a modification required for the assembly of a subset of PP2A holoenzymes that fulfill tumor-suppressive functions in cells. Moreover, reduced PP2A carboxymethylation has been associated with an increased risk of Alzheimer's disease due to reduced PP2A-mediated tau dephosphorylation (Sontag (2014), Front Mol Neurosci. 2014, 7:16; Sontag (2004), J Neuropathol Exp Neurol, 63 (10): 1080-91).

In addition to α-carboxymethylation, the C-terminus of PP2Ac has been reported to be phosphorylated on tyrosine 307 (Tyr³⁰⁷) and threonine 304 (Thr³⁰⁴) (Chen (1992), Science 257, 1261-1264; Ogris (1997), Oncogene 15, 911-917; Schmitz (2010), Nature cell biology 12, 886-893). For many years, erroneous findings according to which PP2Ac phosphorylation, i.e. at Tyr³⁰⁷, inhibits the tumor-suppressive function of PP2A have obscured the significance of carboxymethylation at Leu³⁰⁹ of PP2Ac. Later, it has turned out that PP2Ac phosphorylation has been wrongly implicated in cancer because the binding specificities of the used antibodies were different than assumed. In particular, the rabbit monoclonal antibody clone E155 (catalog #1155-1, Abcam) has been used in at least 112 publications to detect pTyr³⁰⁷. Based on those experiments, high levels of Tyr³⁰⁷ phosphorylation have been interpreted as evidence for PP2A inhibition and were claimed to correlate with poor outcome/overall survival in different human cancer types (Chen (2017), Hum Pathol 66, 93-100; Cristobal (2014), Br J Cancer 111, 756-762; Rincon (2015), Oncotarget 6, 4299-4314). In the context of the present invention, it has been found that commercial pTyr³⁰⁷ antibodies including E155 and mouse monoclonal F-8 from Santa Cruz Biotechnology (SCBT) are not specific for pTyr³⁰⁷ but recognize the non-methylated C-terminus of PP2Ac and are impaired in recognizing carboxymethylated PP2Ac (Frohner (2020) Cell Rep. 30(9):3171-3182; Mazhar (2020) Cell Rep. 30(9):3164-3170). These observations further illustrate that the carboxymethylation of PP2Ac is critical for the tumor-suppressive function of PP2A. Additionally, these findings demonstrate that the validation of the binding specificity of antibodies is crucial and suggest that antibodies which specifically detect carboxymethylated PP2Ac are highly desirable.

However, so far, most analyses of the PP2A α-carboxymethylation state in cells have been done with antibodies that recognize the non-carboxymethylated C-terminus of PP2Ac with a negatively charged α-carboxyl group at Leu³⁰⁹ and which are blocked by α-carboxymethylation of PP2Ac. These antibodies allow to estimate the carboxymethylation level of PP2Ac only indirectly. For instance, the signal increase must be compared between an untreated and a base/NaOH-treated PP2A sample wherein the PP2A α-carboxymethylester is hydrolyzed. In addition, an essential control for the detection of changes in the PP2A α-carboxymethylation state by using non-carboxymethyl specific antibodies is the determination of total PP2Ac expression levels with a PP2Ac antibody that is not impaired by any posttranslational PP2Ac modification. Thus, the indirect determination of carboxymethylated PP2Ac levels is rather error-prone and cumbersome, and anti-carboxymethyl-specific PP2Ac antibodies which directly detect carboxymethylated PP2Ac are desired.

To date, the only commercially available antibody with presumed specificity for the α-carboxymethylated PP2Ac is “2A10” which is sold by several companies including Upstate Biotechnology (now Merck-Millipore), Abcam, Biolegend/Covance, ImmuQuest and Santa Cruz Biotechnology. This 2A10 antibody is, inter alia, employed in Hombauer (2007), PLos Biol Jun 5 (6): e155. Yet, as has been surprisingly been found in context of the present invention, said 2A10 antibody is not specific for carboxymethylated PP2Ac and cross-reacts with carboxymethylated PP4c and also with carboxymethylated PP6c (FIG. 8B+C, Table 5). Furthermore, the carboxymethylation specificity of the 2A10 antibody may be considered as influenced by the phosphorylation of Tyr³⁰⁷ and Thr³⁰⁴.

Furthermore, the generation of a monoclonal antibody, termed 4D9, with alleged “specificity” for the methylated PP2Ac has been reported (Tolstykh (2000), Embo J 19, 5682-5691). The 4D9 antibody has not been commercialized and does not appear to be publicly available. This “4D9” clone, however, has been raised against an amidated C-terminal PP2Ac peptide (299-309). Yet, amidation neutralizes the charge of the α-carboxyl-group and can at most, if at all, mimic the functional consequence of the carboxymethylation (charge neutralization). Accordingly, the 4D9 antibody is to be considered as being not specific for the α-carboxymethylated C-terminus of the PP2Ac subunit since it also binds the amidated C-terminus of PP2Ac, the antigen/epitope against it was apparently raised. Therefore, even if this antibody may have a reactivity to carboxymethylated PP2Ac, it is not a truly anti-methyl-PP2Ac-specific antibody/truly anti-carboxymethylated PP2Ac-specific antibody. The lack of specific anti-carboxymethylated PP2Ac antibodies further hampers the monitoring of protein phosphatase 2A (PP2A) activation and/or activity which is associated with PP2Ac carboxymethylation. Moreover, the lack of specific anti-carboxymethylated PP2Ac antibodies further impedes the development of therapies, i.e. anti-cancer therapies, which aim at increasing PP2Ac carboxymethylation and/or establishing or enhancing target-specific activity. In particular, potential drugs for said therapies include PP2A activators such as phenothiazine derived small molecule PP2A activators (SMAPs) (Allen-Petersen (2019), Cancer Res 79, 209-219; Sangodkar (2017), J Clin Invest. 127, 2081-2090; Gutierrez (2014), J Clin Invest. 124(2), 644-55; Kastrinsky (2015), Bioorg Med Chem. October 1; 23(19):6528-34), drugs that counteract the endogenous PP2A inhibitors SET (I2PP2A, UniProt: Q01105) and CIP2A (UniProt: Q8TCG1-1), drugs that inhibit the Phosphatase Methylesterase PME-1 (UniProt Q9Y570) and drugs which activate PP2A through a yet unknown mechanism as described in O'Connor (2018), Int J Biochem Cell Biol., 96:182-193 and Grossman (2017) Cell Chem Biol. 24(11):1368-1376.

Several SET inhibiting drugs have been described: COG112, OP449, FTY720, OSU-2S, MP07-66 and TGI1002 (Matsuoka (2003) Br. J. Pharmacol. 138, 1303-1312; Neviani (2007) J. Clin. Invest. 117, 2408-2421.; Omar (2011); Hepatology 53, 1943-1958; Zonta (2015), Blood, 125(24):3747-55). Furthermore, several CIP2A inhibiting drugs have been described: Erlotinib, Celastrol, Bortezomib, TD-19, and TD-44 (Chao (2014), J Pharmacol Exp Ther. 351(2):352-8; Chen (2010), Oncogene 29, 6257-6266; Chen (2012) Bioorg. Med. Chem. 20, 6144-6153; Ding (2014) Mol. Med. Rep. 10, 387-392; Hou (2013) Molecules 18, 15398-15411; Liu (2013) Haematologica 98, 729-738; Liu (2014) Carcinogenesis 35, 905-914; Liu (2017) Eur. J. Cancer 72, 46-48; Tseng (2012) Breast Cancer Res. 14(2):R68; Wang (2014) Lung Cancer 85, 152-160; Wu (2017) J. Pharmacol. Sci. 134, 22-28; Yu (2013) Biochem. Pharmacol. 85, 356-366; Yu (2014) Cell Death Dis. 5:e1359). Furthermore, ABL-127 has been described as an PME-1 inhibitor (Bachovchin (2011) PNAS. 108 (17) 6811-6816). Furthermore, Forskolin, Vitamin E analogs, Lanolinamide, Canthardin and morroniside have been described to activate PP2A (Feschenko (2002) J. Pharmacol. Exp. Ther. 302, 111-118; Huang (2009) Carcinogenesis 30, 1125-1131; Kar (2012) Apoptosis 17, 735-747; Neuzil (2002) FASEB J. 15, 403-412; Neviani (2005) Cancer Cell 8, 355-368; Seamon (1981) Proc. Natl. Acad. Sci. U.S.A. 78, 3363-3367; Yang (2016) J Alzheimers Dis 51(1):33-44). However, it remains unclear whether such drugs are effective in enhancing PP2Ac carboxymethylation and/or PP2A activity to dephosphorylate disease-associated hyperphosphorylated substrates.

Furthermore, the lack of specific anti-carboxymethylated PP2Ac antibodies also hampers the diagnostics of diseases that are associated with altered PP2A activity.

Considering the tight regulation of PP2A, i.e. by methylation of the C-terminal carboxyl group of PP2Ac, the important biological roles of PP2A and its implications in many diseases, and the great potential of PP2A as a therapeutic target, it is of utmost importance to have tools available which allow precisely determining the level of carboxymethylated PP2Ac in a biological sample or tissue.

Thus, there is a need for means and methods for specifically and truly detecting carboxymethylated PP2Ac and/or assessing the activation and/or activity of PP2A.

Accordingly, the invention relates to an antibody specifically binding the carboxymethylated catalytic subunit of protein phosphatase 2A (PP2Ac). Therefore, the present invention provides for anti-methyl-PP2Ac-specific antibodies (anti-carboxymethylated PP2Ac-specific antibodies). In the context of the present invention, the expression “specifically binding carboxymethylated PP2Ac” (or equivalents thereof) means that the antibody only specifically binds the carboxymethylated PP2Ac but does neither specifically bind the non-carboxymethylated PP2Ac nor the closely related carboxymethylated PP4c (UniProtKB identifier P60510-1; Brewis (1993), EMBO J. 12(3):987-96; Kloeker (1997), Biochem J. 327 (Pt 2): 481-486) or PP6c (UniProtKB identifier 000743-1; Bastians (1996) J. Cell Sci. 109:2865-2874; Hwang (2016), J Biol Chem. 291(40):21008-21019). Moreover, said expression preferably further means that said antibody binds the carboxymethylated PP2Ac stronger than the respective C terminally α-amidated PP2Ac. In one embodiment, the inventive antibodies do also not (specifically) bind non-methylated PP4c or non-methylated PP6c. The present invention also provides for anti-methyl-PP2Ac-specific antibodies (anti-carboxymethylated PP2Ac-specific antibodies) that do not (specifically) bind to the amidated C-terminus of PP2Ac.

As illustrated in the appended Examples, the inventors provide for the first antibody that specifically binds the methylated carboxyl group of the C-terminal leucine of PP2Ac (Leu³⁰⁹ in human PP2Ac), designated as “7C10-C5”. Therefore, the first truly anti-methyl-PP2Ac-specific antibodies (truly anti-carboxymethylated PP2Ac-specific antibodies) are provided herein. As illustrated in the present invention, this inventive compound does not only specifically bind the methylated carboxyl group of the C-terminal leucine of PP2Ac (Leu³⁰⁹ in human PP2Ac) but it does neither bind non-carboxymethylated PP2Ac nor the closely related carboxymethylated PP4c or PP6c. Furthermore, and as also illustrated in the appended examples, this inventive compound binds the carboxymethylated C-terminal part of PP2Ac 6.15-fold stronger as determined by ELISA (FIG. 4) than the respective amidated C-terminal part of PP2Ac. It was further surprisingly found that the binding of this inventive compound, i.e a binding molecule that comprises at least one CDR of the herein disclosed 7C10-C5, to the carboxymethylated C-terminus of PP2Ac is not impaired by the phosphorylation of nearby amino acids (Tyr³⁰⁷ or Thr³⁰⁴). To achieve this unexpectedly high specificity, the inventors have modified, i.e. expanded, a typical method for generating monoclonal antibodies in an unusual way. First, as illustrated in appended examples, a very short specific immunogen only comprising merely the most C-terminal six amino acids of PP2Ac including the carboxymethylated C-terminal leucine was used for the immunization of mice and secondly, a specific screening approach employing mutant cells was developed in context of this invention which allows a precise assessment of the specific binding of hybridoma supernatants to carboxymethylated PP2Ac.

The appended Examples further provide ample evidence that none of the prior art antibodies does indeed specifically bind carboxymethylated PP2Ac as defined above. In particular, it is demonstrated that said prior art antibodies additionally bind non-carboxymethylated PP2Ac, carboxymethylated PP4c and/or carboxymethylated PP6c. Accordingly, the term “specifically binding” in context of this invention means that there is no, or a much reduced and/or very low, cross-reactivity of the herein disclosed inventive binding molecule to bind non-carboxymethylated PP2Ac, carboxymethylated (and non-carboxymethylated) PP4c and/or carboxymethylated (and non-carboxymethylated) PP6c as detectable via corresponding standard assays as employed herein and as illustrated in the appended examples. In particular, the inventors found by employing surface plasmon resonance analysis that 7C10-C5 binds to carboxymethylated PP2Ac about 12-fold stronger than to carboxymethylated PP4c, and does not bind to non-carboxymethylated PP2Ac to a measureable extent. These findings were confirmed by ELISA which also revealed, e.g., an at least about 10-fold stronger binding to carboxymethylated PP2Ac than to carboxymethylated PP4c. Western blotting further confirmed the high specificity of 7C10-C5 against carboxymethylated PP2Ac and the very low and negligable cross reactivity with PP4c. Furthermore, as also documented herein, available antibodies of the prior art are impaired to various degrees by phosphorylation of the nearby sites Thr304 and Tyr307. Impairment of the binding to carboxymethylated PP2Ac by concurrent phosphorylation of Thr304 and/or Tyr307 precludes the use of such a binding molecule/antibody for precise quantification of carboxymethylated PP2Ac levels.

Of note, as used herein, the term “PP2Ac” refers to the catalytic subunit of PP2A and not the trimeric holoenzyme PP2A (having parts A, B and C) as such. In contrast, the term “PP2A” refers to said trimeric holoenzyme.

Furthermore, as used herein, the terms “PP4c” or “PP6c” refer to the catalytic subunit of PP4 or PP6, respectively, and not the PP4 or PP6 holoenzymes.

Based on the surprising findings illustrated in the appended Examples, it is evident that the epitope of the antibody of the present invention comprises the carboxymethylated C-terminal leucine of PP2Ac.

Preferably, the binding molecule/antibody of the present invention binds specifically the methylated carboxyl group of the C-terminal leucine of PP2Ac. Said C-terminal leucine corresponds to Leu³⁰⁹ of human PP2Ac (UniProtKB identifier P67775-1; Stone, (1988), Nucleic Acids Res. 16 (23), 11365; Arino, Proc. Natl. Acad. Sci. U.S.A. 1988 85 (12), 4252-4256). In particular, the amino acid sequence of PP2Ac (UniProtKB identifier P67775-1; HUMAN Serine/threonine-protein phosphatase 2A catalytic subunit alpha isoform) is:

(SEQ ID NO: 118)
MDEKVFTKELDQWIEQLNECKQLSESQVKSLCEKAKEILTKESNVQEVRC
PVTVCGDVHGQFHDLMELFRIGGKSPDTNYLFMGDYVDRGYYSVETVTLL
VALKVRYRERITILRGNHESRQITQVYGFYDECLRKYGNANVWKYFTDLF
DYLPLTALVDGQIFCLHGGLSPSIDTLDHIRALDRLQEVPHEGPMCDLLW
SDPDDRGGWGISPRGAGYTFGQDISETFNHANGLTLVSRAHQLVMEGYNW
CHDRNVVTIFSAPNYCYRCGNQAAIMELDDTLKYSFLQFDPAPRRGEPHV
TRRTPDYFL.

Furthermore, the term “PP2Ac”, as used herein, may also refer to the UniProtKB identifier P62714 HUMAN Serine/threonine-protein phosphatase 2A catalytic subunit beta isoform with the sequence:

(SEQ ID NO: 119)
MDDKAFTKELDQWVEQLNECKQLNENQVRTLCEKAKEILTKESNVQEVRC
PVTVCGDVHGQFHDLMELFRIGGKSPDTNYLFMGDYVDRGYYSVETVTLL
VALKVRYPERITILRGNHESRQITQVYGFYDECLRKYGNANVWKYFTDLF
DYLPLTALVDGQIFCLHGGLSPSIDTLDHIRALDRLQEVPHEGPMCDLLW
SDPDDRGGWGISPRGAGYTFGQDISETFNHANGLTLVSRAHQLVMEGYNW
CHDRNVVTIFSAPNYCYRCGNQAAIMELDDTLKYSFLQFDPAPRRGEPHV
TRRTPDYFL.

In particular, the amino acid sequence of PP4c (UniProtKB identifier P60510-1; HUMAN Serine/threonine-protein phosphatase 4 catalytic subunit) is:

(SEQ ID NO: 120)
MAEISDLDRQIEQLRRCELIKESEVKALCAKAREILVEESNVQRVDSPVT
VCGDIHGQFYDLKELFRVGGDVPETNYLFMGDFVDRGFYSVETFLLLLAL
KVRYPDRITLIRGNHESRQITQVYGFYDECLRKYGSVTVWRYCTEIFDYL
SLSAIIDGKIFCVHGGLSPSIQTLDQIRTIDRKQEVPHDGPMCDLLWSDP
EDTTGWGVSPRGAGYLFGSDVVAQFNAANDIDMICRAHQLVMEGYKWHFN
ETVLTVWSAPNYCYRCGNVAAILELDEHLQKDFIIFEAAPQETRGIPSKK
PVADYFL.

In particular, the amino acid sequence of PP6c (UniProtKB identifier 000743-1; Human Serine/threonine-protein phosphatase 6 catalytic subunit) is:

(SEQ ID NO: 121)
MAPLDLDKYVEIARLCKYLPENDLKRLCDYVCDLLLEESNVQPVSTPVTV
CGDIHGQFYDLCELFRTGGQVPDTNYIFMGDFVDRGYYSLETFTYLLALK
AKWPDRITLLRGNHESRQITQVYGFYDECQTKYGNANAWRYCTKVFDMLT
VAALIDEQILCVHGGLSPDIKTLDQIRTIERNQEIPHKGAFCDLVWSDPE
DVDTWAISPRGAGWLFGAKVTNEFVHINNLKLICRAHQLVHEGYKFMFDE
KLVTVWSAPNYCYRCGNIASIMVFKDVNTREPKLFRAVPDSERVIPPRTT
TPYFL.

In context of this invention, the terms “α-carboxymethylated PP2Ac”, “carboxymethylated PP2Ac”, “methylated PP2Ac”, “C-terminally methylated PP2Ac”, “methyl-PP2Ac”, “carboxymethyl-PP2Ac” and equivalents thereof, are used interchangeably herein and refer to PP2Ac, or the C-terminal region of PP2Ac, comprising a C-terminal leucine with a methylated carboxyl group (L-O—CH3). In context of this invention the term α-carboxymethylated PP2Ac means that the carboxyl group of said leucine 309 in the human PPAc is esterified with a methyl group, i.e. the present invention is based on the provision of a highly specific binding molecule/antibody for “α-carboxymethylesterified PP2Ac”.

The terms “non-α-carboxymethylated PP2Ac”, “non-carboxymethylated PP2Ac”, “non-methylated PP2Ac”, “unmethylated PP2Ac”, “non-C-terminally methylated PP2Ac”, “non-methyl-PP2Ac” and equivalents thereof, are used interchangeably herein and refer to PP2Ac, or the C-terminal region of PP2Ac, comprising a C-terminal leucine with a non-modified, unmodified or hydroxylated (L-OH) carboxyl group.

The terms “α-amidated PP2Ac”, “amidated PP2Ac”, and “C-terminally amidated PP2Ac”, and equivalents thereof, are used interchangeably herein and refer to PP2Ac, or the C-terminal region of PP2Ac, comprising a C-terminal leucine with an amidated carboxyl group (i.e. L-NH₂).

The C-terminal region of PP2Ac, as used herein, may refer to the 50, 20, 11, 8 or 6 most C-terminal amino acids of PP2Ac.

Because of the absolute conservation of the C-terminal PP2Ac sequence TPDYFL (SEQ ID NO:1) across eukaryotes, the binding molecule/antibody of the invention recognizes the carboxymethylated PP2Ac of all eukaryotic PP2Ac species. Thus, the carboxymethylated PP2Ac bound by the binding molecule/antibody according to the invention may be inter alia the carboxymethylated PP2Ac of a human, mouse, rat, dog, pig, cow, sheep, goat, camel, horse, fish, frog, fly, worm, nematode, sea urchin, plant or yeast, preferably of a human.

Moreover, as used herein, the C-terminal region of PP2Ac comprises the sequence TPDYFL (SEQ ID NO:1), the C-terminal region of PP4c comprises the sequence VADYFL (SEQ ID NO:19) and the C-terminal region of PP6c comprises the sequence TTPYFL (SEQ ID NO:21). These definitions of the corresponding C-termini are based on the human sequences and the respective UniProtKB identifiers for PP2Ac, PP4c and PP6c as provided herein.

Preferably, the binding molecule/antibody of the invention binds specifically an epitope comprised in the carboxymethylated C-terminal region of PP2Ac, wherein said C-terminal region has the sequence TPDYFL (SEQ ID NO:1). As illustrated in the appended examples, said C-terminal region was employed in the generation of the inventive antibody/binding molecule and is comprised in the immunogen. Accordingly, TPDYFL (SEQ ID NO:1) may be employed in context of the present invention to generate an antibody/binding molecule. It has also been employed (with modifications as provided herein and in the appended examples) for the generation of the inventive antibodies, like the monoclonal “7C10-C5”.

As already mentioned above and illustrated in the appended Examples, antibodies of the present invention, like the 7C10-C5 antibody, do not specifically bind the carboxymethylated C-terminal region of PP4c or PP6c. Since the most four C-terminal amino acids of PP2Ac and PP4c are identical (DYFL; SEQ ID NO: 47), it can be safely assumed that in addition to the methylated carboxyl group of the C-terminal leucine, the epitope further comprises the threonine and/or the proline within SEQ ID NO:1.

Thus, the epitope of the antibody of the invention may comprise (i) the methylated carboxyl group of the C-terminal leucine and (ii) the threonine and/or the proline within SEQ ID NO:1.

As described herein, the inventive binding molecule or antibody provided herein has a very strong binding affinity to carboxymethylated PP2Ac and/or a peptide comprising the carboxymethylated C-terminal region of PP2Ac, as described herein.

Moreover, as illustrated in the appended Examples, an exemplary antibody of the invention (7C10-C5) which is characterized by the CDRs and FRs described herein, binds a peptide consisting of the last 11 amino acids of the carboxymethylated C-terminal region of PP2Ac (HVTRRTPDYFL; SEQ ID NO:18), with a dissociation constant (K_{D}) of 20 nM or less, e.g. about 11 nM. In contrast, the prior art antibody 2A10 binds said peptide only with a K_{D} of 300 nM or greater, a.g. about 448 nM, as determined by the same measurement method under similar conditions (see Example 15).

Thus, it was surprisingly found by the inventors that an exemplary antibody of the invention (7C10-C5) binds a peptide consisting of the last 11 amino acids of the carboxymethylated C-terminal region of PP2Ac (HVTRRTPDYFL; SEQ ID NO:18) at least about 15-fold, in particular at least about 40-fold, e.g. about 41-fold, stronger than the prior art antibody 2A10.

Thus, the antibody of the invention may bind carboxymethylated PP2Ac or a peptide consisting of the last 11 amino acids of the carboxymethylated C-terminal region of PP2Ac (HVTRRTPDYFL; SEQ ID NO:18) with a dissociation constant (K_{D}) of 200 nM, 100 nM, 80 nM, 60 nM, 40 nM, 20 nM, 15 nM or less, preferably 40 nM or less, more preferably 20 nM or less, e.g. about 11 nM. Preferably, said antibody binds said peptide having the sequence HVTRRTPDYFL (SEQ ID NO:18) with said K_{D}. Preferably, said peptide is acetylated at the N-terminus.

Preferably herein, the peptides described herein in the context of the binding to an antibody, e.g. an antibody of the present invention, and/or in the context of means or methods for measuring the binding specificity of an antibody to said peptides, e.g. surface plasmon resonance or ELISA, are acetylated at the N-terminus.

Preferably, in context of the invention, the dissociation constant (K_{D}), i.e. of the binding of an antibody, e.g. the antibody of the invention, to an antigen (e.g. a peptide comprising the carboxymethylated C-terminal region of PP2Ac) is determined by surface plasmon resonance, preferably Biacore, more preferably a BiacoreT200 instrument (e.g. from Cytiva, former GE healthcare) and a BiacoreT200 Evaluation Software (i.e. version 3.1), e.g. as described in the appended Examples. Furthermore, in context of the invention, the conditions for determining said dissociation constant, i.e. by employing said surface plasmon resonance, preferably comprise:

a temperature of 25° C.; an antibody concentration of about 50 μg/mL; and/or PBS+0.005% Tween+0.1% BSA as a buffer (e.g. running buffer) that has, in particular, a pH of about 7, preferably 7.4. Preferably, the antibody of interest (e.g. the antibody of the invention) is immobilized on the surface plasmon resonance (e.g. Biacore) chip (preferably the Series S Sensor Chip CM5) by suitable means, e.g., by coating the chip with an antibody that binds to the antibody of interest, for example, an anti-mouse IgG antibody where suitable. For example, the flow cells may be coated with about 30 μg/mL anti-mouse IgG antibody (e.g. in 10 mM sodium acetate pH 5.0 as immobilization buffer) using a mouse antibody capture kit via amine coupling, i.e. to obtain an immobilization level within the specifications (e.g. about 11000 RU such as 11606 RU or 11334 RU), as illustrated in the appended Examples. Thus, the antigen, e.g a peptide comprising the carboxymethylated C-terminal region of PP2Ac as described herein and/or illustrated in the appended Examples, is preferably the analyte. Furthermore, preferably, several measurements are performed by using the antigen, e.g. said peptide, at different concentrations. Since the analyte sample concentration (i.e. antigen/peptide) is preferably within a range of 0.1 to 10 times the K_{D} and equilibrium is preferably reached at all concentrations that are used for K_{D} calculation, and the range of analyte concentrations is ideally wide enough to reveal the full curvature of the plot (i.e. to reach saturation), the antigen/peptide concentrations relate to a parameter that may be adjusted for each antibody-antigen pair. In particular, the value of the equilibrium dissociation constant (K_{D}) may be obtained by fitting a plot of response at equilibrium (Req) against the respective concentration of the analyte, as described herein. Thus, the K_{D} value may refer to the analyte concentration at 50% of the maximum (saturated) response (see the curves in FIG. 32 for this relationship, and the respective K_{D} as vertical line), as described herein.

Thus, the concentrations of a peptide comprising the carboxymethylated C-terminal region of PP2Ac, e.g. a peptide consisting of the sequence HVTRRTPDYFL-CH₃ (SEQ ID NO:18), are, preferably, as follows:

for the antibody of the invention, e.g. 7C10-C5: about 2 nM to 500 nM, preferably at least about 5 nM and about 160 nM, and more preferably further about 10 nM, about 20 nM, about 40 nM, and/or about 80 nM; and

for the 2A10 antibody: about 2 nM to 500 nM, preferably at least about 30 nM and about 480 nM, and more preferably further about 60 nM, about 120 nM, and/or about 240 nM.

For comparing the K_{D} values of different antibodies and/or antigens, preferably, the same measurement conditions, devices and analyse methods (e.g. software) are to be used unless certain parameters are preferably adjusted to obtain a very reliable K_{D} value, e.g. the analyte concentrations.

In particular, the Biacore measurements are to be performed such that all binding curves reach equilibrium and all requirements for a steady state analysis are fulfilled, e.g. as demonstrated in the appended Examples.

The person skilled in the art can easily and reliably reproduce the K_{D} values described herein and in the context of the present invention based on the guidelines, experimental details and data provided herein supplemented with his or her common general knowledge. Thus, the binding specificity of the antibody of the invention can be readily determined and compared to other antibodies.

It is well known in the art that the complementary determining regions (CDRs) determine the binding specificity of the antibody. Suitable methods to determine the sequence of a monoclonal antibody are readily available in the art and further described in the appended Examples. The variable regions of an antibody may be grouped into the CDRs and the framework regions (FRs), in particular by using the Kabat or Chothia numbering schemes. Due to its wide-spread use and reliability, the Kabat numbering system may be preferred.

In a particular embodiment, the binding molecule/antibody of the present invention comprises a heavy chain variable region and a light chain variable region, wherein

(a) the heavy chain variable region comprises at least one complementary determining region selected from a CDR-H3, a CDR-H2 and a CDR-H1, wherein
(i) the CDR-H3 sequence is RFAY (SEQ ID NO:2),
(ii) the CDR-H2 sequence is YISYDGSNNYNPSLKN (SEQ ID NO:3),
(iii) the CDR-H1 sequence is SGYYWN (SEQ ID NO:4), and/or

(b) the light chain variable region comprises at least one complementary determining region selected from a CDR-L3, a CDR-L2 and a CDR-L1, wherein the
(iv) the CDR-L3 sequence is FQGSHVPWT (SEQ ID NO:5),
(v) the CDR-L2 sequence is KVSNRFS (SEQ ID NO:6), and
(vi) the CDR-L1 sequence is RSSQSIVHSNGNTYLE (SEQ ID NO:7).

In a particular embodiment, the binding molecule/antibody of the present invention comprises a heavy chain variable region and a light chain variable region, wherein

(a) the heavy chain variable region comprises a CDR-113, a CDR-H2 and a CDR-H1, wherein
(i) the CDR-H3 sequence is RFAY (SEQ ID NO:2),
(ii) the CDR-H2 sequence is YISYDGSNNYNPSLKN (SEQ ID NO:3),
(iii) the CDR-H1 sequence is SGYYWN (SEQ ID NO:4), and

(b) the light chain variable region comprises a CDR-L3, a CDR-L2 and a CDR-L1, wherein the
(iv) the CDR-L3 sequence is FQGSHVPWT (SEQ ID NO:5),
(v) the CDR-L2 sequence is KVSNRFS (SEQ ID NO:6), and
(vi) the CDR-L1 sequence is RSSQSIVHSNGNTYLE (SEQ ID NO:7).

The CDRs as in SEQ ID NO:2, SEQ ID NO:3, SEQ ID NO:4, SEQ ID NO:5, SEQ ID NO:6 and SEQ ID NO:7 have been determined with the Kabat numbering system.

Preferably, the inventive binding molecule/antibody that is characterized by the CDRs comprised in the heavy chain variable region and/or light chain variable region as described herein, e.g. the CDRs as just described above, has a binding specificity as described herein, e.g. it binds a peptide consisting of the last 11 amino acids of the carboxymethylated C-terminal region of PP2Ac (HVTRRTPDYFL; SEQ ID NO:18) with a dissociation constant (K_{D}) of 200 nM, 100 nM, 80 nM, 60 nM, 40 nM, 20 nM, 15 nM or less, preferably 40 nM or less, more preferably 20 nM or less, e.g. about 11 nM. Furthermore, said inventive binding molecule/antibody preferably binds a peptide comprising the carboxymethylated C-terminal region of PP2Ac, e.g. a peptide consisting of the last 11 amino acids of the carboxymethylated C-terminal region of PP2Ac (HVTRRTPDYFL-CH₃; SEQ ID NO:18) at least 4-, 6-, 8-, 10- or 12-fold, preferably at least 10-fold, e.g. about 12-fold, stronger than a peptide comprising the carboxymethylated C-terminal region of PP4c, e.g. a peptide consisting of the last 11 amino acids of the carboxymethylated C-terminal region of PP4c (PSKKPVADYFL-CH₃; SEQ ID NO:20). Preferably, said binding specificity is determined by surface plasmon resonance (e.g. Biacore), as described herein. Generally, the inventive binding molecules/antibodies (anti-methyl-PP2Ac-specific antibodies/anti-carboxymethylated PP2Ac-specific antibodies) provided and to be used in accordance with the present invention may comprise a CDR sequence having 75% or more (e.g. 80%, more preferably 85%, 90%, most preferably 95%, 96%, 97%, 98%, 99% or more) amino acid identity to one of the specific CDR sequences provided and disclosed herein. It is understood that the identity is assessed/determined over the full length of the CDR sequence. The binding molecules/antibodies of the present invention that comprise CDRs with lower than 100% sequence identity to the specific CDR sequences provided and disclosed herein specifically bind the methylated carboxyl group of the C-terminal leucine of PP2Ac (Leu³⁰⁹ in human PP2Ac). In a further embodiment, these inventive binding molecules/antibodies comprising CDRs with lower than 100% sequence identity to the specific CDR sequences provided and disclosed herein do also neither bind non-carboxymethylated (human) PP2Ac nor the closely related carboxymethylated (human) PP4c and/or carboxymethylated (human) PP6c as defined herein, or at very low and/or much reduced levels compared to prior art antibodies. The binding specificity of the inventive antibody can be readily determined, as described herein, e.g. by surface plasmon resonance, ELISA and/or Western blot, preferably by surface plasmon resonance (e.g. Biacore).

The term “CDR” as employed herein relates to “complementary determining region”, which is well known in the art. The CDRs are parts of immunoglobulins and T cell receptors that determine the specificity of said molecules and make contact with specific ligand. The CDRs are the most variable part of the molecule and contribute to the diversity of these molecules. There are three CDR regions, CDR1, CDR2 and CDR3, in each V domain. CDR-H depicts a CDR region of a variable heavy chain and CDR-L relates to a CDR region of a variable light chain. H means the variable heavy chain and L means the variable light chain. The CDR regions of an Ig-derived region may be determined as described in Kabat (1991), Sequences of Proteins of Immunological Interest, 5th edit., NIH Publication no. 91-3242 U.S. Department of Health and Human Services; Chothia (1987), J. Mol. Biol. 196, 901-917; and Chothia (1989) Nature, 342, 877-883. The CDRs as provided herein above were determined by the Kabat system. Each CDR region of a variable heavy chain is herein interchangeably designated as CDR-H1 or VH-CDR1, CDR-H2 or VH-CDR2, and CDR-H3 or VH-CDR3, respectively. Likewise, each CDR region of a variable light chain is designated herein CDR-L1 or VL-CDR1, CDR-L2 or VL-CDR2, and CDR-L3 or VL-CDR3, respectively.

As discussed above and as used herein, the term “antibody” also relates to binding molecules that comprise CDRs or binding portions of the antibodies described herein.

Generally, the inventive binding molecules/antibodies provided and to be used in accordance with the present invention (anti-methyl-PP2Ac-specific antibodies/anti-carboxymethylated PP2Ac-specific antibodies) may comprise a CDR sequence having 75% or more (e.g. 80%, more preferably 85%, 90%, most preferably 95%, 96%, 97%, 98%, 99% or more) amino acid identity to one of the specific CDR sequences provided and disclosed herein. It is understood that the identity is assessed/determined over the full length of the CDR sequence.

In one aspect, the variable region of the heavy chain of the antibody of this invention comprises a CDR-H3 region having an amino acid sequence as depicted in SEQ ID NO:2. The antibodies may also comprise a CDR sequence having 75% or more (e.g. 80%, more preferably 85%, 90%, most preferably 95%, 96%, 97%, 98%, 99% or more) amino acid identity to one of said CDRs.

In a certain aspect the present invention relates to an antibody specifically binding carboxymethylated PP2Ac, wherein the variable region of the heavy chain of said antibody comprises a CDR-H3 region having an amino acid sequence as depicted in SEQ ID NO:2, or a CDR sequence having 75% or more amino acid identity to said CDR.

The antibody of the present invention (anti-methyl-PP2Ac-specific antibody/anti-carboxymethylated PP2Ac-specific antibody) may comprise

a variable region of the heavy chain comprising a CDR-H1 region having an amino acid sequence as depicted in SEQ ID NO:4, or a CDR sequence having 75% or more amino acid identity to said CDR;

or

a variable region of the heavy chain comprising a CDR-H1 region having an amino acid sequence as depicted in SEQ ID NO:27, or a CDR sequence having 75% or more amino acid identity to said CDR.

The antibody of the present invention (anti-methyl-PP2Ac-specific antibody/anti-carboxymethylated PP2Ac-specific antibody) may comprise

a variable region of the heavy chain comprising a CDR-112 region having an amino acid sequence as depicted in SEQ ID NO:3, or a CDR sequence having 75% or more amino acid identity to said CDR;

or

a variable region of the heavy chain comprising a CDR-112 region having an amino acid sequence as depicted in SEQ ID NO:26, or a CDR sequence having 75% or more amino acid identity to said CDR.

In a certain aspect the present invention relates to a binding molecule/an antibody specifically binding carboxymethylated PP2Ac,

wherein the variable region of the heavy chain of said antibody comprises a CDR-H1 region having an amino acid sequence as depicted in SEQ ID NO:4, a CDR-112 region having an amino acid sequence as depicted in SEQ ID NO:3, and a CDR-113 region having an amino acid sequence as depicted in SEQ ID NO:2, or a CDR sequence having 75% or more amino acid identity to one of said CDRs.

The antibody of the present invention (anti-methyl-PP2Ac-specific antibody/anti-carboxymethylated PP2Ac-specific antibody) may comprise a variable region of the heavy chain comprising a CDR-H1 region having an amino acid sequence as depicted in SEQ ID NO:4, a CDR-H2 region having an amino acid sequence as depicted in SEQ ID NO:3, and a CDR-H3 region having an amino acid sequence as depicted in SEQ ID NO:2.

In a certain aspect the present invention relates to an antibody specifically binding carboxymethylated PP2Ac,

wherein the variable region of the heavy chain of said antibody comprises a CDR-H1 region having an amino acid sequence as depicted in SEQ ID NO:27, a CDR-H2 region having an amino acid sequence as depicted in SEQ ID NO:26, and a CDR-113 region having an amino acid sequence as depicted in SEQ ID NO:25, or a CDR sequence having 75% or more amino acid identity to one of said CDRs.

The antibody of the present invention (anti-methyl-PP2Ac-specific antibody/anti-carboxymethylated PP2Ac-specific antibody) may comprise a variable region of the heavy chain comprising a CDR-H1 region having an amino acid sequence as depicted in SEQ ID NO:27, a CDR-H2 region having an amino acid sequence as depicted in SEQ ID NO:26, and a CDR-H3 region having an amino acid sequence as depicted in SEQ ID NO:25.

The antibody of the present invention (anti-methyl-PP2Ac-specific antibody/anti-carboxymethylated PP2Ac-specific antibody) may comprise a variable region of the light chain comprising a CDR-L1 region having an amino acid sequence as depicted in SEQ ID NO:7, or a CDR sequence having 75% or more amino acid identity to said CDR.

The antibody of the present invention (anti-methyl-PP2Ac-specific antibody/anti-carboxymethylated PP2Ac-specific antibody) may comprise a variable region of the light chain comprising a CDR-L2 region having an amino acid sequence as depicted in SEQ ID NO:6, or a CDR sequence having 75% or more amino acid identity to said CDR.

The antibody of the present invention (anti-methyl-PP2Ac-specific antibody/anti-carboxymethylated PP2Ac-specific antibody) may comprise a variable region of the light chain comprising a CDR-L3 region having an amino acid sequence as depicted in SEQ ID NO:5, or a CDR sequence having 75% or more amino acid identity to said CDR.

The antibody of the present invention (anti-methyl-PP2Ac-specific antibody/anti-carboxymethylated PP2Ac-specific antibody) may comprise a variable region of the light chain comprising a CDR-L1 region having an amino acid sequence as depicted in SEQ ID NO:7, a CDR-L2 region having an amino acid sequence as depicted in SEQ ID NO:6, and a CDR-L3 region having an amino acid sequence as depicted in SEQ ID NO:5, or a CDR sequence having 75% or more amino acid identity to one of said CDRs.

In a certain aspect, the present invention relates to an antibody specifically binding carboxymethylated PP2Ac,

wherein the variable region of the light chain of said antibody comprises a CDR-L1 region having an amino acid sequence as depicted in SEQ ID NO:7, a CDR-L2 region having an amino acid sequence as depicted in SEQ ID NO:6, and a CDR-L3 region having an amino acid sequence as depicted in SEQ ID NO:5, or a CDR sequence having 75% or more amino acid identity to one of said CDRs.

The antibody of the present invention (anti-methyl-PP2Ac-specific antibody/anti-carboxymethylated PP2Ac-specific antibody) may comprise a variable region of the light chain comprising a CDR-L1 region having an amino acid sequence as depicted in SEQ ID NO:7, a CDR-L2 region having an amino acid sequence as depicted in SEQ ID NO:6, and a CDR-L3 region having an amino acid sequence as depicted in SEQ ID NO:5.

In a certain aspect the present invention relates to an antibody specifically binding carboxymethylated PP2Ac,

wherein the variable region of the heavy chain of said antibody comprises a CDR-H1 region having an amino acid sequence as depicted in SEQ ID NO:4, a CDR-112 region having an amino acid sequence as depicted in SEQ ID NO:3, and a CDR-113 region having an amino acid sequence as depicted in SEQ ID NO:2, or a CDR sequence having 75% or more amino acid identity to one of said CDRs;

and

wherein the variable region of the light chain of said antibody comprises a CDR-L1 region having an amino acid sequence as depicted in SEQ ID NO:7, a CDR-L2 region having an amino acid sequence as depicted in SEQ ID NO:6, and a CDR-L3 region having an amino acid sequence as depicted in SEQ ID NO:5, or a CDR sequence having 75% or more amino acid identity to one of said CDRs.

The antibody of the present invention (anti-methyl-PP2Ac-specific antibody/anti-carboxymethylated PP2Ac-specific antibody) may comprise

a variable region of the heavy chain comprising a CDR-H1 region having an amino acid sequence as depicted in SEQ ID NO:4, a CDR-H2 region having an amino acid sequence as depicted in SEQ ID NO:3, and a CDR-H3 region having an amino acid sequence as depicted in SEQ ID NO:2;

and

a variable region of the light chain comprising a CDR-L1 region having an amino acid sequence as depicted in SEQ ID NO:7, a CDR-L2 region having an amino acid sequence as depicted in SEQ ID NO:6, and a CDR-L3 region having an amino acid sequence as depicted in SEQ ID NO:5.

In a certain aspect the present invention relates to an antibody an antibody specifically binding carboxymethylated PP2Ac,

wherein the variable region of the heavy chain of said antibody comprises a CDR-H1 region having an amino acid sequence as depicted in SEQ ID NO:27, a CDR-H2 region having an amino acid sequence as depicted in SEQ ID NO:26, and a CDR-113 region having an amino acid sequence as depicted in SEQ ID NO:25, or a CDR sequence having 75% or more amino acid identity to one of said CDRs;

and

wherein the variable region of the light chain of said antibody comprises a CDR-L1 region having an amino acid sequence as depicted in SEQ ID NO:30, a CDR-L2 region having an amino acid sequence as depicted in SEQ ID NO:29, and a CDR-L3 region having an amino acid sequence as depicted in SEQ ID NO:28, or a CDR sequence having 75% or more amino acid identity to one of said CDRs.

The antibody of the present invention (anti-methyl-PP2Ac-specific antibody/anti-carboxymethylated PP2Ac-specific antibody) may comprise

a variable region of the heavy chain comprising a CDR-H1 region having an amino acid sequence as depicted in SEQ ID NO:27, a CDR-H2 region having an amino acid sequence as depicted in SEQ ID NO:26, and a CDR-H3 region having an amino acid sequence as depicted in SEQ ID NO:25;

and

a variable region of the light chain comprising a CDR-L1 region having an amino acid sequence as depicted in SEQ ID NO:30, a CDR-L2 region having an amino acid sequence as depicted in SEQ ID NO:29, and a CDR-L3 region having an amino acid sequence as depicted in SEQ ID NO:28.

The herein provided antibodies (anti-methyl-PP2Ac-specific antibody/anti-carboxymethylated PP2Ac-specific antibody) may comprise one or more of the heavy or light chain variable sequences above or a sequence at least 75%, 80%, more preferably at least 85%, 90%, even more preferably at least 95%, 96%, 97%, 98%, or most preferably 99% identical thereto.

In one aspect, the variation in the sequences occurs in the framework regions, i.e. outside of the CDR sequences. For example, the antibodies of these aspects contain specific CDR regions above that are not subject to variation. Yet, the framework region of these antibodies can show a variation/identity of 75% or more (or 80%, more preferably at least 85%, 90%, even more preferably at least 95%, 96%, 97%, 98%, or most preferably 99%) to the framework region of the specific variable V_{L}-region(s) and/or variable V_{H}-region(s) as defined above. The framework region(s) can be identified by methods known in the art or may be as described herein in the context of the present invention. As used herein the term “framework region” can refer to the sequence of the variable V_{L}-region(s) and/or the variable V_{H}-region(s) that is outside of the CDR sequences.

In a certain aspect the present invention relates to an antibody specifically binding carboxymethylated PP2Ac,

wherein said antibody comprises a variable V_{H}-region having an amino acid sequence as shown in SEQ ID NO:16, or a variable V_{H}-region having an amino acid sequence which has 75% or more identity to said variable V_{H}-region;

and/or

wherein said antibody comprises a variable V_{L}-region having an amino acid sequence as shown in SEQ ID NO:17, or a variable V_{L}-region having an amino acid sequence which has 75% or more identity to said variable V_{L}-region,

said antibody comprising

a variable region of the heavy chain comprising a CDR-H1 region having an amino acid sequence as depicted in SEQ ID NO:4, a CDR-H2 region having an amino acid sequence as depicted in SEQ ID NO:3, and/or a CDR-113 region having an amino acid sequence as depicted in SEQ ID NO:2;

and/or

a variable region of the light chain comprising a CDR-L1 region having an amino acid sequence as depicted in SEQ ID NO:7, a CDR-L2 region having an amino acid sequence as depicted in SEQ ID NO:6, and/or a CDR-L3 region having an amino acid sequence as depicted in SEQ ID NO:5.

The antibodies/binding molecules of the invention include the antibodies having one or more of the CDRs and/or one or more of the variable regions (V_{H}-region and/or V_{L}-region) and/or one or more of the chains (heavy chain and/or light chain) as disclosed herein as well as variants thereof having 75% or more (for example 80%, more preferably 85%, 90%, most preferably 95%, 96%, 97%, 98%, or 99%) sequence identity to said CDR(s), variable region(s) and/or chains.

In a further embodiment, the binding molecule/antibody of the invention (anti-methyl-PP2Ac-specific antibody/anti-carboxymethylated PP2Ac-specific antibody) is a binding molecule/an antibody wherein

(a) the heavy chain variable region further comprises at least one framework region selected from a H-FR1, a H-FR2, a H-FR3 and a H-FR4, wherein the framework regions are directly adjacent to the CDRs according to the formula (H-FR1)-(CDR-H1)-(H-FR2)-(CDR-H2)-(H-FR3)-(CDR-H3)-(H-FR4), and/or

(b) the light chain variable region further comprises at least one framework region selected from a L-FR1, a L-FR2, a L-FR3 and a L-FR4, wherein the framework regions are directly adjacent to the CDRs according to the formula (L-FR1)-(CDR-L1)-(L-FR2)-(CDR-L2)-(L-FR3)-(CDR-L3)-(L-FR4).

Preferably, said H-FR1 sequence is DVQLQESGPGLVKPSQSLSLTCSVTGYSIT (SEQ ID NO:8),

said H-FR2 sequence is WIRQFPGNKLEWMG (SEQ ID NO:9),

said H-FR3 sequence is RISITRDTSKNQFFLKLNSVTTEDTATYYCAG (SEQ ID NO:10),

said H-FR4 sequence is WGQGTLVTVSA (SEQ ID NO:11),

said L-FR1 sequence is DVLMTQTPLSLPVSLGDQASISC (SEQ ID NO:12),

said L-FR2 sequence is WYLQKPGQSPKLLIY (SEQ ID NO:13),

said L-FR3 sequence is GVPDRFSGSGSGTDFTLKINRVEAEDLGVYYC (SEQ ID NO:14), and

said L-FR4 sequence is FGGGTKLEIK (SEQ ID NO:15).

Alternatively, said H-FR1 sequence may be DVQLQESGPSLVKPSQSLSLTCSVTGYSIT (SEQ ID NO:45).

The present invention also relates to binding molecules/antibodies, i.e. anti-methyl-PP2Ac-specific antibodies/anti-carboxymethylated PP2Ac-specific antibodies wherein, the heavy chain variable region of said binding molecule/antibody has the sequence SEQ ID NO:16 and/or the light chain variable region of said binding molecule/antibody has the sequence SEQ ID NO:17.

In a particular embodiment, the heavy chain variable region of the binding molecule/antibody of the invention has the sequence SEQ ID NO:16 and the light chain variable region of said binding molecule/antibody has the sequence SEQ ID NO:17.

The binding molecules/antibodies, i.e. the anti-methyl-PP2Ac-specific antibodies/anti-carboxymethylated PP2Ac-specific antibodies of the present invention may also comprise (sequence) variations in the herein disclosed heavy chain variable region and/or the herein disclosed light chain variable region. The binding molecules/antibodies of the present invention that comprise heavy and/or light chain variable region with lower than 100% sequence identity to the specific heavy and/or light chain variable regions provided and disclosed herein specifically bind the methylated carboxyl group of the C-terminal leucine of PP2Ac (Leu309 in human PP2Ac). In a further embodiment, these inventive binding molecules/antibodies comprising heavy and/or light chain variable region with lower than 100% sequence identity to the specific heavy and/or light chain variable regions provided and disclosed herein do also neither bind non-carboxymethylated (human) PP2Ac nor the closely related carboxymethylated (human) PP4c and/or carboxymethylated (human) PP6c as defined herein, or at very low and/or much reduced levels compared to prior art antibodies. The binding specificity of the inventive antibody can be readily determined, as described herein, e.g. by surface plasmon resonance, ELISA and/or Western blot, preferably by surface plasmon resonance (e.g. Biacore).

Accordingly, the herein provided binding molecules/antibodies anti-methyl-PP2Ac-specific antibodies/anti-carboxymethylated PP2Ac-specific antibodies may comprise one or more of the heavy or light chain variable sequences above (e.g. SEQ ID NO. 16 and 17, respectively) or a sequence of at least 75%, 80%, more preferably at least 85%, 90%, even more preferably at least 95%, 96%, 97%, 98%, or most preferably 99% identical thereto.

In one aspect, the variation in the sequences occurs in the framework regions, i.e. outside of the CDR sequences. For example, the binding molecules/antibodies of these aspects contain specific CDR regions above that are not subject to variation. Yet, the framework region of these binding molecules/antibodies can show a variation/identity of 75% or more (or 80%, more preferably at least 85%, 90%, even more preferably at least 95%, 96%, 97%, 98%, or most preferably 99%) to the framework region of the specific variable VL-region(s) and/or variable VH-region(s) as defined above. The framework region(s) can be identified by methods known in the art. As used herein the term “framework region” can refer to the sequence of the variable VL-region(s) and/or the variable VH-region(s) that is outside of the CDR sequences.

The antibodies/binding molecules of the invention/anti-methyl-PP2Ac-specific antibodies/anti-carboxymethylated PP2Ac-specific antibodies include the antibodies having one or more of the CDRs and/or one or more of the variable regions (VH-region and/or VL-region) and/or one or more of the chains (heavy chain and/or light chain) as disclosed herein as well as variants thereof having 75% or more (for example 80%, more preferably 85%, 90%, most preferably 95%, 96%, 97%, 98%, or 99%) sequence identity to said CDR(s), variable region(s) and/or chains.

Preferably, the inventive binding molecule/antibody that is characterized by the heavy chain variable region and/or light chain variable region, or the CDRs and framework regions comprised in the heavy chain variable region and/or light chain variable region, as described herein, has a binding specificity as described herein, e.g. as described in the context of the inventive binding molecule/antibody that is characterized by the CDRs comprised in the heavy chain variable region and/or light chain variable region.

As used herein, the terms “identity”, “sequence identity”, “homology” or “sequence homology” (the terms are used interchangeably herein) are used to describe the sequence relationships between two or more amino acid sequences, proteins (or fragments thereof), or polypeptides (or fragments thereof), or corresponding nucleic acid sequences, nucleic acids (or fragments thereof), polynucleotides (or fragments thereof). The terms can be understood in the context of and in conjunction with the terms including: (a) reference sequence, (b) comparison window, (c) sequence identity, (d) percentage of sequence identity, and (e) substantial identity or “homologous”.

A “reference sequence” is a defined sequence used as a basis for sequence comparison. A reference sequence may be a subset of or the entirety of a specified sequence. A “comparison window” includes reference to a contiguous and specified segment of a nucleic acid sequence/polynucleotide sequence or amino acid sequence/polypeptide sequence/protein sequence, wherein the nucleic acid sequence/polynucleotide sequence or amino acid sequence/polypeptide sequence/protein sequence may be compared to a reference sequence. The portion of the nucleic acid sequence/polynucleotide sequence or amino acid sequence/polypeptide sequence/protein sequence in the comparison window may comprise additions, substitutions, or deletions (i.e., gaps) compared to the reference sequence (which does not comprise additions, substitutions, or deletions) for optimal alignment of the two sequences. Generally, the comparison window may be at least about 9 contiguous nucleotides in length (or correspondingly about 3 amino acid residues in length), and optionally can be about 9, 12, 15, 18, 21, 24, 27, 30, 33, 36, 39, 40, 50, or 100, contiguous nucleotides or longer (or correspondingly about 3, 4, 5, 6, 7, 8, 9, 11, 13, 16, or 33 amino acid residues in length or longer). Those of skill in the art understand that to avoid a misleadingly high similarity to a reference sequence due to inclusion of gaps in the polynucleotide or polypeptide sequence a gap penalty is typically introduced and is subtracted from the number of matches. Methods of alignment of sequences for comparison are well-known in the art. Optimal alignment of sequences for comparison may be conducted by the local homology algorithm of Smith and Waterman, Adv. Appl. Math., 2: 482, 1981; by the homology alignment algorithm of Needleman and Wunsch, J. Mol. Biol., 48: 443, 1970; by the search for similarity method of Pearson and Lipman, Proc. Natl. Acad. Sci. USA, 8: 2444, 1988; by computerized implementations of these algorithms, including, but not limited to: CLUSTAL in the PC/Gene program by Intelligenetics, Mountain View, Calif., GAP, BESTFIT, BLAST, FASTA, and TFASTA in the Wisconsin Genetics Software Package, Genetics Computer Group (GCG), 7 Science Dr., Madison, Wisc., USA; the CLUSTAL program is well described by Higgins and Sharp (1988) Gene 73: 237-244; Corpet et al. (1988) Nucleic Acids Research 16:881-90; Huang, et al. (1992) Computer Applications in the Biosciences, 8:1-6; and Pearson, et al. (1994) Methods in Molecular Biology, 24:7-331. The BLAST family of programs which can be used for database similarity searches includes: BLASTN for nucleotide query sequences against nucleotide database sequences; BLASTX for nucleotide query sequences against protein database sequences; BLASTP for protein query sequences against protein database sequences; TBLASTN for protein query sequences against nucleotide database sequences; and TBLASTX for nucleotide query sequences against nucleotide database sequences. See, Current Protocols in Molecular Biology, Chapter 19, Ausubel, et al., Eds., Greene Publishing and Wiley-Interscience, New York, 1995. New versions of the above programs or new programs altogether will undoubtedly become available in the future, and can be used with the present invention.

Unless otherwise stated, sequence identity/similarity values provided herein refer to the value obtained using the BLAST 2.0 suite of programs, or their successors, using default parameters. Altschul et al. (1997) Nucleic Acids Res, 2:3389-3402. It is to be understood that default settings of these parameters can be readily changed as needed in the future.

As those ordinary skilled in the art will understand, BLAST searches assume that proteins or nucleic acids can be modeled as random sequences. However, many real proteins and nucleic acids comprise regions of nonrandom sequences which may be homopolymeric tracts, short-period repeats, or regions enriched in one or more amino acids or nucleic acids. Such low-complexity regions may be aligned between unrelated proteins even though other regions of the protein or nucleic acid are entirely dissimilar. A number of low-complexity filter programs can be employed to reduce such low-complexity alignments. For example, the SEG (Wooten et al. (1993) Comput. Chem. 17:149-163) and XNU (Claverie et al. (1993) Comput. Chem. 17:191-1) low-complexity filters can be employed alone or in combination.

“Sequence identity” or “identity” in the context of two nucleic acid or polypeptide sequences includes reference to the residues in the two sequences which are the same when aligned for maximum correspondence over a specified comparison window, and can take into consideration additions, deletions and substitutions. When percentage of sequence identity is used in reference to proteins it is recognized that residue positions which are not identical often differ by conservative amino acid substitutions, where amino acid residues are substituted for other amino acid residues with similar chemical properties (for example, charge or hydrophobicity) and therefore do not deleteriously change the functional properties of the molecule. Where sequences differ in conservative substitutions, the percent sequence identity may be adjusted upwards to correct for the conservative nature of the substitution. Sequences which differ by such conservative substitutions are said to have sequence similarity. Approaches for making this adjustment are well-known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, for example, according to the algorithm of Meyers and Miller, Computer Applic. Biol. Sci., 4: 11-17, 1988, for example, as implemented in the program PC/GENE (Intelligenetics, Mountain View, Calif., USA).

“Percentage of sequence identity” means the value determined by comparing two optimally aligned sequences over a comparison window, wherein the portion of the polynucleotide or nucleic acid sequence in the comparison window may comprise additions, substitutions, or deletions (i.e., gaps) as compared to the reference sequence (which does not comprise additions, substitutions, or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “homologous” in their various grammatical forms in the context of peptides indicates that a peptide comprises a sequence that has a desired identity, for example, at least 75% sequence identity to a reference sequence, preferably at least 80% sequence identity to a reference sequence, more preferably 85%, even more preferably at least 90% or 95% or even 96%, 97%, 98% or 99% sequence identity to the reference sequence over a specified comparison window. Preferably, optimal alignment is conducted using the homology alignment algorithm of Needleman and Wunsch (1970) J. Mol. Biol., 48:443. An indication that two peptide sequences are substantially identical is that one peptide is immunologically reactive with antibodies raised against the second peptide, although such cross-reactivity is not required for two polypeptides to be deemed substantially identical. Thus, a peptide is substantially identical to a second peptide, for example, where the two peptides differ only by a conservative substitution. Peptides which are “substantially similar” share sequences as noted above except that residue positions which are not identical may differ by conservative amino acid changes.

Accordingly, the present invention provides for binding molecules/antibodies etc specifically binding carboxymethylated PP2Ac which comprise CDRs and/or variable regions and/or heavy/light chains that have an amino acid sequence having at least 75% sequence identity, more preferably at least 80%, even more preferably at least 85%, still more preferably at least 90% and most preferably at least 95%, 96%, 97%, 98% or 99% sequence identity with the amino acid sequence of an antibody (or variable regions thereof or CDRs thereof or heavy/light chains thereof, respectively) that can be obtained or is obtainable from Hybridoma 7C10-C5 deposited under accession number DSM ACC3350 with the depositary institute DSMZ on Jan. 16, 2019.

The present invention also relates to binding molecules/antibodies. i.e., the anti-methyl-PP2Ac-specific antibodies/anti-carboxymethylated PP2Ac-specific antibodies that comprise at least one, at least two, at least three, at least four, at least five, or the 6 CDRs and/or the heavy chain and/or the light chain of an antibody as obtainable from the hybridoma cell line “7C10-C5” deposited under the stipulations of the Budapest treaty under the accession number “DSM ACC3350” with the Leibniz-Institute DSMZ-Deutsche Sammlung von Mikroorgansimen and Zellkulturen GmbH, in Braunschweig/Germany on Jan. 16, 2019 (see appended deposit receipt). The present invention also relates to Hybridoma 7C10-C5 deposited under accession number DSM ACC3350 with the depositary institute DSMZ on Jan. 16, 2019.

Accordingly, the present invention also relates to a binding molecule/antibody that comprises at least one CDR or at least one variable region of the antibody as obtainable from the hybridoma cell line as deposited with the accession number “DSM ACC3350” with the DSMZ (see above and appended deposit receipt). Said binding molecule/antibody comprising at least one CDR or at least one variable region of the antibody as obtainable from the hybridoma cell “7C10-C5”/“DSM ACC3350” is preferably a binding molecule/antibody that specifically binds carboxymethylated catalytic subunit of protein phosphatase 2A (PP2Ac). A preferred antibody of the present invention may also additionally comprise the framework region(s) and/or the Fc region(s) of the antibody as obtainable from the hybridoma cell line “7C10-C5” as deposited under accession number “DSM ACC3350”. Also part of this invention is a full antibody or the full length amino acid sequence of an antibody as obtainable from the hybridoma cell line “7C10-C5” as deposited under accession number “DSM ACC3350”. The CDRs, FRs, variable region, Fc region and/or the full amino acid sequence of said deposited (monoclonal) antibody may be obtained and the corresponding coding nucleic acid sequences or amino acid sequences may be determined by standard means and methods such as inter alia sequencing of the antibody sequence contained in the genome of the single clone hybridoma cell lines and/or mass spectrometry, and/or as described in the appended Examples.

Preferably, the inventive binding molecule/antibody that is characterized by the heavy chain variable region and/or light chain variable region, or the CDRs, or the CDRs and framework regions, comprised in the heavy chain variable region and/or light chain variable region of the antibody that is obtainable from the Hybridoma 7C10-C5 deposited under accession number DSM ACC3350, as described herein, has a binding specificity as described herein, e.g. as described in the context of the inventive binding molecule/antibody that is characterized by the CDRs comprised in the heavy chain variable region and/or light chain variable region.

The antibodies of the present invention may be monoclonal antibodies. Preferably, said monoclonal antibody is a mouse monoclonal antibody. Furthermore, said monoclonal antibody is preferably an IgG1 or IgG2, e.g. IgG2a, antibody, preferably an IgG1 antibody. Preferably, the monoclonal antibody is comprised in the supernatant of a hybridoma clone (singe clone hybridoma cell line) or isolated/purified from the supernatant of a hybridoma clone.

The binding molecule/antibody (i.e. the anti-methyl-PP2Ac-specific antibodies/anti-carboxymethylated PP2Ac-specific antibodies) of the present invention may further be a CDR grafted antibody, a chimeric antibody, a humanized antibody, or a fully human antibody or. An antibody of the present invention, may be of any Ig format and may comprise the IgG, IgA, IgM, etc format. In one embodiment, the antibody of the present invention, i.e an antibody that specifically binds carboxymethylated catalytic subunit of protein phosphatase 2A (PP2Ac) is in an IgG1 or IgG2 format. Yet, without deferring from the gist of the present invention, also other binding molecules/antibody formats may be obtained. Again and preferably, such other binding molecules/antibody format specifically bind the carboxymethylated catalytic subunit of protein phosphatase 2A (PP2Ac) as defined herein. In one embodiment, these other binding molecules/antibody format specifically bind the carboxymethylated catalytic subunit of protein phosphatase 2A (PP2Ac) comprise at least one, at least two, at least three, at least four, at least five and preferably at last six CDRs of the inventive antibody described herein and/or as obtainable from the hybridoma cell line “7C10-C5” as deposited under accession number

The binding molecules/antibodies, i.e., the anti-methyl-PP2Ac-specific antibodies/anti-carboxymethylated PP2Ac-specific antibodies of the present invention may also be an antigen-binding fragment of an antibody, like and in particular a Fab or a F(ab′)₂. Thus, the antibody of the invention may, in one embodiment, lack a constant region (Fc region)

The antibodies/binding molecules of the invention/anti-methyl-PP2Ac-specific antibodies/anti-carboxymethylated PP2Ac-specific antibodies of the present invention also include recombinant molecules, like a recombinant antibody, in particular a single-chain variable fragment (scFv). In one embodiment, said recombinant antibody and/or single-chain variable fragment (scFv) comprises at least one heavy chain variable region and/or at least one light chain variable region of the inventive antibodies as described herein or may comprise at least one, at least two, at least three, at least four, at least five and preferably at last six CDRs of the inventive antibody described herein. In one embodiment, the corresponding heavy chain variable region, the corresponding light chain variable region and/or the corresponding CDRs obtained from the hybridoma cell line “7C10-C5” as deposited under accession number “DSM ACC3350”. In one embodiment, said scFv is a monovalent or a bivalent scFv, preferably a bivalent scFv. Accordingly, the present invention also comprises 3-valent, 4-valent etc scFvs. Also comprised in this invention are bi-specific binding molecules, like bispecific antibodies, bispecific scFvs and the like. In one embodiment, the recombinant antibody further comprises an Fc part, e.g. according to an IgG2 such as an IgG2a, or an IgG1. Again, the binding molecules of the present invention are capable of specifically binding the carboxymethylated catalytic subunit of protein phosphatase 2A (PP2Ac) as defined herein. Again, the binding molecules of the present invention may have a binding specificity as described herein, e.g. as described in the context of the inventive binding molecule/antibody that is characterized by the CDRs comprised in the heavy chain variable region and/or light chain variable region. Accordingly, a corresponding “bispecific binding molecule” comprises at least one binding specificity for carboxymethylated catalytic subunit of protein phosphatase 2A (PP2Ac). Again, as described herein above and in one embodiment of the inventive bispecific binding molecules of the present invention may comprise at least one, at least two, at least three, at least four, at least five and preferably at last six CDRs of the inventive antibody described and defined herein. In one embodiment, the corresponding heavy chain variable region, the corresponding light chain variable region and/or the corresponding CDRs obtained from the hybridoma cell line “7C10-C5” as deposited under accession number “DSM ACC3350”.

In one embodiment of the invention, the heavy chain variable region of the antibodies/binding molecules of the invention/anti-methyl-PP2Ac-specific antibodies/anti-carboxymethylated PP2Ac-specific antibodies has the sequence SEQ ID NO:16, and the light chain variable region of said binding molecule antibody has the sequence SEQ ID NO:17. Said antibody may also be in another format than an immunoglobulin and/or a full antibody molecule, as discussed herein. Such binding molecules may comprise single-chain variable fragments (scFv) and the like. The embodiments as described herein above apply, mutatis mutantis, for such binding molecules.

In all the aspects described herein, the antibody/binding molecule of the present invention may be a full antibody (immunoglobulin), an antibody fragment such as a F(ab)-fragment, a F(ab)2-fragment or an epitope-binding fragment, as well as a single-chain antibody. The antibodies/binding molecules of the invention may be a monoclonal antibody, a recombinantly produced antibody, a chimeric antibody, a humanized antibody, a human antibody, a fully human antibody, a CDR-grafted antibody, a bivalent antibody-construct, a synthetic antibody or a cross-cloned antibody, a diabody, a triabody, a tetrabody, a single chain antibody, a bispecific single chain antibody, etc. The antibody may also be a multispecific antibody, including a bi-specific antibody. The antibodies of the invention may be multifunctional, i.e. they may exert their effects via more than one mode of action, such as for example by activating ADCC or CDC pathways, if desired. The antibodies/binding molecules of the present invention have in common that they specifically bind carboxymethylated (human) PP2Ac as defined and illustrated herein.

Thus, the antibodies of the invention include, but are not limited to, synthetic antibodies, monoclonal antibodies, recombinantly produced antibodies, multispecific antibodies (including bi-specific antibodies), human antibodies, humanized antibodies, chimeric antibodies, single-chain Fvs (scFv) (including bi-specific scFvs), single chain antibodies Fab fragments, F(ab′) fragments, disulfide-linked Fvs (sdFv), and anti-idiotypic (anti-Id) antibodies, and epitope-binding fragments of any of the above.

“Single-chain Fv” or “scFv” antibody fragments have, in the context of the invention, the VII and VL domains of an antibody, wherein these domains are present in a single polypeptide chain. Generally, the scFv polypeptide further comprises a polypeptide linker between the VII and VL domains which enables the scFv to form the desired structure for antigen binding. Techniques described for the production of single chain antibodies are described, e.g., in Plückthun in The Pharmacology of Monoclonal Antibodies, Rosenburg and Moore eds. Springer-Verlag, N.Y. (1994), 269-315. A “Fab fragment” as used herein is comprised of one light chain and the CH1 and variable regions of one heavy chain. The heavy chain of a Fab molecule cannot form a disulfide bond with another heavy chain molecule. An “Fc” region contains two heavy chain fragments comprising the CH2 and CH3 domains of an antibody. The two heavy chain fragments are held together by two or more disulfide bonds and by hydrophobic interactions of the CH3 domains. A “Fab′ fragment” contains one light chain and a portion of one heavy chain that contains the VH domain and the CH1 domain and also the region between the CH1 and CH2 domains, such that an interchain disulfide bond can be formed between the two heavy chains of two Fab′ fragments to form a F(ab′)2 molecule. A “F(ab′)2 fragment” contains two light chains and two heavy chains containing a portion of the constant region between the CH1 and CH2 domains, such that an interchain disulfide bond is formed between the two heavy chains. A F(ab′)2 fragment thus is composed of two Fab′ fragments that are held together by a disulfide bond between the two heavy chains. The “Fv region” comprises the variable regions from both the heavy and light chains, but lacks the constant regions.

Techniques for the production of antibodies and the elicitation of an immune response against a specific antigen are well known in the art and described, e.g. in Howard and Bethell (2000) Basic Methods in Antibody Production and Characterization, Crc. Pr. Inc.

In particular, antibodies of the present invention include immunoglobulin molecules and immunologically active portions of immunoglobulin molecules, i.e., molecules that contain an antigen binding site that immunospecifically binds carboxymethylated PP2Ac as defined herein. In one aspect, the antibodies are humanized or human and/or deimmunized. More preferably, the antibodies are humanized and most preferably the antibodies are fully humanized/human.

The present invention also relates to a process or method for the production of the inventive binding molecule/antibody as defined and provided herein, said process comprising

(a) culturing a host expressing nucleic acid molecule expressing at least one CDR and/or at least one heavy and/or light chain variable region as defined herein; or the hybridoma cell “7C10-C5”/“DSM ACC3350” under conditions allowing the expression of the antibody; and

(b) recovering the produced antibody from the culture.

Accordingly, the present invention also provides for methods/processes for the production of anti-methyl-PP2Ac-specific antibodies/anti-carboxymethylated PP2Ac-specific antibodies.

The invention also provides for a composition comprising the inventive binding molecule/antibody as defined herein above or as produced by the processes provided herein, a nucleic acid molecule. The invention also provides for a composition that comprises the hybridoma “7C10-C5”/“DSM ACC3350”. The composition of the present invention and comprising an inventive binding molecule/antibody may, further comprise a secondary antibody that is specifically binding to the inventive binding molecule/antibody that binds carboxymethylated PP2Ac. Such a composition may be, inter alia, useful as a kit for research or as a diagnostic kit. Such a diagnostic composition/kit may further comprise, optionally, means and methods for detection. The binding molecules/antibodies as well as the compostions of the present invention may also be useful in biological and/or medical screenings, e.g. in diagnostics, as well as in the evaluation of drugs and/or medicaments.

Furthermore, the inventive kit provided herein, i.e. the kit comprising an antibody specifically binding the carboxymethylated catalytic subunit of protein phosphatase 2A (PP2Ac) as described herein, may further comprise a brochure or leaflet with instructions for carrying out at least one of methods of the present invention, e.g. the method for specifically detecting the carboxymethylated catalytic subunit of protein phosphatase 2A (PP2Ac) in a biological sample, the in vitro method for prognosing the outcome of a cancer in a patient, the in vitro method for diagnosing whether a cancer is metastatic or prone to metastasize, the method of detecting an abnormal level of carboxymethylated PP2Ac in a sample from a patient, the in vitro method for prognosing the responsiveness of a cancer in a patient to treatment with an antiandrogen, the method for prognosing the progression of a disease in a subject, the method for predicting the responsiveness of a subject suffering from a disease to the treatment with a PP2A modulator, the method for determining the responsiveness of a subject suffering from a disease to the treatment with a PP2A modulator, the method for evaluating whether a test agent modulates PP2Ac carboxymethylation, the method for identifying agents that modulate PP2Ac carboxymethylation, the method for evaluating whether a test agent modulates the activity of PP2A, the method for identifying an agent that modulates the activity of PP2A, the method for identifying a PP2A modulator, the screening method for evaluating whether a molecule modulates the activity of PP2A, and/or the method for screening for (a) medicament(s) and/or drug(s), as described herein.

As illustrated in the appended Examples, both, an exemplary antibody according to the invention (7C10-C5) as well as recombinant monovalent or bivalent scFVs generated from the variable region of 7C10-C5, as well as a recombinant antibody comprising a Fc-region, bind carboxymethylated PP2Ac in a highly specifical manner. In context of this invention, “binding in highly specifical manner” means that it does not bind, under standard conditions and conditions as employed in the appended examples to non-methylated catalytic subunit of PP2A (PP2Ac). Furthermore, the inventive binding molecules/antibodies do not bind to carboxymethylated PP4c and/or to carboxymethylated PP6c under standard assay conditions, or at very low and/or much reduced levels compared to prior art antibodies. The term “specific binding” is known to the skilled artisan and is also further illustrated herein below and in the appended examples.

Preferably, the binding molecule/antibody of the present invention is a monoclonal antibody as described herein, or a scFv as described and provided herein and as employed in the appended examples.

As described above, the inventive anti-methyl-PP2Ac-specific antibodies/anti-carboxymethylated PP2Ac-specific antibodies (binding molecule/antibody of the present invention) does not specifically bind the non-methylated catalytic subunit of PP2A (PP2Ac). This means that said binding molecule/antibody binds the non-methylated PP2Ac at most at background levels which is much weaker than the specific binding to carboxymethylated PP2Ac. In particular, the binding to non-methylated PP2Ac is considered to be at most at background levels, when the binding to non-carboxymethylated PP2Ac is at least 4, 8, 16, 20, 24, 26, 32, 40, 50, 100, 200 or 500-fold, preferably at least 100-fold, weaker than the binding to carboxymethylated PP2Ac as determined by Western blotting, and/or the binding to a peptide comprising the non-methylated C-terminal region of PP2Ac is at least 4, 8, 16, 24, 26, 32, 40 or 48-fold, preferably at least 26-fold, weaker than the binding to a corresponding peptide comprising the carboxymethylated C-terminal region of PP2Ac as determined by ELISA. Furthermore, the binding to non-methylated PP2Ac may considered to be at most at background levels, when no reliable KD value can be determined by surface plasmon resonance experiments (e.g. Biacore), as illustrated in the appended Examples, i.e. because impermissibly high analyte concentration would have to be used to obtain a signal.

Assays for determining the strength of the binding of an antibody to antigen are well known in the art and further described in the Examples. For example, the binding strength may be determined inter alia by ELISA, Western blotting, quantitative immunostaining/immunohistochemistry/immunocytochemistry, (intracellular) flow cytometry, surface plasmon resonance, for example inter alia Biacore, and/or Microscale Thermophoresis (NanoTemper Technologies), preferably surface plasmon resonance and/or ELISA, more preferably surface plasmon resonance as described herein. The binding strength may be further expressed as affinity and/or avidity.

The differential affinity of the inventive anti-methyl-PP2Ac-specific antibodies/anti-carboxymethylated PP2Ac-specific antibodies, like antibody 7C10-C5, to carboxymethylated PP2A catalytic subunit versus non-carboxymethylated PP2A catalytic subunit may be determined by ELISA using peptides corresponding to the carboxymethylated or non-methylated carboxy-terminus of PP2A catalytic subunit. This can be done by immobilizing a dilution series of the antigen/peptide (e.g. in six 4-fold dilution steps from about 10 μg/ml to about 0.002 ug/ml) to the ELISA plate surface and incubating with a constant concentration of the antibody (1 μg/ml). It may further be done by immobilizing a constant concentration of the antigen (10 μg/ml) to the ELISA plate surface and incubation with a dilution series of the antibody (e.g. in five 4-fold dilution steps from about 2 μg/ml to about 0.002 μg/ml). By blotting the absorbance of the measurements against the antigen or antibody concentration, respectively, the antibody binding strength/affinity can be calculated. The steps of said ELISA assay may be combined with the step/specifications of the ELISA as described herein and/or in the appended Examples.

Specifically, the differential affinity of the inventive anti-methyl-PP2Ac-specific antibodies/anti-carboxymethylated PP2Ac-specific antibodies, like 7C10-C5, to carboxymethylated PP2A catalytic subunit versus non-carboxymethylated PP2A catalytic subunit may be determined by ELISA using peptides corresponding to the carboxymethylated or non-methylated carboxy-terminus of PP2A catalytic subunit. This can be done by immobilizing a dilution series of the antigen/peptide (e.g. in six 4-fold dilution steps from about 10 μg/ml to about 0.002 μg/ml (e.g. 002441406 μgimp) to the ELISA plate surface and incubating with a constant concentration of the antibody (1 μg/ml). Said antibody can be detected with a secondary antibody that is conjugated to a fluorescent or chromogenic compound (e.g. inter alia peroxidase). It may further be done by immobilizing a constant concentration of the antigen (10 μg/ml) to the ELISA plate surface and incubation with a dilution series of the antibody (e.g. in five 4-fold dilution steps from about 2 μg/ml to about 0.002 μg/ml (e.g. 0.001953125 μg/ml)). Also there, said antibody can be detected with a secondary antibody that is conjugated to a fluorescent or chromogenic compound (e.g. inter alia peroxidase). The respective signal of said compound (e.g. fluorescence, luminescence, chromatic enzyme substrate) can then be quantified. By blotting the signal against the antigen or antibody concentration, respectively, the antibody binding strength/affinity can be calculated. Furthermore, the ratio of two signals (or calculated binding strengths/affinities) generated by ELISAs employing the same antibody but two different peptides can be calculated, thereby determining how much stronger/weaker (fold-change) said antibody binds to the one peptide compared to the other peptide. Furthermore, the ratio of the signals (or calculated binding strengths/affinities) generated by ELISAs employing two different antibodies but the same peptide can be calculated, thereby determining how much stronger/weaker (fold-change) said peptide is by bound by the one antibody compared to the other antibody.

The differential affinity of the inventive anti-methyl-PP2Ac-specific antibodies/tanti-carboxymethylated PP2Ac-specific antibodies, like antibody 7C10-C5, to carboxymethylated PP2A catalytic subunit versus noncarboxy methylated PP2A catalytic subunit can be further determined by surface plasmon resonance, for example inter alia Biacore, using peptides corresponding to the carboxymethylated or non-methylated carboxy-terminus of PP2A catalytic subunit, as described herein in the context of K_{D} values, and as illustrated in the appended Examples.

Furthermore, for reference purposes only, and not in relation to the K_{D} values described herein and in the appended Examples in context of the present invention, a Fab fragment of 7C10-C5 can be prepared by digesting the antibody with Ficin (using e.g. Pierce™ Mouse IgG1 Fab and F(ab′)2 Preparation Kit, catalog number 44980). Peptides can be immobilized via their N-terminus to the Biacore dextrane chip surface by N-Hydroxysuccinimide ester or similar chemistry. The affinity of antibody 7C10-C5 can be determined by incubation of the immobilized peptides with solutions of different Fab fragment concentrations, ranging from e.g. 50 nmol/l to 800 nmol/l, and using the Biacore software to calculate Kₐ, K_{d} and K_{D}. Alternatively, the monovalent affinity of the scFv fragment can be determined by the same procedure.

In particular, the binding molecules/antibodies of the invention bind carboxymethylated PP2Ac at least 4, 8, 16, 20, 24, 26, 32, 40, 50, 100, 200 or 500-fold, preferably at least 100-fold stronger than non-carboxymethylated PP2Ac.

Binding may be measured (and fold-changes determined) by methods known in the art, by the measurement methods provided herein, and/or as illustrated in the appended Examples, i.e. by Western blotting, ELISA and/or surface plasmon resonance.

In particular, the binding molecule/antibodies of the invention (the anti-methyl-PP2Ac-specific antibodies/anti-carboxymethylated PP2Ac-specific antibodies, like antibody 7C10-C5)) may bind a peptide comprising the carboxymethylated C-terminal region of PP2Ac at least 4, 8, 16, 24, 26, 32, 40 or 48-fold, preferably at least 26-fold, stronger than a corresponding peptide comprising the non-methylated C-terminal region of PP2Ac, wherein said C-terminal region of PP2Ac has the sequence TPDYFL (SEQ ID NO:1), e.g. as determined by an ELISA. Preferably, both peptides (the carboxymethylated and the non-methylated peptides) have the same length. Preferably, the C-terminal region of PP2Ac comprised in said peptide(s) has the sequence HVTRRTPDYFL (SEQ ID NO:18).

As described above, the binding molecule/antibodies of the invention (the anti-methyl-PP2Ac-specific antibodies/anti-carboxymethylated PP2Ac-specific antibodies, like antibody 7C10-C5) do not bind, specifically bind or cross-react with the carboxymethylated catalytic subunit of PP4 (PP4c) and/or the carboxymethylated catalytic subunit of PP6 (PP6c). This means that said binding molecule/antibody binds the carboxymethylated PP4c or PP6c at most at background levels and/or with a much higher K_{D}, i.e. a binding which is much weaker than the specific binding to carboxymethylated PP2Ac. In particular, the binding to carboxymethylated PP4c or PP6c is considered to be at most at background levels, when the binding to carboxymethylated PP4c or PP6c is at least 4, 8, 16, 20, 24, 26, 32, 40, 50, 100, 200 or 500-fold, preferably at least 100-fold, weaker than the binding to carboxymethylated PP2Ac as determined by Western blotting, and/or the binding to a peptide comprising the carboxymethylated C-terminal region of PP4c or PP6c is at least 4, 8, 10, 16, 24, 32, 40 or 48-fold, preferably at least 10-fold, weaker than the binding to a corresponding peptide comprising the carboxymethylated C-terminal region of PP2Ac as determined by ELISA. The (non-desired) binding to carboxymethylated PP6c may be at most at background levels, when the binding to carboxymethylated PP6c is at least 4, 8, 16, 20, 24, 26, 32, 40, 50, 100, 200 or 500-fold, preferably at least 100-fold, weaker than the binding to carboxymethylated PP2Ac as determined by Western blotting, and/or the binding to a peptide comprising the carboxymethylated C-terminal region of PP6c may be at least 4, 8, 10, 16, 24, 32, 40 or 48-fold, preferably at least 16-fold, weaker than the binding to a corresponding peptide comprising the carboxymethylated C-terminal region of PP2Ac as determined by ELISA.

In particular, the binding molecule/antibodies of the invention (the anti-methyl-PP2Ac-specific antibodies/anti-carboxymethylated PP2Ac-specific antibodies, like antibody 7C10-C5) may be further considered to not bind, specifically bind or cross-react with the carboxymethylated catalytic subunit of PP4 (PP4c), when the K_{D} of the binding to a peptide comprising the carboxymethylated C-terminal region of PP4c, e.g. a peptide consisting of the last 11 amino acids of the carboxymethylated C-terminal region of PP4c (PSKKPVADYFL-CH₃; SEQ ID NO:20) is at least 4-, 6-, 8-, 10- or 12-fold, preferably at least 10-fold, e.g. about 12-fold, greater than the K_{D} of the binding to a peptide comprising the carboxymethylated C-terminal region of PP2Ac, e.g. a peptide consisting of the last 11 amino acids of the carboxymethylated C-terminal region of PP2Ac (HVTRRTPDYFL-CH₃; SEQ ID NO:18), in particular as described herein in the context of the dissociation constant (K_{D}) determined by surface plasmon resonance.

Thus the inventive antibody provided herein preferably binds a peptide consisting of the last 11 amino acids of the carboxymethylated C-terminal region of PP2Ac (HVTRRTPDYFL-CH₃; SEQ ID NO:18) at least 4-, 6-, 8-, 10- or 12-fold, preferably at least 10-fold, e.g. about 12-fold, stronger than a peptide consisting of the last 11 amino acids of the carboxymethylated C-terminal region of PP4c (PSKKPVADYFL-CH₃; SEQ ID NO:20). Preferably, said binding is determined by surface plasmon resonance (e.g. Biacore) as described herein, i.e. in the context of the dissociation constant (K_{D}). Preferably each peptide is employed at different concentrations, e.g at least 4 different concentrations. In particular, for the carboxymethylated peptide having the SEQ ID NO:18 one (e.g the lowest) concentration may be about 5 nM and another (e.g. the highest) concentration may be about 160 nM; wherein for the carboxymethylated peptide having the SEQ ID NO:20 one (e.g. the lowest) concentration may be about 50 nM and another (e.g. the highest) concentration may be about 800 nM.

Furthermore, the binding molecule/antibodies of the invention (the anti-methyl-PP2Ac-specific antibodies/anti-carboxymethylated PP2Ac-specific antibodies, like antibody 7C10-C5) may bind a peptide (PP2Ac peptide) comprising the C-terminal region of the carboxymethylated PP2Ac (SEQ ID NO:1) at least 4, 8, 10, 16, 24, 32, 40 or 48-fold, preferably at least 10-fold, stronger than a peptide (PP4c peptide) comprising the C-terminal region of the catalytic subunit of protein phosphatase 4 (PP4c), wherein the C-terminal region of PP4c has the sequence VADYFL (SEQ ID NO:19), e.g. as determined by an ELISA. Preferably both peptides (the PP2Ac and the PP4c peptides) have the same length. Preferably, the C-terminal region of PP2Ac comprised in the PP2Ac peptide has the sequence HVTRRTPDYFL (SEQ ID NO:18) and the C-terminal region of PP4c comprised in the PP4c peptide has the sequence PSKKPVADYFL (SEQ ID NO:20). Preferably, the carboxyl group of the C-terminal leucine of both peptides (the PP2Ac and the PP4c peptides) comprising either the C-terminal region of PP2Ac (PP2Ac peptide) or PP4c (PP4c peptide) is methylated.

Furthermore, the binding molecule/antibody of the invention may bind a peptide (PP2Ac peptide) comprising the C-terminal region of the carboxymethylated PP2Ac (SEQ ID NO:1) at least 4, 8, 10, 16, 24, 32, 40 or 48-fold, preferably at least 10-fold, stronger than a peptide (PP6c peptide) comprising the C-terminal region of the catalytic subunit of protein phosphatase 6 (PP6c), wherein the C-terminal region of PP6c has the sequence TTPYFL (SEQ ID NO:21), e.g. as determined by an ELISA. Preferably both peptides (the PP2Ac and the PP6c peptides) have the same length. Preferably, the C-terminal region of PP2Ac comprised in the PP2Ac peptide has the sequence HVTRRTPDYFL (SEQ ID NO:18) and the C-terminal region of PP6c comprised in the PP6c peptide has the sequence IPPRTTTPYFL (SEQ ID NO:22). Preferably, the carboxyl group of the C-terminal leucine of both peptides (the PP2Ac and the PP6c peptides) comprising either the C-terminal region of PP2Ac (PP2Ac peptide) or PP6c (PP6c peptide) is methylated.

As described above, the binding molecule/antibody of the present invention preferably binds the carboxymethylated catalytic subunit of PP2A (PP2Ac) stronger than the respective C-terminally amidated PP2Ac. In particular, the binding to carboxymethylated PP2Ac may be considered to be stronger, when the binding to a peptide comprising the carboxymethylated C-terminal region of PP2Ac is at least 4, 6, 8, 16, 24, 32, 40 or 48-fold, preferably at least 6-fold, stronger than the binding to a corresponding peptide comprising the amidated C-terminal region of PP2Ac as determined by an ELISA.

In particular, the binding molecule/antibody of the invention may bind a peptide comprising the carboxymethylated C-terminal region of PP2Ac at least 4, 6, 8, 16, 24, 32, 40 or 48-fold, preferably at least 6-fold, stronger than a corresponding peptide comprising the amidated C-terminal region of PP2Ac, wherein said C-terminal region of PP2Ac has the sequence TPDYFL (SEQ ID NO:1). Preferably, both peptides (the carboxymethylated and the amidated peptides) have the same length. Said length may be 6 to 16, preferably 9 to 13, preferably 11 amino acids.

In one embodiment, the peptide comprising either the C-terminal region of PP2Ac, PP4c or PP6c consists of 6 to 16, preferably 9 to 13, preferably 11 amino acids.

In some preferred embodiments, the binding molecule/antibody of the invention binds a peptide comprising the carboxymethylated C-terminal region of PP2Ac:
(i) at least 4, 8, 16, 24, 26, 32, 40 or 48-fold, preferably at least 26-fold, stronger than a corresponding peptide comprising the non-methylated C-terminal region of PP2Ac,
(ii) at least 4, 6, 8, 16, 24, 32, 40 or 48-fold, preferably at least 6-fold, stronger than a corresponding peptide comprising the amidated C-terminal region of PP2Ac,
(iii) at least 4, 8, 10, 16, 24, 32, 40 or 48-fold, preferably at least 10-fold, stronger than a peptide comprising the carboxymethylated C-terminal region of the catalytic subunit of protein phosphatase 4 (PP4c), and/or
(iv) at least 4, 8, 10, 16, 24, 32, 40 or 48-fold, preferably at least 10-fold or 16-fold, preferably at least 16-fold, stronger than a peptide comprising the carboxymethylated C-terminal region of the catalytic subunit of protein phosphatase 6 (PP6c),

wherein said C-terminal region of PP2Ac has the sequence TPDYFL (SEQ ID NO:1),

wherein the C-terminal region of PP4c has the sequence VADYFL (SEQ ID NO:19), and

wherein the C-terminal region of PP6c has the sequence TTPYFL (SEQ ID NO:21); e.g. as determined by an ELISA.

Preferably, said peptides comprising either the C-terminal region of PP2Ac, PP4c or PP6c, have the same length. Preferably, said peptides have the sequences HVTRRTPDYFL (SEQ ID NO:18), PSKKPVADYFL (SEQ ID NO:20) and IPPRTTTPYFL (SEQ ID NO:22), respectively. Preferably, the peptide comprising either the C-terminal region of PP2Ac, PP4c or PP6c consists of 6 to 16, preferably 9 to 13, preferably 11 amino acids.

In further preferred embodiments, the binding molecule/antibody of the invention binds a peptide consisting of the last 11 amino acids of the carboxymethylated C-terminal region of PP2Ac (HVTRRTPDYFL-CH₃; SEQ ID NO:18):
(i) with a dissociation constant (K_{D}) of 200 nM, 100 nM, 80 nM, 60 nM, 40 nM, 20 nM, 15 nM or less, preferably 40 nM or less, more preferably 20 nM or less, e.g. about 11 nM,
(ii) at least 10-, 20-, 50-, 100-, 200-, 300-, 400-, 500-, or 600-fold, preferably at least 100-fold or 400-fold, stronger than a peptide consisting of the last 11 amino acids of the non-methylated C-terminal region of PP2Ac (HVTRRTPDYFL-OH; SEQ ID NO:18), and/or
(iii) at least 4-, 6-, 8-, 10- or 12-fold, preferably at least 10-fold, e.g. about 12-fold, stronger than a peptide consisting of the last 11 amino acids of the carboxymethylated C-terminal region of PP4c (PSKKPVADYFL-CH₃; SEQ ID NO:20);

preferably as determined by surface plasmon resonance (e.g. Biacore) under the conditions described herein, e.g. in context of the K_{D}.

In particular, the binding strength of the antibodies/binding molecules of the invention/the inventive anti-methyl-PP2Ac-specific antibodies/the inventive anti-carboxymethylated PP2Ac-specific antibodies to a peptide comprising the carboxymethylated C-terminal region of PP2Ac (SEQ ID NO:1 or SEQ: ID NO:18) is preferably not affected when the tyrosine in said C-terminal region of PP2Ac is phosphorylated. Said tyrosine also corresponds to tyrosine 307 (Tyr³⁰⁷) of human PP2Ac as described herein.

In particular, the binding strength of the binding molecule/antibody of the invention to a peptide comprising the carboxymethylated C-terminal region of PP2Ac (SEQ ID NO:1 or SEQ: ID NO:18) is preferably not affected, or is at most 4-fold, preferably at most 3-fold, preferably at most 2-fold higher, when the most C-terminal threonine in said C-terminal region of PP2Ac is phosphorylated. Said threonine also corresponds to threonine 304 (Thr³⁰⁴) of human PP2Ac as described herein.

In the context of the invention, a certain fold-change is considered to be met, if said fold-change is within an error range. In particular, the binding strength is considered to be not affected when the fold-change is 1+/−an error range. As used herein, the error range is at most +/−50%, preferably at most +/−30%, preferably at most +/−10%.

Thus, in some particularly preferred embodiments, the binding molecule/antibody of the invention (anti-methyl-PP2Ac-specific antibody/anti-carboxymethylated PP2Ac-specific antibody) binds a peptide comprising the carboxymethylated C-terminal region of PP2Ac
(i) at least 4, 8, 16, 24, 26, 32, 40 or 48-fold, preferably at least 26-fold, stronger than a corresponding peptide comprising the non-methylated C-terminal region of PP2Ac,
(ii) at least 4, 6, 8, 16, 24, 32, 40 or 48-fold, preferably at least 6-fold, stronger than a corresponding peptide comprising the amidated C-terminal region of PP2Ac,
(iii) at least 4, 8, 10, 16, 24, 32, 40 or 48-fold, preferably at least 10-fold, stronger than a peptide comprising the carboxymethylated C-terminal region of the catalytic subunit of protein phosphatase 4 (PP4c),
(iv) at least 4, 8, 10, 16, 24, 32, 40 or 48-fold, preferably at least 10-fold or 16-fold, preferably at least 16-fold, stronger than a peptide comprising the carboxymethylated C-terminal region of the catalytic subunit of protein phosphatase 6 (PP6c),
wherein the binding strength of said antibody to a peptide comprising the carboxymethylated C-terminal region of PP2Ac is
(v) not affected when the tyrosine in said C-terminal region of PP2Ac is phosphorylated, and
(vi) not affected, or is at most 4-fold, preferably at most 3-fold, preferably at most 2-fold higher, when the most C-terminal threonine in said C-terminal region of PP2Ac is phosphorylated, and

wherein said C-terminal region of PP2Ac has the sequence TPDYFL (SEQ ID NO:1),

wherein the C-terminal region of PP4c has the sequence VADYFL (SEQ ID NO:19), and

wherein the C-terminal region of PP6c has the sequence TTPYFL (SEQ ID NO:21);

e.g. as determined by an ELISA.

Preferably, said peptides comprising either the C-terminal region of PP2Ac, PP4c or PP6c, have the same length. Preferably, said peptides have the sequences HVTRRTPDYFL (SEQ ID NO:18), PSKKPVADYFL (SEQ ID NO:20) and IPPRTTTPYFL (SEQ ID NO:22), respectively. Preferably, the peptide comprising either the C-terminal region of PP2Ac, PP4c or PP6c consists of 6 to 16, preferably 9 to 13, preferably 11 amino acids.

The binding strength of the binding molecule/antibody of the invention to a certain peptide may be determined or validated by an enzyme-linked immunosorbent assay (ELISA), preferably wherein said binding strength corresponds to the signal intensity determined by said ELISA. ELISAs may be carried out, for example, inter alia as described in the appended Examples. For example, the ELISA comprises the steps of coating a plate with said certain peptide, incubating the coated plated with said antibody, detecting said antibody with a secondary antibody conjugated to a fluorescent or chromogenic compound (e.g. inter alia peroxidase), and quantifying the respective signal of said compound (e.g. fluorescence, luminescence, chromatic enzyme substrate).

The ratio of two signals generated by ELISAs employing the same antibody but two different peptides can be calculated, thereby determining how much stronger/weaker (fold-change) said antibody binds to the one peptide compared to the other peptide. Furthermore, the ratio of the signals generated by ELISAs employing two different antibodies but the same peptide can be calculated, thereby determining how much stronger/weaker (fold-change) said peptide is by bound by the one antibody compared to the other antibody.

Furthermore, the binding strength of the binding molecule/antibody of the invention to a certain protein (i.e. PP2Ac, PP4c or PP6c) may be determined or validated by western blotting (immunoblotting). Western blotting may be carried out, for example, inter alia as described in the appended Examples. For example, Western blotting comprises the steps of immunoprecipitating a tagged protein (e.g. HA-PP2Ac) from a lysate of cells expressing said tagged protein, performing an SDS-PAGE with said protein, transferring the protein to a nitrocellulose membrane, incubating the nitrocellulose membrane with said antibody, detecting said antibody with a secondary antibody conjugated to a fluorescent or chromogenic compound (e.g. inter alia peroxidase), and quantifying the respective signal of said compound (e.g. fluorescence, luminescence, chromatic enzyme substrate).

The ratio of two signals generated by Western blotting employing the same antibody but two different proteins can be calculated, thereby determining how much stronger/weaker (fold-change) said antibody binds to the one protein compared to the other protein. Furthermore, the ratio of the signals generated by Western blotting employing two different antibodies but the same peptide can be calculated, thereby determining how much stronger/weaker (fold-change) said peptide is by bound by the one antibody compared to the other antibody.

In particular, the binding molecule/antibody of the invention. i.e. the inventive anti-methyl-PP2Ac-specific antibodies/anti-carboxymethylated PP2Ac-specific antibodies, like antibody 7C10-C5, may bind carboxymethylated PP2Ac at least 10, 20, 50, 100, 200 or 500-fold stronger, preferably at least 100-fold stronger than carboxymethylated PP4c and/or carboxymethylated PP6c, wherein said binding strength may be determined by Western blotting.

Furthermore, the binding, binding strength, affinity, avidity of the binding molecule/antibody of the invention to a certain protein or peptide may be, inter alia, determined by surface plasmon resonance and other methods known to the skilled artisan. Such methods comprise, but are not limited to inter alia Biacore or by Microscale Thermophoresis (NanoTemper Technologies).

In particular, the binding strength, affinity or avidity of an antibody binding to antigen may be specified by the equilibrium dissociation constant (K_{D}), i.e. as described herein in context of surface plasmon resonance (e.g. Biacore) measurements, e.g., employing the inventive antibody provided herein, and/or as illustrated in the appended Examples. Preferably, the value of the equilibrium dissociation constant (K_{D}) may be obtained by fitting a plot of response at equilibrium (Req) against the respective concentration of the analyte. Thus, the dissociation constant (K_{D}) may be determined from the relationship between the equilibrium binding level (response) and the analyte concentration, and thus may refer, in particular, to the analyte concentration at 50% of the maximum (saturated) response (see the curves in FIG. 32 for this relationship, and the respective K_{D} as vertical line). Furthermore, the term K_{D} may refer to the ratio of the off-rate (k_{off}) constant over the on-rate (IQ constant: (K_{D}=k_{off}/kₒₙ). In other words, the smaller the dissociation constant (K_{D}), the stronger the binding of the antibody to antigen.

Binding molecules/antibodies of the invention, i.e. the inventive anti-methyl-PP2Ac-specific antibodies/anti-carboxymethylated PP2Ac-specific antibodies, like antibody 7C10-C5, may be suitable for quantifying the amount of methyl-PP2AC, a prerequisite for trimeric complex formation. Accordingly, a binding molecule/antibody of the present invention may also detect heterotrimeric PP2A holoenzyme and/or active PP2A in a biological sample since carboxymethylation of PP2Ac is a prerequisite for formation of certain trimeric holoenzymes.

As is illustrated herein below, the amount of heterotrimeric PP2A holoenzyme and/or active PP2A may be quantified by employing the binding molecule/antibody of the invention.

In one embodiment, the binding molecule/antibody of the invention, i.e. the inventive anti-methyl-PP2Ac-specific antibodies/anti-carboxymethylated PP2Ac-specific antibodies, like antibody 7C10-C5, is raised against an immunogen as described herein and in the following in the context of the method for producing an antibody according to the invention. Also this immunogen is part of this invention.

Accordingly, the invention further relates to a method for producing a binding molecule/an antibody specifically binding the carboxymethylated catalytic subunit of protein phosphatase 2A (PP2Ac), wherein said method comprises the steps of
(a) immunizing a non-human mammal, preferably a mouse, with a peptide comprising the sequence of the 5, preferably 6, C-terminal amino acids of the carboxymethylated PP2Ac,
(b) generating hybridoma clones from immune cells of said non-human animal,
(c) selecting a hybridoma clone whereof the supernatant binds at least at least 4, 8, 16, 20, 24, 26, 32, 40, 50, 100, 200 or 500-fold, preferably at least 100-fold stronger to the PP2Ac of a cell which contains a PP2A specific methyltransferase (PPM1/LCMT-1) than to the PP2Ac of a cell which lacks said PP2A specific methyltransferase, and
(d) obtaining said antibody from said selected hybridoma clone.

Preferably, the immunization peptide/immunogen in step (a) and/or the immunization peptide/immunogen according to the invention has the sequence TPDYFL (SEQ ID NO:1), PDYFL (SEQ ID NO:23), or RTPDYFL (SEQ ID NO:24), preferably SEQ ID NO:1, wherein the carboxyl group of the C-terminal leucine of said peptide is methylated/methylesterified, and preferably wherein said sequence further includes at the N-terminus a cysteine followed by a β-alanine. Preferably, said peptide is coupled to a carrier protein, preferably to keyhole limpet hemocyanin (KLH) via said cysteine. Said KLH may be maleimide activated before coupling to said cysteine.

Thus, the immunization peptide/immunogen according to the invention consists in one preferred embodiment of the six C-terminal amino acids of the carboxymethylated PP2Ac (SEQ ID NO:1) in addition to a cysteine followed by a β-alanine at the N-terminus. Preferably said peptide is coupled via the cysteine to KLH.

Thus, in particularly preferred embodiments, the immunization peptide/immunogen according to the invention has the sequence KLH-Cys-βAla-Thr-Pro-Asp-Tyr-Phe-Leu-CH₃ (SEQ ID NO:48), wherein Leu-CH₃ is the carboxymethylated C-terminal leucine.

Without being bound by theory, the particularly short immunization peptide only comprising six amino acids (SEQ ID NO:1) of PP2Ac including the C-terminal carboxymethylated leucine, promotes the production of an antibody according to the invention, because the immunized non-human animal tends to raise antibodies against an epitope comprising the C-terminal carboxymethylated leucine.

It has been found in the context of the present invention, and as illustrated in the appended Examples, that hybridoma supernatants/antibodies can be screened against genetically modified cell lines/strains having different levels of carboxymethylated PP2Ac. Particularly useful are cells expressing high levels of PP2Ac (or an orthologue of PP2Ac), preferably because a recombinant PP2Ac which is preferably tagged (e.g. HA), has been introduced into those cells.

Using a such a cell line/strain but further lacking PPM1 (or the mammalian orthologue leucine carboxyl methyltransferase 1; LCMT1) is particularly useful for determining the specificity of a putative anti-carboxymethylated PP2Ac antibody. In particular, the specific epitope of the putative anti-carboxymethylated PP2Ac antibody is not present (or only present at very low levels) in said cell line/strain in contrast to other potential epitopes with which said antibody may potentially cross-react (i.e. the non-carboxymethylated PP2Ac). This is particularly useful for assessing the unspecific/background binding of the putative anti-carboxymethylated PP2Ac antibody.

Furthermore, it may be further useful to assess the binding of said antibody to PP2Ac of a cell line/strain lacking the PP2A specific demethylase PPE1 (or the mammalian orthologue protein phosphatase methylesterase 1, PPME1/PME1). In particular, the binding of said antibody to PP2Ac of a WT cell line/strain (having normal levels of PPM1/LCMT1 and PPE1/PPME1) may be compared to the binding of said antibody to a cell line/strain lacking PPM1/LCMT1 or a cell line/strain lacking PPE-1/PPME1. With this approach, the specific binding to carboxymethylated PP2Ac can be confirmed (signal increase with the cell line/strain lacking PPE1/PPME1 compared to WT), and the absence of cross-reactivity with non-methylated PP2Ac can be ensured (absence of a signal/very low signal with the cell line/strain lacking PPM1/LCMT1 compared to WT).

Of note, the screening can be done inter alia in yeast or human cells because of the absolute conservation of the C-terminus of PP2Ac (TPDYFL) (SEQ ID NO:1) and the conservation of the enzymes regulating demethylation and methylation of PP2Ac.

Thus, in certain embodiments of the production method according to the invention, the cell containing said PP2A specific methyltransferase lacks the PP2A specific demethylase (PPE-1/PPME1).

In preferred embodiments of the production method, the cells, in particular the cells lacking PPM1/LCMT1, further express PP4c (or the yeast orthologue PPH3) and/or PP6c (or the yeast orthologue SIT4), preferably at high levels, preferably because a recombinant PP4c/PPH3 and/or PP6c/SIT has been introduced in to said cells, and in the context of said preferred embodiments, said cells are preferably mouse cells, preferably a mouse fibroblast cell line.

Preferably, the cells for assaying the binding of the hybridoma supernatant are not limited to a certain organism, and may be for example, inter alia yeast cells or mammalian cell such as inter alia mouse or human cell lines. Preferably, said cells are yeast cells.

The assessment of the binding of a hybridoma clone supernatant/antibody to PP2Ac of a cell may further comprise the isolation of said PP2Ac from said cell and/or the identification of said PP2Ac within said cell or a lysate thereof. For example, the PP2Ac may be immunoprecipitated/isolated/purified with an antibody, in particular when it is tagged (e.g. with HA). Furthermore, the proteins of a lysate of said cell may be separated by SDS-PAGE, and the band of the size of PP2Ac may be identified and the binding of said hybridoma clone supernatant/antibody to said band may be evaluated in subsequent western blotting.

In preferred embodiments of the production method according to the invention, the PP2Ac of a cell is contained in the lysate of said cell. Preferably the binding of the hybridoma supernatant to said PP2Ac is determined by western blotting.

In one embodiment, the binding molecule/antibody of the invention is obtainable by the method for producing an antibody according to the invention.

The binding molecules/antibodies of the present invention are characterized in that they specifically bind the carboxymethylated catalytic subunit of (human) protein phosphatase 2A (PP2Ac) as defined herein. In particular, the inventive binding molecules/antibodies specifically bind the methylated carboxyl group of the C-terminal leucine/Leu309 of (human) PP2Ac. As illustrated herein above and in the appended examples, this is in contrast to antibodies mentioned in the prior art (or partially available from commercial resources, like “2A10” (Upstate Biotechnology (now Merck-Millipore), Abcam, Biolegend/Covance, ImmuQuest and Santa Cruz Biotechnology). Binding molecules/antibodies of the present invention do not cross-react (or at most at background levels or very low and/or much reduced levels as described herein above) with α-carboxymethylated catalytic subunits of protein phosphatase 4 (PP4c) and protein phosphatase 6 (PP6c) (for example in Western Blots as described herein and in the appended examples). Furthermore, binding molecules/antibodies of the present invention may preferably bind α-carboxymethylated PP2Ac C-terminal peptides with an at least 10-fold higher signal intensity than the α-carboxymethylated PP4c and PP6c C-terminal peptides (for example in ELISAs as described herein and in the appended examples, and/or as determined by surface plasmon resonance, as described herein). Furthermore, binding molecules/antibodies of the present invention (inventive anti-methyl-PP2Ac-specific antibodies/anti-carboxymethylated PP2Ac-specific antibodies) may bind α-carboxymethylated PP2Ac C-terminal peptides with an at least 16-fold higher signal intensity than the α-carboxymethylated PP6c C-terminal peptides (for example in ELISAs as described herein and in the appended examples). In addition, the inventive binding molecules/antibodies are preferably characterized in that their specific binding to the carboxymethylated catalytic subunit of (human) protein phosphatase 2A (PP2Ac) is not impaired by the concurrent phosphorylation of tyrosine 307 or threonine 304 of PP2Ac, i.e. there is no impairment of 7C10-C5 binding specificity by concurrent phosphorylation of tyrosine 307 or threonine 304 of PP2Ac.

These highly specific features, taken alone or in individual combinations, of the inventive binding molecules/antibodies render these inventive anti-methyl-PP2Ac-specific antibodies/anti-carboxymethylated PP2Ac-specific antibodies extremely valuable, not only as research tools, but also as “diagnostic” tools in human and animal disorders and or diseases. For example, the binding molecules/antibodies of the present invention may be particularly useful in the specific detection and/or specific quantification of α-carboxymethylated PP2Ac in a biological sample. Specific detection and/or specific quantification of α-carboxymethylated PP2Ac may be a useful marker system in the evaluation/assessment of diseases and/or disease status. Accordingly, detected and/or quantified (human) α-carboxymethylated PP2Ac in a biological sample may serve as marker (biomarker) in or for cancer and other diseases. The specific detection of α-carboxymethylated PP2Ac and/or the detection of the expression level of α-carboxymethylated PP2Ac using the binding molecules/antibodies of the present invention may serve as a prognostic biomarker in cancer and other diseases. As also discussed in the appended examples, the assembly of trimeric tumor-suppressive PP2A holoenzymes is promoted and/or facilitated by PP2Ac α-carboxymethylation. The PP2Ac α-carboxymethylation levels correlate positively with the levels of methylation-sensitive trimeric PP2A holoenzymes. Accordingly, the binding molecules/antibodies of the present invention may be employed in the determination and/or evaluation of α-carboxymethylated PP2Ac levels in normal and disease tissue. Such an evaluation may comprise the use of immunohistological methods (immunohistochemistry (IHC)). Medical conditions to be assessed may comprise cancer (like prostate, lung adenocarcinoma, lung squamous carcinoma, breast cancer, colon cancer and leukemias), heart diseases (like cardiac fibrosis, non-ischemic/ischemic human heart failure), neurological disorders (like Alzheimers disease), and/or metabolic disorders (like diabetes).

Furthermore and as discussed in the present invention, without deferring form the gist of the present invention, the binding molecules/antibodies of the present invention (anti-methyl-PP2Ac-specific antibodies/anti-carboxymethylated PP2Ac-specific antibodies) as well as the herein defined compositions and kits may be employed as diagnostics/diagnostic compositions in biological and/or medical screenings as well as in drug screenings.

The diagnostic composition of the present invention and/or the kit of the present invention may comprise a binding molecule/antibody of the present invention and may further comprise, optionally, means and methods for detection. In accordance with the present invention, suitable detectable labels or markers include, but are not limited to, a radioisotope, a nanoparticle, a fluorescent compound, a bioluminescent compound, chemiluminescent compound, a metal chelator or an enzyme. In general, a “label” or a “detectable moiety” is a compound that when linked with the antibody of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

The invention further relates to a method for specifically detecting the carboxymethylated catalytic subunit of protein phosphatase 2A (PP2Ac) in a biological sample, wherein said method comprises a step of contacting said biological sample with the binding molecule/antibody of the invention.

Of note, a pH greater than 8.0, in particular greater than 8.5 may lead to hydrolysis of the carboxymethylation of PP2Ac, thus artificially generating non-methylated PP2Ac (as also illustrated in the appended Examples by NaOH). To obtain an accurate non-falsified carboxymethylation signal, it is preferably avoided to treating or contact a sample with a liquid having a pH greater than 8.0 or 8.5, i.e. before and/or during contacting said sample with the binding molecule/antibody of the invention.

Thus, the sample/biological sample as described herein does preferably not have a pH greater than 8.0, preferably 8.5, and/or said sample is not treated with a liquid having a pH greater than 8.0, preferably 8.5. Preferably said sample is not treated with said liquid before and/or during contacting with said antibody.

In one illustrative embodiment, if lysates are prepared under native conditions, a PME-1 inhibitor, for example ABL127 (e.g. at 2 μM final concentration in buffer), may be added. This avoids undesired demethylation of PP2Ac in the lysate by PME1 (Yabe (2018) FEBS Open Bio. 8(9):1486-1496; see, e.g., for PME1 activity at 0 degrees).

ABL127, as used herein, refers to dimethyl (3R)-3-cyclopentyl-4-oxo-3-phenyldiazetidine-1,2-dicarboxylate (Bachovchin (2011) J Med Chem.;54(14):5229-36).

Further, if lysates are prepared under native conditions, phosphatase and protease inhibitors, for example S-adenosylhomocysteine (SAH; e.g. at a concentration of 100 μM) may be added to said lysates to inhibit LCMT1. This avoids undesired methylation of PP2Ac in the lysate by LCMT1. Reference for SAH inhibition of LCMT1: Sontag (2007) J Neurosci.;27(11):2751-2759.

As described already above, the binding molecule/antibody of the invention (anti-methyl-PP2Ac-specific antibody/anti-carboxymethylated PP2Ac-specific antibody) may be used in prognostic, predictive, and/or diagnostic methods, e.g. for diagnosing the state, stage and/or subtype of a disease in a subject and/or prognosing the progression of a disease in a subject. Prognostic, predictive and/or diagnostic methods according to the invention are described in the following.

Thus, the invention further relates to a method for diagnosing the state, stage and/or subtype of a disease, wherein said method comprises detecting or quantifying carboxymethylated PP2Ac in a sample using the binding molecule/antibody of the invention (anti-methyl-PP2Ac-specific antibody/anti-carboxymethylated PP2Ac-specific antibody/an antibody specifically binding carboxymethylated PP2Ac) provided herein.

Furthermore, the present invention relates to a method for diagnosing the state, stage and/or subtype of a disease in a subject, wherein said method comprises detecting or quantifying carboxymethylated PP2Ac in a biological sample from said subject using the herein provided inventive binding molecule/antibody of the invention (anti-methyl-PP2Ac-specific antibody/anti-carboxymethylated PP2Ac-specific antibody/antibody specifically binding carboxymethylated PP2Ac).

In addition, the present invention relates to a method for prognosing the progression of a disease, wherein said method comprises detecting or quantifying carboxymethylated PP2Ac in a sample using the herein provided inventive binding molecule/antibody of the invention (anti-methyl-PP2Ac-specific antibody/anti-carboxymethylated PP2Ac-specific antibody/antibody specifically binding carboxymethylated PP2Ac).

Furthermore, the present invention relates to a method for prognosing the progression of a disease in a subject, wherein said method comprises detecting or quantifying carboxymethylated PP2Ac in a biological sample from said subject using the herein provided inventive binding molecule/antibody of the invention (anti-methyl-PP2Ac-specific antibody/anti-carboxymethylated PP2Ac-specific antibody/antibody specifically binding carboxymethylated PP2Ac).

The herein provided diagnostic, prognostic and/or predictive methods are preferably in vitro methods. In particular, the disease of which the state, stage and/or subtype is diagnosed, the disease of which the progression is prognosed, and/or the disease of which the outcome is prognosed, is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A).

Hyperphosphorylation, as used herein, refers to an abnormally high phosphorylation of a protein, i.e. a component of a signalling pathway, wherein the normal state may be determined with healthy tissue of a series of subjects.

Hypophosphorylation, as used herein, refers to an abnormally low phosphorylation of a protein, i.e. a component of a signalling pathway, wherein the normal state may be determined with healthy tissue of a series of subjects.

In certain embodiments of the diagnostic, prognostic, and/or predictive methods according to the present invention, the disease of which the state, stage and/or subtype is diagnosed, the disease of which the progression is prognosed, and/or the disease of which the outcome is prognosed, is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

In one embodiment, said disease may be selected from the group consisting of cancer, a neurodegenerative disorder, diabetes and a heart disease. Preferably, said disease is associated with and/or is caused by a deregulated signaling pathway, wherein said deregulation comprises the hyperphosphorylation of at least one component of said signaling pathway, and wherein said component can be dephosphorylated by protein phosphatase 2A (PP2A). Preferably, said disease is cancer which is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

In preferred embodiments of the diagnostic, prognostic, and/or predictive methods according to the present invention, the disease is cancer which is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

As already indicated above, a variety of diseases, such as inter alia cancer, is associated with and/or caused by hyperphosphorylation of a signaling pathway component which may be associated with abnormal activity, i.e. hyperactivity, of the signaling pathway. Evidently, it is of paramount importance for cells that the signaling pathways which control essential cell properties such as inter alia growth, proliferation, apoptosis, differentiation and metabolism, are tightly controlled, have normal activity, and normally respond to external/internal cues such as inter alia cytokines/growth factors and feed-back control. In particular, when signaling pathways are hyperactive (or hypoactive), cells might not behave adequately. Specifically, the hyperactivity of certain signaling pathways comprising a tyrosine kinase receptor, an androgen receptor, Bcl2, PI3K, AKT, S6K, MAP, ERK, β-catenin and/or c-MYC is associated with diseases such as inter alia cancer.

In many cases, the hyperphosphorylated signaling pathway component is a substrate of the phosphatase PP2A which is capable of dephosphorylating said component. However, the effectivity of PP2A in (re)establishing a normal, non-pathological, phosphorylation state of such a signaling pathway component depends on the level and/or activity of the respective active substrate-specific PP2A holoenzyme. Usually, the level and/or activity of such a PP2A holoenzyme, i.e. a tumor-suppressive PP2A holoenzyme, depends on the carboxymethylation of its catalytic subunit (PP2Ac).

Thus, if a subject suffering from a disease as described herein requires or favors high PP2A activity, i.e. high levels of carboxymethylation dependent PP2A holoenzymes to stop, slow-down or reverse the progression of said disease, high methyl-PP2Ac levels detected in a biological sample from said subject by the inventive antibody provided herein, may indicate a favorable progression of said disease. In contrast, if the methyl-PP2Ac levels detected in a biological sample from said subject are rather low, said the progression of said disease may be rather unfavorable.

As illustrated in the appended Examples, the carboxymethylated PP2Ac levels detected by the inventive antibody provided herein, were much lower in metastatic prostate cancer tissue than in respective non-metastatic prostate cancer. This demonstrates that the antibody of the invention is suitable for diagnosing different disease state, stage and/or subtype of cancer, in particular, metastatic vs. non-metastatic cancer, late stage vs. early stage cancer, and/or a methyl-PP2Ac-associated cancer. Moreover, those findings strongly suggest that the detection of certain methyl-PP2Ac levels in a tissue which are associated with a certain state and/or stage of a disease may indicate the progression towards that state and/or stage, or the maintenance of that state and/or stage. For example, if carboxymethylated PP2Ac levels are very low in a non-metastatic tumor sample (and such low levels more often occur in metastatic tumor samples), it may be likely that said non-metastatic tumor becomes metastatic and the progression may thus be rather unfavourable. In contrast, very high methyl-PP2Ac levels would rather indicate that the tumor may not very soon become metastatic and that the progression is thus rather favourable. Thus, when very low (e.g. an Allred Score <2) carboxymethylated PP2Ac levels are detected in a non-metastatic tumor sample, there is a high likelihood that said tumor will metastasize, and hence it maybe prognosed that the outcome of the cancer is negative and/or the progression is unfavourable, in particular when such very low carboxymethylated PP2Ac levels are associated with and/or found in corresponding metastatic tumor samples. In contrast, when higher (e.g. an Allred Score of at least 2) carboxymethylated PP2Ac levels are detected in a non-metastatic tumor sample, there is a low likelihood that said tumor will metastasize, and hence it maybe prognosed that the outcome of the cancer is positive and/or the progression is favourable.

Of note, the diagnostic, prognostic and/or predictive methods of the invention critically rely on antibodies/binding molecules of the invention (/anti-methyl-PP2Ac-specific antibodies/anti-carboxymethylated PP2Ac-specific antibodies), i.e. an antibody specifically binding carboxymethylated PP2Ac because a precise detection or quantification of carboxymethylated PP2Ac levels is desired or required. As described herein, allegedly PP2Ac specific antibodies of the prior art, in fact, also bind non-methylated PP2Ac, PP2Ac with other modifications, cross-react with PP4c and/or PP6c and/or are impaired by concurrent phosphorylation near the epitope comprising the C-terminal carboxymethylated leucine of PP2Ac and are therefore predisposed for giving erroneous results, and/or, provide, at best, a much lower accuracy, sensitivity and/or specificity if any meaningful results can be obtained at all.

In one embodiment, the diagnostic, prognostic and/or predictive methods according to the present invention comprise a step of determining the level of carboxymethylated PP2Ac in a sample by contacting said sample with the inventive antibody provided herein, i.e. an antibody specifically binding carboxymethylated PP2Ac. Preferably, said step of determining the level of carboxymethylated PP2Ac is an in vitro method. Such a method may comprise, but is not limited to, immunohistochemistry employing the inventive antibodies specifically binding carboxymethylated PP2Ac as disclosed herein. Accordingly, the present invention also relates to a method for prognosing the progression of a disease in a subject, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with an inventive antibody specifically binding carboxymethylated PP2Ac, and
(b) evaluating whether the disease progression is favorable or unfavorable, and
   indicating a favorable progression if
      said disease is associated with hyperphosphorylation of said signaling pathway component and the level of said carboxymethylated PP2Ac is elevated, or
      said disease is associated with hypophosphorylation of said signaling pathway component and the level of said carboxymethylated PP2Ac is reduced, and/or
   indicating an unfavorable progression if
      said disease is associated with hyperphosphorylation of said signaling pathway component and the level of said carboxymethylated PP2Ac is reduced, or
      said disease is associated with hypophosphorylation of said signaling pathway component and the level of said carboxymethylated PP2Ac is elevated.

Preferably, as used herein, an elevated level of carboxymethylated PP2Ac may correspond to an Allred Score of 2 or greater, and/or a reduced level of carboxymethylated PP2Ac may correspond to an Allied Score lower than 2, preferably as described herein, e.g., in context of the in vitro method for prognosing the outcome of a cancer in a patient.

As used in context of this invention, the term “antibody specifically binding carboxymethylated PP2Ac” relates to anti-methyl-PP2Ac-specific antibodies/anti-carboxymethylated PP2Ac-specific antibodies as disclosed and provided herein.

Furthermore, the invention relates to methods for prognosing the progression of a disease in a subject, wherein said disease may be selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with an inventive antibody specifically binding carboxymethylated PP2Ac, and
(b) evaluating whether the disease progression is favorable or unfavorable, and
   indicating a favorable progression if
      high PP2A activity is beneficial and the level of said carboxymethylated PP2Ac is elevated, or
      low PP2A activity is beneficial and the level of said carboxymethylated PP2Ac is reduced, and/or
   indicating an unfavorable progression if
      high PP2A activity is beneficial and the level of said carboxymethylated PP2Ac is reduced, or
      low PP2A activity is beneficial and the level of said carboxymethylated PP2Ac is elevated.

In one particular but not limiting aspect, the invention relates to a method for diagnosing the state of a disease in a subject, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with an inventive antibody specifically binding carboxymethylated PP2Ac, and
(b) evaluating whether the disease state is rather benign or malign, and indicating a rather benign disease state if the level of said carboxymethylated PP2Ac is elevated, and/or
   indicating a rather malign disease state if the level of said carboxymethylated PP2Ac is reduced.

It is evident that the present invention is also useful in methods for diagnosing the state and/or the status of a disease in a subject, wherein said disease is influenced either directly or indirectly and/or either positively or negatively by the carboxymethylation status of PP2Ac. Non-limiting examples of such diseases comprise, diseases selected from the group consisting of cancer, a neurodegenerative disorder, diabetes and a heart disease. Preferably, said disease is associated with and/or is caused by a deregulated signaling pathway, wherein said deregulation comprises the hyperphosphorylation of at least one component of said signaling pathway, and wherein said component can be dephosphorylated by protein phosphatase 2A (PP2A). Preferably, said disease is cancer which is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

A rather benign disease state, as used herein, means that the disease is less deleterious than what would be expected when only knowing the disease but not the diagnosis according to the present invention. For example, when it is found by employing the diagnostic method according to the invention that a patient suffering from prostate cancer has elevated levels of carboxymethylated PP2Ac, i.e. in the prostate cancer tissue, said prostate cancer may be diagnosed to be rather benign.

In contrast, a rather malign disease state, as used herein, means that the disease is more deleterious than what would be expected when only knowing the disease but not the diagnosis according to the present invention. For example, when it is found by employing the diagnostic method according to the invention that a patient suffering from prostate cancer has reduced levels of carboxymethylated PP2Ac, i.e. in the prostate cancer tissue, said prostate cancer may be diagnosed to be rather malign, and may, for example, may have a high susceptibility to metastasize.

In one aspect, the invention relates to a method for diagnosing the stage of a disease in a subject, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with the inventive antibody provided herein or an antibody specifically binding carboxymethylated PP2Ac, and
(b) evaluating whether the disease is in a rather early or advanced stage, and
   indicating a rather early stage if the level of said carboxymethylated PP2Ac is elevated, and/or
   indicating a rather advanced stage if the level of said carboxymethylated PP2Ac is reduced.

Preferably, said disease is selected from the group consisting of cancer, a neurodegenerative disorder, diabetes and a heart disease. Preferably, said disease is associated with and/or is caused by a deregulated signaling pathway, wherein said deregulation comprises the hyperphosphorylation of at least one component of said signaling pathway, and wherein said component can be dephosphorylated by protein phosphatase 2A (PP2A). Preferably, said disease is cancer which is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

A rather early disease stage, as used herein, means that the disease is in an earlier stage than what would be expected when only knowing the disease stage by other methods but not the diagnosis according to the present invention.

In contrast, a rather advanced disease state, as used herein, means that the disease is in a more advanced stage than what would be expected when only knowing the disease stage by other methods but not the diagnosis according to the present invention. It is well known in the art how diseases, i.e. certain cancers, may be staged by other means and methods.

In one aspect, the invention relates to a method for prognosing the progression of a disease in a subject, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with the inventive antibody provided herein or an antibody specifically binding carboxymethylated PP2Ac, and
(b) evaluating whether the disease progression is favorable or unfavorable, and indicating a favorable progression if the level of said carboxymethylated PP2Ac is elevated, and/or
   indicating an unfavorable progression if the level of said carboxymethylated PP2Ac is reduced.

Preferably, said disease is selected from the group consisting of cancer, a neurodegenerative disorder, diabetes and a heart disease. Preferably, said disease is associated with and/or is caused by a deregulated signaling pathway, wherein said deregulation comprises the hyperphosphorylation of at least one component of said signaling pathway, and wherein said component can be dephosphorylated by protein phosphatase 2A (PP2A). Preferably, said disease is cancer which is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

A certain progression of a disease may be expected based on empirical evidence and further characteristics of the disease. This may be used as a reference. Thus, if the level of said carboxymethylated PP2Ac is elevated as determined according to the prognostic method of the invention, the progression may be rather favorable compared to what would be expected without employing the present invention. Furthermore, if the level of said carboxymethylated PP2Ac is reduced as determined according to the prognostic method of the invention, the progression may be rather unfavorable compared to what would be expected without employing the present invention.

In particular, as illustrated in the appended Examples, the progression of cancer, i.e. prostate cancer, can be prognosed by assessing the carboxymethylated PP2Ac levels in cancer/tumor samples from patients. As demonstrated by the inventors, prostate cancer samples of a certain stage (determined by the Gleason score) can be classified based on their carboxymethylated PP2Ac level determined by the inventive antibody provided herein and, for example, by scoring with the Allred score. Surprisingly, although all prostate cancer samples had the same Gleason score, the actual cancer progression was not the same, but was linked to the carboxymethylated PP2Ac levels/Allred score (FIG. 25). In particular, patients whose carboxymethylated PP2Ac levels in the cancer sample were relatively high (Allred score of at least 2) survived in average much longer than patients whose carboxymethylated PP2Ac levels in the cancer sample were low (Allred score below 2). Prognosing disease progression, i.e. cancer progression, by employing the inventive antibody provided herein in conventional methods such as immunohistochemistry, Western blotting or ELISA, may thus be a highly useful, simple, and cost-effective diagnostic method, which may be combined with further diagnostic methods to further increase the accuracy of the method. It is evident that an accurate diagnostic method is critical for identifying the optimal therapy.

Thus, the invention further relates to an in vitro method for prognosing the outcome of a cancer in a patient, wherein said method comprises the steps of
(a) determining the level of carboxymethylated PP2Ac in a sample from said patient by contacting said sample with an anti-carboxymethylated PP2Ac-specific antibody according to the invention;
(b) comparing the level of carboxymethylated PP2Ac to a threshold value and/or reference level; and
(c) prognosing the outcome of said cancer, wherein
   (i) a positive outcome is prognosed when the level of carboxymethylated PP2Ac is equal to or greater than said threshold value and/or reference level,
   and/or
   (ii) a negative outcome is prognosed when the level of carboxymethylated PP2Ac is lower than said threshold value and/or reference level.

In some embodiments, instead of prognosing the outcome of said cancer, the outcome of any one of the diseases described herein in context of the further diagnostic, prognostic and/or predictive methods described herein may be prognosed, e.g. a disease that is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway,

wherein said deregulation comprises the hyperphosphorylation or hypophosphorylation of at least one component of said signaling pathway, and wherein said component can be dephosphorylated by protein phosphatase 2A (PP2A).

Furthermore, a positive outcome may further refer to a favorable progression, as described herein. A negative outcome may further refer to an unfavorable progression, as described herein. A level of carboxymethylated PP2Ac that is equal to or greater than the threshold value and/or reference level may further refer to an “elevated” level, as described herein. A level of carboxymethylated PP2Ac that is lower said threshold value and/or reference level may further refer to an “reduced” level, as described.

A positive outcome, as used herein, may refer to survival of the patient, preferably recurrence free survival, for at least about 0.5, 1, 2, 3, 4, 5, 6, 7, 8, 9, 10, 11, 12, 13, 14, 15 or 20 years, preferably at least 5 or 6 years, more preferably at least 10 years. Furthermore, a positive outcome may also refer to the prevention and/or elimination of metastases, and/or a metastase-free state, in particular for said period of time.

A negative outcome, as used herein, may refer to death and/or recurrence of the cancer in less than about 0.5, 1, 2, 3, 4, 5, 6, 7, 8, 9, 10, 11, 12, 13, 14, 15 or 20 years, e.g. less than about 10 years, or less than about 5 or about 6 years. Furthermore, a negative outcome may also refer to the development, maintenance or recurrence of metastases, in particular for said period of time.

Therefore, the invention further relates to an in vitro method for diagnosing whether a cancer is metastatic or prone to metastasize, wherein said method comprises the steps of
(a) determining the level of carboxymethylated PP2Ac in a sample from said patient by contacting said sample with an anti-carboxymethylated PP2Ac-specific antibody according to the invention;
(b) comparing the level of carboxymethylated PP2Ac to a threshold value and/or reference level; and
(c) diagnosing whether the cancer is metastatic or prone to metastasize, wherein
   (i) it is diagnosed that said cancer is not metastatic or prone to metastasize when the level of carboxymethylated PP2Ac is equal to or greater than said threshold value and/or reference level,
   and/or
   (ii) it is diagnosed that said cancer is metastatic or prone to metastasize when the level of carboxymethylated PP2Ac is lower than said threshold value and/or reference level.

In particular, the threshold value, as used herein, e.g. in context of the method for prognosing the outcome of a cancer in a patient or the method for diagnosing whether a cancer is metastatic or prone to metastasize may refer to an Allred Score of 2 determined by immunohistochemistry staining with the anti-carboxymethylated PP2Ac-specific antibody of the invention. In particular, an Allred Score of equal to or higher than 2 means that some cells, e.g. at least 0.1%, 0.5% or 1%, preferably at least 0.5% of the cells in the sample show some staining, e.g. a weak, intermediate or highstaining. Thus, an Allied Score of lower than 2 means, in particular, that essentially no cells in the sample, e.g. less than 0.1%, preferably less than 0.01%, more preferably no cells (0%), show at most a weak staining, preferably no staining. In other words, an Allred Score of lower than 2 may mean that there are essentially no carboxmethylated PP2Ac positive cells detected in the sample by immunohistochemistry staining with the anti-carboxymethylated PP2Ac-specific antibody of the invention.

Preferably herein, the reference level, e.g. in context of the method for prognosing the outcome of a cancer in a patient or the method for diagnosing whether a cancer is metastatic or prone to metastasize may be determined by analyzing the level of carboxymethylated PP2Ac in samples from a plurality of reference patients diagnosed with the respective disease, e.g. a cancer (and optionally additional healthy subjects), by contacting said samples with an anti-carboxymethylated PP2Ac-specific antibody according to the invention, wherein it is known whether the outcome of the disease, e.g. cancer, of said reference patients has been positive or negative. In particular, the reference level allows to separate the reference patients with a positive outcome from the reference patients with a negative outcome in an optimal way. Preferably, the level of carboxymethylated PP2Ac in the samples from the reference patients is determined by the same measurement method that is employed in said step (a) of said methods.

Preferably, the disease, e.g. cancer, of the reference patients is the same type of disease or cancer from which the patient to be diagnosed is suffering from, or from which the patient to be diagnosed is suspected of suffering.

It is known in the art that the absolute level of a biomarker (herein carboxymethylated PP2Ac) may depend on the exact measurement method and measurement conditions. However, the relationship between two variables X (e.g. carboxymethylated PP2Ac level) and Y (e.g. the outcome and/or progression of the disease, e.g. cancer) may be robust, independent of variations in the measurement method, at least when a truly specific anti-carboxymethylated PP2Ac antibody of the invention is employed. For example, when multiple samples (e.g. from reference patients) with known properties (e.g. the outcome of the disease, e.g. cancer) are measured with a certain measurement method and condition and graphed, then the same properties (e.g. the outcome of the disease, e.g. cancer) can be determined for an unknown sample (e.g. from the subject or patient to be diagnosed) that is measured with the same measurement method and condition, e.g. by interpolation of the graph.

Furthermore, a threshold (i.e. the reference level) may be determined based on the data from the reference patients, e.g. as described herein with respect to the Allied Score, wherein said threshold value allows to separate the reference patients with a positive outcome from those with a negative outcome in a useful and/or optimal way.

Thus, reference patients allow to establish a standard curve and/or a threshold value for different data sets and/or in different laboratories.

Furthermore, the invention relates to a method of detecting an abnormal level of carboxymethylated PP2Ac in a sample from a patient, wherein said method comprises
(a) measuring the level of carboxymethylated PP2Ac in a sample from said patient by contacting said sample with an anti-carboxymethylated PP2Ac-specific antibody according to the invention; and
(b) determining whether the sample is abnormal, wherein the sample is determined to be abnormal if the level of carboxymethylated PP2Ac is at least about 20% lower than the amount determined for a reference sample.

Preferably, the sample may be determined to be abnormal if the level of carboxymethylated PP2Ac is at least about 30%, about 40%, about 50%, about 60%, about 70%, or about 80%, preferably at least about 60%, more preferably at least about 80%, e.g. about 80%, lower than the amount determined for the reference sample.

Furthermore, the method of detecting an abnormal level of carboxymethylated PP2Ac in a sample from a patient further may further comprises:
(c) reporting to said patient whether said sample is determined to be abnormal or normal.

In particular, said patient may be suffering from a cancer, as described herein, preferably a prostate cancer.

Furthermore, the reference sample may be derived from at least one patient not suffering from a disease selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A).

In particular, the reference sample may be derived from at least one patient not suffering from a cancer.

In a particulary preferred embodiment, the reference sample is derived from at least one patient not suffering from a metastatic cancer, preferably a metastatic prostate cancer, wherein said patient, however, preferably suffers from a non-metastatic (localized) cancer, preferably a non-metastatic (localized) prostate cancer.

Furthermore, a patient having reported an abnormal level of carboxymethylated PP2Ac may be also reported to expect death and/or recurrence of the cancer in less than about 0.5, 1, 2, 3, 4, 5, 6, 7, 8, 9, 10, 11, 12, 13, 14, 15 or 20 years, e.g. in less than about 10 years, or less than about 5 or about 6 years; whereas a patient having reported a normal level of carboxymethylated PP2Ac may be also reported to expect survival, preferably recurrence free survival, for at least 0.5, 1, 2, 3, 4, 5, 6, 7, 8, 9, 10, 11, 12, 13, 14, 15 or 20 years, preferably at least 5 or 6 years, more preferably at least 10 years.

In preferred embodiments, the patient for whom the analysis, diagnosis or prognosis is made, is suspected of having a metastatic cancer or developing a metastatic cancer.

Furthermore, in preferred embodiments of the diagnostic, prognostic and/or predictive methods of the invention, the sample is a cancer sample, e.g. a prostate cancer sample. Furthermore, the cancer, as used herein, e.g. in context of the diagnostic, prognostic and/or predictive methods of the invention, may be associated with and/or caused by hyperphosphorylation of androgen receptor, c-MYC, ERK, AKT, S6K, β-catenin, and/or Bcl2, e.g. androgen receptor.

Furthermore, reporting an abnormal or normal level of carboxymethylated PP2Ac may indicate a corresponding treatment of the disease, e.g. cancer.

Thus, the invention further relates to a method of treating cancer, e.g. prostate cancer, in a patient, wherein said method comprises administering to the patient a PP2A activating drug (i.e. a PP2A activator as described herein, for example, but not limited to is DT-061 or forskolin), wherein said patient was reported as having an abnormal level of carboxymethylated PP2Ac, i.e. by employing an anti-carboxymethylated PP2Ac-specific antibody according to the invention, and wherein said level of carboxymethylated PP2Ac in a sample obtained from said patient was determined to be at least about 20%, preferably at least about 30%, about 40%, about 50%, about 60%, about 70%, or about 80%, more preferably at least about 60%, most preferably at least about 80%, e.g. about 80%, lower than the amount determined for a reference sample. As regards the patient, the abnormal level of carboxymethylated PP2Ac, the sample and the reference sample, the same applies as described in the context of the inventive method of detecting an abnormal level of carboxymethylated PP2Ac in a sample from a patient provided herein.

In particulary preferred embodiments, the prognosis of the progression of cancer, prognosing the outcome of a cancer in a patient, and/or diagnosing whether a cancer is metastatic or prone to metastasize, may be performed on tissue from a patient at an intermediate stage of cancer, i.e. characterized by a Gleason score of 6 or 7, preferably 7.

Furthermore, the association of carboxymethylated PP2Ac levels in patient tissue with patient survival, as illustrated for example in the appended Examples, may be highly useful for generating a suitable disease associated reference range/score of carboxymethylated PP2Ac levels.

In one further aspect, the invention relates to a method for diagnosing the subtype of a disease in a subject, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with the inventive antibody provided herein or an antibody specifically binding carboxymethylated PP2Ac, and
(b) determining that said disease is a subtype that is associated with carboxymethylated PP2Ac levels, if the carboxymethylated PP2Ac levels are reduced.

Preferably, said disease is selected from the group consisting of cancer, a neurodegenerative disorder, diabetes and a heart disease. Preferably, said disease is associated with and/or is caused by a deregulated signaling pathway, wherein said deregulation comprises the hyperphosphorylation of at least one component of said signaling pathway, and wherein said component can be dephosphorylated by protein phosphatase 2A (PP2A). Preferably, said disease is cancer which is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

A certain disease, for example, a certain cancer, such as prostate cancer, may have many origins/pathological histories. For targeted and/or personalized medicine it is critical to determine certain particularities of the said prostate cancer, i.e. to decide for an appropriate therapy. Thus, if the level of carboxymethylated PP2Ac is reduced in the cancer of a patient, administration of drug which increases PP2Ac levels and/or activates PP2A may be a suitable means for therapy of said patient.

Furthermore, in the context of the prognostic, predictive or diagnostic methods according to the invention, the elevation or reduction of carboxymethylated PP2Ac levels, and/or the presence of low vs. high carboxymethylated PP2Ac levels, may be determined by comparison with a suitable reference. A reference may be, for example, a biological sample of a subject not suffering from the respective disease. Furthermore, a plurality of samples (i.e. healthy and diseased) may be assessed for carboxymethylated PP2Ac levels to establish a reference range/score. Furthermore, a catalogue of carboxymethylated PP2Ac levels in different tissues may be prepared and each tissue may be associated with a certain disease outcome. The disease outcome may be evaluated by patient data, i.e. survival rate, disease-free time etc. and/or by classification of empirical data. For example, if the methyl-PP2Ac data of the sample (e.g. low methyl-PP2Ac levels) better correspond to the methyl-PP2Ac levels of a certain cancer which is associated with an unfavorable outcome than to a related cancer associated with a rather favorable outcome, then it may be prognosed that the progression is rather unfavorable.

Preferably, i.e. in the context of the prognostic, predictive or diagnostic methods according to the invention, the carboxymethylated PP2Ac levels determined with the inventive antibody provided herein or an antibody specifically binding carboxymethylated PP2Ac may be scored, ranked and/or categorized. Particularly suitable for such scoring/ranking/categorization is the Allred score, in particular when the disease is a cancer. Preferably, the Allred score is applied of immunohistochemistry samples wherein carboxymethylated PP2Ac is detected by the inventive antibody provided herein or an antibody specifically binding carboxymethylated PP2Ac.

Allred scoring allows to categorize a cancer, for example a prostate cancer, based on its methyl-PP2Ac status into (1) the group of cancers with a high risk to metastasize, a prognosed negative outcome, a prognosed unfavorable progression and/or cancers associated with a lower patient survival rate, and (2) the group of cancers with a low risk to metastasize, a prognosed positive outcome, a prognosed favorable progression and/or cancers associated with a higher patient survival rate.

In particular, the Allred score is the sum of the Proportion score and the Intensity score, wherein the Proportion score is based on the number of methyl-PP2Ac cells, and the Intensity score is based on the intensity of the immunostaining, i.e. the intensity of the staining of the methyl-PP2Ac positive cells. One advantage of the Allred score is that it can cope with both heterogeneity in and intensity of staining. Specifically, the Allred scoring system is based on the percentage of cells that stain by immunohistochemistry for carboxymethylated PP2Ac (on a scale of 0 to 5) and the intensity of that staining (on a scale of 0 to 3), for a possible total score of 8. In particular, the Allred score is calculated the following way:

Proportion Score

0—No cells or essentially no cells (e.g. ≤0.01%) are methyl-PP2Ac+

1—≤1% (e.g. 0.5-1%) of cells are methyl-PP2Ac+

2—1-10% (i.e. >1% and ≤10%; e.g. 2-10%) of cells are methyl-PP2Ac+

3—11-33% of cells are methyl-PP2Ac+.

4—34-66% of cells are methyl-PP2Ac+.

5—67-100% of cells are methyl-PP2Ac+.

Intensity Score

0—Negative methyl-PP2Ac staining.

1—Weak methyl-PP2Ac staining.

2—Intermediate methyl-PP2Ac staining.

3—Strong methyl-PP2Ac staining.

For example, when 0.5% positive cells in the sample are methyl-PP2Ac positive (+), and their staining intensity is weak, the Allred Score is 2 (1+1).

Furthermore, when no methyl-PP2Ac positive (+) cells are detected in the sample, the Allied Score is 0 (0+0), because the staining intensity is inherently also 0.

When virtually no methyl-PP2Ac positive (+) cells are detected in the sample, e.g. 0.01%, and only with weak staining intensity, the Allred Score may be considered 1 (0+1), i.e. below 2.

Furthermore, when some positive cells are detected, e.g. 5% (proportion score: 2), the Allied Score is normally at least 1 point higher than the proportion score, e.g. 3 (2+1) when the cells show only a weak staining intensity, or, e.g. 4 (2+2) when the cells show an intermediate staining intensity.

In particular, Allied scores are linked to the progression of the disease, i.e. cancer, and/or the survival of patients, i.e. cancer patients.

In particular, an Allied score of 0 or 1 (<2), e.g. 0, indicates a high probability of a cancer to spread/metastasize and is associated with a positive outcome, a favorable progression and/or a lower patient survival rate, and an Allred score of at least 2 indicates a low/lower probability of a cancer to spread/metastasize and is associated with a positive outcome, a favorable progression and/or a higher patient survival rate.

In particular, if the Allred score is below 2, a 50% reduced survival rate 10 years post biopsy may be expected compared to samples with an Allied score of at least 2.

To determine which staining intensity is considered negative, weak, intermediate or strong, suitables negative and positive controls may be required. For example, a suitable negative control is a respective tissue sample which does not contain relevant carboxymethylated PP2Ac levels (e.g. because the gene(s) for the PP2Ac specific demethylase has/have been deleted). For example, a suitable positive control is a respective tissue sample wherein a PP2Ac methyltransferase is overexpressed. Furthermore, it is preferable to obtain/provide a collection of respective healthy and disease samples to set the Allred intensity scores 0 and based on the staining intensities in that collection.

In principle, carboxymethylated PP2Ac levels are associated with high levels of certain, i.e. tumor-suppressive, PP2A holoenzymes. Those PP2A holoenzymes dephosphorylate their substrates, wherein those substrates may be components of certain signaling pathways as described herein. Many diseases, as described herein, i.e. cancer, are associated with and/or caused by hyperphosphorylation of such a signaling pathway. For the treatment of this kind of diseases, i.e. cancer, high PP2A activity, i.e. tumor-suppressive activity, is beneficial. Thus, for example, when the carboxymethylated PP2Ac levels are elevated in a biological sample (indicating high PP2A activity) from a patient suffering from a disease (i.e. cancer) that is associated with hyperphosphorylation of a signaling pathway component (a substrate of PP2A), then high PP2A activity is beneficial, and the disease progression is rather favorable.

The inventors have further surprisingly found, as illustrated in the appended Examples, that reduced levels of LCMT1 (the only enzyme which methylates the C-terminus of PP2Ac) and concomitant low levels of carboxymethylated PP2Ac, as determined by the inventive antibody provided herein (e.g. 7C10-C5) are associated with increased expression of androgen receptor, increased phosphorylation of androgen receptor, increased phosphorylation of c-MYC, and resistance to androgen deprivation (antiandrogen) therapies, e.g. the treatment with enzalutamide.

Thus, the invention further relates to an in vitro method for prognosing the responsiveness of a cancer in a patient to treatment with an antiandrogen, wherein said method comprises the steps of
(a) determining the level of carboxymethylated PP2Ac in a sample from said patient by contacting said sample with an anti-carboxymethylated PP2Ac-specific antibody according to the invention;
(b) comparing the level of carboxymethylated PP2Ac to a reference level; and
(c) prognosing whether the cancer is responsive, wherein
(i) it is prognosed that said cancer is responsive when the level of carboxymethylated PP2Ac is equal to or greater than said reference level,
and/or
(ii) it is prognosed that said cancer is not responsive when the level of carboxymethylated PP2Ac is lower than said reference level.

In particular, a response may correspond to
(i) elimination of the cancer,
(ii) prevention and/or elimination of metastases,
(iii) a reduction of the cancer volume by at least 30%
(iv) survival of the cancer patient for at least about 0.5, 1, 2, 3, 4, 5, 6, 7, 8, 9, 10, 11, 12, 13, 14, 15 or 20 years, preferably at least 5 years; and/or
(v) a decline of at least one cancer tumor marker, e.g. prostate-specific antigen (PSA), by at least 50%.

Preferably, said cancer is a prostate cancer.

In particular, said reference level may be determined by analyzing the level of carboxymethylated PP2Ac in samples from a plurality of reference patients diagnosed with a cancer by contacting said sample with an anti-carboxymethylated PP2Ac-specific antibody according to the invention, wherein it is known whether the cancer of said reference patients has been responsive to an antiandrogen treatment or not.

In particular, the reference level allows to separate the reference patients with a responsive cancer from the reference patients with an unresponsive cancer in an optimal way. Preferably, the level of carboxymethylated PP2Ac in the samples from said reference patients is determined by the same measurement method that is employed in said step (a).

Preferably herein and in context of the invention, the antiandrogen comprises an antagonist of androgen receptor signaling, preferably an androgen receptor antagonist, more preferably enzalutamide.

Antiandrogens, and/or androgen receptor signaling antagonists (i.e. androgen receptor antagonists), as used herein, are, for example, Enzalutamide (Xtandi), apalutamide (Erleada), darolutamide (Nubeqa), Flutamide (Eulexin), Bicalutamide (Casodex), or Nilutamide (Nilandron), preferably enzalutamide.

The binding molecule/antibody of the invention may be also used for further prognostic, predictive, and/or diagnostic methods, i.e. to stratify subjects based on their predicted or determined responsiveness or response to the treatment with a PP2A modulator.

Thus, the invention further relates to a method for predicting the responsiveness of a subject suffering from a disease to the treatment with a PP2A modulator, wherein said method comprises detecting or quantifying carboxymethylated PP2Ac in a sample using the herein provided antibody or an antibody specifically binding carboxymethylated PP2Ac.

Furthermore, the invention relates to a method for determining the responsiveness or response of a subject suffering from a disease to the treatment with a PP2A modulator, wherein said method comprises detecting or quantifying carboxymethylated PP2Ac in a sample using the herein provided antibody or an antibody specifically binding carboxymethylated PP2Ac.

The invention also relates to a method for predicting the responsiveness of a subject suffering from a disease to the treatment with a PP2A modulator, wherein said method comprises detecting or quantifying carboxymethylated PP2Ac in a biological sample from said subject using the herein provided antibody or an antibody specifically binding carboxymethylated PP2Ac.

Furthermore, the invention relates to a method for determining the responsiveness or response of a subject suffering from a disease to the treatment with a PP2A modulator, wherein said method comprises detecting or quantifying carboxymethylated PP2Ac in a sample from said subject using the herein provided antibody or an antibody specifically binding carboxymethylated PP2Ac.

In particular, the subject whose responsiveness or response to the treatment with a PP2A modulator is predicted or determined is suffering from a disease selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A).

Preferably, said disease is selected from the group consisting of cancer, a neurodegenerative disorder, diabetes and a heart disease. Preferably, said disease is associated with and/or is caused by a deregulated signaling pathway, wherein said deregulation comprises the hyperphosphorylation of at least one component of said signaling pathway, and wherein said component can be dephosphorylated by protein phosphatase 2A (PP2A). Preferably, said disease is cancer which is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

Furthermore, the invention relates to a method for predicting the responsiveness of a subject suffering from a disease to the treatment with a PP2A modulator, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with the inventive antibody provided herein or an antibody specifically binding carboxymethylated PP2Ac, and
(b) evaluating whether said subject will be responsive to the treatment with said PP2A modulator, and
   indicating that said subject will be responsive if
      said disease is associated with hyperphosphorylation of said signaling pathway component, said PP2A modulator activates PP2A, and the level of said carboxymethylated PP2Ac is reduced, or
      said disease is associated with hypophosphorylation of said signaling pathway component, said PP2A modulator inhibits PP2A, and the level of said carboxymethylated PP2Ac is elevated, and/or
   indicating that said subject will not be responsive if
      said disease is associated with hyperphosphorylation of said signaling pathway component, said PP2A modulator activates PP2A, and the level of said carboxymethylated PP2Ac is elevated, or
      said disease is associated with hypophosphorylation of said signaling pathway component, said PP2A modulator inhibits PP2A, and the level of said carboxymethylated PP2Ac is reduced.

In one aspect, the invention relates to a method for predicting the responsiveness of a subject suffering from a disease to the treatment with a PP2A modulator, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with the inventive antibody provided herein or an antibody specifically binding carboxymethylated PP2Ac, and
(b) evaluating whether said subject will be responsive to the treatment with said PP2A modulator, and
   indicating that said subject will be responsive if the level of said carboxymethylated PP2Ac is reduced, and/or indicating that said subject will not be responsive if the level of said carboxymethylated PP2Ac is elevated.

Preferably, said disease is selected from the group consisting of cancer, a neurodegenerative disorder, diabetes and a heart disease. Preferably, said disease is associated with and/or is caused by a deregulated signaling pathway, wherein said deregulation comprises the hyperphosphorylation of at least one component of said signaling pathway, and wherein said component can be dephosphorylated by protein phosphatase 2A (PP2A). Preferably, said disease is cancer which is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

In particular, if the subject or patient has already high carboxymethylated PP2Ac levels, as determined according to the predictive method of the invention, an PP2A activator is rather unlikely to provide a therapeutic benefit and said patient may not respond to a therapy with such a PP2A activator. In contrast, if the carboxymethylated PP2Ac levels, as determined according to the predictive method of the invention, are rather low, said patient may benefit from increasing carboxymethylated PP2Ac levels and/or activating PP2A, and thus may respond to a therapy with such a PP2A activator.

The invention also relates to a method for predicting the responsiveness of a subject suffering from a disease to the treatment with a PP2A modulator, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with the inventive antibody provided herein or an antibody specifically binding carboxymethylated PP2Ac, and
(b) evaluating whether said subject will be responsive to the treatment with said PP2A modulator, and
   indicating that said subject will be responsive if
      high PP2A activity is beneficial, said PP2A modulator activates PP2A, and the level of said carboxymethylated PP2Ac is reduced, or
      low PP2A activity is beneficial, said PP2A modulator inhibits PP2A, and the level of said carboxymethylated PP2Ac is elevated, and/or
   indicating that said subject will not be responsive if
      high PP2A activity is beneficial, said PP2A modulator activates PP2A, and the level of said carboxymethylated PP2Ac is elevated, or
      low PP2A activity is beneficial, said PP2A modulator inhibits PP2A, and the level of said carboxymethylated PP2Ac is reduced.

Furthermore, the invention relates to a method for determining the responsiveness or response of a subject suffering from a disease to the treatment with a PP2A modulator, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with the inventive antibody provided herein or an antibody specifically binding carboxymethylated PP2Ac, wherein said biological sample is derived from a tissue that has been contacted in said subject with said PP2A modulator,
(b) determining that said subject responds to the treatment with said PP2A modulator, if the level of carboxymethylated PP2Ac in said sample is altered.

Preferably, in the context of the prognostic, predictive and/or diagnostic methods of the invention, the sample is a sample from the subject to be assessed, in particular a biological sample. In particular, said biological sample comprises at least one cell, preferably a cell population or a tissue comprising cells, or a lysate thereof. Preferably, the biological sample is, or has been derived or obtained from a diseased and/or pathogenic tissue from said subject.

Preferably herein, i.e. in the context of the prognostic, predictive and/or diagnostic methods of the invention, the subject is a human. In particular, said subject is suffering or is suspected to suffer from a disease as described herein in the context of the prognostic, predictive, and/or diagnostic methods of the invention.

In particular, the carboxymethylated PP2Ac level in said sample may be compared to the carboxymethylated PP2Ac level in a reference or control sample, thereby determining if the carboxymethylated PP2Ac level is reduced or increased. The person skilled in the art is well aware how to select an appropriate reference or control sample, and suitable reference samples are described herein.

As used herein, a modulator may be an activator or an inhibitor. In particular, a PP2A modulator, as used herein, may be a PP2A activator or a PP2A inhibitor.

A PP2A activator, as used herein may activate PP2A, enhance PP2A activity, inhibit PP2A deactivation, and/or elevate active PP2A levels.

A PP2A inhibitor, as used herein may reduce PP2A activity, inhibit PP2A activation, enhance PP2A deactivation, and/or reduce active PP2A levels.

Preferably, as used herein, the PP2A activity refers to the target or substrate-specific activity of PP2A.

In the context of the present invention, the PP2A modulator is preferably a PP2A activator. Preferably, said PP2A activator is a small molecule, preferably a modified phenothiazine and/or a small molecule derived from phenothiazine. In a particular embodiment, the PP2A activator is DT-061 (CAS No.: 1809427-19-7 or CAS No.: 1809427-18-6) or forskolin (Colforsin; (3R,4aR,5S,6S,6aS,10S,10aR,10bS)-dodecahydro-5,6,10,10b-tetrahydroxy-3,4a,7,7,10a-pentamethyl-1-oxo-3-vinyl-1H-naphtho[2,1-b]pyran-5-yl acetat), preferably DT-061. Further PP2A activators have been described herein further above, in particular, SET targeting inhibiting drugs (such as inter alia COG112, OP449, FTY720, OSU-2S, MP07-66 and, TGI1002; CIP2A inhibiting drugs (such as inter alia Erlotinib, Celastrol, Bortezomib, TD-19, and TD-44), PME-1 inhibitors (such as inter alia ABL-127), and drugs acting through an unknown mechanism (i.e. Forskolin, Vitamin E analogs, Lanolinamide and Canthardin).

Preferably, the deregulation in the context of a deregulated signaling pathway, as described herein, comprises the hyperphosphorylation of a component of said signaling pathway, wherein said component can be dephosphorylated by protein phosphatase 2A (PP2A). In particular, said signaling pathway may comprise a tyrosine kinase receptor, an androgen receptor, Bcl2, BCR-ABL1, PI3K, AKT, S6K, MAP, ERK, β-catenin, STAT5 and/or c-Myc, preferably wherein the tyrosine kinase receptor is EGFR. In particular, the component that can be dephosphorylated by PP2A is selected from c-MYC, ERK, AKT, S6K β-catenin, androgen receptor, Bcl2 and STAT5.

Preferably herein, i.e. in the context of the prognostic, predictive and/or diagnostic methods of the invention, the disease is cancer, a neurodegenerative disorder, diabetes and/or a heart disease, preferably cancer. Preferably, the cancer is myeloid leukemia (acute and chronic), non-small-cell lung carcinoma (including lung adenocarcinoma and lung squamous cell carcinoma), breast cancer, colon cancer or prostate cancer, and/or the neurodegenerative disorder is Alzheimer's disease. In some preferred embodiments, the cancer is prostate cancer.

In a particular embodiment, the disease is a cancer that is associated with and/or caused by a hyperactive Akt, S6K and/or ERK/MAP signaling pathway(s) and/or the hyperphosphorylation of at least one component of said signaling pathways(s), wherein said component can be dephosphorylated by PP2A.

In a further particular embodiment, the disease is a cancer that is associated with and/or caused by abnormal androgen receptor signaling and/or hyperphosphorylation of androgen receptor. Preferably said cancer is associated with and/or caused by increased androgen receptor expression and/or hyperphosphorylation, most preferably hyperphosphorylation of androgen receptor.

As described already above, the binding molecule/antibody of the invention may be used in screening/test methods, i.e. to identify/evaluate agents which modulate PP2Ac carboxymethylation and/or PP2A activity. Screening/test methods according to the invention are described in the following.

Therefore, and as illustrated in the appended Examples, screening methods for potentially useful pharmaceuticals are provided in context of this invention. Such pharmaceuticals and/or medicaments may, inter alia, comprise activators of protein phosphatase 2A (PP2A). Without being bound by theory, such medicaments (i.e. activators of PP2A) are in particular useful in context of the present invention, since the protein phosphatase 2A complex is capable of stabilizing and/or increasing the methylation status of PP2Ac, in particular the stabilization of alpha-carboxymethylation of PP2Ac as described herein. Such a “stabilization” of said alpha-carboxymethylation of PP2Ac may lead to advantageous effects in disorders wherein it is desired to de-phosphorylate a hyperactive/hyperphosphorylated component of a disorder-related signaling pathway, like a disordered and/or modified signaling pathway in cancer. Accordingly, binding molecules as described in context of this invention, in particular the antimethyl-PP2Ac-specific antibody are particularly useful in drug screenings. One illustrative example of such a drug screening is provided herein below. In this context, a known activator of protein phosphatase 2A (PP2A) is employed as standard control, i.e. as a drug that fulfils this desired function. Such an activator may be DT-061, an activator of protein phosphatase 2A (PP2A) and proposed in the therapy of KRAS-mutant and MYC-driven tumorigenesis; see inter alia, Kauko (2018) “PP2A inhibition is a druggable MEK inhibitor resistance mechanism in KRAS-mutant lung cancer cells”. Sci Transl Med. 18; 10(450); McClinch (2018) Cancer Res.;78(8):2065-2080. The “read-out” of such drug screening methods may be the increase of methyl PP2Ac levels in response to the test compound, inter alia, versus (a) control compound(s). Control compounds may comprise negative controls, like compounds that do not lead to an increase of methyl PP2Ac levels and positive controls, like compounds that are known to lead to an increase of methyl PP2Ac levels (like activator of protein phosphatase 2A (PP2A), like DT-061 (CAS NO 1809427-19-7)) Other “positive controls may comprise known drugs that lead to an increase and/or stabilization of carboymehtylated PP2Ac, like PP2A activators such as phenothiazine derived small molecule PP2A activators (SMAPs) (Allen-Petersen (2019), Cancer Res 79, 209-219; Sangodkar (2017), J Clin Invest. 127, 2081-2090; Gutierrez (2014), J Clin Invest. 124(2), 644-55; Kastrinsky (2015), Bioorg Med Chem. October 1; 23(19):6528-34), drugs that counteract the endogenous PP2A inhibitors SET (I2PP2A, UniProt: Q01105) and CIP2A (UniProt: Q8TCG1-1). In the description herein above, further potential drugs are described. In such drug screenings, the inventive antibodies being specific for methyl-PP2Ac, in particular specific alpha-carboxymethylated PP2Ac, are particularly useful.

Also an illustrative in vitro drug screening assay is provided in the appended Examples wherein the binding molecules/antibodies of the present invention are useful.

Accordingly, the present invention also relates to a method for evaluating whether a test agent modulates PP2Ac carboxymethylation, wherein said method comprises (a) treating/contacting a sample with said test agent, and (b) subsequently detecting/quantifying carboxymethylated PP2Ac in said sample using the herein provided antibody or an antibody specifically binding carboxymethylated PP2Ac.

Furthermore, the present invention relates to a method for identifying agents that modulate PP2Ac carboxymethylation, wherein said method comprises (a) treating a sample with a certain test agent, and (b) subsequently detecting/quantifying carboxymethylated PP2Ac in said sample using the herein provided antibody or an antibody specifically binding carboxymethylated PP2Ac.

In particular, in the context of the screening/test methods according to the invention, detecting/quantifying carboxymethylated PP2Ac in said sample comprises contacting said sample with the herein provided antibody or an antibody specifically binding carboxymethylated PP2Ac after treating/contacting said sample with a test agent.

In particular, the test method according to the invention further comprises a step of determining that a test agent modulates PP2Ac carboxymethylation or that said test agent does not modulate PP2Ac carboxymethylation.

Preferably, said determination comprises comparing the carboxymethylated PP2Ac level in the sample treated with said test agent with the carboxymethylated PP2Ac level in a suitable control sample, for example, a sample which is only treated with the solvent of said test agent or an untreated sample. In particular, if the level of carboxymethylated PP2Ac is not significantly altered and/or is altered by at most +/−50%, preferably at most +/−30%, preferably at most +/−10%, said test agent is determined to not modulate PP2Ac carboxymethylation.

In contrast, if the level of carboxymethylated PP2Ac is significantly increased and/or is increased by at least +/−10%, preferably at least +/−30%, preferably at least +/−50%, said test agent is determined to modulate, i.e. increase, PP2Ac carboxymethylation.

Furthermore, if the level of carboxymethylated PP2Ac is significantly reduced and/or is reduced by at least +/−10%, preferably at least +/−30%, preferably at least +/−50%, said test agent is determined to modulate, i.e. reduce, PP2Ac carboxymethylation.

Of note, if the carboxymethylated PP2Ac is altered by at most +/−50% and increased by at least +/−10%, said test agent is determined to modulate, i.e. increase, PP2Ac carboxymethylation.

Of note, if the carboxymethylated PP2Ac is altered by at most +/−50% and reduced by at least +/−10%, said test agent is determined to modulate, i.e. decrease, PP2Ac carboxymethylation.

In particular, the sample to be treated/contacted in the context of the screening/test methods of the invention is comprised in a methylation assay, wherein said methylation assay further contains PP2Ac and
(i) a PP2Ac methylase enzyme and/or a PP2Ac demethylase enzyme, and/or
(ii) an A and B subunit of PP2A.

An activator of said PP2Ac methylase enzyme, i.e. LCMT-1 is suspected to increase carboxymethylated PP2Ac levels. In contrast, an inhibitor of said PP2Ac demethylase enzyme, i.e. PME-1, such as ABL-127 (Bachovchin (2011) PNAS. 108 (17) 6811-6816), is suspected to reduce carboxymethylated PP2Ac levels.

Furthermore, an agent which promotes formation of the heterotrimeric PP2A holoenzyme (comprising a A, B and C subunit of PP2A) is suspected to increase carboxymethylated PP2Ac levels. In particular, SMAPS such as DT-061, may function that way. Without being bound by theory, SMAPs such as inter alia DT-061 may promote the formation of the PP2Ac carboxymethylation-dependent heterotrimeric PP2A holoenzyme and thereby sequester the carboxymethylated catalytic subunit of PP2A (PP2Ac) within the holoenzyme such that it is less susceptible to demethylation by a PP2A demethylase, thereby increasing carboxymethylated PP2Ac levels.

Thus, candidate test agents may be employed in the test/screening methods according to the present invention to evaluate whether they indeed and truly modulate PP2Ac carboxymethylation.

Of note, the test/screening methods of the invention critically rely on the antibody of the invention or an antibody specifically binding carboxymethylated PP2Ac because a precise detection/quantification of carboxymethylated PP2Ac levels is essential. As described above in the context of the prognostic/predictive/diagnostic methods of the invention, other allegedly PP2Ac specific antibodies in the prior art may be predisposed for giving erroneous results.

Thus, the invention also relates to a method for evaluating whether a test agent modulates PP2Ac carboxymethylation, wherein said method comprises the steps of
(a) assessing effects of said test agent on PP2Ac carboxymethylation status in a PP2Ac methylation assay, wherein said assay contains PP2Ac and
   (i) a PP2Ac methylase enzyme and/or a PP2Ac demethylase enzyme, and/or
   (ii) an A and B subunit of PP2A, and
wherein the PP2Ac carboxymethylation status is determined by the inventive antibody provided herein or an antibody specifically binding carboxymethylated PP2Ac; and
(b) determining based on the assessed effects that said test agent modulates PP2Ac carboxymethylation or that said test agent does not modulate PP2Ac carboxymethylation.

Furthermore, the invention relates to a method for identifying agents that modulate PP2Ac carboxymethylation, wherein said method comprises the steps of
(a) providing a plurality of candidate test agents;
(b) assessing effects of an individual candidate test agent on PP2Ac carboxymethylation status in a PP2Ac methylation assay, wherein said assay contains PP2Ac and
   (i) a PP2Ac methylase enzyme and/or a PP2Ac demethylase enzyme; and/or
   (ii) an A and B subunit of PP2A, and
wherein the PP2Ac carboxymethylation status is determined by the inventive antibody provided herein or an antibody specifically binding carboxymethylated PP2Ac; and
(c) identifying, based on the assessed effects, at least one agent that modulates PP2Ac methylation.

The effects on PP2Ac carboxymethylation status may comprise an increase or reduction of PP2Ac carboxymethylation compared to a reference and/or control.

In particular embodiments, i.e. in the context of the screening/test method of the invention, the PP2Ac methylation assay comprises a cell and/or cell lysate and/or is performed in a cell and/or cell lysate. Said cell/cell lysate may be a cell/cell lysate as described herein and/or as illustrated in the appended Examples.

In further embodiments, i.e. in the context of the screening/test method of the invention, the PP2Ac methylation assay is performed in vivo in a non-human test animal. In particular, said methylation assay comprises a xenograft tumor in said test animal. Suitable test animals, i.e. comprising a xenograft tumor, are known in the art and further illustrated in the appended Examples.

Preferably, i.e. in the context of the screening/test method of the invention, assessing the effects of a test agent on the PP2Ac carboxymethylation status comprises the steps of
(b′) contacting the PP2Ac methylation assay with said test agent, and subsequently
(b″) contacting said PP2Ac methylation assay with the inventive antibody provided herein or an antibody specifically binding carboxymethylated PP2Ac, thereby determining the PP2Ac carboxymethylation status.

Thus, in certain embodiments of the screening/test method of the invention, assessing the effects of a test agent on the PP2Ac carboxymethylation status comprises the steps of
(b′) contacting a cell and/or cell lysate with said test agent, and subsequently
(b″) contacting said cell/cell lysate with the inventive antibody provided herein or an antibody specifically binding carboxymethylated PP2Ac, thereby determining the PP2Ac carboxymethylation status.

In particular, said cell/cell lysate contains PP2Ac and
(i) a PP2Ac methylase enzyme and/or a PP2Ac demethylase enzyme; and/or
(ii) an A and B subunit of PP2A.

In further embodiments of the screening/test method of the invention, assessing the effects of a test agent on the PP2Ac carboxymethylation status comprises the steps of
(b′) contacting a tissue within a non-human test animal with said test agent, and subsequently
(b″) contacting said tissue with the inventive antibody provided herein or an antibody specifically binding carboxymethylated PP2Ac, thereby determining the PP2Ac carboxymethylation status.

Preferably, said tissue is a xenograft tumor, preferably comprising human cancer cells, for example as illustrated in the appended Examples.

Drug screening with the inventive antibody as provided herein and a xenograft animal model may be particularly useful for evaluating/identifying (potential) anti-tumor/cancer drugs because it allows determining the carboxymethylated PP2Ac levels in response to the test drug in human cells and in an in vivo environment including aspects like drug delivery/availability and potential feed-back mechanisms between cells/tissues.

The invention further relates to a method for evaluating whether a test agent modulates the activity of PP2A, wherein said method comprises the steps of
(a) evaluating whether said test agent modulates PP2Ac carboxymethylation according to the method for evaluating whether a test agent modulates PP2Ac carboxymethylation according to the invention, and
(b) determining that said test agent modulates the activity of PP2A, if said test agent modulates PP2Ac carboxymethylation.

The invention also relates to a method for identifying an agent that modulates the activity of PP2A, wherein said method comprises the steps of
(a) identifying an agent that modulates PP2Ac carboxymethylation according to the method for identifying agents that modulate PP2Ac carboxymethylation according to the invention, and
(b) selecting said identified agent.

In particular, i.e in the context of the screening/test method of the invention, the agent/test agent activates PP2A and/or enhances the activity of PP2A, if said agent/test agent increases PP2Ac carboxymethylation.

In particular, i.e in the context of the screening/test method of the invention, the agent/test agent inhibits the activity of PP2A and/or inhibits the activation of PP2A, if said agent/test agent reduces PP2Ac carboxymethylation.

In particular, the method for identifying agents that modulate PP2Ac carboxymethylation according to the invention may be used for identifying an agent that activates PP2A and/or enhances the activity of PP2A, wherein said method further comprises the steps of
(a) identifying an agent that increases PP2Ac carboxymethylation according to the method for identifying agents that modulate PP2Ac carboxymethylation according to the invention, and
(b) selecting said identified agent.

In particular, the method for identifying agents that modulate PP2Ac carboxymethylation according to the invention may be used for identifying an agent that inhibits the activity of PP2A and/or inhibits the activation of PP2A, wherein said method comprises the steps of
(a) identifying an agent that reduces PP2Ac carboxymethylation according to the method for identifying agents that modulate PP2Ac carboxymethylation according to the invention, and
(b) selecting said identified agent.

In particular, i.e. in the context of the screening/test method of the invention, the agent/test agent is a PP2A activator, if said agent/test agent
(i) activates the PP2Ac methylase enzyme and/or enhances the activity of the PP2Ac methylase enzyme; and/or
(ii) inhibits the activity of the PP2Ac demethylase enzyme and/or inhibits the activation of the PP2Ac demethylase enzyme.

In particular, i.e. in the context of the screening/test method of the invention, the agent/test agent is a PP2A activator, if said agent/test agent increases the amount of the trimeric PP2A holoenzyme.

In particular, i.e. in the context of the screening/test method of the invention, the agent/test agent is a PP2A inhibitor, if said agent/test agent
(i) activates the PP2Ac demethylase enzyme and/or enhances the activity of the PP2Ac demethylase enzyme; and/or
(ii) inhibits the activity of the PP2Ac methylase enzyme and/or inhibits the activation of the PP2Ac methylase enzyme.

In particular, i.e. in the context of the screening/test method of the invention, the agent/test agent is a PP2A inhibitor, if said agent/test agent reduces the amount of the trimeric PP2A holoenzyme.

In a particular embodiment, if said agent/test agent is a PP2A activator, said agent/test agent is DT-061 (CAS No.: 1809427-19-7 or CAS No.: 1809427-18-6) or forskolin (Colforsin; (3R,4aR,5S,6S,6aS,10S,10aR,10bS)-dodecahydro-5,6,10,10b-tetrahydroxy-3,4a,7,7,10a-pentamethyl-1-oxo-3-vinyl-1H-naphtho[2,1-b]pyran-5-yl acetat), preferably DT-061.

In certain embodiments of the test/screening methods according to the invention, further the total PP2Ac levels are determined by using an anti-total PP2Ac antibody. Thus, if the total-PP2Ac levels are found to be unchanged but the carboxymethylated PP2Ac levels are increased, it suggests that the respective test agent leads to the carboxymethylation of PP2Ac and does not increase both, non-methylated and carboxymethylated PP2Ac levels. Likewise, if the total-PP2Ac levels are found be unchanged but the carboxymethylated PP2Ac levels are decreased, it suggests that the respective test agent leads to the demethylation of PP2Ac and does not decrease both, non-methylated and carboxymethylated PP2Ac levels.

In certain embodiments of the test/screening methods according to the invention, the levels of androgen receptor (which may be determined for example by using the androgen receptor antibody Abcam Cat #ab74272) may be further determined subsequently to treating/contacting the sample with the test agent. Since androgen receptor is a substrate of PP2A and unstable in its dephosphorylated form, reduced levels of androgen receptor may further indicate a high PP2A activity.

In a particular embodiment, the screening method according to the invention further comprises a step of obtaining the identified agent.

Thus, the invention also relates to an PP2A activator obtained by the screening method according to the invention for use in treating a disease selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

Thus, the invention also relates to an PP2A inhibitor obtained by the screening method according to the invention for use in chemotherapy and/or radiotherapy.

Further screening/test methods according to the invention are provided in the following:

Thus, the invention further relates to a method for identifying a PP2A modulator, wherein said method comprises the steps of
(a) contacting a cell or cell lysate containing an A, B and C subunit of PP2A with a candidate PP2A modulator,
(b) contacting said cell or cell lysate with the inventive antibody provided herein or an antibody specifically binding carboxymethylated PP2Ac, thereby determining the level of carboxymethylated PP2Ac in said cell/cell lysate, and
(c) selecting said candidate PP2A modulator if the level of carboxymethylated PP2Ac in said cell/cell lysate is altered.

Furthermore, the invention relates to a screening method for evaluating whether a molecule modulates the activity of PP2A, wherein said method comprises the steps of
(a) contacting a cell or cell lysate containing an A, B and C subunit of PP2A with said molecule,
(b) contacting said cell or cell lysate with the inventive antibody provided herein or an antibody specifically binding carboxymethylated PP2Ac, thereby determining the level of carboxymethylated PP2Ac in said cell/cell lysate, and
(c) indicating that said molecule modulates the activity of PP2A if the level of carboxymethylated PP2Ac in said cell/cell lysate is altered.

In a particular embodiment in the context of the screening/test method of the invention, said PP2A modulator activates PP2A and/or enhances the activity of PP2A, said modulation is the activation of PP2A and/or the enhancement of the PP2A activity, and said alteration is an elevated carboxymethylated PP2Ac level.

In a particular embodiment in the context of the screening/test method of the invention said PP2A modulator/activator is DT-061 (CAS No.: 1809427-19-7 or CAS No.: 1809427-18-6) or forskolin (Colforsin; (3R,4aR,5S,6S,6aS,10S,10aR,10bS)-dodecahydro-5,6,10,10b-tetrahydroxy-3,4a,7,7,10a-pentamethyl-1-oxo-3-vinyl-1H-naphtho[2,1-b]pyran-5-yl acetat), preferably DT-061.

In accordance with the above, the inventive binding molecules/antibodies may also be useful and may be employed in drug screenings as described in the following.

Drugs may be screened for PP2A activation in vitro and in vivo. In vitro screening for drugs that increase PP2Ac methylation may be done by a methylation assay employing the core components of the PP2A holoenzyme, the catalytic subunit PP2Ac, the structural A and the B′56alpha subunit, as well as the enzymes regulating the methylations state LCMT1 and PME1. Read-out may be the methylation level of PP2Ac as determined by 7C10-C5, for example, in an ELISA.

In vivo screening may be done in mouse xenograft models and increase of PP2Ac methylation may be determined by IHC and western blot analyses with 7C10-C5 and a PP2Ac antibody that determines the total PP2Ac levels in cells. PP2A activators superior to the existing DT-061 PP2A activating drug may be selected by 2 selection criteria: Faster (than with DT-061) kinetics of methyl PP2Ac increase in response to novel compound and greater (than with DT-061) increase of methyl PP2Ac levels in response to novel compound compared to DT-061 and vehicle control and determined by IHC of formalin-fixed tumor tissue and by western blot analysis with 7C10-C5 and an antibody specific for total PP2Ac such as mouse monoclonal H-8, with non-methyl specific mouse monoclonal antibody 1D7 and a loading control antibody specific for actin or tubulin.

Accordingly, the present invention also provides a pharmaceutical composition comprising a medicament/drug screened and/or assessed with the herein provided means and methods, i.e. with the antibodies/binding molecules of the present invention.

The invention is also characterized by the following figures, figure legends and the following non-limiting examples.

### BRIEF DESCRIPTION OF THE FIGURES

FIG. 1: Western blot screening of mouse antisera. 400 μg of yeast protein lysates of the indicated strains was separated by 10% SDS-PAGE and transferred to nitrocellulose membrane (0.2 μm, GE Healthcare). The membranes were blocked in 3% non-fatty dry milk (NFDM) in PBS-Tween for 1 h at RT and incubated in a Miniblotter 28 channel unit (Immunetics) with the indicated mouse sera diluted 1:500 in 0.5% NFDM in PBS-T o/n at 4° C. Crude hybridoma cell culture supernatant of anti PP2Ac catalytic subunit antibody, clone 1D7 (diluted 1:100) which is specific for non-methylated PP2Ac, and anti-HA tag, clone 16B12 (Covance, diluted 1:10,000) were used as positive controls. After washing 3×5 min in PBS-T, incubation with anti-mouse HRP-coupled secondary antibody for 1 h at RT and 3×10 min washing in PBS-T, ECL was performed with GE Healthcare ECL reagents (RPN2106). Signals were detected by exposure of X-ray films. Molecular weights are indicated in kDa. M1-M4: mouse 1-mouse 4; p: pre-immune sera; 3.: 3rd bleed sera

FIG. 2: Western blot screening of mix clone hybridoma cell culture supernatants. 400 μg of yeast protein lysates of the indicated strains was separated by 10% SDS-PAGE and transferred to nitrocellulose membrane (0.2 μm, GE Healthcare). The membranes were blocked in 3% non-fatty dry milk (NFDM) in PBS-Tween for 1 h at RT and incubated in a Miniblotter 28 channel unit (Immunetics) with mix clone hybridoma cell culture supernatants o/n at 4° C. to search for clones displaying differential recognition of carboxy-methylated vs. unmethylated PP2A catalytic subunit. Anti-HA tag, clone 16B12 (Covance, diluted 1:10,000) was used as positive control. After washing 3×5 min in PBS-T, incubation with anti-mouse HRP-coupled secondary antibody for 1 h at RT and 3×10 min washing in PBS-T, ECL was performed with GE Healthcare ECL reagents (RPN2106). Signals were detected by exposure of X-ray films. Molecular weights are indicated in kDa. Yeast strain BY4741 expressing HA-tagged yeast PP2Ac (PPH21) was used as a background. The BY4741 ppe1Δ strain contained high levels of carboxymethylated PP2Ac. The BY4741 ppm1Δ strain lacked carboxymethylated PP2Ac.

FIG. 3: Methyl-PP2Ac specificity of 7C10-C5 confirmed by Western blot analysis. 50 μl lysate corresponding to 180 μg of protein from a BY4741 yeast strain expressing HA-tagged PPH21 and lacking the PP2A methylesterase, PPE1 (ppe1Δ) was treated with 20 of 2M NaOH (resulting in a pH ˜9-10) or with 6 μl of 2M NaOH (resulting in a pH >11) for 5 or 15 minutes on ice. Lysates were then neutralized with HCl to pH ˜7 and boiled with Laemmli buffer. 30 μg of the indicated lysates were loaded per lane. Lysates of the HA-PPH21 expressing BY4741 wt strain or a strain lacking the PP2A methyltransferase, PPM1 (ppm1Δ) were loaded as controls. Proteins were separated by 10% SDS-PAGE and transferred to nitrocellulose membrane (0.2 μm, GE Healthcare). The membranes were blocked in 3% non-fatty dry milk (NFDM) in PBS-Tween for 1 h at RT and incubated with 7C10-C5 hybridoma cell culture supernatant diluted 1:50 in 0.5% NFDM in PBS-T o/n at 4° C. (upper panels), or with anti-HA tag, clone 16B12 (Covance, diluted 1:10,000, lower panel). After washing 3×5 min in PBS-T, incubation with anti-mouse HRP-coupled secondary antibody for 1 h at RT and 3×10 min washing in PBS-T, ECL was performed with GE Healthcare ECL reagents (RPN2106). Signals were detected by exposure (30 s or 60 min) of X-ray films. Molecular weights are indicated in kDa.

FIG. 4: Monoclonal antibody 7C10-C5 is specific for the α-carboxymethylated terminal leucine 309 of PP2Ac. Nunc Medisorp 96-well ELISA flat-bottom plates were coated with peptides L309 (ac-HVTRRTPDYFL) (SEQ ID NO:18), meL309 (ac-HVTRRTPDYFL-CH₃) (SEQ ID NO:18) or amL309 (ac-HVTRRTPDYFL-NH₂) (SEQ ID NO:18) at 2 μg/ml in TBS at 4° C. over-night. After washing once with TBS, the wells were incubated with 2% BSA in TBS for 1 h at RT. After washing once with TBS, the wells were incubated with single clone hybridoma cell culture supernatant clone 7C10-C5 1:50 (˜1 μg/ml) in 1% BSA/TBS for 1 h at RT. After washing twice with TBS, wells were incubated with anti-mouse HRP-coupled secondary antibody for 1 h at RT, followed by washing 3× in PBS. Bound antibodies were detected by colorimetric reaction with 3,3′,5,5′-Tetramethylbenzidine as substrate, and absorbance was measured at 450 nm. Values were normalized to meL309 which was set to 1. Average and standard deviation of N=8 experiments are shown. Statistical significance was assessed using ANOVA followed by Tukey's HSD as a post-hoc test. P values are indicated with *, **, ***, which correspond to values of <0.05, <0.01, and <0.001.

FIG. 5: Monoclonal antibody 7C10-C5 is specific for the α-carboxymethylated PP2Ac. (A) Immunoblotting of lysates from untreated or NaOH-treated HAP1 and HEK293Trex cells using indicated antibodies. The panel originates from 2 different blotting membranes, the H8 blot was reincubated with a pan-actin antibody as loading control. The blots are representative of N=4 (HAP1) or N=3 (HEK) independent immunoblotting experiments. (B) Immunoblotting of lysates of HAP1 wild type or Lcmt-1 knock-out cells (Lcmt-1-KO) were analyzed by immunoblotting with the indicated antibodies. Blots originate from 3 different blotting membranes and are representative of N=5 independent experiments. The label “7C10” refers to the 7C10-C5 monoclonal antibody.

FIG. 6: The methyl-PP2Ac specificity of 7C10-C5 is not impaired by the concurrent phosphorylation of tyrosine 307 or threonine 304 of PP2Ac. Nunc Medisorp 96-well ELISA flat-bottom plates were coated with peptides L309 (ac-HVTRRTPDYFL) (SEQ ID NO:18), meL309 (ac-HVTRRTPDYFL-CH₃) (SEQ ID NO:18), pT304-L309 (ac-HVTRRpTPDYFL) (SEQ ID NO:116) pT304-meL309 (ac-HVTRRpTPDYFL-CH₃) (SEQ ID NO:116), pY307-L309 (ac-HVTRRTPDpYFL) (SEQ ID NO:117) and pY307-meL309 (ac-HVTRRTPDpYFL-CH₃) (SEQ ID NO:117) at 2 μg/ml in TBS at 4° C. over-night. After washing once with TBS, the wells were incubated with 2% BSA in TBS for 1 h at RT. After washing once with TBS, the wells were incubated with single clone hybridoma cell culture supernatant, clone 7C10-C5 1:50 for 1 h at RT. After washing twice with TBS, wells were incubated with anti-mouse HRP-coupled secondary antibody for 1 h at RT, followed by washing 3× in PBS. Bound antibodies were detected by colorimetric reaction with 3,3′,5,5′-Tetramethylbenzidine as substrate, and absorbance was measured at 450 nm. Values were normalized to meL309 which was set to 1. Average and standard deviation of N=8 experiments are shown. Statistical significance was assessed using ANOVA followed by Tukey's HSD as a post-hoc test. P values are indicated with *, **, ***, which correspond to values of <0.05, <0.01, and <0.001.

FIG. 7: The methyl-PP2Ac specificity of 7C10-C5 is not impaired by concurrent phosphorylation of tyrosine 307 or threonine 304 of PP2Ac. Nunc Medisorp 96-well ELISA flat-bottom plates were coated with peptides Y307-L309 (ac-HVTRRTPDYFL) (SEQ ID NO:18), Y307-meL309 (ac-HVTRRTPDYFL-CH₃) (SEQ ID NO:18), pT304-meL309 (ac-HVTRRpTPDYFL-CH3) (SEQ ID NO:116) pY307-meL309 (ac-HVTRRTPDpYFL-CH₃) (SEQ ID NO:117), at a 4 fold serial dilution from 8 μg/ml to 7.8125 ng/ml in TBS at 4° C. over-night. After washing once with TBS, the wells were incubated with 2% BSA in TBS for 1 h at RT. After washing once with TBS, the wells were incubated with hybridoma cell culture supernatant, clone 7C10-C5 1:50 for 1 h at RT. After washing twice with TBS, wells were incubated with anti-mouse HRP-coupled secondary antibody for 1 h at RT, followed by washing 3× in TBS. Bound antibodies were detected by colorimetric reaction with 3,3′,5,5′-Tetramethylbenzidine as substrate, and absorbance was measured at 450 nm. Values were normalized to meL309 which was set to 1. Average and standard deviation of N=3 experiments are shown.

FIG. 8: Monoclonal antibody 7C10-C5 does not cross-react with α-carboxymethylated catalytic subunits of protein phosphatase 4 (PP4c) and protein phosphatase 6 (PP6c) in Western blots. (A) Alignment of the C-termini of mammalian catalytic PP2Ac, PP4c and PP6c subunits (B) Immunoblotting of anti HA-immunoprecipitates of NIH3T3 cells either infected with retroviral supernatants of pBabe hygro (control) or pBabe hygro HA-PP2Ac (label “HA-PP2A), pBabe hygro HA-PP4c (label “HA-PP4”) or pBabe hygro HA-PP6c (label HA-PP6) using indicated antibodies. The label “7C10” refers to the 7C10-C5 monoclonal antibody. To equilibrate HA levels, 2 times more of the HA-PP4c and 3 times more of the HA-PP6c immunoprecipitates was loaded than HA-PP2Ac. The panel originates from 3 different blotting membranes for each antibody indicated in the panel. The blots are representative of N=3 independent immunoprecipitation experiments. The 2A10 and 7C10-C5 signals were quantified from N=3 independent experiments, normalized to the HA levels and the HA-PP2Ac signals were set to 1. Average and standard deviation (error bars) are shown. Statistical significance was assessed using ANOVA followed by Tukey's HSD as a post-hoc test. P values are indicated with *, **, ***, which correspond to values of <0.05, <0.01, and <0.001 (C) Immunoblotting of lysates (not immunoprecipitated) and anti HA-immunoprecipitates (HA-IP) of NIH3T3 cells either infected with retroviral supernatants of pBabe hygro HA-PP2Ac (label “HA-PP2A”) or pBabe hygro HA-PP4c (label “HA-PP4”) (+/− NaOH treatment as indicated) using indicated antibodies. Blots originate from 3 different blotting membranes and are representative of N=3 independent immunoprecipitation experiments. Of note, the lower band in the HA-PP2Ac lysate and the band in the HA-PP4c lysate relates to endogenous PP2Ac.

FIG. 9: Monoclonal antibody 7C10-C5 detects α-carboxymethylated PP2Ac C-terminal peptides with >10-fold higher signal intensity than the α-carboxymethylated PP4c and PP6c C-terminal peptides in ELISA. Nunc Medisorp 96-well ELISA flat-bottom plates were coated with PP2Ac peptides L309 (ac-HVTRRTPDYFL) (SEQ ID NO:18), meL309 (ac-HVTRRTPDYFL-CH3) (SEQ ID NO:18), PP4c peptides L307 (ac-PSKKPVADYFL) (SEQ ID NO:20), meL307 (ac-PSKKPVADYFL-CH₃) (SEQ ID NO:20), PP6c peptides L305 (ac-IPPRTTTPYFL) (SEQ ID NO:22) or meL305 (ac-IPPRTTTPYFL-CH₃) (SEQ ID NO:22) at 2 μg/ml in TBS or TBS only (Neg) at 4° C. over-night. After washing once with TBS, the wells were incubated with 2% BSA in TBS for 1 h at RT. After washing once with TBS, the wells were incubated with single clone hybridoma cell culture supernatant, clone 7C10-C5 1:50 for 1 h at RT. After washing twice with TBS, wells were incubated with anti-mouse and anti rabbit HRP-coupled secondary antibody for 1 h at RT, followed by washing 3× in PBS. Bound antibodies were detected by colorimetric reaction with 3,3′,5,5′-Tetramethylbenzidine as substrate, and absorbance was measured at 450 nm. Values were normalized to meL309 which was set to 1. Average and standard deviation of N=7 experiments are shown. PP2Ac peptides are labeled “PP2A”, PP4c peptides are labeled “PP4”, and PP6c peptides are labeled “PP6”. Statistical significance was assessed using ANOVA followed by Tukey's HSD as a post-hoc test. P values are indicated with *, **, ***, which correspond to values of <0.05, <0.01, and <0.001

FIG. 10: Single chain variable fragments (scFv) of antibody 7C10-C5. Schemes of the recombinant (A) monovalent and (B) bivalent single chain variable fragments (scFv). Monovalent scFv: The Signal Peptide is followed by the variable domain of the 7C10 light chain, a 19 amino acid long linker, the variable domain of the 7C10 heavy chain, 8 amino acids of the constant part of the heavy chain, a 6 times His Tag and an HA tag. Bivalent scFv: The Signal Peptide is followed by the variable domain of the 7C10 light chain, a 19 amino acid long linker, the variable domain of the 7C10 heavy chain, 8 amino acids of the constant part of the heavy chain, a 15 times Glycine Serine linker, the variable domain of the 7C10 light chain, a 19 amino acid long linker, the variable domain of the 7C10 heavy chain, 8 amino acids of the constant part of the heavy chain, a 6 times His Tag and an HA tag. C) Recombinant mono- and bivalent single chain variable fragment antibodies are specific for the α-carboxymethylated PP2Ac. 100 ng of BSA crosslinked with peptides (L309: CGEPHVTRRTPDYFL (SEQ ID NO:49), Y307F: CGEPHVTRRTPDFFL, pY307: CGEPHVTRRTPDpYFL or meL309: CGEPHVTRRTPDYFL-CH₃ (SEQ ID NO:49)) was separated by 10% SDS-PAGE and transferred to nitrocellulose membrane (0.2 μm, GE Healthcare). The membranes were blocked in 3% non-fatty dry milk (NFDM) in TBS-Tween for 1 h at RT and incubated with single clone hybridoma cell culture supernatant, clone 7C10-C5 1:100 (7C10 X63), recombinant single chain variable fragment of antibody 7C10-C5, undiluted (7C10 scFvs), recombinant bivalent single chain variable fragment 7C10-C5, undiluted (7C10 bi-scFvs) or single clone hybridoma cell culture supernatant, clone 1D7 1:100 in 0.5% NFDM in TBS-T o/n at 4° C. After washing 3×5 min in TBS-T, blots incubated with recombinant antibodies were incubated with monoclonal antibody 16B12 (recognizing the hemagglutinin tag on the recombinant antibodies), 1:10000 in 0.5% NFDM in TBS-T 2h at room temperature. After washing 3×5 min in TBS-T, incubation with anti-mouse-HRP coupled secondary antibody for 1 h at RT and 3×10 min washes in TBS-T was performed. ECL was performed with GE Healthcare ECL reagents (RPN2106) mixed 1:3 with Clarity™ Western ECL Substrate, 500 ml #1705061 (Biorad). Signals were detected by exposure of X-ray films. Molecular weights are indicated in kDa.

FIG. 11: Recombinant mono- and bivalent single chain variable fragment antibodies are specific for the α-carboxymethylated PP2Ac. Nunc Medisorp 96-well ELISA flat-bottom plates were coated with peptides either free or linked to BSA, unmodified/L309 (CGEPHVTRRTPDYFL) (SEQ ID NO:49), Y307F (CGEPHVTRRTPDFFL), pY307 (CGEPHVTRRTPDpYFL) or meL309 (CGEPHVTRRTPDYFL-CH₃) (SEQ ID NO:49), on the ELISA plate at 8 μg/ml in TBS (free) or 1 μg/ml in TBS (linked to BSA) at 4° C. over night. After washing twice with TBS, the wells were incubated with 2% BSA in TBS for 1 h at RT. After washing once with TBS, the wells were incubated with single clone hybridoma cell culture supernatant, clone 7C10-C5 1:50 (7C10 X63), recombinant single chain variable fragment of antibody 7C10-C5, undiluted (7C10 scFvs), recombinant bivalent single chain variable fragment of antibody 7C10-C5 undiluted (7C10 bi-scFvs). After washing 3× with TBS, wells incubated with recombinant antibodies were incubated with monoclonal antibody 16B12, 1:10000 (recognizing the hemagglutinin tag on the recombinant antibody) in 1% BSA TBS 1 h at room temperature. After washing 3× in TBS, incubation with anti-mouse-HRP coupled secondary antibody for 1 h at RT was performed. Bound antibodies were detected by colorimetric reaction with 3,3′,5,5′-Tetramethylbenzidine as substrate, and absorbance was measured at 450 nm. Values were normalized to meL309 which was set to 1. Average and standard deviation of N=3 experiments are shown.

FIG. 12: Schematic of PP2A holoenzyme maturation. The PP2A core enzyme consists of A and C subunit. PP2Ac is methylated by LCMT-1. The methylated PP2A core enzyme associates with B, B′ or B″ subunits. The unmethylated core enzyme with Striatin, DNA tumor virus antigens SV40 ST, PyST or MT.

FIG. 13: Monoclonal antibody 7C10-C5 detects a SMAP (DT-061)-induced, dose dependent increase of the α-carboxymethylation of PP2Ac. (A) Immunohistochemical analysis of xenograft treated tumors probed for mL309 (left) with the 7C10-C5 monoclonal antibody or tPP2A-C (right) with the antibody Abcam Cat #ab106262 (polyclonal antibody with epitope in the N terminus of PP2Ac: FTKELDQWIEQLNEC) (SEQ ID NO:52) images shown are 100× magnification and representative from each time point (n=4-6), demonstrates an increase in mL309 at early time points following DT-061 treatment. (B) Densitometric quantification of western blots probed for methyl-L309 PP2A-C (mL309), total-PP2Ac (tPP2A-C) and vinculin with the antibodies 7C10-C5, Abcam Cat #ab106262 and Santa Cruz Cat #13901S, respectively, from tumors treated with a single dose of DT-061 (5 mg/kg) for the designated times (n=5-13 tumors per group), mirrors the IHC data whereby DT-061 treatment enhances mL309 between 1-3 hours after treatment followed by a return to baseline. Individual data points represent ratios, mL309/tPP2A-C or tPP2A-C/Vinculin, all normalized to a single tumor from vehicle control group included on all gels. Box-whiskers represent average ±s.d. one way parametric ANOVA with Dunnett multiple comparison test presented comparing treated groups to vehicle control, ***=p<0.001. (C) Correlation of DT-061 serum concentration and detected mL309 level from individual tumors. Linear regression with r=0.64 and significantly positive slope, p<0.0001 (See also FIG. 51). (D) Immunohistochemical c-MYC staining with the antibody Abcam Cat #ab32072 of single dose vehicle or DT-061 treated xenograft tumors, demonstrates PP2A activation by DT-061 in vivo, which is temporally in line with and inversely proportional to mL309 levels, images are 100× magnification. (E) Peptide competition with methylated peptide (binding/neutralizing the 7C10-C5 antibody) show diminished staining in comparison with unmodified peptide (not binding/neutralizing the 7C10-C5 antibody). The label “7C10” indicates staining with the 7C10-C5 monoclonal antibody. (F) Incubation with NaOH results in decreased α-carboxymethylation and decreased α-carboxymethyl-PP2Ac staining.

FIG. 14: Monoclonal antibody 7C10-C5 detects a SMAP (DT-061)-induced, dose dependent increase of the α-carboxymethylation of PP2Ac. (A) DT-061 increases methylation of L309 on the PP2A-C subunit (Related to FIG. 13). (A-G) Complete western blots for PP2Ac L309 methylation, total PP2Ac, and vinculin in control or DT-061 treated groups determined by the antibodies as in FIG. 13. (H) High magnification image (40×) of methylC, totalC. IHC from representative tumors. (I) Quantification of methyl-C staining intensity from IHC performed on single dose time-course xenograft tumors (n for each time point=4-6) (J) DT-061 half-life obtained from serum determined to be 6.3 hours. (K) Correlative analysis between tPP2Ac 10 densitometry and DT-061 serum concentrations, no correlation observed.

FIG. 15: Immunohistochemical analysis of prostate cancer tissue microarray. Tissue microarray analysis in localized and metastatic prostate tissues and quantification of their Allred scores. Monoclonal antibody 7C10-C5 detects significantly lower levels of α-carboxymethylated PP2Ac in metastatic than in non-metastatic prostate cancer, whereas E155 antibody or anti-total PP2Ac antibody YE351 (ab32065, abcam) are unable to discriminate between these disease states. (A) Allred score of staining with 7C10-C5 antibody and high magnification image (20×); (B) Allred score of staining with E155 antibody and high magnification image (20×).; (C) Allred score of staining with anti-total PP2Ac antibody YE351 (ab32065, abcam) and high magnification image.

FIG. 16: Mouse monoclonal antibody 4B7 is impaired in the recognition of PP2Ac by α-carboxymethylation at Leu309 but also by phosphorylation at position Thr304. Nunc Medisorp 96-well ELISA flat-bottom plates were coated with peptides L309 (ac-HVTRRTPDYFL) (SEQ ID NO:18), meL309 (ac-HVTRRTPDYFL-CH₃) (SEQ ID NO:18), pT304-L309 (ac-HVTRRpTPDYFL) (SEQ ID NO:116) pT304-meL309 (ac-HVTRRpTPDYFL-CH3) (SEQ ID NO:116), pY307-L309 (ac-HVTRRTPDpYFL) (SEQ ID NO:117) and pY307-meL309 (ac-HVTRRTPDpYFL-CH₃) (SEQ ID NO:117) on the ELISA plate at 2 μg/ml in TBS at 4° C. over night. After washing once with TBS, the wells were incubated with 2% BSA in TBS for 1 h at RT. After washing once with TBS, the wells were incubated with monoclonal antibody 4B7 (SCBT, sc-13601, lot 0716) 1 μg/ml for 1 h at RT. After washing twice with TBS, wells were incubated with anti-mouse HRP-coupled secondary antibody for 1 h at RT, followed by washing 3× in PBS. Bound antibodies were detected by colorimetric reaction with 3,3′,5,5′-Tetramethylbenzidine as substrate, and absorbance was measured at 450 nm. Values were normalized to L309 which was set to 1. Average and standard deviation of N=6 experiments are shown. Statistical significance was assessed using ANOVA followed by Tukey's HSD as a post-hoc test. P values are indicated with *, **, ***, which correspond to values of <0.05, <0.01, and <0.001. In addition, fold-changes between conditions are shown as indicated by the bottom brackets.

FIG. 17: Mouse monoclonal antibody 1D7 is impaired in the recognition of PP2Ac by α-carboxymethylation at Leu309 but also by phosphorylation at position Thr304. Nunc Medisorp 96-well ELISA flat-bottom plates were coated with peptides L309 (ac-HVTRRTPDYFL) (SEQ ID NO:18), meL309 (ac-HVTRRTPDYFL-CH₃) (SEQ ID NO:18), pT304-L309 (ac-HVTRRpTPDYFL) (SEQ ID NO:116) pT304-meL309 (ac-HVTRRpTPDYFL-CH₃) (SEQ ID NO:116), pY307-L309 (ac-HVTRRTPDpYFL) (SEQ ID NO:117) and pY307-meL309 (ac-HVTRRTPDpYFL-CH₃) (SEQ ID NO:117) on the ELISA plate at 2 μg/ml in TBS at 4° C. over night. After washing once with TBS, the wells were incubated with 2% BSA in TBS for 1 h at RT. After washing once with TBS, the wells were incubated with single clone hybridoma cell culture supernatant, clone 1D7 1:10 for 1 h at RT. After washing twice with TBS, wells were incubated with anti-mouse HRP-coupled secondary antibody for 1 h at RT, followed by washing 3× in PBS. Bound antibodies were detected by colorimetric reaction with 3,3′,5,5′-Tetramethylbenzidine as substrate, and absorbance was measured at 450 nm. Values were normalized to meL309 which was set to 1. Average and standard deviation of N=6 experiments are shown. Statistical significance was assessed using ANOVA followed by Tukey's HSD as a post-hoc test. P values are indicated with *, **, ***, which correspond to values of <0.05, <0.01, and <0.001. In addition, fold-changes between conditions are shown as indicated by the bottom brackets.

FIG. 18: Rabbit monoclonal antibody E155 is impaired in the recognition of PP2Ac by α-carboxymethylation at Leu309 but also by phosphorylation at position Thr304. Nunc Medisorp 96-well ELISA flat-bottom plates were coated with peptides L309 (ac-HVTRRTPDYFL) (SEQ ID NO:18), meL309 (ac-HVTRRTPDYFL-CH₃) (SEQ ID NO:18), pT304-L309 (ac-HVTRRpTPDYFL) (SEQ ID NO:116) pT304-meL309 (ac-HVTRRpTPDYFL-CH3) (SEQ ID NO:116), on the ELISA plate at 2 μg/ml in TBS at 4° C. over night. After washing once with TBS, the wells were incubated with 2% BSA in TBS for 1 h at RT. After washing once with TBS, the wells were incubated commercial E155 (Abcam E155 #ab32104 lot GR17965-24) at 1 μg/ml in 1% BSA/TBS for 1 h at RT. After washing twice with PBS, wells were incubated with anti-mouse HRP-coupled secondary antibody for 1 h at RT, followed by washing 3× in TBS. Bound antibodies were detected by colorimetric reaction with 3,3′,5,5′-Tetramethylbenzidine as substrate, and absorbance was measured at 450 nm. Values were normalized to meL309 which was set to 1. Average and standard deviation of 4 experiments are shown. Statistical significance was assessed using ANOVA followed by Tukey's HSD as a post-hoc test. P values are indicated with *, **, ***, which correspond to values of <0.05, <0.01, and <0.001. In addition, fold-changes between conditions are shown as indicated by the bottom brackets.

FIG. 19: Detection of PP2Ac with rabbit monoclonal antibody E155 is less impaired by α-carboxymethylation of Leu309, if Tyr307 is concurrently phosphorylated. Nunc Medisorp 96-well ELISA flat-bottom plates were coated with peptides L309 (ac-HVTRRTPDYFL) (SEQ ID NO:18), meL309 (ac-HVTRRTPDYFL-CH₃) (SEQ ID NO:18), pY307-L309 (ac-HVTRRTPDpYFL) (SEQ ID NO:117) pY307-meL309 (ac-HVTRRTPDpYFL-CH₃) (SEQ ID NO:117), on the ELISA plate over night at 2 μg/ml in TBS at 4° C. o/n. After washing once with TBS, the wells were incubated with 2% BSA in TBS for 1 h at RT. After washing once with TBS, the wells were incubated commercial E155 (Abcam E155 #ab32104 lot GR17965-24) at 1 μg/ml in 1% BSA/TBS for 1 h at RT. After washing twice with PBS, wells were incubated with anti-mouse HRP-coupled secondary antibody for 1 h at RT, followed by washing 3× in TBS. Bound antibodies were detected by colorimetric reaction with 3,3′,5,5′-Tetramethylbenzidine as substrate, and absorbance was measured at 450 nm. Values were normalized to L309 which was set to 1. Average and standard deviation of N=3 experiments are shown. Statistical significance was assessed using ANOVA followed by Tukey's HSD as a post-hoc test. P values are indicated with *, **, ***, which correspond to values of <0.05, <0.01, and <0.001. In addition, fold-changes between conditions are shown as indicated by the bottom brackets.

FIG. 20: Rabbit monoclonal antibody E155 is impaired in the recognition of PP2Ac by α-carboxymethylation at Leu309 but also by phosphorylation at position Thr304. Nunc Medisorp 96-well ELISA flat-bottom plates were coated with peptides Y307-L309 (ac-HVTRRTPDYFL) (SEQ ID NO:18), Y307-meL309 (ac-HVTRRTPDYFL-CH₃) (SEQ ID NO:18) and either pT304-L309 (ac-HVTRRpTPDYFL) (SEQ ID NO:116) pT304-meL309 (ac-HVTRRpTPDYFL-CH₃) (SEQ ID NO:116) (top panel) or pY307-L309 (ac-HVTRRTPDpYFL) (SEQ ID NO:117) pY307-meL309 (ac-HVTRRTPDpYFL-CH₃) (SEQ ID NO:117) (bottom panel), on the ELISA plate at a 4 fold serial dilution from 8 μg/ml to 7.8125 ng/ml in TBS at 4° C. over night. After washing once with TBS, the wells were incubated with 2% BSA in TBS for 1 h at RT. After washing once with TBS, the wells were incubated with E155 (Abcam E155 #ab32104 lot GR17965-24) at indicated concentrations in 1% BSA/TBS for 1 h at RT. After washing twice with TBS, wells were incubated with anti-mouse HRP-coupled secondary antibody for 1 h at RT, followed by washing 3× in PBS. Bound antibodies were detected by colorimetric reaction with 3,3′,5,5′-Tetramethylbenzidine as substrate, and absorbance was measured at 450 nm. Values were normalized to L309 which was set to 1. Average and standard deviation of 3 experiments are shown.

FIG. 21: IHC detection of PP2Ac in cancer tissues by rabbit monoclonal E155 does not inversely correlate with the detection of α-carboxymethylated PP2Ac by monoclonal antibody 7C10-C5. (A) Prostate cancer tissue. High magnification image (20×). (B) Lung adenocarcinoma tissue. High magnification image (20×). (C) Lung squamous cancer tissue. High magnification image (20×).

FIG. 22: Western blot analysis with the 7C10-C5 monoclonal antibody or antibodies that possess a preference for non-methylated PP2Ac (4B7, 1D7, E155 and F-8). Immunoblotting of lysates from untreated or NaOH treated HAP1 wild type cells or HAP1 Lcmt1⁻ cells using indicated antibodies. The label “7C10” refers to the 7C10-C5 monoclonal antibody. A dilution series of NaOH treated HAP1 cells were loaded for western quantification. The Blots were incubated with monoclonal antibody E155 (Abcam Abcam E155 #ab32104 lot GR17965-24) 1:5000, 4B7 (SCBT, sc-13601, lot 0716E155) 1:500) F8 (SCBT sc-271903, lot C1617), 1:500, single clone hybridoma cell culture supernatants, clone 1D7 1:200, clone 7C10-C5 1:100, clone 118 1:100 and pan Actin 1:200, diluted in 0.5% NFDM TBS 0.5% Tween, for 1h at room temperature. After washing 3× for 5 min with TBS 0.5% Tween, the blots were incubated with anti-mouse or anti-rabbit HRP-coupled secondary antibody for 1 h at RT 0.5% NFDM TBS 0.5% Tween, followed by washing 3× in TBS 0.5% Tween. Bound antibodies were detected by ECL.

FIG. 23. Graphical summary of detection properties of C-terminal PP2Ac antibodies. Monoclonal antibody 7C10-C5 is truly specific for the α-carboxymethylated PP2Ac. Monoclonal antibody 4D9 is potentially specific for the uncharged, amidated C-terminus but not the α-carboxymethylated C-terminus of PP2Ac subunit. Antibodies 4B7, 1D7, F8, E155 and 1D6 are impaired by α-carboxymethylation at Leu309 and thus recognize non-methylated PP2Ac.

FIG. 24. Distribution of Allred scores for methyl-PP2Ac IHC staining using the 7C10-C5 antibody in a panel of human dysplastic and cancer tissues. The number of tissues stained is indicated above each pie chart and the percentage of patients that fall into each of the Allred scores is indicated. Monoclonal antibody 7C10-C5 detects significantly lower levels of α-carboxymethylated PP2Ac in tissue microarrays (TMAs) of metastasizing prostate cancer than in TMAs of lung adenocarcinoma, lung squamous carcinoma, and barren's esophagus. Allred score of 7C10-C5 antibody and high magnification image (20×). The majority of metastatic prostate cancers have no to low levels of methylated PP2A.

FIG. 25. Positive correlation of carboxymethylated PP2Ac levels in prostate cancer patient samples (as determined by the anti-carboxymethylated PP2Ac-specific antibody, here the 7C10-C5 antibody) and survival of patients. A) Carboxymethylated PP2Ac (Methyl-C) levels in differently staged prostate cancers (according to the Gleason score) were determined using the 7C10-C5 antibody. The Allied scores (categorical values) are jittered around the actual value for display purposes. B) The carboxymethylated PP2Ac (Methyl-C) levels were analyzed in Gleason 7 scored tumors from 50 individual patients by immunostaining with the 7C10-C5 antibody and Allied scoring of the staining, and the survival of the patients was monitored. Specifically, Kaplan-Meier curves of a prostate cancer cohort with Gleason Score 7 (n=50) stained for 7C10-C5 and scored using Allied scoring are shown. Patients for which an Allred score of at least 2 (low/intermediate to high carboxymethylated PP2Ac levels) has been determined survived longer, i.e. without recurrence of the cancer, than patients for which an Allied score below 2 (very low carboxymethylated PP2Ac levels/no carboxymethylated PP2Ac) has been determined (p=0.0329; Log-rank (Mantel-Cox) test).

FIG. 26. Analysis of LCMT1 expression in prostate cancer tissue and Enzalutamide resistant prostate cancer cells. A) Proteomic profiling of benign, localized, and metastatic prostate cancer reveals a negative correlation of AR expression with LCMT1 expression (Pearson r=−0.3385, P=0.0141). B,C) Enzalutamide resistant cell lines derived by long-term exposure to enzalutamide showed B) decreased LCMT1 mRNA expression and C) decreased LCMT1 protein levels.

FIG. 27. LCMT1 alters methylation of PP2Ac in prostate cancer cells. A) Stable knockdown of LCMT1 in LNCaP resulted in decreased methylation of PP2Ac as assessed by western blotting with the 7C10-C5 antibody. B) Stable knockdown of LCMT1 resulted in increased phosphorylation of AR and cMYC.

FIG. 28. LCMT1 knockdown attenuates Enzalutamide response. A) Representative images and quantification of colony formation in response to Enzalutamide in LCMT1 knockdown lines. B) Cell titer glo assay in LCMT1 knockdown lines treated with Enzalutamide after 96 hours.

FIG. 29. Schemes of (A) recombinant Light chain variable fragment and mIgCK of antibody 7C10-C5 and (B) recombinant Heavy chain variable fragment and mIgG2a of antibody 7C10-C5. A) The Signal Peptide L1 is followed by the variable domain of the 7C10-C5 light chain and the mIgCK constant region provided by the pTrioz vector. B) The Signal Peptide IL2 is followed by the variable domain of the 7C10-C5 heavy chain and the mIgG2a constant region provided by the pTrioz vector.

FIG. 30. Recombinant 7C10-C5 antibody is specific for the α-carboxymethylated PP2Ac as determined by ELISA. Nunc Medisorp 96-well ELISA flat-bottom plates were coated with peptides either free or linked to BSA, L309 (CGEPHVTRRTPDYFL-OH) (SEQ ID NO:49) or meL309 (CGEPHVTRRTPDYFL-CH₃) (SEQ ID NO:49), on the ELISA plate at 0.5 μg/ml in TB S (free) or 1 μg/ml in TB S (linked to BSA) at 4° C. over night. After washing twice with TB S, the wells were incubated with 2% BSA in TBS for 1 h at RT. After washing once with TBS, the wells were incubated with single clone hybridoma cell culture supernatant, clone 7C10-C5 1:50 (7C10 X63), or recombinant antibody 7C10-C5, undiluted (rec 7C10). After washing 3× with TBS, incubation with anti-mouse-HRP coupled secondary antibody for 1 h at RT was performed. Bound antibodies were detected by colorimetric reaction with 3,3′,5,5′-Tetramethylbenzidine as substrate, and absorbance was measured at 450 nm. Values were normalized to meL309 incubated with 7C10 X63 which was arbitrarily set to 1. Average and standard deviation of N=3 experiments are shown.

FIG. 31: Recombinant antibody 7C10-C5 is specific for the α-carboxymethylated PP2Ac as determined by Western blotting. Immunoblotting of lysates from untreated or NaOH treated HEK293T cells using indicated antibodies. The panel originates from 4 different blotting membranes. The Blots were incubated with single clone hybridoma cell culture supernatant, clone H8 1:50, 1D7 (1:100), 7C10-C5 (1:200), or recombinant 7C10-C5 HEK293T cell culture supernatant (rec 7c10 (undiluted)). The ponceau stain of the rec 7C10-C5 blot is shown as loading control.

FIG. 32: Monoclonal antibody 7C10-C5 detects α-carboxymethylated PP2Ac C-terminal peptides with >40.7-fold higher affinity than monoclonal antibody 2A10. (A) Saturation curve of the titration of the α-carboxymethylated PP2Ac C-terminal peptide (SEQ ID NO:18) on 7C10-C5 or 2A10 immobilized on a Series S Sensor Chip CM5 (GE Healthcare, 10296958). Peptide binding curves to the indicated antibodies were fit to a steady-state affinity model and yielded a K_{D} value of 11 nM for 7C10-C5 (left curve) and a K_{D} value of 448 nM for 2A10 (right curve). (B) Saturation curve of the titration of the α-carboxymethylated PP4c C-terminal peptide (SEQ ID NO:20) on 7C10-C5 or 2A10 immobilized on a Series S Sensor Chip CM5 (GE Healthcare, 10296958). Peptide binding curves to the indicated antibodies were fit to a steady-state affinity model and yielded a K_{D} value of 132 nM for 7C10-C5 (left curve) and a K_{D} value of 1130 nM for 2A10 (right curve). The K_{D} values were obtained using the fitting tool of the BiacoreT200 Evaluation Software (version 3.1).

Other aspects and advantages of the invention will be described in the following examples, which are given for purposes of illustration and not by way of limitation. Each publication, patent, patent application or other document cited in this application is hereby incorporated by reference in its entirety.

### EXAMPLES

Methods and materials are described herein for use in the present disclosure; other, suitable methods and materials known in the art can also be used. The materials, methods, and examples are illustrative only and not intended to be limiting.

### Example 1. Generation of the α-Carboxymethylation Specific Monoclonal Antibody 7C10-C5

The single clone hybridoma cell line “7C10-C5” (mouse Balb/c origin) as described herein in the Examples producing the α-carboxymethylation specific monoclonal antibody 7C10-C5 as characterized herein in the Examples, i.e. in Examples 2 and 3, has been deposited at the International Depository Authority “Leipniz Institute DSMZ-German Collection of Microorganisms and Cell Cultures, Inhoffenstr. 7B, D-38124 Braunschweig” under the accession number “DSM ACC3350”. The Leipniz Institute DSMZ-German Collection of Microorganisms and Cell Cultures, Inhoffenstr. 7B, D-38124 Braunschweig, has received said “7C10-C5” cell line with the accession number “DSM ACC3350” on 2019-01-16.

Immunization of Mice

To generate an antibody that specifically recognizes the α-carboxymethylated C-terminal leucine at position 309 (Leu³⁰⁹) of the a and f3 isoforms of the PP2A catalytic subunit (PP2Ac) mice were immunized with a synthetic peptide spanning amino acid 304 to 309 of the carboxyl terminus of the human PP2Ac subunit (sequence identical between α and β isoforms), in which the α-carboxyl group of the C-terminal leucine was methylated.

To elicit an immune response towards the α-carboxymethylated peptide, the peptide was crosslinked to the carrier protein keyhole limpet hemocyanin (KLH) via cysteine which was linked to the PP2Ac peptide by the non-proteinogenic amino acid β-alanine.

The Immunogen

Specifically, a peptide of the sequence C(βA)TPDYFL (SEQ ID NO:48), of which the last six amino acids correspond to the C-terminal 6 amino acids of mammalian or yeast PP2A catalytic subunit (PP2Ac) and which contains a carboxy-terminal methyl-esterified leucine (L-O-CH3) was synthesized by standard peptide synthesis by PiChem GmbH (Graz, Austria) and coupled via the amino-terminal cysteine to maleimide activated keyhole limpet hemocyanine (KLH). As a result, an immunogen of the sequence KLH-Cys-βAla-Thr-Pro-Asp-Tyr-Phe-Leu-CH3 (SEQ ID NO: 48) was obtained (the C-terminal 6 amino acid sequence of the mammalian or yeast PP2Ac including the carboxy-terminal methyl-esterified leucine is shown in bold).

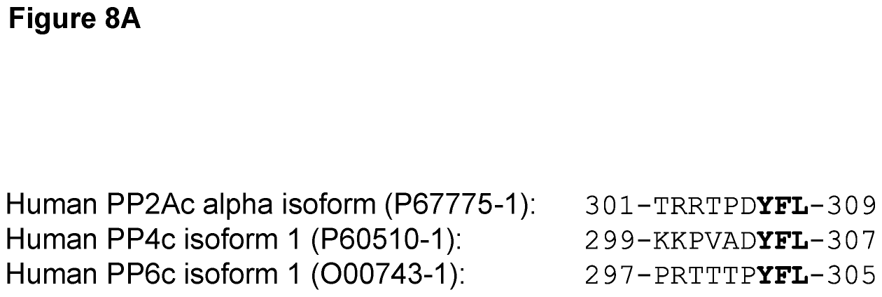

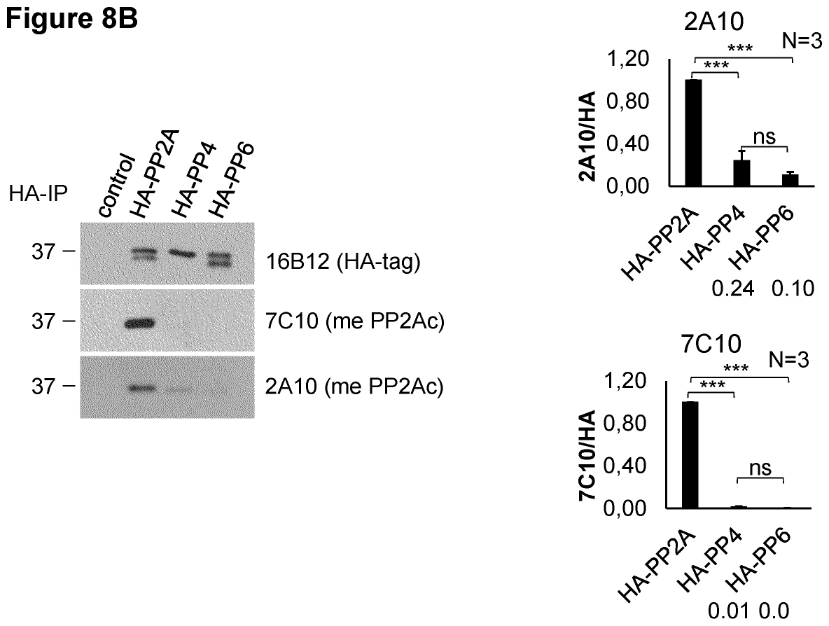

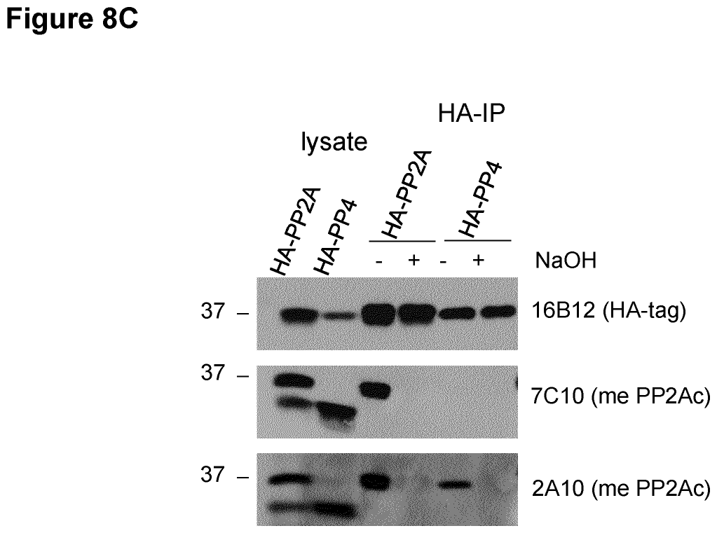

The short PP2Ac specific sequence of only 6 amino acids was used to minimize the chance of obtaining antibodies that recognize the PP2Ac C-terminus independently of the methylated C-terminal leucine. Of note, for the generation of an earlier antibody (clone 2A10) binding to α-carboxymethyl-PP2Ac but cross-reacting with α-carboxymethyl-PP4 and weakly with α-carboxymethyl-PP6 (Tables 4 and 5 and FIG. 8B+C), an 8 amino acid peptide spanning amino acid 302 to 309 of the carboxyl terminus of the human PP2Ac subunit was used.

Immunization

50 μg of said KLH-coupled peptide at a concentration of 0.5 μg/μl in PBS was mixed with adjuvant. In particular, the aqueous antigen solution and the adjuvant oil were emulsified by repeated cycles of sucking-up and pushing-out the oil-water mixture through a 23G (0.6 mm diameter) needle until a stable emulsion was formed. Blood samples were collected from the tail veins of four female cByJ.RBF-Rb(8.12)5Bnr/J mice at the age of 10 weeks (“pre-immune sera”), incubated for 1 h at 37° C. and centrifuged for 5 min at 22° C. at 14,000 rpm in a Beckman & Coulter Microfuge 18. The cleared blood sera were collected, sodium azide was added to a final concentration of 0.02% w/v, and the sera were stored at 4° C. Immediately after the collection of blood, the mice were immunized with 200 μl of antigen-adjuvant emulsion per mouse which was injected subcutaneously at the abdomen. 14 days after the first immunization (day 15), the mice were boosted with 100 μl of said KLH-coupled peptide at a concentration of 0.5 μg/μl in PBS emulsified with 100 μl adjuvant per mouse which was injected subcutaneously at the abdomen. 35 days after the first immunization (day 36), the mice were boosted a second time with 100 μl of said KLH-coupled peptide at a concentration of 0.5 μg/μl in PBS emulsified with 100 μl adjuvant per mouse which injected subcutaneously at the abdomen. 13 days after the second boost (day 49), blood samples of all mice were taken (“immune sera”, 2^{nd} bleed) from the tail veins, incubated for 1 h at 37° C. and centrifuged for 5 min at 22° C. at 14,000 rpm in a Beckman & Coulter Microfuge 18. 95 days after the first immunization (day 96), the mice were boosted a third time with 90 μl of said KLH-coupled peptide at a concentration of 0.5 μg/μl in PBS mixed with 90 μl adjuvant per mouse which was injected subcutaneously at the abdomen. 10 days after the third boost (day 106), blood samples of all mice were taken (“immune sera”, 3^{rd} bleed) from the tail veins, incubated for 1 h at 37° C. and centrifuged for 5 min at 22° C. at 14,000 rpm in a Beckman & Coulter Microfuge 18.

162 days after the first immunization (day 163), mouse 4 (used for the generation of hybridoma cells) received a final boost with 40 μg of KLH-coupled peptide in 100 μl of PBS injected intravenously into the tail vein. 88 hours post injection (day 167) the mouse was sacrificed by cervical dislocation and the spleen removed surgically.

Screening of Polyclonal Antisera and Hybridoma Clone Supernatants

To identify antibodies specifically binding the carboxymethylated catalytic subunit of protein phosphatase 2A (PP2Ac) but not non-carboxymethylated PP2Ac, lysates of different yeast strains having either high levels of carboxymethylated PP2Ac or lacking carboxymethylated PP2Ac were employed for screening.

Yeast Strains

Yeast strain BY4741 expressing HA-tagged yeast PP2Ac (PPH21) was used as a background. Of note, the 6 C-terminal amino acids of mammalian/human PP2Ac are identical to the yeast PP2A catalytic subunit PPH21. The BY4741 wildtype (WT) strain contained carboxymethylated PP2Ac and non-carboxymethylated PP2Ac. The BY4741 ppe1Δ strain contained high levels of carboxymethylated PP2Ac because the ppe1 gene was deleted. The BY4741 ppm1Δ strain lacked carboxymethylated PP2Ac because the ppm1 gene was deleted. Said yeast strains were further used for the characterization of monoclonal antibodies, i.e. 7C10-C5, as described further below. PPE1/ppe1: Phosphoprotein Phosphatase Methylesterase; PPM1/ppm1: Protein Phosphatase Methyltransferase; HA: hemagglutinin.

Preparation of Yeast Cell Lysates for Immunoblot Analysis

Yeast cells were grown at 30° C. to exponential growth phase in drop-out complete medium (2.3 g/l Bacto yeast nitrogen base [Difco, 233520], 20 mg/l adenine, 20 mg/l L-arginine, 15 mg/l L-tyrosine, 15 mg/l L-isoleucine, 25 mg/l L-phenylalanine, 50 mg/l L-glutamic acid, 50 mg/l L-aspartic acid, 100 mg/l L-threonine, 200 mg/l L-serine, 75 mg/l L-valine, 75 mg/l L-methionine, 90 mg/l L-lysine, 20 mg/l uracil, 30 mg/l L-histidine, 0.05 M ammoniumsulfate) lacking L-leucine and containing 2% w/v glucose. Yeast cells were collected by centrifugation at 3,500 rpm at 4° C. in a Beckmann GS-6R centrifuge, washed with ice-cold water and the optical density at 600 nm (0D⁶⁰⁰) was measured in a Hitachi U-2000 spectrophotometer. Yeast cells equivalent to 50 OD⁶⁰⁰ were resuspended in 600 μl of yeast IP buffer (50 mM Tris pH 7.6, 150 mM NaCl, 1% Triton X-100, 1 mM EDTA, 2 mM phenylmethylsulfonyl fluoride, 0.015-0.105 Trypsin Inhibitor Units per ml [TIU/ml] aprotinin [Sigma, A-6279], Complete protease inhibitor cocktail [Roche, 11836145001]), 400 μl of glass beads (0.5 mm diameter, Roth, N030.1) were added, and the cells were lysed in a MP Fastprep-24 at 6 m/s for 40 sec. Lysates were cleared by two times centrifugation for 10 min at 14,000 rpm at 4° C. in a Beckman & Coulter Microfuge 18 centrifuge. The protein concentration was determined with the Protein Assay Dye Reagent (Biorad, 500-0006) and measurement of absorbance at 595 nm in a Hitachi U-2000 spectrophotometer. For SDS-PAGE and Western blotting protein loading buffer was added to a final concentration of 0.112 M dithiothreitol, 2.22% w/v SDS and 11.1% glycerol, and protein samples were denatured by incubation for 5 min at 95° C.

Characterization of Polyclonal Antisera

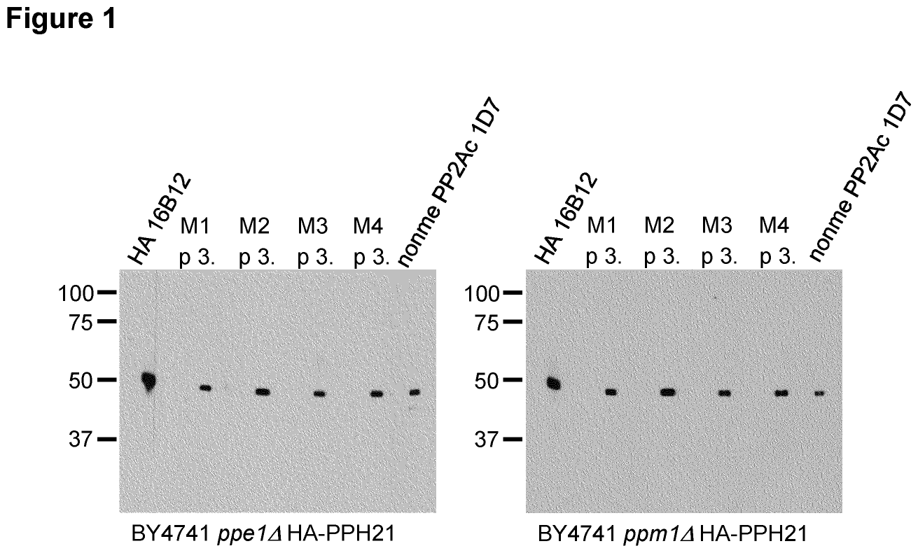

To discriminate between carboxymethyl-specific versus carboxymethyl-unspecific polyclonal PP2Ac antibodies, the polyclonal antisera of the immunized mice were screened by Western blot analysis (FIG. 1) on lysates from the BY4741 ppe1Δ strain or the BY4741 ppm1Δ strain.

Specifically, the cleared blood sera were collected, sodium azide was added to a final concentration of 0.02% w/v, and the sera were tested for the presence of mouse PP2Ac specific IgG antibodies by immunoblotting (Western blot analysis) against 10% SDS-PAGE separated NIH3T3 mouse fibroblast whole cell lysate (prepared as in Example 2), as well as yeast lysates from the BY4741 ppe1Δ strain or the BY4741 ppm1Δ strain. 85×73 mm SDS polyacrylamide gels with 1 mm thick preparative combs (Bio-Rad, 165-2928) were casted with Bio-Rad Mini PROTEAN II electrophoresis cell systems. Whole cell lysate equivalent to 400 μg of total protein was mixed with denaturing buffer to a final concentration of 0.11 mol/l Dithiothreitol (DTT), 2.2% sodium dodecyl sulfate (SDS) and 11% (v/v) glycerol. Proteins were denatured by incubation at 95° C. for 5 min and separated in 0.025 M Tris/0.2 M glycine/0.01% w/v SDS pH 8.5 running buffer at a constant voltage of 100 V at 22° C. Proteins were transferred to Protran BA 83 nitrocellulose membrane (Whatman, 10401396) in 0.025 M Tris/0.19 M glycine/20% methanol pH 8.5 transfer buffer for 3h at a constant current of 0.5 A at 4° C. in Hoefer TE-Series Transphor Electrophoresis Units (Pharmacia Biotech, TE42). Membranes were washed with deionized water and stained with Ponceau S (2.97 mmol/l Ponceau S [Serva, 33429], 0.184 mol/l trichloroacetic acid [AppliChem, A1431], 0.137 mol/l sulfosalicylic acid [Merck, 1.00691]) for 2 min. Excess dye was washed off with deionized water and membranes were stored dry between two 3MM paper sheets at 22° C. Prior to usage, membranes were rehydrated by incubation for 2 min at 22° C. in PBS+0.1% Tween-20 (PBS-T). Membranes were blocked by incubation for 1 h at 22° C. in PBS-T+3% w/v skim milk powder (Merck, 1.15363). Blots were incubated with pre-immune or immune sera diluted 1:500 in PBS-T+0.5% skim milk powder in a Miniblotter system 28 channels dual blot MN28 unit (Immunetics, 168830) over night at 4° C. Membranes were washed 3×5 min with PBS-T at 22° C. For detection of primary mouse antibodies, membranes were incubated with peroxidase-conjugated AffiniPure goat anti-mouse IgG Fcγ fragment specific (Jackson ImmunoResearch, 115 008) secondary antibody diluted 1:10,000 in PBS-T+0.5% skim milk powder for 1 h at 22° C. Membranes were washed three times 10 min with PBS-T at 22° C. and bound antibodies were visualized by enhanced chemoluminescence with ECL Western Blotting Detection Reagents (GE Healthcare, RPN2106) and exposure of Fuji Medical X-ray films (FUJIFILM Corporation, Super HR-E, 47410 08471).

All four mice showed a robust immune response against mammalian (NIH3T3 mouse fibroblast) as well as yeast PP2A catalytic subunit (yeast strains). However, the polyclonal antisera of the immunized mice detected the PP2Ac subunit independent of its carboxymethylation state (FIG. 1) indicating that the immune response had primarily produced carboxymethyl-unspecific PP2Ac antibodies and that any carboxymethyl-PP2Ac specific clone might rather be a rare species that can only be isolated by the monoclonalization of hybridoma cells.

Generation of Hybridoma Mix Clones

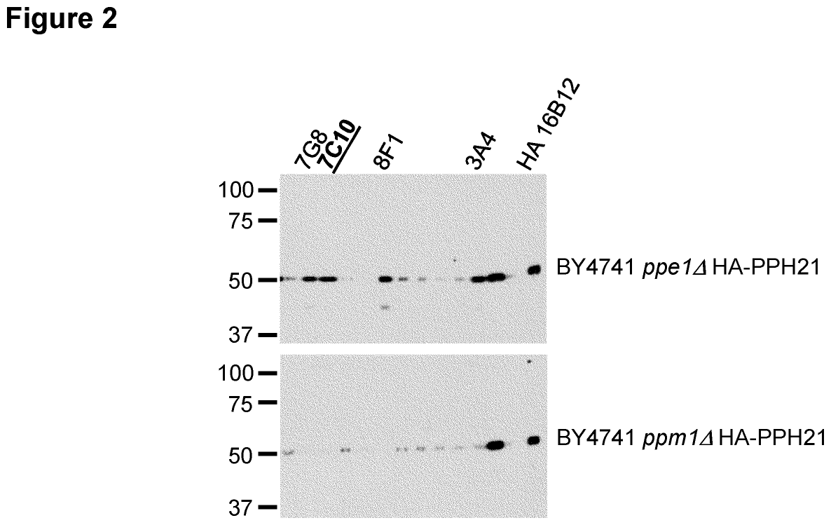

First, splenocytes were fused with mouse myeloma cells to generate hybridoma cells. Specifically, the spleen of mouse 4 was placed in 10 ml of 37° C. warm Dulbecco's Modified Eagle's medium (DMEM; Sigma, D5671), cut in small pieces with a sterile pair of scissors and grinded between two sterile frosted microscope slides (Menzel Glaser Superfrost Plus, Thermo Scientific, J1800AMNZ) until no macroscopic pieces of splenic tissue were visible. The cell suspension was filtered through a 100 μm nylon cell strainer (BD Falcon, Ref. 352360) and the filter was washed two times with 10 ml of 37° C. warm DMEM. Cells were centrifuged for 5 min at 1200 rpm in a Heraeus Megafuge 1.0 at 22° C., resuspended in 3 ml of ice-cold red blood cell lysis buffer (Sigma, R7757) and incubated for 90 sec. The cell suspension was filled up to 30 ml with 37° C. warm DMEM and centrifuged for 5 min at 1200 rpm in Heraeus Megafuge 1.0 at 22° C. The splenocytes were counted with a 0.0025 mm² glass counting chamber (0,100 mm depth; Bikker, Labor Optik). X63-Ag8.653 mouse myeloma cells were grown at 37° C. in a 5% CO2 atmosphere on Vents Nunclon TC 140/20 petri dishes (Nunc, 168381) for a minimum of 3 passages after thawing in DMEM+10% fetal bovine serum (Sigma, F7524)+2 mM Glutamax (Gibco, 35050-038)+100 units/ml Penicillin/0.1 mg/ml Streptomycin (Sigma, P4333)+1 mM sodium pyruvate (Sigma, S8636). X63-Ag8.653 cells were harvested by rinsing off the petri dish, centrifuged for 5 min at 1200 rpm in a Heraeus Megafuge 1.0 at 22° C., resuspended in 30 ml of 37° C. warm DMEM, counted with a 0.0025 mm² glass counting chamber (0,100 mm depth; Burker, Labor Optik) and centrifuged again for 5 min at 1200 rpm in a Heraeus Megafuge 1.0 at 22° C. Splenocytes and myeloma cells were mixed at a ratio of 2.5:1, centrifuged for 5 min at 1200 rpm and fused by resuspending and incubating for 90 sec at 37° C. the cells in 1 ml of polyethylenglycol (PEG) 1450 (50% w/v solution in PBS; Sigma, P7181). After 90 sec, the cell suspension was diluted stepwise with 1 ml of 37° C. warm DMEM, followed by 5 ml of 37° C. warm DMEM and followed again by 10 ml of 37° C. warm DMEM and was then incubated at 37° C. for 5 min. Cells were centrifuged for 5 min at 1200 rpm in a Heraeus Megafuge 1.0 at 22° C. and were resuspended in DMEM+10% HyClone Fetal Clone I (Thermo Scientific, SH30080.03)+2 mM Glutamax+100 units/ml Penicillin/0.1 mg/ml Streptomycin+1 mM sodium pyruvate+5% BM Condimed H1 Hybridoma Cloning Supplement (Roche, 11088947001)+0.1 mM hypoxanthine/0.4 μM aminopterin/16 μM thymidine (provided as HAT 50× stock; Life Technologies, 21060-017). 10⁵ cells per well were seeded onto 96-well petri-dishes (TC Microwell 96F, Nunc, 167000). Cells were grown for 7 days at 37° C. in a 5% CO₂ atmosphere and the supernatants were tested for the presence of carboxymethyl PP2Ac specific IgG antibodies by immunoblotting and ELISA of above-described yeast cell lysates (FIG. 2).

Specifically, the supernatants were first tested by immunoblotting against SDS-PAGE separated yeast BY4741 wildtype strain lysate to determine the presence of PP2Ac specific IgG antibodies. Secondly, positive hybridoma supernatants were tested similarly against SDS-PAGE separated lysates from the BY4741 ppe1Δ strain or the BY4741 ppm1Δ strain to determine the presence of antibodies which are specific for carboxymethylated PP2Ac (FIG. 2). The immunoblotting was performed as described above for the screening of polyclonal antibody sera except that after blocking, the blots were incubated with undiluted supernatants for 150 minutes at 22° C. and bound antibodies were visualized by enhanced chemoluminescence with Western Lightning Plus-ECL reagents (PerkinElmer, NEL102001EA).

In parallel to the immunoblotting screening, hybridoma supernatants were tested for the presence of TPDYFL-CH3 (SEQ ID NO:1) specific IgG antibodies by ELISA. Specifically, flat-bottom Nunc-Immuno Medisorp 96-well ELISA plates (Thermo; 467320) were coated over night at 4° C. with 50 μl of either a peptide of the sequence C(βA)TPDYFL (SEQ ID NO:48) at a concentration of 3 μg/ml in 10 mM sodium phosphate buffer pH 7.0 or with a peptide of the sequence C(βA)TPDYFL-CH₃ (SEQ ID NO:48) at a concentration of 3 μg/ml in 10 mM sodium phosphate buffer pH 7.0. The wells were washed once with PBS, blocked with 180 μl of 2% bovine serum albumin (Sigma, A9647) in PBS for 1 hour at RT and washed again once with PBS. The wells were incubated with 50 μl of undiluted splenic fusion hybridoma supernatants for 2 hours at RT, washed 3 times with PBS, incubated for 1 hour at RT with 50 μl peroxidase-conjugated AffiniPure goat anti-mouse IgG Fcγ fragment specific (Jackson ImmunoResearch, 115-035-008) secondary antibody diluted 1:10,000 in PBS and washed 3 times with PBS. Bound antibodies were detected colorimetrically by addition of 50 μl of 33 μg/ml 3′,5′,5′,5′-tetramethylbenzidine (TMB) (Sigma, T2885) in 0.1 M sodium acetate pH 6.0 and 0.01% H₂O₂ (Sigma, H1009). The colorimetric reaction was stopped after 28 min by addition of 50 μl 0.5 M H₂SO₄, and absorbance was measured at 450 nm with a Perkin Elmer Wallac Victor 2 1420 Multilabel Counter.

It was found by Western blotting that the supernatant of hybridoma mix clones 7G8, 7C10, 8F1 and 3A4 detected PP2Ac only in the BY4741 ppe1Δ strain that contains high levels of carboxymethylated PP2Ac subunit but not in the BY4741 ppm1Δ strain that lacks carboxymethylated PP2Ac (FIG. 2).

Furthermore, it was found by ELISA that supernatants of hybridoma mix clones 5A4, 5A5, 5H8, 6F12, 6H11, 7C10, 7G1, 7G8 and 8F1 detected the carboxymethylated peptide (C(βA)TPDYFL-CH₃) (SEQ ID NO:48) but did not detect the non-carboxymethylated peptide (C(βA)TPDYFL) (SEQ ID NO:48).

Those results indicated that mix clones 7G8, 7C10, 8F1, 3A4, 5A4, 5A5, 5H8, 6F12, 6H11 and 7G1 were specific for the carboxymethylated PP2Ac. Since the specificity of mix clone 7C10 was determined by Western blotting and ELISA, 7C10 was selected for further monoclonalization.

Generation of the Single Clone 7C10-C5

Mix clone 7C10 was monoclonalized and single clone 7C10-C5 was isolated. Specifically, hybridoma cells growing in tissue culture 96-well containing supernatant that was tested positive for the presence of antibodies specific for carboxymethylated PP2Ac were resuspended in DMEM+10% HyClone Fetal Clone I (Thermo Scientific, SH30080.03)+2 mM Glutamax +100 units/ml Penicillin/0.1 mg/ml Streptomycin+1 mM sodium pyruvate+5% BM Condimed H1 Hybridoma Cloning Supplement (Roche, 11088947001)+0.1 mM hypoxanthine/0.4 μM aminopterin/16 μM thymidine (referred to as “Hybridoma growth medium”) and counted with a 0.0025 mm² glass counting chamber (0,100 mm depth; Bürker, Labor Optik). The appropriate volume of cell suspension was diluted in 30 ml of Hybridoma growth medium to yield a concentration of 1 cell in 200 μl of Hybridoma growth medium, and 300 μl of cell suspension per well were seeded onto 96-well petri-dishes (TC Microwell 96F, Nunc, 167000). Cells were grown for 7 days at 37° C. in a 5% CO₂ atmosphere and the supernatants were tested for the presence of anti-carboxymethylated PP2Ac subunit IgG antibodies by immunoblotting against lysates from the BY4741 ppe1Δ strain as described above for the screening of the hybridoma supernatants. Wells containing supernatant that was tested positive for the presence of anti-carboxymethylated PP2Ac antibodies were further examined under the microscope for the number of hybridoma clones growing. One well with a single clone growing was selected for expansion and further propagation to obtain the 7C10-C5 monoclonal antibody.

### Example 2. Characterization of the Binding Specificity of the 7C10-C5 Monoclonal Antibody

Confirmation of the Carboxymethylation Specificity of 7C10-C5

7C10-C5 Western Blotting with Yeast Strain Lysates

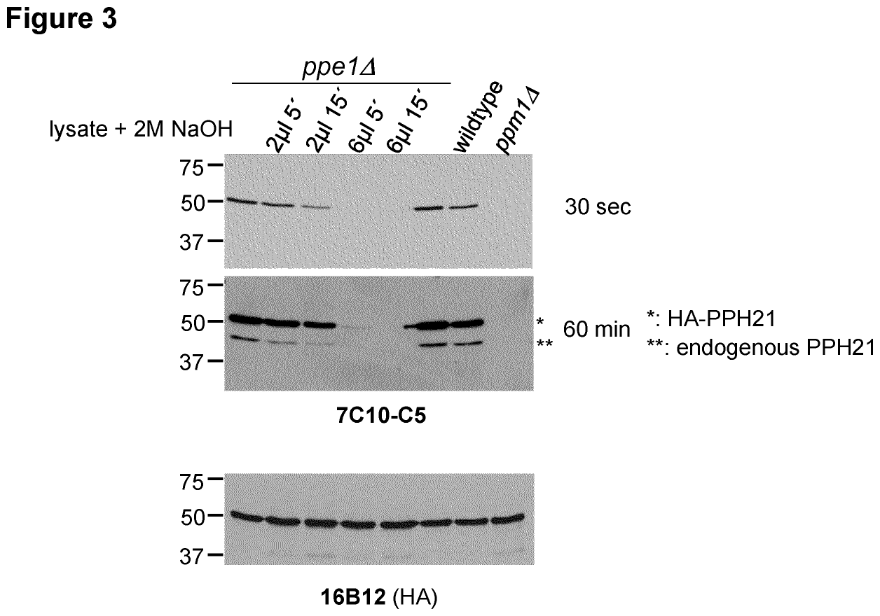

The carboxymethyl-PP2Ac specificity of the 7C10-C5 monoclonal antibody was confirmed by Western blot analysis of lysates from the wildtype BY4741 strain, the BY4741 ppe1Δ strain and the BY4741 ppm1Δ strain. Furthermore, lysates from the BY4741 ppe1Δ strain were analyzed in which the methylation of the C-terminal carboxyl group of PP2Ac was chemically removed (hydrolyzed) by NaOH treatment (FIG. 3).

Specifically, in one assay 50 μl of protein lysate corresponding to 180 μg of protein from the BY4741 ppe1Δ strain was incubated with 2 μl of 2 M NaOH, resulting in a pH ˜9-10 as determined with pH-indicator paper pH 1-14 (Merck, 1.10232.0001) for either 5 min or 15 min on ice. Lysates were then neutralized by addition of 4 μl 1 M HCl plus 14 μl 1 M Tris pH 6.8 to pH ˜7 as determined with pH-indicator paper pH 1-14 and boiled with Laemmli buffer. In a further assay, said protein lysate was incubated with 6 μl of 2M NaOH, resulting in a pH >11 as determined with pH-indicator paper pH 1-14 for either 5 min or 15 min on ice. Lysates were then neutralized by addition of 12 μl 1 M HCl plus 36 μl 1 M Tris pH 6.8 to pH ˜7 as determined with pH-indicator paper pH 1-14 and boiled with Laemmli buffer.

In particular, it was confirmed that the 7C10-C5 antibody only detected carboxymethylated PP2Ac (ppe1Δ strain) but not non-carboxymethylated PP2Ac (ppm1Δ strain; ppe1Δ strain +NaOH, 6 μl; FIG. 3).

7C10-C5 ELISA with Peptides

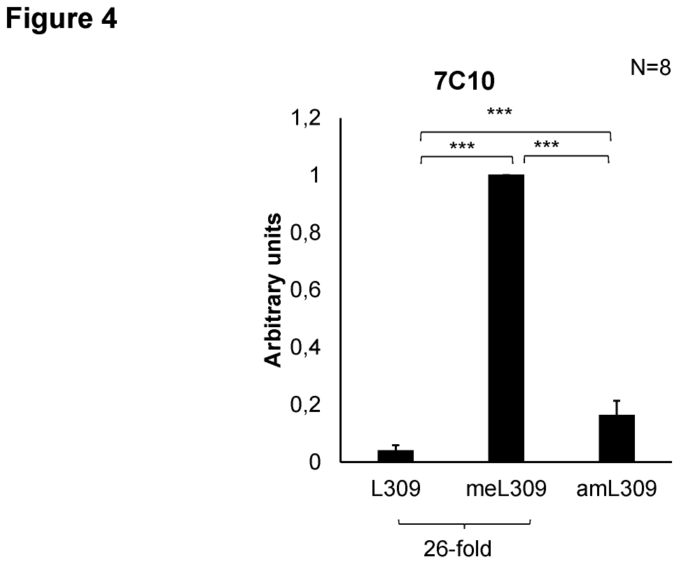

7C10-C5 was tested for its specificity to the unmodified, methylated or amidated carboxyl group of Leu³⁰⁹ at the carboxy-terminus of PP2Ac by ELISA with 3 different undecapeptides (11-mers) (FIG. 4). Methylated and amidated peptides were compared because at physiological pH the α-carboxyl-group of Leu³⁰⁹ is negatively charged and either the methylation or amidation of the α-carboxyl-group neutralizes its negative charge. This allowed the inventors to discriminate between a specificity for the amidated charge-neutralized PP2Ac C-terminus or the carboxymethylated charge-neutralized PP2Ac C-terminus.

Specifically, flat-bottom Nunc-Immuno Medisorp 96-well ELISA plates (Thermo; 467320) were coated o/n at 4° C. with 50 μl of either a peptide of the sequence ac-HVTRRTPDYFL-OH (L309; unmodified) (SEQ ID NO:18) at a concentration of 2 μg/ml, a peptide of the sequence ac-HVTRRTPDYFL-CH₃ (meL309; methylated) (SEQ ID NO:18) at a concentration of 2 μg/ml in TBS (137 mM NaCl (AppliChem, #131659, 2.7 mM KCl (AppliChem, #131494), 24.8 mM Tris (AppliChem, #A1086), pH 7.4 with HCl) or a peptide of the sequence ac-HVTRRTPDYFL-NH₂ (amL309; amidated) (SEQ ID NO:18) at a concentration of 2 μg/ml in TBS. Of note, the “ac-” in the peptides described here and in other Examples refers to an acetylated N-terminus. Said acetylation neutralizes the positive charge at the N-terminus, and the free charged N-terminus is thereby removed.

Then, the wells were washed once with TBS, blocked with 200 μl of 2% bovine serum albumin (BSA) (Sigma, A9647) in TBS for 1 hour at RT and washed again once with TBS. The wells were incubated with 50 μl of hybridoma supernatants 7C10-C5, 1:50 dilution in of 1% BSA in TBS for 1 hour at RT, washed 3 times with TBS, incubated for 1 hour at RT with 50 μl peroxidase-conjugated AffiniPure goat anti-mouse IgG Fcγ fragment specific (Jackson ImmunoResearch, 115-035-008) secondary antibody diluted 1:10,000 in TBS and washed 3 times with TBS. Bound antibodies were detected colorimetrically by addition of 50 μl of 33 μg/ml 3′,5′,5′,5′-tetramethylbenzidine (TMB) (Sigma, T2885) in 0.1 M sodium acetate pH 6.0 and 0.01% H₂O₂ (Sigma, H1009). The colorimetric reaction was stopped after 10 min by addition of 50 μl 0.5 M H₂SO₄, and measured at 450 nm and 560 nm with a Perkin Elmer VICTOR® Nivo™ microplate reader. The results from 560 nm read were subtracted from the 450 nm read.

It was found that the 7C10-C5 antibody detects the corresponding α-carboxymethylated PP2Ac peptide with 26-fold higher signal intensity than the non-methylated peptide. Furthermore, the peptide with the methylated α-carboxyl-group was recognized with 6-fold higher signal intensity than the amidated peptide (FIG. 4) indicating high specificity of 7C10-C5 for the methylated α-carboxyl-group of Leu³⁰⁹. The preference of the 7C10-C5 monoclonal antibody for the methylated α-carboxyl-group differentiates it from another monoclonal antibody, termed 4D9, that was raised against an amidated (and not methylated) C-terminal PP2Ac peptide (299-309) (Tolstykh (2000), Embo J 19, 5682-5691). Said 4D9 antibody also binds amidated PP2Ac and is not specific for the carboxymethylated PP2Ac.

Preparation of Mammalian Cell Lysates for Immunoblot Analysis

NIH3T3 mouse fibroblasts and human embryonic kidney cells (HEK293Trex) were grown in DMEM+10% FCS+2 mM L-glutamine (Sigma, G2150)+100 units/ml Penicillin/0.1 mg/ml Streptomycin at 37° C. in a 7.5% CO₂ atmosphere. HAP1 wild type and HAP1 Lcmt-1-KO cells (Leucine carboxyl methyltransferase 1 deleted by CRISPR/Cas9; Horizon Discovery, #C631 and #HZGHC004373c001) were grown in Iscove's Modified Dulbecco's Medium (IMDM, Thermo Fischer Scientific, Life Technologies #12440053) supplemented with 10% (v/v) Fetal Calf Serum (FCS) (Sigma #F7524, lot 104M3333), GlutaMAX™ (Thermo Fischer Scientific, Life Technologies #35050-38, lot 1895829), and Penicillin-Streptomycin solution (Sigma, #P4333, lot 125M4781V) in a 5% CO₂ atmosphere. Of note, HAP1 is a near-haploid human cell line that was derived from the male chronic myelogenous leukemia (CML) cell line KBM-7 (Carette (2011), Nature 477(7364):340-3). For cell lysis, exponentially growing cells were washed once with ice-cold PBS, once with ice-cold IP-Wash buffer (20 mmol/l Tris pH 8.0, 135 mmol/l NaCl, 10% glycerol) and lysed on the petri-dish in ice-cold IP-Lyse buffer (18 mmol/l Tris pH 8.0, 122 mmol/l NaCl, 9% glycerol, 1% w/v NP-40) for 20 min at 4° C. rocking. Lysed cells were transferred to Eppendorf 1.5 ml tubes and centrifuged for 15 min at 14,000 rpm at 4° C. in a Beckman & Coulter Microfuge 18 centrifuge. The protein concentration was determined with the Protein Assay Dye Reagent (Biorad, 500-0006) and measurement of absorbance at 595 nm in a Hitachi U-2000 spectrophotometer. For SDS-PAGE and Western blotting, protein loading buffer was added to a final concentration of 0.112 M dithiothreitol, 2.22% w/v SDS and 11.1% glycerol, and protein samples were denatured by incubation for 5 min at 95° C.

7C10-C5 Western Blotting with Mammalian Cells

To confirm the ELISA results with methylated and non-methylated full-length PP2Ac, the carboxy-terminal methyl group was chemically removed from cellular PP2Ac by treating lysates of two different human cell lines, HAP1, and HEK293Trex (HEK) with NaOH as described in Favre (1994), J Biol Chem 269, 16311-16317. In brief, 100 μl of HAP1 wild type or HEK293T cell lysates corresponding to 200 μg of protein was mixed with 1M NaOH to a final concentration of 0.2M and incubated for 10 min at RT. The reaction was neutralized by adding HCL to a final concentration of 0.2M and diluted to 200 μl with IP Lyse. The control reaction was treated with preneutralization solution (0.2M NaOH and 0.2M HCL) for 10 min at room temperature and diluted to 200 μl with IP Lyse. For immunoblot analysis, protein loading buffer was added to the protein samples and proteins were denatured by incubation at 95° C. for 5 min.

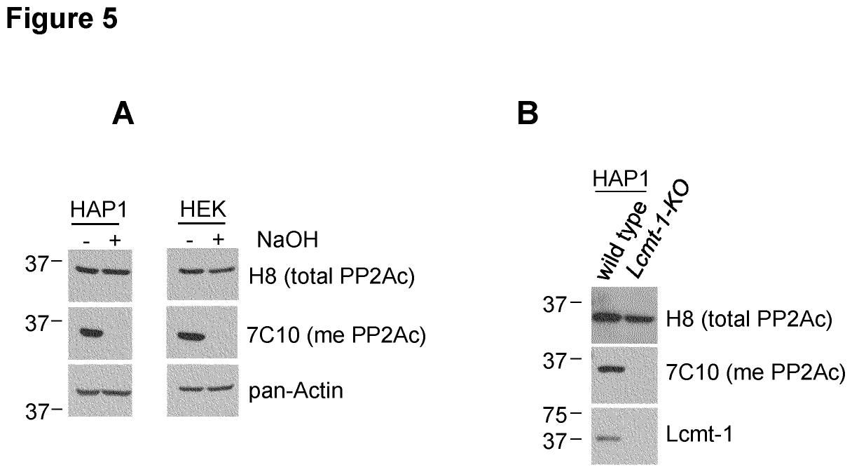

The methyl-PP2Ac specific antibody 7C10-C5 only detected methylated PP2Ac in the untreated cell lysates but not in the NaOH treated lysates, whereas actin and total PP2Ac levels (detected by the anti-total PP2Ac antibody H8) remained unchanged by the NaOH treatment (FIG. 5A).

It has been reported previously that the deletion of the PP2A methyltransferase LCMT-1 in mouse embryonic fibroblasts decreases the carboxymethylation of PP2Ac by >95% (Hwang (2016), J Biol Chem 291, 21008-21019). In agreement with the results of the NaOH experiments, no carboxymethylated PP2Ac specific signals were detected with the 7C10-C5 antibody in HAP1 cells that lack the PP2A methyltransferase LCMT-1 (Lcmt-1-KO), further confirming the specificity of the 7C10-C5 antibody for carboxymethylated PP2Ac (FIG. 5B).

No Impairment of 7C10-C5 Binding Specificity by Co-Cocurrent Phosphorylation of Tyrosine 307 or Threonine 304 of PP2Ac

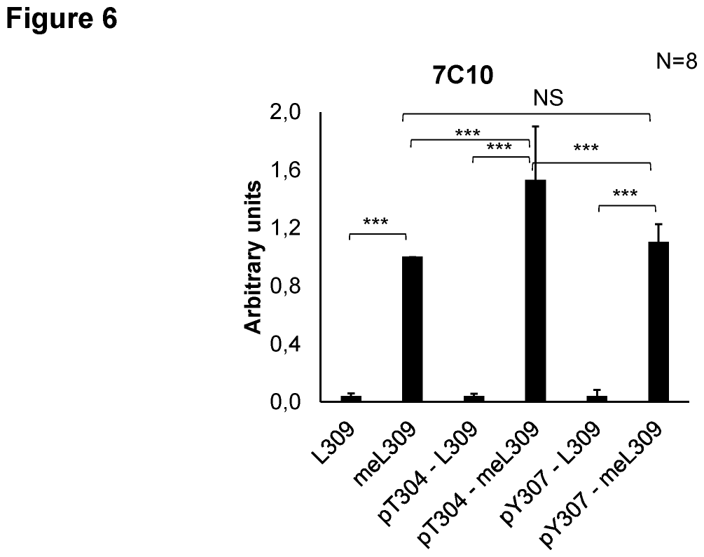

Additional post-translational modifications to the C-terminus of PP2Ac include two reported phosphorylation sites at threonine 304 (Thr³⁰⁴) and tyrosine 307 (Tyr³⁰⁷). To test whether the carboxymethyl-specific binding of 7C10-C5 is affected by these modifications, ELISAs were performed with 6 different undecapeptides (11-mers). (FIG. 6). Specifically, flat-bottom Nunc-Immuno Medisorp 96-well ELISA plates (Thermo; 467320) were coated o/n at 4° C. with 50 μl of either a peptide of the sequence ac-HVTRRTPDYFL-OH (L309; unmodified) (SEQ ID NO:18) at a concentration of 2 μg/ml, a peptide of the sequence ac-HVTRRTPDYFL-CH₃ (meL309; C-terminus carboxymethylated) (SEQ ID NO:18) at a concentration of 2 μg/ml in TBS (137 mM NaCl (AppliChem, #131659, 2.7 mM KCl (AppliChem, #131494.), 24.8 mM Tris (AppliChem, #A1086), pH 7.4 with HCl), with a peptide of the sequence ac-HVTRRp̅T̅PDYFL-OH (pT304-L309; non-carboxymethylated+phosphorylated at T304) (SEQ ID NO:116) at a concentration of 2 μg/ml in TBS, with a peptide of the sequence ac-HVTRRpTPDYFL-CH₃ (pT304-meL309; C-terminus carboxymethylated+phosphorylated at T304) (SEQ ID NO:116) at a concentration of 2 μg/ml in TBS, with a peptide of the sequence ac-HVTRRTPDpYFL-OH (pY307-L309; non-carboxymethylated+phosphorylated at Y307) (SEQ ID NO:117) at a concentration of 2 μg/ml in TBS, or with a peptide of the sequence ac-HVTRRTPDpYFL-CH₃ (pY307-meL309; C-terminus carboxymethylated+phosphorylated at Y307) (SEQ ID NO:117) at a concentration of 2 μg/ml in TBS.

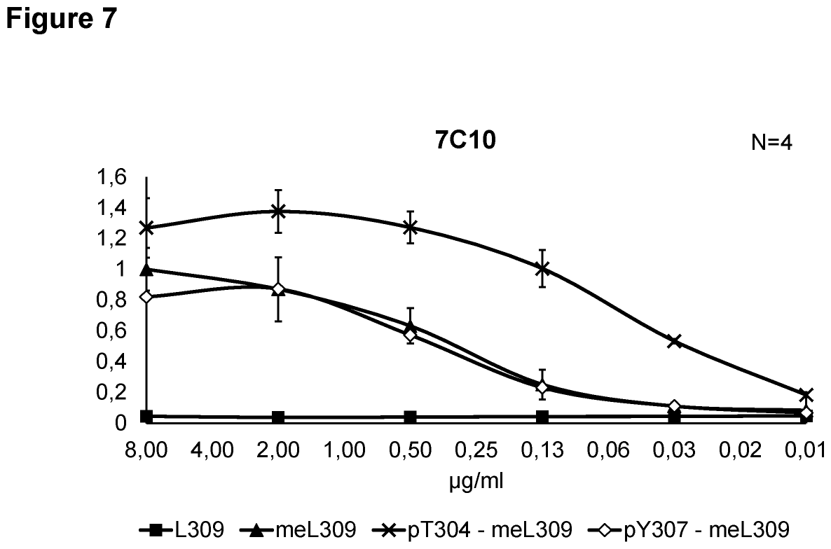

To further determine if the binding of the 7C10-C5 antibody depends on the peptide concentration, ELISAs were performed with 4 different undecapeptides. Specifically, flat-bottom Nunc-Immuno Medisorp 96-well ELISA plates (Thermo; 467320) were coated o/n at 4° C. with 50 μl of either a peptide of the sequence ac-HVTRRTPDYFL-OH (L309) (SEQ ID NO:18) at a concentration of 8, 2, 0.5, 0.125, 0.03125 or 0.0078125 μg/ml in TBS (137 mM NaCl (AppliChem, #131659, 2.7 mM KCl (AppliChem, #131494), 24.8 mM Tris (AppliChem, #A1086), pH 7.4 with HCl), a peptide of the sequence ac-HVTRRTPDYFL-CH₃ (meL309) (SEQ ID NO:18) at a concentration of 8, 2, 0.5, 0.125, 0.03125 or 0.0078125 μg/ml in TBS, with a peptide of the sequence ac-HVTRRpTPDYFL-CH₃ (pT304-meL309) (SEQ ID NO:116) at a concentration of 8, 2, 0.5, 0.125, 0.03125 or 0.0078125 μg/ml in TBS or with a peptide of the sequence ac-HVTRRTPDpYFL-CH₃ (pY307-meL309) (SEQ ID NO:117) at a concentration of 8, 2, 0.5, 0.125, 0.03125 or 0.0078125 μg/ml in TBS (FIG. 7).

The wells were washed once with TBS, blocked with 200 μl of 2% bovine serum albumin (BSA) (Sigma, A9647) in TBS for 1 hour at RT and washed again once with TBS. The wells were incubated with 50 μl of 7C10-C5 hybridoma supernatant 1:50 diluted in 1% BSA in TBS for 1 hour at RT, washed 3 times with TBS, incubated for 1 hour at RT with 50 μl peroxidase-conjugated AffiniPure goat anti-mouse IgG Fcγ fragment specific (Jackson ImmunoResearch, 115-035-008) secondary antibody diluted 1:10,000 in TBS and washed 3 times with TBS. Bound antibodies were detected colorimetrically by addition of 50 μl of 33 μg/ml 3′,5′,5′,5′-tetramethylbenzidine (TMB) (Sigma, T2885) in 0.1 M sodium acetate pH 6.0 and 0.01% H₂O₂ (Sigma, H1009). The colorimetric reaction was stopped after 10 min by addition of 50 μl 0.5 M H₂SO₄, and absorbance was measured at 450 nm and 560 nm with a Perkin Elmer VICTOR® Nivo™ microplate reader. The results from 560 nm read were subtracted from the 450 nm read.

It was found that the 7C10-C5 antibody detects the α-carboxymethylated PP2Ac peptide with 1.6-fold higher signal intensity, when Thr³⁰⁴ is phosphorylated. (FIG. 6). At lower peptide concentrations the detection difference between the carboxymethylated and the carboxymethylated+Thr³⁰⁴ phosphorylated peptide became more apparent and increased to 4-fold suggesting an increased affinity of 7C10-C5 for carboxymethyl-PP2Ac when Thr³⁰⁴ is concurrently phosphorylated (FIG. 7). Of note, the data also demonstrate that the specific binding of the 7C10-C5 antibody to the carboxymethylated C-terminus of PP2Ac (Leu³⁰⁹) is not impaired by concurrent phosphorylation of the nearby threonine (Thr³⁰⁴). Furthermore, the specific binding of the 7C10-C5 antibody to the carboxymethylated C-terminus of PP2Ac (Leu³⁰⁹) was not significantly altered by concurrent phosphorylation of the nearby tyrosine (Tyr³⁰⁷) (FIG. 6+7). These results indicate that detection of a α-carboxymethylated peptide from the PP2Ac C-terminus is not impaired by the concurrent phosphorylation of threonine at position 304 or tyrosine at position 307, but rather slightly increased when Thr³⁰⁴ is simultaneously phosphorylated.

No Cross-Reactivity of 7C10-C5 with PP4c or PP6c

It was found that the monoclonal antibody 7C10-C5 detects α-carboxymethylated PP2Ac with at least 10-fold higher signal intensity than α-carboxymethylated PP4c or α-carboxymethylated PP6c. In particular, the monoclonal antibody 7C10-C5 detected α-carboxymethylated PP2Ac with at least 16-fold higher signal intensity than α-carboxymethylated PP6c.

7C10-C5 Western Blot with Mammalian Cells

The catalytic subunits of protein phosphatase 4 (PP4c) and 6 (PP6c) are closely related to PP2Ac with approximately 50% sequence identity. PP2Ac, PP4c and PP6c share the C-terminal DYFL or YFL motif, respectively (FIG. 8A). Therefore, a potential cross-reactivity of 7C10-C5 with α-carboxymethylated PP4c and/or PP6c was examined and compared to another anti-carboxymethyl-PP2Ac antibody 2A10. 2A10 is a carboxymethyl-PP2Ac specific antibody that has been raised against a larger C-terminal PP2Ac peptide (amino acids 302-309, carboxymethylated Leu³⁰⁹) and that has been shown before to cross-react with α-carboxymethylated PP4c (Lee (2014), Biochem Biophys Res Commun 452, 42-47). HA tagged human PP2Ac a, mouse HA PP4c or mouse HA PP6c from NIH3T3 mouse fibroblasts stably expressing these HA-tagged proteins were immunoprecipitated. The precipitated proteins were then analyzed by immunoblotting with anti-carboxymethyl-PP2Ac antibodies 7C10-C5 or 2A10.

Specifically, 500 μl corresponding to 1 mg of whole cell protein lysates of NIH3T3 mouse fibroblasts were incubated with anti-hemagglutinin (HA) antibody (clone 12CA5) crosslinked to BSA-coated protein A-Sepharose beads (GE-Healthcare) to immunoprecipitate HA-epitope tagged PP2Ac, PP4c or PP6c for 1 h. The Immune complexes were washed once with 1 ml with IP-Lyse, and 3 times with 1 ml with Tris-buffered saline (25 mM Tris, 135 mM NaCl, 2.6 mM KCL pH=7.4 with HCl). For immunoblot analysis protein loading buffer was added to the immunoprecipitate and proteins were denatured by incubation at 95° C. for 5 min. IP-Lyse and protein loading buffers were as described above for the preparation of mammalian cell lysates for immunoblot analysis.

It was found that 7C10-C5 did not detect PP4c or PP6c, and thus is specific for carboxymethylated PP2Ac, whereas clone 2A10 cross-reacted with PP4c or PP2Ac. Quantification of the signal strength revealed that 2A10 bound PP4ac with about 25% of the strength compared to PP2Ac, and PP6c with about 10% of the strength compared to PP2Ac (FIG. 8B). In other words, 2A10 might, for example, yield a similar signal with 8 μg/ml carboxymethylated PP4c than with 2 μg/ml carboxymethylated PP2Ac.

Since no discernable PP4c or PP6c signal was obtained with the 7C10-C5 antibody, the data suggest that 7C10-C5 might bind carboxymethylated PP2Ac 100-fold or even 500-fold stronger than carboxymethylated PP4c or PP6c. Consistent with what we observed for the PP2Ac, both antibodies recognized their targets in a carboxymethylation dependent manner because neither 7C10-C5 nor 2A10 detected PP2Ac or PP4Ac when the carboxymethylation was chemically removed by NaOH treatment (FIG. 8C). Of note, PP2Ac might be detected as a double band (FIG. 8B; see e.g. Ogris (1997). Oncogene 15, 911-917). Furthermore, the used mouse fibroblasts also express endogenous PP2Ac (the lower band in the HA-PP2A lysate in FIG. 8C) which is downregulated by forced expression of HA-PP2Ac. In consequence, the 7C10-C5 and 2A10 antibodies detected the endogenous PP2Ac stronger in the HA-PP4 lysate than the HA-PP2A lysate. However, 7C10-C5 did not detect a signal (in contrast to 2A10) with the immunoprecipitated PP4c, demonstrating that the detected band in the HA-PP4 lysate is not PP4c (but endogenous PP2Ac). In conclusion, these experiments demonstrate that only 7C10-C5 binds specifically carboxymethylated PP2Ac since 2A10 also substantially binds carboxymethylated PP4c and PP6c.

7C10-C5 ELISA with Peptides

To further characterize the carboxymethyl-PP2Ac specificity of monoclonal antibody 7C10-C5 ELISAs were performed on carboxymethylated versus non-methylated PP2Ac, PP4c and PP6c C-terminal undecapeptides.

Specifically, flat-bottom Nunc-Immuno Medisorp 96-well ELISA plates (Thermo; 467320) were coated o/n at 4° C. with 50 μl of either a peptide of the sequence ac-HVTRRTPDYFL-OH (L309) (SEQ ID NO:18) at a concentration of 2 μg/ml in TBS (137 mM NaCl (AppliChem, #131659, 2.7 mM KCl (AppliChem, #131494), 24.8 mM Tris (AppliChem, #A1086), pH 7.4 with HCl), a peptide of the sequence ac-HVTRRTPDYFL-CH₃ (meL309) (SEQ ID NO:18) 2 μg/ml in TBS, with a peptide of the sequence (ac-PSKKPVADYFL (L307) (SEQ ID NO:20) at a concentration of 2 μg/ml in TBS or with a peptide of the sequence ac-PSKKPVADYFL-CH3 (meL307) (SEQ ID NO:20) at a concentration of 2 μg/ml in TBS, with a peptide of the sequence ac-IPPRTTTPYFL (L305) (SEQ ID NO:22) at a concentration of 2 μg/ml in TBS or with a peptide of the sequence ac-IPPRTTTPYFL-CH₃ (meL305) (SEQ ID NO:22) at a concentration of 2 μg/ml in TBS. The wells were washed once with TBS, blocked with 200 μl of 2% bovine serum albumin (BSA) (Sigma, A9647) in TBS for 1 hour at RT and washed again once with TBS. The wells were incubated with 50 μl of hybridoma supernatant 7C10-C5, 1:50 diluted in TBS with 1% BSA for 1 hour at RT, washed 3 times with TBS, incubated for 1 hour at RT with 50 μl peroxidase-conjugated AffiniPure goat anti-mouse IgG Fcγ fragment specific (Jackson ImmunoResearch, 115-035-008) secondary antibody diluted 1:10,000 in TBS and washed 3 times with TBS. Bound antibodies were detected colorimetrically by addition of 50 μl of 33 μg/ml 3′,5′,5′,5′-tetramethylbenzidine (TMB) (Sigma, T2885) in 0.1 M sodium acetate pH 6.0 and 0.01% H₂O₂ (Sigma, H1009). The colorimetric reaction was stopped after 10 min by addition of 50 μl 0.5 M H₂SO₄, and absorbance was measured at 450 nm and 560 nm with a Perkin Elmer VICTOR® Nivo™ microplate reader. The results from 560 nm read were subtracted from the 450 nm read.

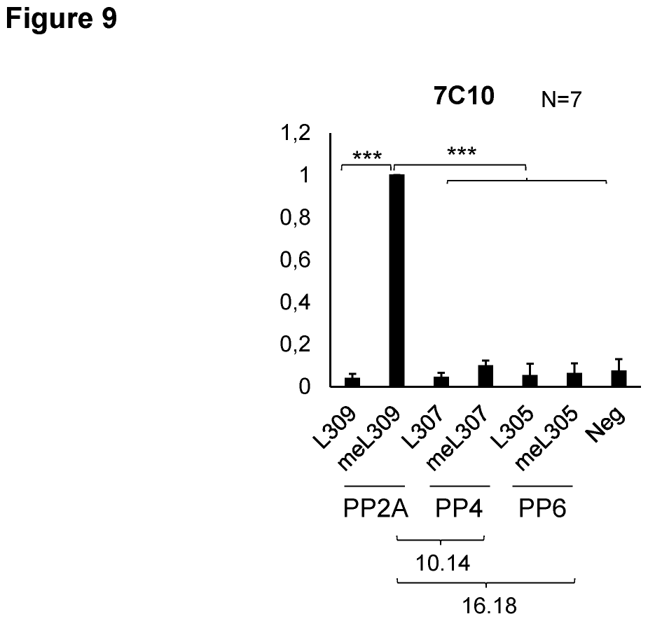

These experiments further confirmed the high specificity of 7C10-C5 for carboxymethylated PP2Ac (FIG. 9). In the ELISA, 7C10-C5 detected the corresponding α-carboxymethylated PP2Ac peptide with at least 10-fold higher signal intensity than the α-carboxymethylated PP4c and PP6c peptides, whose detection signals were close to the negative control signals. In particular, in the ELISA, 7C10-C5 detected the corresponding α-carboxymethylated PP2Ac peptide with at least 16-fold higher signal intensity than the α-carboxymethylated PP6c peptide. No significant difference was observed for the carboxymethylated PP4c or PP6c peptides compared to the non-carboxymethylated PP4c or PP6c peptides, respectively.

### Example 3. Structural Characterization of the 7C10-C5 Monoclonal Antibody

Isotyping of the 7C10-C5 Monoclonal Antibody

The heavy chain and light chain isotypes of clone 7C10-C5 were determined with the ImmunoPure Monoclonal Antibody Isotyping Kit II (Pierce, 37502) by following the protocol for antigen-independent procedure for isotype determination as instructed by the manufacturer's manual on flat-bottom Nunc-Immuno Medisorp 96-well ELISA plates (Thermo; 467320). Instead of the alkaline phosphatase-conjugated goat-anti-rabbit IgG secondary antibody provided in the Isotyping kit, peroxidase-conjugated AffiniPure goat anti-rabbit IgG Fc fragment specific (Jackson ImmunoResearch, 111-035-008) secondary antibody diluted 1:10,000 was used. Instead of the PNPP substrate solution provided in the Isotyping kit, 50 μl of 33 μg/ml TMB (Sigma, T2885) in 0.1 M sodium acetate pH 6.0 and 0.01% 14202 (Sigma, H1009) was used for the colorimetric detection. The colorimetric reaction was stopped by addition of 50 μl 0.5 M H₂SO₄, and absorbance was measured at 450 nm with a Perkin Elmer Wallac Victor 3 1420 Multilabel Counter.

It was determined that the isotype of the 7C10-C5 monoclonal antibody is IgG1.

Determination of the 7C10-C5 Heavy and Light Chain Variable Regions and Complementary Determining Regions (CDRs)

RNA was isolated from 5×10⁷ cells of the carboxymethyl-PP2Ac specific hybridoma cell line clone 7C10-C5 using the RNeasy Mini Kit (Qiagen) following the protocol as instructed by the manufacturer's manual. The purified RNA was resuspended in 50 μl RNase free water and 800 ng RNA was taken for cDNA synthesis using the Accu Script 1^{st} Strand cDNA Synthesis Kit (Agilent) in a 20 μl reaction mix using random hexamers following the protocol as instructed by the manufacturer's manual. For cloning of the variable region of the antibody, the heavy and light chain variable regions were amplified by Polymerase chain reaction (PCR). Specifically, a forward primer mix binding to the V(D)J leader sequences of the heavy chain and a reverse primer binding to the constant region of the heavy chain were used to amplify the heavy chain variable region, and a forward primer mix binding to the V(D)J leader sequences of the light chain and a reverse primer binding to the constant region of the light chain were used to amplify the light chain variable region as demonstrated in von Boehmer (2016), Nat Protoc 11, 1908-1923. The used primers (ordered from SigmaAldrich-Merck) are shown in Table 1:

TABLE 1
Primer sets for the PCR of the heavy and light chain regions

| FORWARD HEAVY 5′→3′ | | FORWARD LIGHT 5′→3′ | |
|---|---|---|---|
| 1MFH_I | AGGAACTGCAGGTGTCC (SEQ ID | 1mFK_I | RGTGCAGATTTTCAGCTTCCTGCT |
| | NO: 53) | | (SEQ ID NO: 65) |
| 1MFH_II | CAGCTACAGGTGTCCACTCC | 1mFK_II | TGGACATGAGGGCYCCTGCTCAGT |
| | (SEQ ID NO: 54) | | (SEQ ID NO: 66) |
| 1MFH_III | TGGCAGCARCAGCTACAGG (SEQ | 1mFK_III | CTSTGGTTGTCTGGTGTTGAYGGA |
| | ID NO: 55) | | (SEQ ID NO: 67) |
| 1MFH_IV | CTGCCTGGTGACATTCCCA (SEQ | 1mFK_IV | GTTGCTGCTGCTGTGGCTTACA (SEQ |
| | ID NO: 56) | | ID NO: 68) |
| 1MFH_V | CCAAGCTGTGTCCTGTC (SEQ ID | 1mFK_V | GTATCTGGTACCTGTGG (SEQ ID |
| | NO: 57) | | NO: 69) |
| 1MFH_VI | TTTTAAAAGGTGTCCAGKGT | 1mFK_VI | TGCCTGTTAGGCTGTTGGTGCT (SEQ |
| | (SEQ ID NO: 58) | | ID NO: 70) |
| 1MFH_VII | CCTGTCAGTAACTRCAGGTGTCC | 1mFK_VII | GCTCAGTTCCTTGGTCTCCTGTTGC |
| | (SEQ ID NO: 59) | | (SEQ ID NO: 71) |
| IMFH_VIII | TTTTAAAAGGGGTCCAGTGT | 1mFK_VIII | TGGGTGCTGCTGCTCTGGGT (SEQ ID |
| | (SEQ ID NO: 60) | | NO: 72) |
| 1MFH_IX | CGTTCCTGGTATCCTGTCT (SEQ | 1mFK_IX | CAGTTCCTGTTTCTGTTARTGCTCTGG |
| | IDNO: 61) | | (SEQ ID NO: 73) |
| 1MFH_X | ATGAAGTTGTGGYTRAACTGG | 1mFK_X | TGCTCTGGTTATATGGTGCTGATGGG |
| | (SEQ ID NO: 62) | | (SEQ ID NO: 74) |
| 1MFH_XI | TGTTGGGGCTKAAGTGGG (SEQ | | |
| | ID NO: 63) | | |
| REVERSE HEAVY 5′→3′ | | REVERSE LIGHT 5′→3′ | |
| 1MRG | AGAAGGTGTGCACACCGCTGGAC | 1mRK | ACTGAGGCACCTCCAGATGTT (SEQ |
| | (SEQ ID NO: 64) | | ID NO: 75) |

The PCR consisted of 2 μl cDNA from the 200 Accu Script reaction volume, 0.60 forward primer mix (10 μM stock) and 0.40 reverse primer (10 μM stock), 100 2× OneTaq® Quick-Load 2× Master Mix (New England BioLabs NEB, #M0482) and 7 μl nuclease free water (Sigma-Aldrich #W4502). Reactions were done in a Biometra TRIO thermocycler starting with a DNA denaturation step at 95° C. for 5 minutes followed by 35 cycles of denaturation of the DNA at 94° C. for 30 sec., annealing at 52° C. for 30 sec., and elongation at 72° C. for 1 minutes. A 10 minutes 72° C. step completed the reaction. The PCR products were separated on a 1.5% TAE (25 mM Tris (AppliChem #A1086), 0.114% Glacial Acetic Acid (AppliChem #A3701), 0.1 mM EDTA pH 8.0 (AppliChem #2937) agarose gel (Sigma, A9539) and stained with ethidium bromide (Sigma #E1510). Agarose gel slices containing the PCR products corresponding to the light chain variable fragments (about 350 bp) and the heavy chain variable fragments (about 400 bp) were cut out with a sterile scalpel blade and purified using a Wizard® DNA Clean Up Kit (Promega, #A9282), following the protocol as instructed by the manufacturer's manual, and eluted with 50 μl nuclease free water (Sigma-Aldrich, #W4502). The purified DNA fragments were cloned into the pJet 1.2 cloning vector following the protocol of the CloneJET PCR Cloning Kit (Thermo, #K1231). 2.5 μl of the ligation mixture was used for bacterial transformation using heat shock competent bacteria. E. coli HB101 cells were grown in Leuria Broth (LB) medium (1% (wt/vol) Tryptone (AppliChem #A1553), 0.5% (wt/vol) Yeast extract (AppliChem #A1552), 0.5% (wt/vol) NaCl (AppliChem #131659) to the early exponential phase (0D600 0.3-0.4) followed by centrifugation at 1000 g for 10 minutes at 4° C. The pellet was then resuspended at one-tenth of its volume in ice cold transformation and storage buffer (1% (wt/vol) Trypton, 0.5% (wt/vol) Yeast extract, 0.5% (wt/vol) NaCl, 10% (wt/vol) PEG (Mw 3350), 5% (vol/vol) 1M MgCl (Merk #105833), 5% (vol/vol) DMSO (added after autoclavation), and pH 6.5 HCl), aliquoted, and frozen at −20° C. 100 μl of this heat shock competent E. coli were thawed on ice, the DNA was added and the cells were kept on ice for another 30 minutes. Bacteria were then heat shocked at 42° C. for 1 minute, put on ice again for 10 minutes, resuspended in 500 μl LB medium without antibiotics for recovery, incubated at 37° C. for 30 minutes, plated out on LB agar plates (LB medium inclusive 1.5% Agar (AppliChem #A0949) with 0.1 mg/ml Ampicillin (GERBU, #1046) and stored over night at 37° C. Individual bacterial colonies were screened for inserted light or heavy chain DNA fragments by colony PCR as described in the CloneJET PCR Cloning Kit (Thermo, #K1231) with the enclosed pJet primers. Positive clones were subsequently inoculated in 5 ml LB with Ampicillin and grown over night at 37° C. Plasmid DNA was prepared with the Qiagen Miniprep Kit (Qiagen, #27106) as instructed by the manufacturer's manual, and after purification eluted with 50 μl nuclease free water (Sigma-Aldrich, #W4502). The DNA was prepared for sequencing as demanded by the sequencing company (LGC genomics). 10 μl with 100 ng/μ1 DNA was mixed with 4 μl primer (10 μM) using either the pJET1.2 forward sequencing primer (5′-CGACTCACTATAGGGAGAGCGGC-3′) (SEQ ID NO:76) or the pJET1.2 reverse sequencing primer (5′-AAGAACATCGATTTTCCATGGCAG-3′) (SEQ ID NO:77) to get sequences from both sides. The obtained sequences for the heavy and light variable fragments were checked with the V-Quest tool from the IMGT homepage (www.imgt.org; Brochet (2008), Nucleic Acids Res 36, W503-508; Giudicelli (2011), Cold Spring Harb Protoc 2011, 695-715).

Heavy and light chain variable regions were again cloned as a single cell variable fragment (scFv) as described below in Example 4. The sequences of the heavy and light chain variable regions of the 7C10-C5 were determined as described in Example 3 and Example 4. Although the sequence determined according to Example 3 could have also been used, the sequence of the scFv plasmid described in Example 4 below was actually used for annotating the heavy and light chain variable regions, i.e. the CDRs and FRs. Specifically, the obtained sequences for the heavy and light chain variable regions were checked with the V-Quest tool from the IMGT homepage (www.imgt.org) and position and length of the CDRs were additionally analyzed with the sequence annotation tool on the abysis hompage (http://www.abysis.org/) which uses inter alia the Chothia, Kabat, and IMGT numbering (Tables 2 and 3).

The nucleotide sequence of the 7C10-C5 heavy chain variable region was determined to be:

(SEQ ID NO: 39)
GATGTACAGCTTCAGGAGTCAGGACCTGGCCTCGTG
AAACCTTCTCAGTCTCTGTCTCTCACCTGCTCTGT
CACTGGCTACTCCATCACCAGTGGTTATTACTGGA
ACTGGATCCGGCAGTTTCCAGGAAACAAACTGGAA
TGGATGGGCTACATAAGCTACGACGGTAGCAATAA
CTACAACCCATCTCTCAAAAATCGAATCTCCATCA
CTCGTGACACATCTAAGAACCAGTTTTTCCTGAAG
TTGAATTCTGTGACTACTGAGGACACAGCTACATA
TTACTGTGCTGGACGGTTTGCTTACTGGGGCCAAG
GGACTCTGGTCACTGTCTCTGCA;

the amino acid sequence of the 7C10-C5 heavy chain variable region was determined to be:

(SEQ ID NO: 16)
DVQLQESGPGLVKPSQSLSLTCSVTGYSITSGYYWNWTRQ
FPGNKLEWMGYISYDGSNNYNPSLKNRISITRDTSKNQFF
LKLNSVTTEDTATYYCAGREAYWGQGTLVTVSA;

the nucleotide sequence of the 7C10-C5 light chain variable region was determined to be:

(SEQ ID NO: 40)
GATGTTTTGATGACCCAAACTCCACTCTCCCTGCC
TGTCAGTCTTGGAGATCAAGCCTCCATCTCTTGCA
GATCTAGTCAGAGCATTGTACATAGTAATGGAAAC
ACCTATTTAGAATGGTACCTGCAGAAACCAGGCCA
GTCTCCAAAGCTCCTGATCTACAAAGTTTCCAACC
GATTTTCTGGGGTCCCAGACAGGTTCAGTGGCAGT
GGATCAGGGACAGATTTCACACTCAAGATCAACAG
AGTGGAGGCTGAGGATCTGGGAGTTTATTACTGCT
TTCAAGGTTCACATGTTCCGTGGACGTTCGGTGG
AGGCACCAAGCTGGAAATCAAA;

and

the amino acid sequence of the 7C10-C5 light chain variable region was determined to be:

(SEQ ID NO: 17)
DVLMTQTPLSLPVSLGDQASISCRSSQSIVHSNGNTYLE
WYLQKPGQSPKLLIYKVSNRFSGVPDRFSGSGSGTDFTL
KINRVEAEDLGVYYCFQGSHVPWTFGGGTKLEIK;

wherein in the amino acid sequences, underlined letters indicate the CDRs according to Kabat and bold letters indicate the CDRs according to Chotia; and in the nucleotide sequences italic letters indicate the joining (J) region. The J region is annotated according to the IMGT nomenclature.

Alternatively, the amino acid sequence of the 7C10-C5 heavy chain variable region may comprise a serine instead of a glycine at position 10.

The complementary determining regions (CDRs) and the framework regions (FRs) of the heavy and light chain variable regions according to Kabat, Chotia, AbM, Contact and IMGT numbering systems/definitions are depicted in Tables 2 and 3. Of note, the Kabat definition is based on sequence variability and is the most commonly used, the Chothia definition is based on the location of the structural loop regions, the AbM definition is a compromise between the two used by Oxford Molecular's AbM antibody modelling software, and the contact definition is based on an analysis of the available complex crystal structures (also see http://www.bioinf.org.uk/abs/; and Swindells (2017), J Mol Biol. 2017; 429(3):356-364). The IMGT definition is according to Brochet (2008), Nucleic Acids Res 36, W503-508; and Giudicelli (2011), Cold Spring Harb Protoc 2011, 695-715.

TABLE 2
Complementary determining regions (CDRs)
and the framework regions (FRs) of the
heavy chain variable region of 7C10-C5.

| | Defini- | | | |
|---|---|---|---|---|
| Region | tion | Sequence Fragment | Residues | Length |
| HFR1 | Chothia | DVQLQESGPGLVKPSQSLS | 1-25 | 25 |
| | | LTCSVT----- | | |
| | | (SEQ ID NO: 31) | | |
| | AbM | DVQLQESGPGLVKPSQSLS | 1-25 | 25 |
| | | LTCSVT----- | | |
| | | (SEQ ID NO: 31) | | |
| | Kabat | DVQLQESGPGLVKPSQSLS | 1-30 | 30 |
| | | LTCSVTGYSIT | | |
| | | (SEQ ID NO: 8) | | |
| | Contact | DVQLQESGPGLVKPSQSLS | 1-29 | 29 |
| | | LTCSVT | | |
| | | GYSI- | | |
| | | (SEQ ID NO: 78) | | |
| | IMGT | DVQLQESGPGLVKPSQSLS | 1-25 | 25 |
| | | LTCSVT | | |
| | | (SEQ ID NO: 31) | | |
| CDR- | Chothia | GYSITSGY--- | 26-33 | 8 |
| H1 | | (SEQ ID NO: 27) | | |
| | AbM | GYSITSGYYWN | 26-36 | 11 |
| | | (SEQ ID NO: 79) | | |
| | Kabat | -----SGYYWN | 31-36 | 6 |
| | | (SEQ ID NO: 4) | | |
| | Contact | ----TSGYYWN | 30-36 | 7 |
| | | (SEQ ID NO: 80) | | |
| | IMGT | GYSITSGYY-- | 26-34 | 9 |
| | | (SEQ ID NO: 81) | | |
| HFR2 | Chothia | YWNWIRQFPGNKLEWMGYI | 34-52 | 19 |
| | | (SEQ ID NO: 32) | | |
| | AbM | ---WIRQFPGNKLEWMG-- | 37-50 | 14 |
| | | (SEQ ID NO: 9) | | |
| | Kabat | ---WIRQFPGNKLEWMG-- | 37-50 | 14 |
| | | (SEQ ID NO: 9) | | |
| | Contact | ---WIRQFPGNKLE----- | 37-47 | 11 |
| | | (SEQ ID NO: 82) | | |
| | IMGT | -WNWIRQFPGNKLEWMGY- | 35-51 | 17 |
| | | (SEQ ID NO: 83) | | |
| CDR- | Chothia | -----SYDGS--------- | 53-57 | 5 |
| H2 | | (SEQ ID NO: 26) | | |
| | AbM | ---YISYDGSNN------- | 51-59 | 9 |
| | | (SEQ ID NO: 84) | | |
| | Kabat | ---YISYDGSNNYNPSLKN | 51-66 | 16 |
| | | (SEQ ID NO: 3) | | |
| | Contact | WMGYISYDGSNN------- | 48-59 | 12 |
| | | (SEQ ID NO: 85) | | |
| | IMGT | ----ISYDGSN-------- | 52-58 | 7 |
| | | (SEQ ID NO: 86) | | |
| HFR3 | Chothia | NNYNPSLKNRISITRDTS | 58-98 | 41 |
| | | KNQFFLKLNSVTTEDTAT | | |
| | | YYCAG | | |
| | | (SEQ ID NO: 33) | | |
| | AbM | --YNPSLKNRISITRDTSK | 60-98 | 39 |
| | | NQFFLKLNSVTTEDTATYY | | |
| | | CAG | | |
| | | (SEQ ID NO: 87) | | |
| | Kabat | ---------RISITRDTSK | 67-98 | 32 |
| | | NQFFLKLNSVTTEDTATYY | | |
| | | CAG | | |
| | | (SEQ ID NO: 10) | | |
| | Contact | --YNPSLKNRISITRDTSK | 60-96 | 37 |
| | | NQFFLKLNSVTTEDTATYY | | |
| | | C-- | | |
| | | (SEQ ID NO: 88) | | |
| | IMGT | -NYNPSLKNRISITRDTSK | 59-96 | 38 |
| | | NQFFLKLNSVTTEDTATYY | | |
| | | C-- | | |
| | | (SEQ ID NO: 89) | | |
| CDR- | Chothia | --RFAY | 99-102 | 4 |
| H3 | | (SEQ ID NO: 25) | | |
| | AbM | --RFAY | 99-102 | 4 |
| | | (SEQ ID NO: 2 or 25) | | |
| | Kabat | --RFAY | 99-102 | 4 |
| | | (SEQ ID NO: 2) | | |
| | Contact | AGRFA- | 97-101 | 5 |
| | | (SEQ ID NO: 90) | | |
| | IMGT | AGRFAY | 97-102 | 6 |
| | | (SEQ ID NO: 91) | | |
| HFR4 | Chothia | -WGQGTLVTVSA | 103-113 | 11 |
| | | (SEQ ID NO: 34) | | |
| | AbM | -WGQGTLVTVSA | 103-113 | 11 |
| | | (SEQ ID NO: 11/34) | | |
| | Kabat | -WGQGTLVTVSA | 103-113 | 11 |
| | | (SEQ ID NO: 11) | | |
| | Contact | YWGQGTLVTVSA | 102-113 | 12 |
| | | (SEQ ID NO: 92) | | |
| | IMGT | -WGQGTLVTVSA | 103-113 | 11 |
| | | (SEQ ID NO: 11/34) | | |

Alternatively, the HFR1 of 7C10-C5 may comprise a serine instead of a glycine at position 10 and thus read:

| | Defini- | | | |
|---|---|---|---|---|
| Region | tion | Sequence Fragment | Residues | Length |
| HFR1 | Chothia | DVQLQESGPSLVKPSQ | | |
| | | SLSLTCSVT----- | | |
| | | (SEQ ID NO: 46) | 1-25 | 25 |
| | AbM | DVQLQESGPSLVKPS | | |
| | | QSLSLTCSVT----- | | |
| | | (SEQ ID NO: 46) | 1-25 | 25 |
| | Kabat | DVQLQESGPSLVKPS | | |
| | | QSLSLTCSVTGYSIT | | |
| | | (SEQ ID NO: 45) | 1-30 | 30 |
| | Contact | DVQLQESGPSLVKPS | | |
| | | QSLSLTCSVTGYSI- | | |
| | | (SEQ ID NO: 93) | 1-29 | 29 |
| | IMGT | DVQLQESGPSLVKPS | | |
| | | QSLSLTCSVT----- | | |
| | | (SEQ ID NO: 46) | 1-25 | 25 |

TABLE 3
Complementary determining regions (CDRs)
and the framework regions (FRs) of the
light chain variable region of 7C10-C5.

| | Defini- | | | |
|---|---|---|---|---|
| Region | tion | Sequence Fragment | Residues | Length |
| LFR1 | Chothia | DVLMTQTPLSLPVSLGD | 1-23 | 23 |
| | | QASISC------ | | |
| | | (SEQ ID NO: 35) | | |
| | AbM | DVLMTQTPLSLPVSLGD | 1-23 | 23 |
| | | QASISC------ | | |
| | | (SEQ ID NO: 12/35) | | |
| | Kabat | DVLMTQTPLSLPVSLGD | 1-23 | 23 |
| | | QASISC------ | | |
| | | (SEQ ID NO: 12) | | |
| | Contact | DVLMTQTPLSLPVSLGD | 1-29 | 29 |
| | | QASISCRSSQSI | | |
| | | (SEQ ID NO: 94) | | |
| | IMGT | DVLMTQTPLSLPVSLGD | 1-26 | 26 |
| | | QASISCRSS--- | | |
| | | (SEQ ID NO: 95) | | |
| CDR-L1 | Chothia | RSSQSIVHSNGNTYLE-- | 24-39 | 16 |
| | | (SEQ ID NO: 30) | | |
| | AbM | RSSQSIVHSNGNTYLE-- | 24-39 | 16 |
| | | (SEQ ID NO: 7/30) | | |
| | Kabat | RSSQSIVHSNGNTYLE-- | 24-39 | 16 |
| | | (SEQ ID NO: 7) | | |
| | Contact | ------VHSNGNTYLEWY | 30-41 | 12 |
| | | (SEQ ID NO: 96) | | |
| | IMGT | ---QSIVHSNGNTY---- | 27-37 | 11 |
| | | (SEQ ID NO: 97) | | |
| LFR2 | Chothia | --WYLQKPGQSPKLLIY | 40-54 | 15 |
| | | (SEQ ID NO: 36) | | |
| | AbM | --WYLQKPGQSPKLLIY | 40-54 | 15 |
| | | (SEQ ID NO: 13/36) | | |
| | Kabat | --WYLQKPGQSPKLLIY | 40-54 | 15 |
| | | (SEQ ID NO: 13) | | |
| | Contact | ----LQKPGQSPK---- | 42-50 | 9 |
| | | (SEQ ID NO: 98) | | |
| | IMGT | LEWYLQKPGQSPKLLIY | 38-54 | 17 |
| | | (SEQ ID NO: 99) | | |
| CDR-L2 | Chothia | ----KVSNRFS | 55-61 | 7 |
| | | (SEQ ID NO: 29) | | |
| | AbM | ----KVSNRFS | 55-61 | 7 |
| | | (SEQ ID NO: 6/29) | | |
| | Kabat | ----KVSNRFS | 55-61 | 7 |
| | | (SEQ ID NO: 6) | | |
| | Contact | LLIYKVSNRF- | 51-60 | 10 |
| | | (SEQ ID NO: 100) | | |
| | IMGT | ----KVS---- | 55-57 | 3 |
| LFR3 | Chothia | -----GVPDRFSGSGSGTDF | 62-93 | 32 |
| | | TLKINRVEAEDLGVYYC | | |
| | | (SEQ ID | | |
| | | NO: 37) | | |
| | AbM | -----GVPDRFSGSGSGTDF | 62-93 | 32 |
| | | TLKINRVEAEDLGVYYC | | |
| | | (SEQ ID | | |
| | | NO: 14/37) | | |
| | Kabat | -----GVPDRFSGSGSGTDF | 62-93 | 32 |
| | | TLKINRVEAEDLGVYYC | | |
| | | (SEQ ID | | |
| | | NO: 14) | | |
| | Contact | ----SGVPDRFSGSGSGTDF | 61-93 | 33 |
| | | TLKINRVEAEDLGVYYC | | |
| | | (SEQ ID | | |
| | | NO: 101) | | |
| | IMGT | NRFSGVPDRFSGSGSGTDFT | 58-93 | 36 |
| | | LKINRVEAEDLGVYYC | | |
| | | (SEQ | | |
| | | ID NO: 102) | | |
| CDR-L3 | Chothia | FQGSHVPWT | 94-102 | 9 |
| | | (SEQ ID NO: 28) | | |
| | AbM | FQGSHVPWT | 94-102 | 9 |
| | | (SEQ ID NO: 5/28) | | |
| | Kabat | FQGSHVPWT | 94-102 | 9 |
| | | (SEQ ID NO: 5) | | |
| | Contact | FQGSHVPW- | 94-101 | 8 |
| | | (SEQ ID NO: 103) | | |
| | IMGT | FQGSHVPWT | 94-102 | 9 |
| | | (SEQ ID NO: 5/28) | | |
| LFR4 | Chothia | -FGGGTKLEIK | 103-112 | 10 |
| | | (SEQ ID NO: 38) | | |
| | AbM | -FGGGTKLEIK | 103-112 | 10 |
| | | (SEQ ID NO: 15/38) | | |
| | Kabat | -FGGGTKLEIK | 103-112 | 10 |
| | | (SEQ ID NO: 15) | | |
| | Contact | TFGGGTKLEIK | 102-112 | 11 |
| | | (SEQ ID NO: 104) | | |
| | IMGT | -FGGGTKLEIK | 103-112 | 10 |
| | | (SEQ ID NO: 15/38) | | |

The CDRs and FRs in Tables 2 and 3 are shown for illustrative purposes with IMGT according to Brochet (2008), loc. cit.; and Giudicelli (2011) loc cit. In the context of the invention, the CDRs and FRs of the inventive antibody/antibodies are defined according to the Kabat numbering system.

Therefore, the CDRs of 7C10-C5 according to Kabat numbering were determined as following:

the CDR-H3 sequence is RFAY (SEQ ID NO:2),

the CDR-H2 sequence is YISYDGSNNYNPSLKN (SEQ ID NO:3),

the CDR-H1 sequence is SGYYWN (SEQ ID NO:4),

the CDR-L3 sequence is FQGSHVPWT (SEQ ID NO:5),

the CDR-L2 sequence is KVSNRFS (SEQ ID NO:6), and

the CDR-L1 sequence is RSSQSIVHSNGNTYLE (SEQ ID NO:7).

Accordingly, the FRs of 7C10-C5 according to Kabat numbering were determined as following:

the H-FR1 sequence is preferably DVQLQESGPGLVKPSQSLSLTCSVTGYSIT (SEQ ID NO:8) or alternatively DVQLQESGPSLVKPSQSLSLTCSVTGYSIT (SEQ ID NO:45),

the H-FR2 sequence is WIRQFPGNKLEWMG (SEQ ID NO:9),

the H-FR3 sequence is RISITRDTSKNQFFLKLNSVTTEDTATYYCAG (SEQ ID NO:10),

the H-FR4 sequence is WGQGTLVTVSA (SEQ ID NO:11),

the L-FR1 sequence is DVLMTQTPLSLPVSLGDQASISC (SEQ ID NO:12),

the L-FR2 sequence is WYLQKPGQSPKLLIY (SEQ ID NO:13),

the L-FR3 sequence is GVPDRFSGSGSGTDFTLKINRVEAEDLGVYYC (SEQ ID NO:14), and

the L-FR4 sequence is FGGGTKLEIK (SEQ ID NO:15).

Accordingly, the CDRs of 7C10-C5 according to Chothia numbering were determined as following:

the CDR-H3 sequence is RFAY (SEQ ID NO:25),

the CDR-H2 sequence is SYDGS (SEQ ID NO:26),

the CDR-H1 sequence is GYSITSGY (SEQ ID NO:27),

the CDR-L3 sequence is FQGSHVPWT (SEQ ID NO:28),

the CDR-L2 sequence is KVSNRFS (SEQ ID NO:29), and

the CDR-L1 sequence is RSSQSIVHSNGNTYLE (SEQ ID NO:30).

Accordingly, the FRs of 7C10-C5 according to Chothia numbering were determined as following:

the H-FR1 sequence is preferably DVQLQESGPGLVKPSQSLSLTCSVT (SEQ ID NO:31) or alternatively DVQLQESGPSLVKPSQSLSLTCSVT (SEQ ID NO:46),

the H-FR2 sequence is YWNWIRQFPGNKLEWMGYI (SEQ ID NO:32),

the H-FR3 sequence is NNYNPSLKNRISITRDTSKNQFFLKLNSVTTEDTATYYCAG (SEQ ID NO:33),

the H-FR4 sequence is WGQGTLVTVSA (SEQ ID NO:34),

the L-FR1 sequence is DVLMTQTPLSLPVSLGDQASISC (SEQ ID NO:35),

the L-FR2 sequence is WYLQKPGQSPKLLIY (SEQ ID NO:36),

the L-FR3 sequence is GVPDRFSGSGSGTDFTLKINRVEAEDLGVYYC (SEQ ID NO:37), and

the L-FR4 sequence is FGGGTKLEIK (SEQ ID NO:38).

### Example 4. Generation and Characterization of Single-Chain Variable Fragments (scFvs) of 7C10-C5

Cloning of the Single-Chain Variable Fragments (scFv)

For cloning of the single-chain variable fragments (scFv), the sequences of the first 20 base pairs (bp) of the heavy or light chain variable regions (SEQ ID NO:39 and SEQ ID NO:40, respectively) were taken as forward primers. As reverse primer for the light chain variable fragment a sequence in the joining region and for the heavy chain a sequence at the beginning of the constant region was used.

Additionally, the light chain forward primer contained a part of a signal peptide. Of note, the signal peptide is thought to improve expression of the antibody (Haryadi (2015); PLoS One 10, e0116878). The sequence of the light chain forward primer was: 5′-CCTGGGGCTCCTGCTGCTCTGGCTCTCAGGTGCCAGATGT-gatgttttgatgacccaaac-3′ (SEQ ID NO:105), with the signal peptide (overhang) in capital letters.

The light chain reverse primer additionally contained the linker sequence between the heavy and light chain variable fragments. The sequence of the light chain reverse primer was: 5′-GGAAGATCTAGAGGAACCACCCCCACCACCGCCCGAGCCACCGCCACCAGAGG-atttgatttccagcttggtgcc-3′) (SEQ ID NO:106), with the linker sequence (overhang) in capital letters.

The heavy chain forward primer also contained the linker sequence between the heavy and light chain variable fragments and had the sequence 5′-GGTGGTTCCTCTAGATCTTCCCTCgatgtacagcttcaggagtc-3′ (SEQ ID NO:107), with the linker sequence (overhang) in capital letters.

The heavy chain reverse primer additionally contained the sequence for a FseI restriction enzyme cutting site and had the sequence

(SEQ ID NO: 108)
5’-CTGGCCGGCCTGGCCACTAGT
gacagatgggggtgtcgttttggc-3’,

with the overhang in capital letters and the FseI restriction site underlined.

For the PCR, 2 μl cDNA (derived from the single hydridoma clone 7C10-C5) from the 20 μl Accu Script reaction volume (prepared as described above in Example 3) were mixed with 2 μl forward primer (10 μM) and 2 μl reverse primer (10 μM), 10 μl 5×Q5 Reaction Buffer (NEB #B9027), 0.41 dNTPs (10 μM), 33 μl nuclease free water (Sigma-Aldrich, #W4502) and 0.5 μl Q5® High-Fidelity DNA Polymerase (Neb, #M0491L). Reactions were performed in a Biometra TRIO thermocycler starting with a DNA denaturation step at 98° C. for 1 min followed by 30 cycles of denaturation of the DNA at 98° C. for 15 sec. annealing at 56° C. for 15 sec., and elongation at 72° C. for 30 sec. A 3 minutes 72° C. step completed the reaction. The PCR products were separated on a 1.5% TAE (for 1 Liter: 4.84 g Tris (AppliChem #A1086), 1.14 ml Glacial Acetic Acid, 2 ml 0.5M EDTA pH 8.0) agarose gel (Sigma, A9539) and stained with ethidium bromide. The bands corresponding to the light chain variable fragment and the heavy chain variable fragment were cut out and purified using a Wizard® DNA Clean Up Kit (Promega, #A9282), following the protocol as instructed, and eluted in 50 μl nuclease free water (Sigma, #W4502).

Next, the light and heavy chain variable fragments were joined by fusion PCR. 100 ng of the respective light chain variable fragment with a part of the signal peptide on the 5′ end and the overlapping linker sequence at the 3 ‘-end and 100 ng of the respective heavy variable fragment with the overlapping linker sequence at the 5’-end were mixed with 2 μl signal peptide forward primer (10 μM) containing a BlpI restriction enzyme cutting site (5′-ATGGACATGAGGGTCCCTGCTCAGCTCCTGGGGCTCCTGCTGCTCTGGCTC-3′) (SEQ ID NO:109) and 2 μl heavy chain reverse primer containing a FseI restriction enzyme cutting site (5′-GAGGAGGAGGAGGAGGAGCCTGGCCGGCCTGGCCACTAGTG-3′) (SEQ ID NO:110) (10 μM), 10 μl 5× Q5 Reaction Buffer (NEB #B9027), 0.2 μl dNTPs (10 μM), 33 μl nuclease free water (Sigma-Aldrich, #W4502) and 0.5 μl Q5® High-Fidelity DNA Polymerase (Neb, #M0491L). Reactions were done in a Biometra TRIO thermocycler starting with a DNA denaturation step at 98° C. for 1 min followed by 30 cycles of denaturation of the DNA at 98° C. for 15 sec. annealing at 56° C. for 15 sec., and elongation at 72° C. for 30 sec. A 3 minutes 72° C. step completed the reaction. The PCR products were separated on a 1.5% TAE agarose gel (Sigma, A9539) stained with ethidium bromide. The corresponding band (about 862 bp) was cut out and purified using a Wizard® DNA Clean Up Kit (Promega, #A9282), following the protocol as instructed, and eluted in 50 μl nuclease free water (Sigma-Aldrich, #W4502). The purified fragments were cloned into the pJet 1.2 cloning vector following the protocol of the CloneJET PCR Cloning Kit (Thermo, #K1231). 2.5 μl of the ligation mixture was used for bacterial transformation. 100 μl of heat shock competent E. coli HB101 were thawed on ice, the DNA was added and the cells were kept for another 30 minutes on ice. A heat shock for 1 minute at 42° C. was performed and after 10 minutes on ice the bacteria were resuspended in 500 μl LB medium without antibiotics for recovery. After 30 minutes at 37° C. bacteria were plated out on LB plates with Ampicillin and incubated over night at 37° C. Colonies were screened for insertions by colony PCR as described in the CloneJET PCR Cloning Kit with the enclosed pJet primers. Positive clones were subsequently inoculated in 5 ml LB with Ampicillin, grown over night and DNA was eluted in 50 μl nuclease free water (Sigma-Aldrich, #W4502) after purification with a Qiagen Miniprep Kit by following the manufacture's manual (Qiagen, #27106). The gained DNA was prepared for sequencing as demanded by the sequencing company (LGC genomics). 10 μl with 100 ng/μl DNA was mixed with 4 μl primer (10 μM) using either the pJET1.2 forward sequencing primer (5′-CGACTCACTATAGGGAGAGCGGC-3′) (SEQ ID NO:76) or the pJET1.2 reverse sequencing primer (5′-AAGAACATCGATTTTCCATGGCAG-3′) (SEQ ID NO:77) to get sequences from both sides.

Cloning of Single-Chain Variable Fragments (scFvs) of 7C10-C5 into a Mammalian Expression Vector

Monovalent 7C10-C5 scFv

For mammalian expression, the monovalent 7C10-C5 scFv was cloned into the pcDNA3.1(+)/Hygro vector (Thermo Fisher) already containing the Signal Peptide with the BlpI restriction site and a FseI restriction site before the 6×Histidine (His) Tag and hemagglutinin (HA) tag. The pcDNA3.1(+) vector was cut with BlpI and FseI. 3 μg plasmid was mixed with 50 NEB CutSmart® buffer (10×), 0.50 BlpI (NEB R0585S) and FseI (NEB R0588L), filled up with nuclease free water (Sigma-Aldrich, #W4502) to 50 μl, and incubated at 37° C. overnight. The monovalent 7C10-C5 scFv obtained with the fusion PCR described above was cut by mixing 1 μg of the PCR with 50 10×CutSmart® buffer (NEB B7204S), 0.50 BlpI (NEB R0585S) and FseI (NEB R0588L), filled up with nuclease free water to 500, and incubated at 37° C. over night. The restriction digest was separated on a 1% TAE (25 mM Tris (AppliChem #A1086), 0.114% Glacial Acetic Acid (AppliChem #A3701), 0.1 mM EDTA pH 8.0 (AppliChem #2937) agarose gel (Sigma, A9539) and stained with ethidium bromide (Sigma #E1510). The correct bands (5761 bp for the pcDNA 3 vector backbone and 819 bp for the 7C10-C5 scFv) were cut out and purified using a Wizard® DNA Clean Up Kit (Promega #A9282), following the protocol as instructed, and eluted in 30 μl nuclease free water (Sigma #W4502). Ligation was performed mixing 50 ng of the vector, 21 ng of the 7C10-C5 scFv, 20 of T4 DNA Ligase Buffer (NEB) and 1 μl of T4 DNA Ligase (NEB M0202L), filled up with nuclease free water to 20 μl and incubated at 22° C. for 1 hour. 10 μl of the Ligation mixture was used for bacterial transformation. 100 μl of heat shock competent E. coli HB101 were thawed on ice, the DNA was added and the cells were kept for another 30 minutes on ice. A heat shock for 1 minute at 42° C. was performed and after 10 minutes on ice the bacteria were resuspended in 500 μl LB medium without antibiotics for recovery. After 30 minutes at 37° C. bacteria were plated out on LB plates with Ampicillin and incubated over night at 37° C. 3 Clones were subsequently inoculated in 5 ml LB with Ampicillin, grown over night and DNA was eluted in 50 μl nuclease free water after purification with a Qiagen Miniprep Kit by following the manufacture's manual (Qiagen, #27106). The gained DNA was prepared for sequencing as demanded by the sequencing company (LGC genomics). 10 μl with 100 ng/μ1 DNA was mixed with 4 μl primer (10 μM) using either the pcDNA3 forward sequencing primer (5′-TAATACGACTCACTATAGGG-3′) (SEQ ID NO:111) or the pcDNA3 reverse sequencing primer (5′-TAGAAGGCACAGTCGAGG-3′) (SEQ ID NO:112) to get sequences from both sides. The obtained sequences for the heavy and light variable fragments were checked with previously obtained sequences.

Bivalent 7C10-C5 scFv

To obtain a bivalent 7C10-C5 scFV, two scFvs were linked together by amplifying the scFvs from the pcDNA3.1(+)/Hygro 7C10-C5 by PCR. The 5′-terminal primer of the first scFv was binding the signal peptide encoding sequence (3′-ATGGACATGAGGGTCCCTGCTCAGCTCCTGGGGCTCCTGCTGCTCTGGCTC-5′) (SEQ ID NO:109) and the 3′-terminal primer at the heavy chain constant region comprised an additional sequence for a (Gly-Ser)×15 linker between the two scFvs (5′-CCTGAACCGGACCCAGATCCGCTGCCACTACCAGACCCTGATCCGGAGCCAGAAC CGACAGATGGGGGTGTCG-3′) (SEQ ID NO:113). The 5′-terminal primer of the second scFv started with the overlapping linker sequence, comprised the rest of the linker sequence, and was binding the scFv encoding sequence at the 5′-end of the variable fragment (5′-GGATCTGGGTCCGGTTCAGGCTCAGGAAGTGGGAGCGGATCAGGGTCCGGGTCA GATGTTTTGATGACCCAAAC-3′) (SEQ ID NO:114). The 3′-terminal primer reverse primer contained a FseI restriction enzyme cutting site (5′-GAGGAGGAGGAGGAGGAGCCTGGCCGGCCTGGCCACTAGTG-3′) (SEQ ID NO:110). 50 ng pcDNA3.1(+)/Hygro 7C10-C5 vector, 10 μl 5×Q5 Reaction Buffer (NEB #B9027), 0.41 dNTPs (10 μM) nuclease free water filled up to 50 μl (Sigma-Aldrich, #W4502) and 0.5 μl Q5® High-Fidelity DNA Polymerase (Neb, #M0491L) were mixed. Reactions were done in a Biometra TRIO thermocycler starting with a DNA denaturation step at 98° C. for 1 min followed by 30 cycles of denaturation of the DNA at 98° C. for 15 sec. annealing at 56° C. for 15 sec., and elongation at 72° C. for 30 sec. A 3 minutes 72° C. step completed the reaction. The PCR products were separated on a 1% TAE agarose gel stained with ethidium bromide. The correct bands (about 878 for the first and 851 bp for the second scFv) were cut out and purified using a Wizard® DNA Clean Up Kit (Promega #A9282), following the protocol as instructed, and eluted in 50 μl nuclease free water (Sigma-Aldrich, #W4502). For linking of the two scFv, 100 ng of the first and 100 ng of the second scFv encoding DNA fragments were mixed with 2 μl of the 5′-terminal primer of the first scFv (10 μM), 2 μl of the 3′ terminal primer of the second scFv (10 μM), 10 μl Q5 buffer (5×), 0.2 μl of dNTPs (10 mM) and filled up with nuclease free water (Sigma-Aldrich, #W4502) to 50 μl. Reactions were done in a X thermocylcer starting with 1 minutes 98° C. followed by 20 cycles of denaturation of the DNA at 98° C. for 15 sec., annealing at 56° C. for 15 sec., and elongation at 72° C. for 45 seconds. A 3 minutes 72° C. step completed the reaction. The PCR products were separated on a 1% TAE agarose gel (Sigma, A9539) stained with ethidium bromid (X). The band corresponding to the combined bivalent scFv DNA fragment (about 1709 bp) was cut out and purified using a Wizard® DNA Clean Up Kit (Promega #A9282), following the protocol as instructed, and eluted in 50 μl nuclease free water (Sigma-Aldrich, #W4502).

The bivalent scFv 7C10-C5 was cloned into the pcDNA3.1(+)/Hygro vector (Thermo Fisher) containing the signal peptide containing the BlpI restriction site and a FseI restriction site before the 6× Histidine (His) Tag and hemagglutinin (HA) tag. The pcDNA3.1(+) vector was cut with BlpI and FseI. 3 μg plasmid was mixed with 5 μl NEB CutSmart® buffer (10×), 0.5 μl BlpI (NEB R0585S) and FseI (NEB R0588L), filled up with nuclease free water (Sigma-Aldrich, #W4502) to 50 μl, and incubated at 37° C. overnight. The bivalent 7C10-C5 scFv obtained with the fusion PCR described above was cut by mixing 1 μg of the PCR with 5 μl 10× CutSmart® buffer (NEB B7204S), 0.5 μl BlpI (NEB R0585S) and FseI (NEB R0588L), filled up with nuclease free water to 50 μl, and incubated at 37° C. overnight. The restriction digests were separated on a 1% TAE (25 mM Tris (AppliChem #A1086), 0.114% Glacial Acetic Acid (AppliChem #A3701), 0.1 mM EDTA pH 8.0 (AppliChem #2937) agarose gel (Sigma, A9539) and stained with ethidium bromide (Sigma #E1510). The correct bands (5761 bp for the pcDNA 3 vector backbone and 1665 bp for the 7C10-C5 bivalent scFv) were cut out and purified using a Wizard® DNA Clean Up Kit (Promega #A9282), following the protocol as instructed, and eluted in 30 μl nuclease free water (Sigma #W4502). Ligation was performed mixing 50 ng of the vector, 43 ng of the 7C10-C5 bivalent scFv, 2 μl of T4 DNA Ligase Buffer (NEB) and 1 μl of T4 DNA Ligase (NEB M0202L), filled up with nuclease free water to 20 μl and incubated at 22° C. for 1 hour. 10 μl of the Ligation mixture was used for bacterial transformation. 100 μl of heat shock competent E. coli HB101 were thawed on ice, the DNA was added and the cells were kept for another 30 minutes on ice. A heat shock for 1 minute at 42° C. was performed and after 10 minutes on ice the bacteria were resuspended in 500 μl LB medium without antibiotics for recovery. After 30 minutes at 37° C. bacteria were plated out on LB plates with Ampicillin and incubated overnight at 37° C. 3 Clones were subsequently inoculated in 5 ml LB with Ampicillin, grown over night and DNA was eluted in 50 μl nuclease free water after purification with a Qiagen Miniprep Kit by following the manufacture's manual (Qiagen, #27106). The gained DNA was prepared for sequencing as demanded by the sequencing company (LGC genomics). 10 μl with 100 ng/μ1 DNA was mixed with 4 μl primer (10 μM) using either the pcDNA3 forward sequencing primer (5′-TAATACGACTCACTATAGGG-3′) (SEQ ID NO:111) or the pcDNA3 reverse sequencing primer (5′-TAGAAGGCACAGTCGAGG-3′) (SEQ ID NO:112) to get sequences from both sides. The obtained sequences for the heavy and light variable fragments were checked with previously obtained sequences.

Expression of Recombinant Mono- and Bivalent scFv 7C10-C5 Constructs.

For mammalian transfection, the clones with the correct expression constructs for the monovalent or bivalent 7C10-C5 recombinant antibodies were subsequently inoculated in 100 ml LB with Ampicillin, grown overnight and DNA was eluted in 200 μl nuclease free water after purification with a Qiagen Midiprep Kit by following the manufacture's manual (Qiagen, #12943).

HEK293T cells were grown in DMEM+10% FCS+2 mM L-glutamine (Sigma, G2150)+100 units/ml Penicillin/0.1 mg/ml Streptomycin in a humidified incubator controlled at 37° C. with 7.5% CO2. For subculturing, cells were passaged every 2 to 3 days at a ratio of 1:3 to 1:8. 1.3×106 HEK 293T were seeded in 6 cm dishes in 5 ml growth media the day before transfection. Cells were transfected by mixing 600 μl of OptiMEM (Gibco™ 31985047, #11524456) with 5 μg of DNA (monovalent or bivalent 7C10-C5 recombinant antibody constructs) 2 seconds of vortex-mixing, addition of 7.5 μl of TurboFectin 8.0 (Origin #TF81001) mixing by 5×inverting the tube, incubating the transfection mix for 15 min at room temperature and adding it to the cells. 72 hours later the cell supernatant was centrifuged at 1200 rpm in a Beckman SC6R table top centrifuge, and the supernatant was used for the ELISA and Western blot experiments.

Structural Characterization of Recombinant Mono- and Bivalent scFv 7C10-C5 Antibodies

Monovalent scFv Sequence

The recombinant monovalent scFv antibody was determined to have the following nucleotide and amino acid sequences:

(SEQ ID NO: 41)
ATGGACATGAGGGTCCCTGCTCAGCTCCTGGGGCT
CCTGCTGCTCTGGCTCTCAGGTGCCAGATGTGAT
GTTTTGATGACCCAAACTCCACTCTCCCTGCCTGT
CAGTCTTGGAGATCAAGCCTCCATCTCTTGCAGAT
CTAGTCAGAGCATTGTACATAGTAATGGAAACACC
TATTTAGAATGGTACCTGCAGAAACCAGGCCAGTC
TCCAAAGCTCCTGATCTACAAAGTTTCCAACCGAT
TTTCTGGGGTCCCAGACAGGTTCAGTGGCAGTGGA
TCAGGGACAGATTTCACACTCAAGATCAACAGAGT
GGAGGCTGAGGATCTGGGAGTTTATTACTGCTTTC
AAGGTTCACATGTTCCGTGGACGTTCGGTGGAGGC
ACCAAGCTGGAAATCAAATCCTCTGGTGGCGGTG
GCTCGGGCGGTGGTGGGGGTGGTTCCTCTAGATCT
TCCCTCGATGTACAGCTTCAGGAGTCAGGACCTGG
CCTCGTGAAACCTTCTCAGTCTCTGTCTCTCACCT
GCTCTGTCACTGGCTACTCCATCACCAGTGGTTAT
TACTGGAACTGGATCCGGCAGTTTCCAGGAAACAA
ACTGGAATGGATGGGCTACATAAGCTACGACGGTA
GCAATAACTACAACCCATCTCTCAAAAATCGAATC
TCCATCACTCGTGACACATCTAAGAACCAGTTTTT
CCTGAAGTTGAATTCTGTGACTACTGAGGACACAG
CTACATATTACTGTGCTGGACGGTTTGCTTACTGG
GGCCAAGGGACTCTGGTCACTGTCTCTGCAGCCAA
AACGACACCCCCATCTGTCACTAGTGGCCAGGCCG
GCCAGCACCATCACCATCACCATGGCGCATACCCG
TACGACGTTCCGGACTACGCTTCTTAG;
(SEQ ID NO: 42)
MDMRVPAQLLGLLLLWLSGARCDVLMTQTPLSLPV
SLGDQASISCRSSQSIVHSNGNTYLEWYLQKPGQS
PKLLIYKVSNRFSGVPDRFSGSGSGTDFTLKINRV
EAEDLGVYYCFQGSHVPWTFGGGTKLEIKSSGGG
GSGGGGGGSSRSSLDVQLQESGPGLVKPSQSLSLT
CSVTGYSITSGYYWNWIRQFPGNKLEWMGYISYDG
SNNYNPSLKNRISITRDTSKNQFFLKLNSVTTEDT
ATYYCAGRFAYWGQGTLVTVSAAKTTPPSVTSGQA
GQHHHHHHGAYPYDVPDYAS*;

wherein the signal peptide is in italics, the light chain variable region in bold, the linker double-underlined, the heavy chain variable region single-underlined, and the 3′ or C-terminal part including an 6× His-tag and a HA-tag in standard font. The * denotes the stop codon.

Bivalent scFv Sequence

The recombinant bivalent scFv antibody was determined to have the following nucleotide and amino acid sequences:

(SEQ ID NO: 43)
ATGGACATGAGGGTCCCTGCTCAGCTCCTGGGGCTCCTGCTGCTCTGGCTCTCAGGTG
CCAGATGTGATGTTTTGATGACCCAAACTCCACTCTCCCTGCCTGTCAGTCTT
GGAGATCAAGCCTCCATCTCTTGCAGATCTAGTCAGAGCATTGTACATAGTA
ATGGAAACACCTATTTAGAATGGTACCTGCAGAAACCAGGCCAGTCTCCAAA
GCTCCTGATCTACAAAGTTTCCAACCGATTTTCTGGGGTCCCAGACAGGTTC
AGTGGCAGTGGATCAGGGACAGATTTCACACTCAAGATCAACAGAGTGGAG
GCTGAGGATCTGGGAGTTTATTACTGCTTTCAAGGTTCACATGTTCCGTGGA
CGTTCGGTGGAGGCACCAAGCTGGAAATCAAATCCTCTGGTGGCGGTGGCTCG
GGCGGTGGTGGGGGTGGTTCCTCTAGATCTTCCCTCGATGTACAGCTTCAGGAGT
CAGGACCTGGCCTCGTGAAACCTTCTCAGTCTCTGTCTCTCACCTGCTCTGTCACT
GGCTACTCCATCACCAGTGGTTATTACTGGAACTGGATCCGGCAGTTTCCAGGAA
ACAAACTGGAATGGATGGGCTACATAAGCTACGACGGTAGCAATAACTACAACC
CATCTCTCAAAAATCGAATCTCCATCACTCGTGACACATCTAAGAACCAGTTTTTC
CTGAAGTTGAATTCTGTGACTACTGAGGACACAGCTACATATTACTGTGCTGGAC
GGTTTGCTTACTGGGGCCAAGGGACTCTGGTCACTGTCTCTGCAGCCAAAACGAC
ACCCCCATCTGTC [symbol] [symbol] [symbol] [symbol]
GATGTTTTGA
TGACCCAAACTCCACTCTCCCTGCCTGTCAGTCTTGGAGATCAAGCCTCCAT
CTCTTGCAGATCTAGTCAGAGCATTGTACATAGTAATGGAAACACCTATTTA
GAATGGTACCTGCAGAAACCAGGCCAGTCTCCAAAGCTCCTGATCTACAAAG
TTTCCAACCGATTTTCTGGGGTCCCAGACAGGTTCAGTGGCAGTGGATCAGG
GACAGATTTCACACTCAAGATCAACAGAGTGGAGGCTGAGGATCTGGGAGT
TTATTACTGCTTTCAAGGTTCACATGTTCCGTGGACGTTCGGTGGAGGCACC
AAGCTGGAAATCAAATCCTCTGGTGGCGGTGGCTCGGGCGGTGGTGGGGGTGGT
TCCTCTAGATCTTCCCTCGATGTACAGCTTCAGGAGTCAGGACCTGGCCTCGTGA
AACCTTCTCAGTCTCTGTCTCTCACCTGCTCTGTCACTGGCTACTCCATCACCAGT
GGTTATTACTGGAACTGGATCCGGCAGTTTCCAGGAAACAAACTGGAATGGATGG
GCTACATAAGCTACGACGGTAGCAATAACTACAACCCATCTCTCAAAAATCGAAT
CTCCATCACTCGTGACACATCTAAGAACCAGTTTTTCCTGAAGTTGAATTCTGTGA
CTACTGAGGACACAGCTACATATTACTGTGCTGGACGGTTTGCTTACTGGGGCCA
AGGGACTCTGGTCACTGTCTCTGCAGCCAAAACGACACCCCCATCTGTCACTAGT
GGCCAGGCCGGCCAGCACCATCACCATCACCATGGCGCATACCCGTACGACGTTC
CGGACTACGCTTCTTAG;
(SEQ ID NO: 44)
MDMRVPAQLLGLLLLWLSGARCDVLMTQTPLSLPVSLGDQASISCRSSQSIVHSNGN
TYLEWYLQKPGQSPKLLIYKVSNRFSGVPDRFSGSGSGTDFTLKINRVEAEDLGV
YYCFQGSHVPWTFGGGTKLEIKSSGGGGSGGGGGGSSRSSLDVQLQESGPGLVKPS
QSLSLTCSVTGYSITSGYYWNWIRQFPGNKLEWMGYISYDGSNNYNPSLKNRISITRD
TSKNQFFLKLNSVTTEDTATYYCAGRFAYWGQGTLVTVSAAKTT [symbol] [symbol]
DVLMTQTPLSLPVSLGDQASISCRSSQSIVHSNGNTY
LEWYLQKPGQSPKLLIYKVSNRFSGVPDRFSGSGSGTDFTLKINRVEAEDLGVYY
CFQGSHVPWTFGGGTKLEIKSSGGGGSGGGGGGSSRSSLDVQLQESGPGLVKPSQS
LSLTCSVTGYSITSGYYWNWIRQFPGNKLEWMGYISYDGSNNYNPSLKNRISITRDTS
KNQFFLKLNSVTTEDTATYYCAGRFAYWGQGTLVTVSAAKTTPPSVTSGQAGQHHH
HHHGAYPYDVPDYAS*;

wherein the signal peptide is in italics, the light chain variable region in bold, linker A double-underlined, the heavy chain variable region single-underlined, linker B double-underlined and in italics, and the remainder (the 3′ or C-terminal part including an 6×His-tag and a HA-tag; and a small part before linker B) in standard font. The * denotes the stop codon.

Carboxymethylation Specificity of the Recombinant scFv 7C10-C5 Antibodies

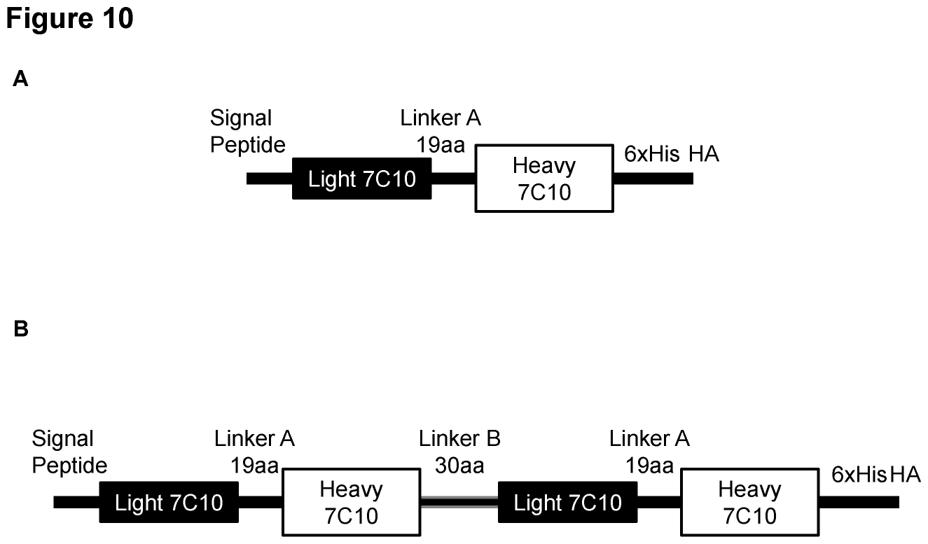

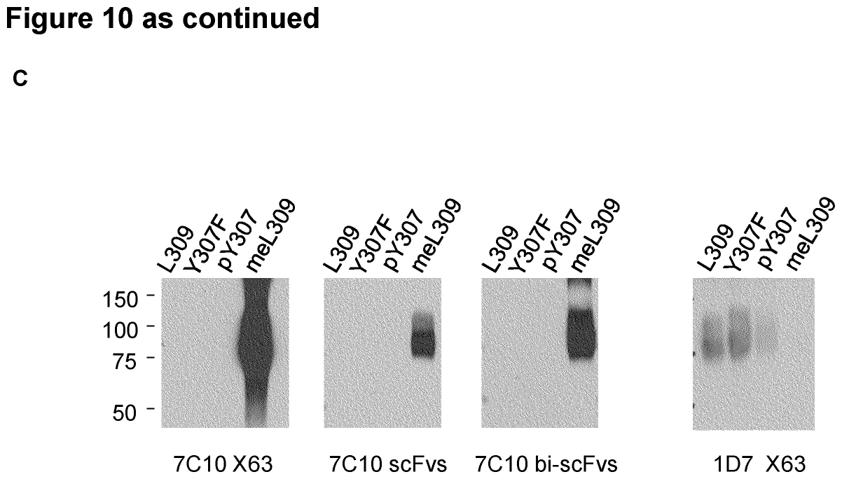

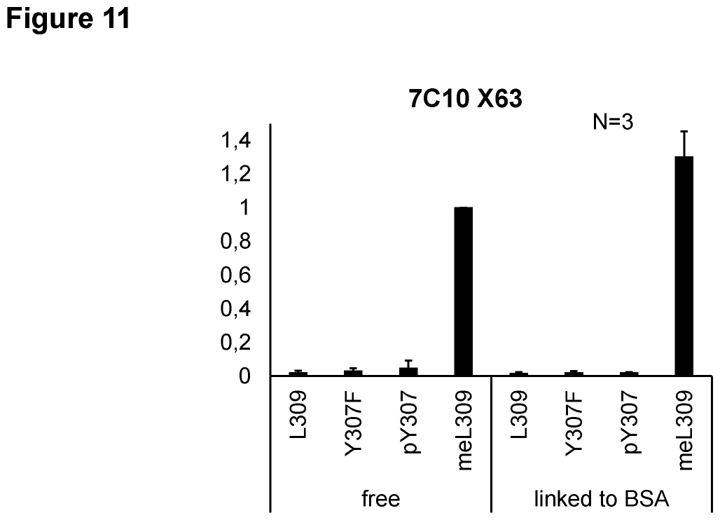

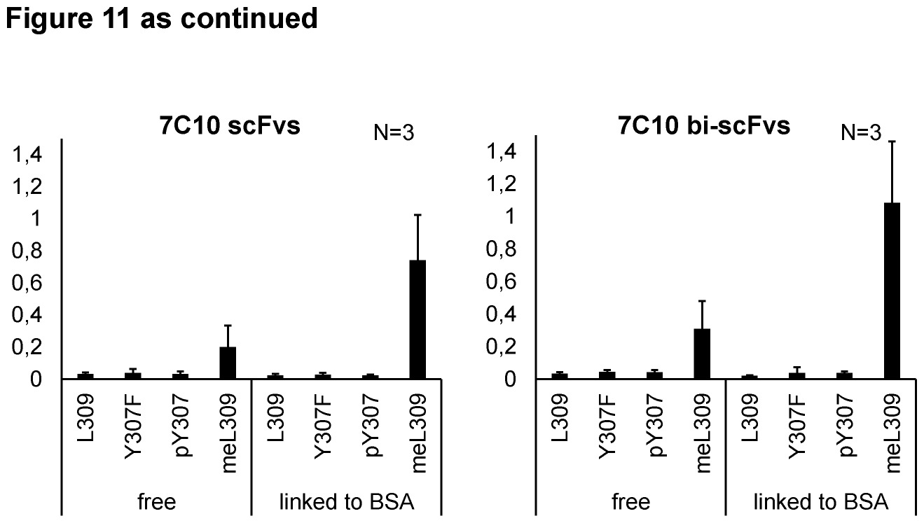

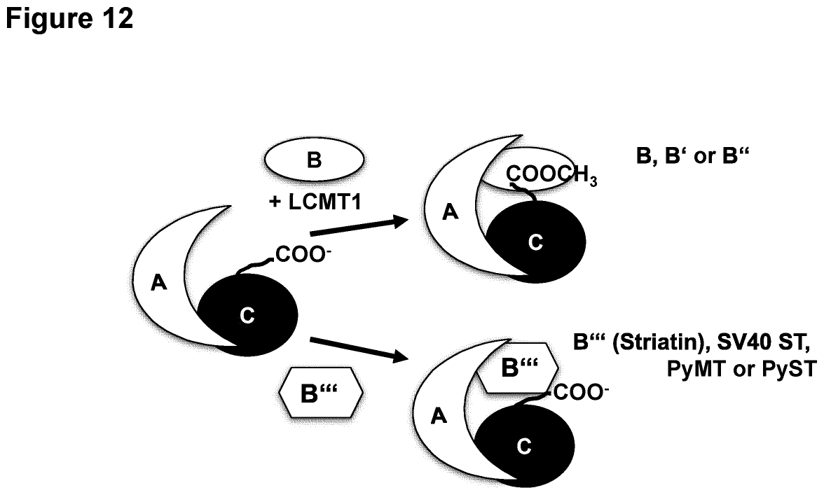

The recombinant anti-carboxymethyl PP2Ac antibodies (mono- or bivalent scFv 7C10-C5) were tested for carboxymethyl-PP2Ac specificity by Western blot analysis and ELISA (FIG. 10C and FIG. 11).

ScFv 7C10-C5 Western Blotting

100 ng of BSA crosslinked with peptides (L309: CGEPHVTRRTPDYFL (SEQ ID NO:49), Y307F: CGEPHVTRRTPDFFL (SEQ ID NO.50), pY307: CGEPHVTRRTPDpYFL (SEQ ID NO:51) or meL309: CGEPHVTRRTPDYFL-CH₃) (SEQ ID NO:49) was separated by 10% SDS-PAGE and transferred to nitrocellulose membrane (0.2 μm, GE Healthcare). Of note, the Y307F peptide cannot be phosphorylated at position 307 due to the presence of a phenylalanine. The membranes were blocked in 3% non-fatty dry milk (NFDM) in TBS-Tween for 1 h at RT and incubated with single clone hybridoma cell culture supernatant, clone 7C10-C5 diluted 1:100 in 0.5% NFDM in TBS-T (7C10 X63), recombinant single chain variable fragment of antibody 7C10-C5, undiluted (7C10 scFvs), recombinant bivalent single chain variable fragment 7C10-C5, undiluted (7C10 bi-scFvs) or single clone hybridoma cell culture supernatant, clone 1D7 (binding to non-carboxymethylated PP2Ac) 1:100 in 0.5% NFDM in TBS-T o/n at 4° C. After washing 3×5 min in TBS-T, blots incubated with recombinant antibodies were incubated with mouse anti-HA monoclonal antibody 16B12 (recognizing the hemagglutinin (HA) tag on the recombinant antibody), 1:10000 in 0.5% NFDM in TBS-T 2 h at room temperature. After washing 3×5 min in TBS-T, incubation with anti-mouse-HRP coupled secondary antibody for 1 h at RT and 3×10 min washes in TBS-T was performed. ECL was performed with GE Healthcare ECL reagents (RPN2106) mixed 1:3 with Clarity™ Western ECL Substrate, 500 ml #1705061 (Biorad). Signals were detected by exposure of X-ray films. Molecular weights in FIG. 10C are indicated in kDa.

It was found that, similarly to the monoclonal antibody 7C10-C5 from the hybridoma supernatant, both recombinant monovalent or bivalent scFv 7C10-C5 antibodies specifically recognized the carboxymethylated meL309 peptide, but none of the non-carboxymethylated peptides, regardless of phosphorylation at position 307. In contrast, the anti-non-carboxymethylated PP2Ac antibody 1D7 did not recognize the meL309 peptide.

Scfv 7C10-C5 ELISA

Nunc Medisorp 96-well ELISA flat-bottom plates were coated with C-terminal PP2Ac peptides either free or linked to BSA. Specifically, the peptides were: unmodified L309 (CGEPHVTRRTPDYFL) (SEQ ID NO:49), Y307F (CGEPHVTRRTPDFFL) (SEQ ID NO:50), pY307 (CGEPHVTRRTPDpYFL) (SEQ ID NO:51) or carboxymethylated meL309 (CGEPHVTRRTPDYFL-CH₃) (SEQ ID NO:49), on the ELISA plate at 8 μg/ml in TBS (free) or 1 μg/ml in TBS (linked to BSA) at 4° C. overnight. After washing twice with TBS, the wells were incubated with 2% BSA in TBS for 1 h at RT. After washing once with TBS, the wells were incubated with single clone hybridoma cell culture supernatant, clone 7C10-C5 1:50 (7C10 X63), recombinant single chain variable fragment of antibody 7C10-C5, undiluted (7C10 scFvs), or recombinant bivalent single chain variable fragment of antibody 7C10-C5, undiluted (7C10 bi-scFvs). After washing 3× with TBS, wells incubated with recombinant antibodies were incubated with monoclonal antibody 16B12, 1:10000 (recognizing the hemagglutinin tag on the recombinant antibody) in 1% BSA TBS 1 h at room temperature. After washing 3× in TBS, incubation with anti-mouse-HRP coupled secondary antibody for 1 h at RT was performed. Bound antibodies were detected by colorimetric reaction with 3,3′,5,5′-Tetramethylbenzidine as substrate, and absorbance was measured at 450 nm. Values were normalized to meL309 which was arbitrarily set to 1. Average and standard deviation of N=3 experiments are shown.

It was found that, similarly to the monoclonal antibody 7C10-C5 from the hybridoma supernatant, both recombinant monovalent or bivalent scFv 7C10-C5 antibodies specifically recognized the carboxymethylated meL309 peptide, but none of the non-carboxymethylated peptides, regardless of phosphorylation at position 307. Of note, the bivalent scFv has bound stronger to the meL309 peptide than the monovalent scFv.

In conclusion, both recombinant monovalent or bivalent scFvs of 7C10-C5 are highly specific for the α-carboxymethylated PP2Ac. This confirms that the constant region is not required for the anti-carboxymethylated PP2Ac binding specificity of 7C10-C5.

### Example 5. Carboxymethylated PP2Ac Levels Detected by a Truly Specific Anti-Methyl-PP2Ac Antibody (7C10-C5) Indicate PP2A Activity

Small molecule activators of PP2A (SMAPs) have been previously developed, with the lead drug DT-061 that shows anti-tumor efficacy in cancer mouse models (Allen-Petersen (2019), Cancer Res 79, 209-219; Kastrinsky (2015), Bioorganic & medicinal chemistry 23, 6528-6534; McClinch (2018), Cancer Res 78, 2065-2080; Sangodkar (2017), The Journal of clinical investigation 127, 2081-2090; Tohme (2019), JCI insight 4). DT-061 binds to and activates PP2A holoenzymes such as the AB56αC holoenzyme (Allen-Petersen (2019), Cancer Res 79, 209-219), whose assembly and activation in vivo is regulated by the LCMT-1 catalysed α-carboxymethylation of PP2Ac. The inventors have therefore assessed if the DT-061-mediated stabilization/activation of PP2A holoenzymes can be determined by monitoring the methylation state of the PP2A catalytic subunit in cells/tissues from an in vivo DT-061 treated xenograft tumor model, using the 7C10-C5 antibody.

Xenograft Tumor Model

Specifically, for xenograft tumor formation and in vivo DT-061 treatment studies, 5×10⁶ H358 (NCI-H358; ATCC® CRL-5807™) cells were resuspended in a 1:1 mix of RPMI:Matrigel and subcutaneously injected into the right flank of 6-8 week old Balb/c nu/nu mice. Tumors were monitored and measured twice a week until study enrollment. For the DT-061 time-course analysis, tumors were grown to 300 mm³, then randomized into a control N,N-Dimethylacetamide (DMA; solvent for DT-061) 24 hour treatment group or a DT-061 (5 mg/kg BID) treatment group which included a 1 hour, 2 hour, 3 hour, 6 hour, 12 hour, and 24 hour group (7 total groups for randomization). After the respective treatment incubation, mice were sacrificed, and serum and tumor tissue were harvested. Tumor tissue was both formalin fixed for IHC and snap frozen, along with collected serum, in liquid nitrogen for immunoblotting or molecule exposure analysis, respectively. Serum was analyzed by Agilux Laboratories Inc. to determine serum concentrations of DT-061. For in vivo treatment studies, DT-061 was dissolved in 10% DMA, 10% solutol, and 80% water.

Immunostaining of Xenograft Tumors

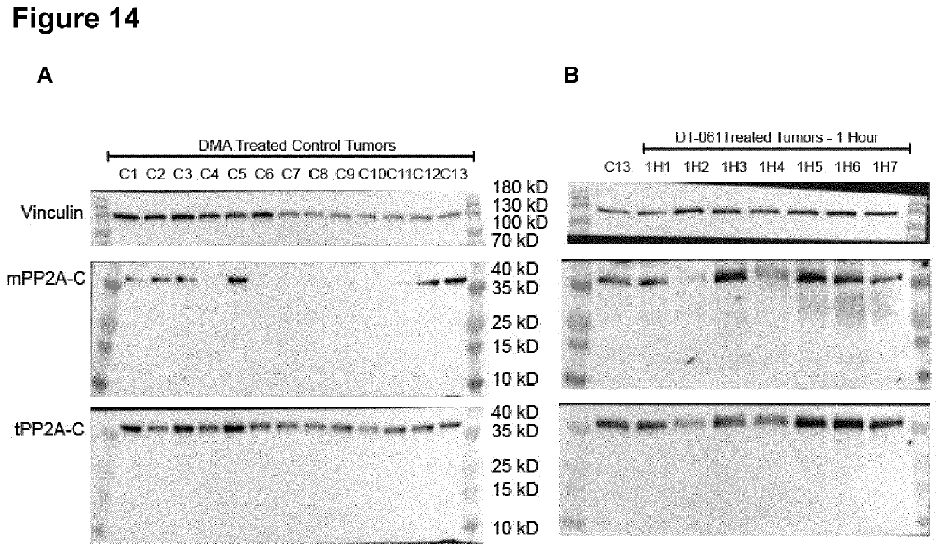

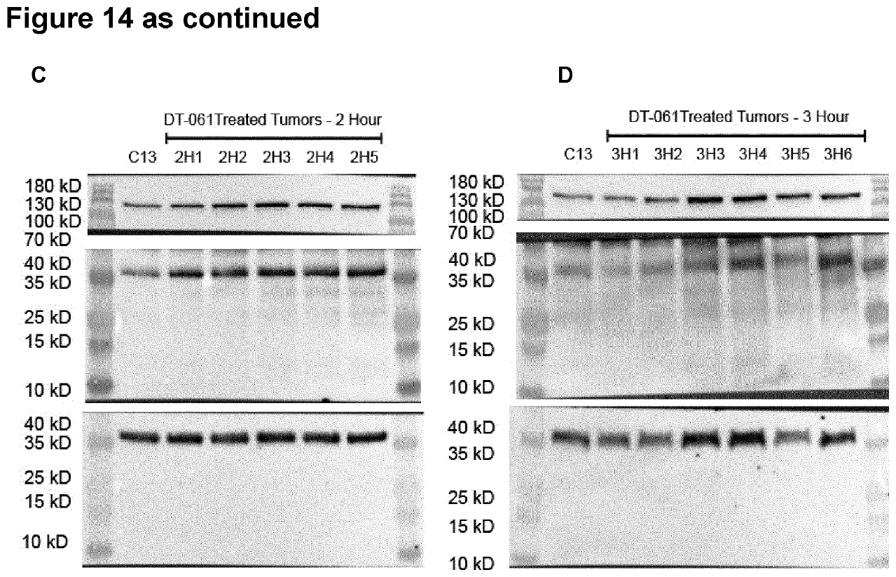

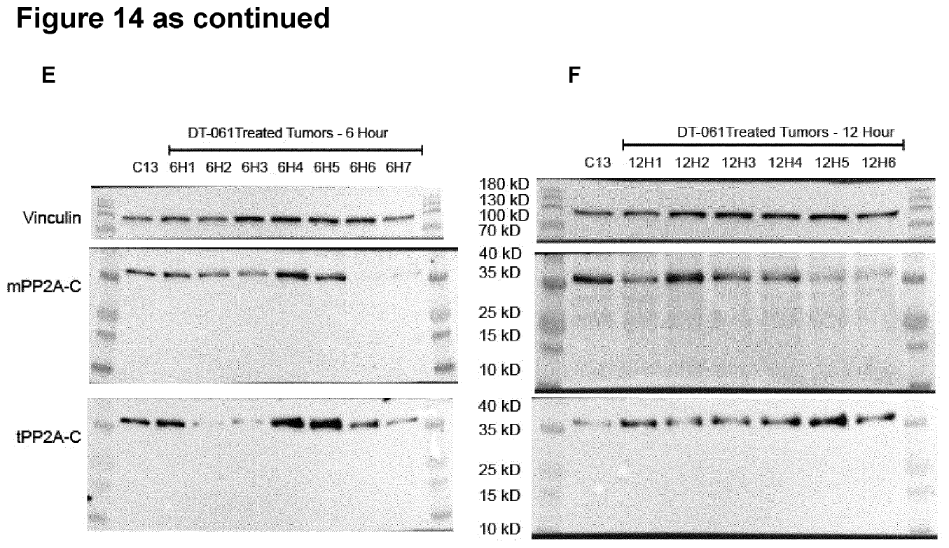

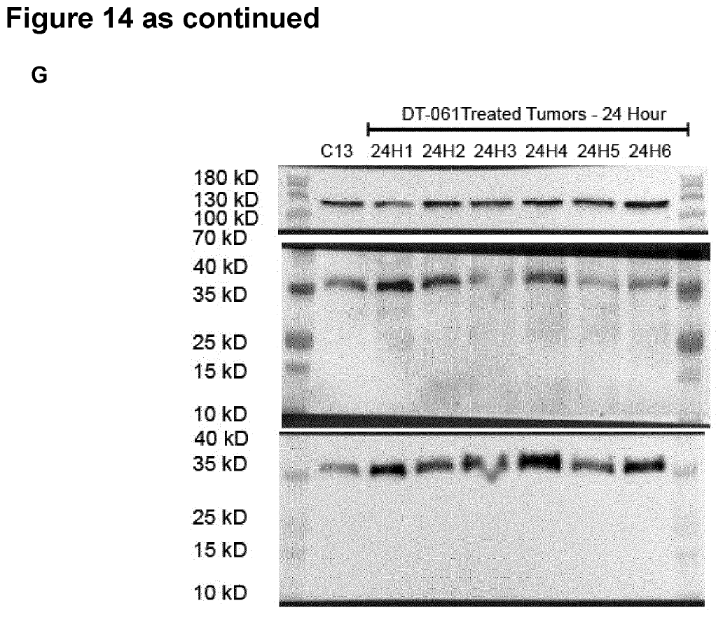

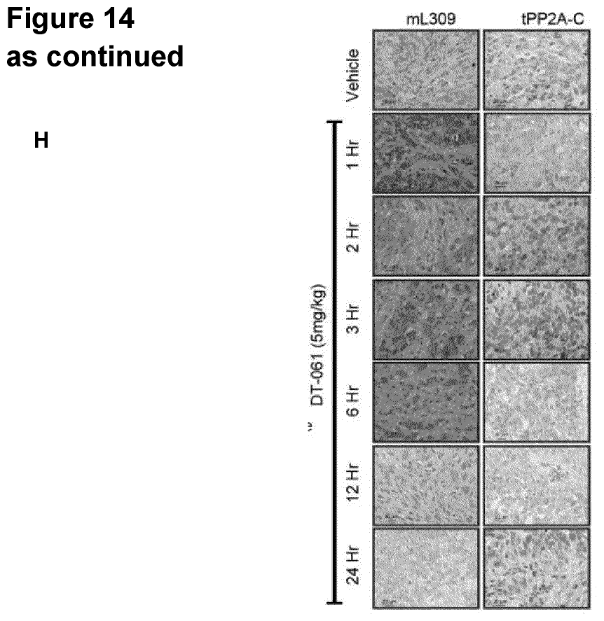

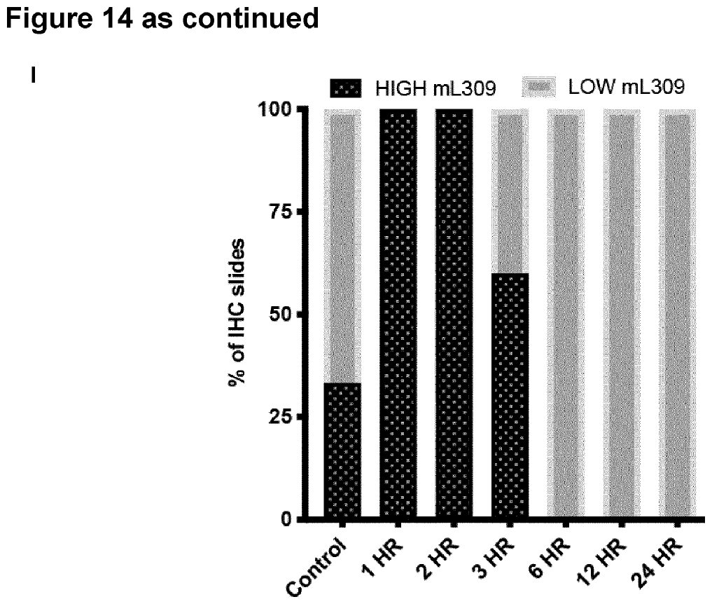

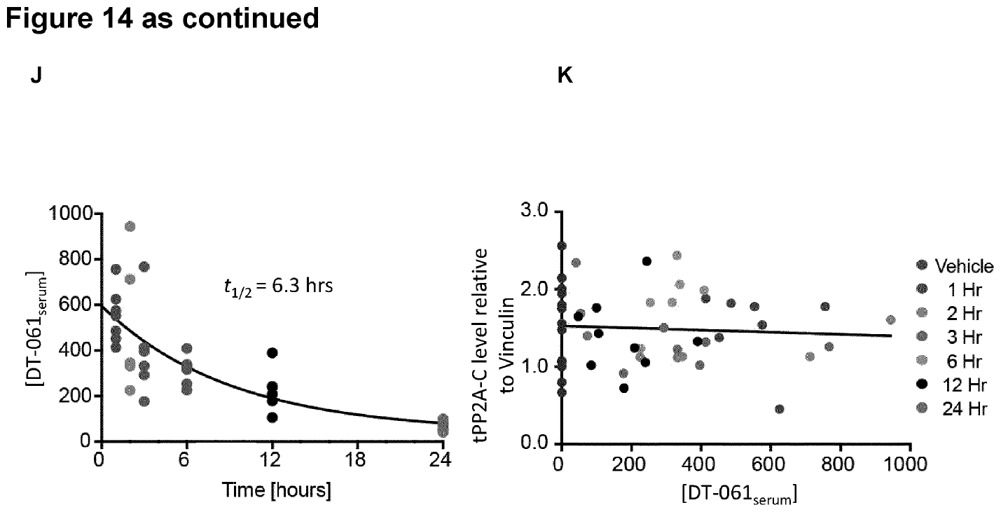

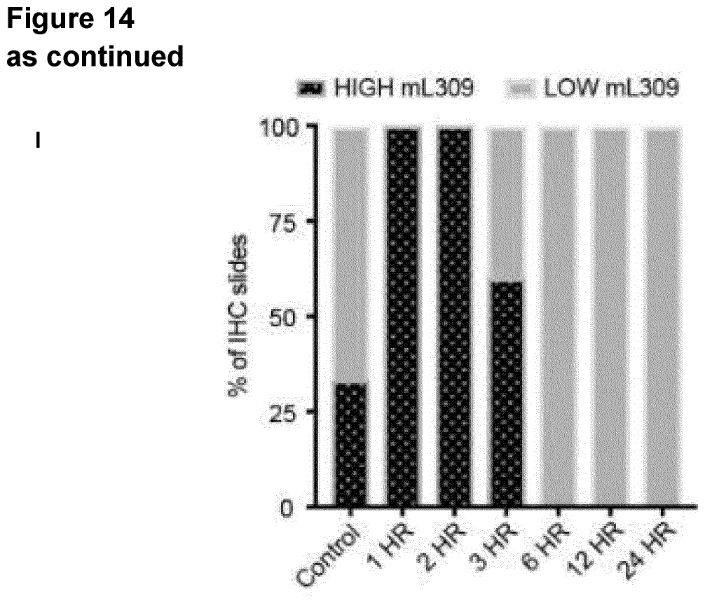

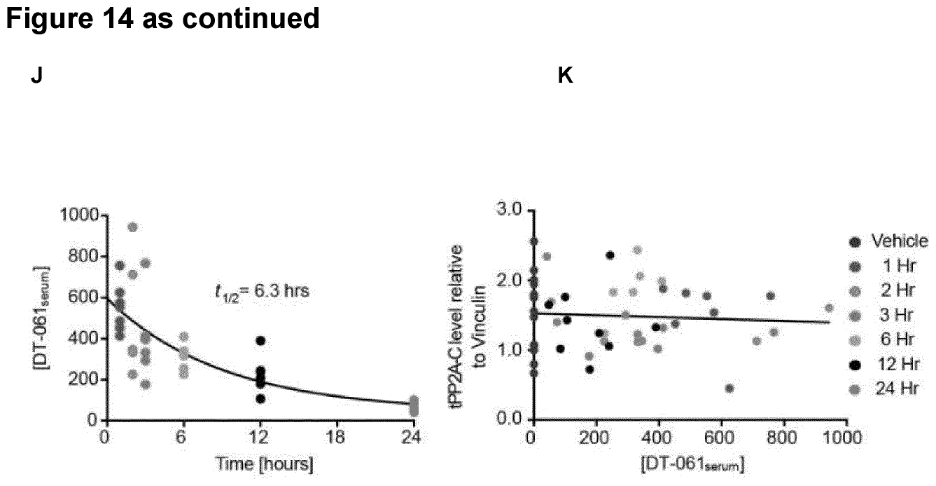

Next, the immunohistochemical analysis of xenograft tumors was performed. In brief, five micron sections from formalin-fixed paraffin-embedded (FFPE) specimens were deparaffinized in Histo-Clear and ethanol, exposed to 0.3% hydrogen peroxide to block endogenous peroxidase activity, and incubated for antigen retrieval with a citrate-based antigen unmasking solution (H-3300, Vector Laboratories) in a pressure cooker. Tumor sections were blocked with Mouse Ig Blocking Reagent (BMK-2202, Vector Laboratories), and incubated with anti-carboxymethyl PP2Ac specific antibody (7C10-C5) or an antibody recognizing total PP2Ac (ab106262) at 4° C. for two nights. Washes were performed in PBS. Slides were subsequently developed using secondary anti-mouse immunoglobulins/HRP (K1497, Agilent), DAB peroxidase substrate as the chromagen, and hematoxylin for counterstaining nuclei. Slides were sealed, and randomized for subsequent blinded review. Two independent researchers scored the level of carboxymethylated PP2Ac staining (with 7C10-C5) in one section per tumor (min. 6 tumors per treatment/control group). Specifically, each IHC sample was scored in a blinded fashion where each slide was categorized the following way: little to no staining (0), less than 50% of the slide had a moderate to low intensity stain (1), or more than 50% of the slide had a moderate to high intensity stain (2). The scores per slide were then combined so that each slide had a grade from 0-4 (2×0-2). Scores from 0-2 are considered low staining, 2-4 as moderate, and 4-6 as high intensity staining. Based on these scores the data was plotted as the percentage of slides from each time point that fell into each group (FIG. 14I).

Immunohistochemical Analysis of Rabbit Monoclonal E155 and Mouse Monocolonal 7C10-C5

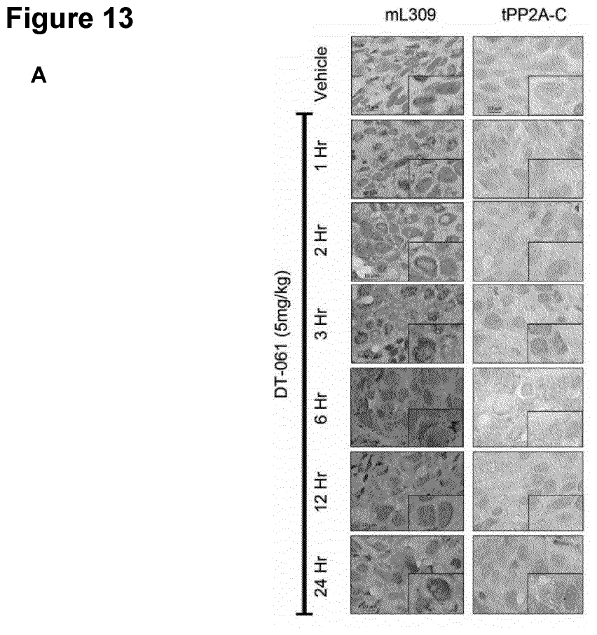

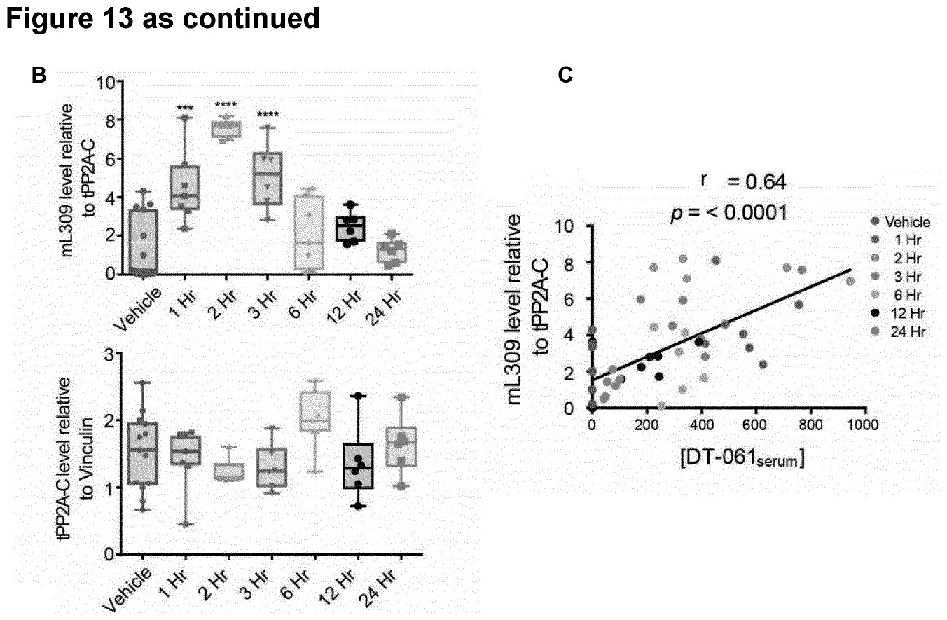

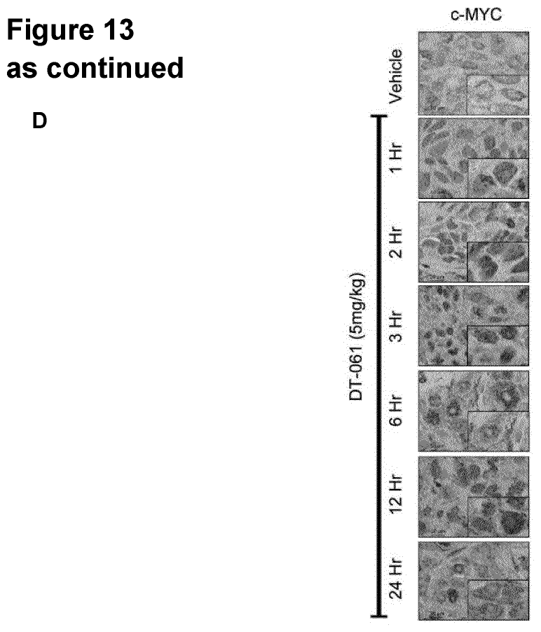

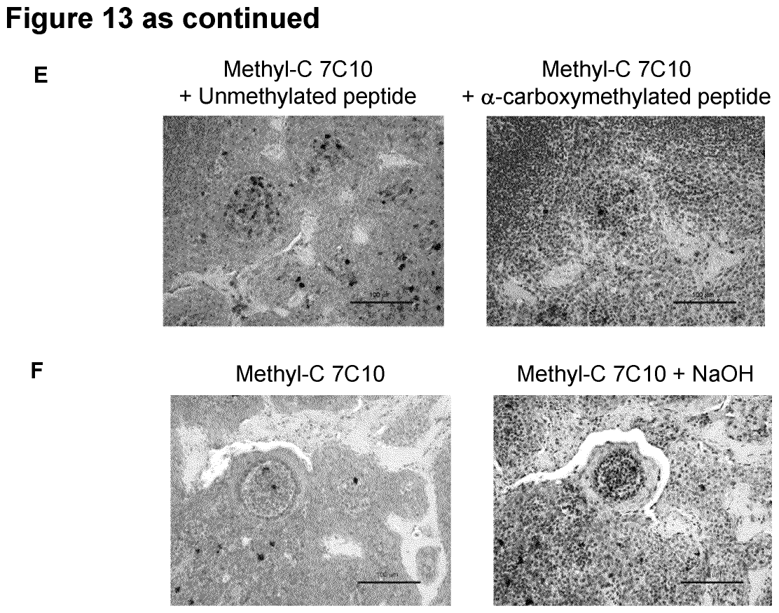

Immunohistochemical staining was performed on the DAKO Autostainer (DAKO, Carpinteria, Calif.) using Envision+ or liquid streptavidin-biotin and diaminobenzadine (DAB) as the chromogen. De-paraffinized sections were labeled with mouse monoclonal antibody 7C10-C5 (1:200) overnight at 4 C. Microwave 10 mM Tris HCl, pH9 containing 1 mM EDTA epitope retrieval was used prior to staining. Appropriate negative (no primary antibody) and positive controls (pancreas) were stained in parallel with each set of slides studied. Peptide competition studies were performed using 2 peptides (DPAPRRGEPHVTRRTPDYFL*) (SEQ ID NO:115) differing in the methylation status at position L (marked with a *); FIG. 13E: Peptides were incubated at ambient temperature for 60 mins at 10-1000× molar ratios before being applied to the slide. FIG. 13F: Before being stained, slides were incubated in freshly made 200 mM NaOH for 10 mins at 4° C. and then neutralized by several washes in TBST.

Stained slides were scanned using an APERIO AT2 (Leica) and the digital images imported into QuPath (Bankhead et al., 2017). TMA cores were analyzed for percentage of cells staining and intensity. In FIGS. 13E and F, higher visibility of the dark cell nuclei indicates weaker α-carboxymethylated PP2Ac staining.

Analysis of DT-061 Treated Xenograft Tumors

Immunohistochemical (IHC) analysis of xenograft DT-061 treated tumors with an anti-methyl-PP2Ac antibody of the invention (i.e here 7C10-C5) revealed an increase in α-carboxymethylated PP2Ac at early time points (one, two, and three hours) after single dose DT-061 treatment followed by a return to basal levels by 12 hours post treatment (FIG. 13A+B). Peptide competition with non-methylated or α-carboxymethylated peptides confirmed the specificity of 7C10-C5 for the α-carboxymethylated PP2Ac also in IHC applications (FIG. 13E). Additional validation of the specificity of the anti-methyl-PP2Ac antibody of the invention (i.e here 7C10-C5) for the α-carboxymethylated PP2Ac in IHC applications was performed with NaOH treatment to chemically remove the methyl group from the methylated PP2A-C. Immunohistochemical staining with the 7C10-C5 antibody showed diminished staining upon treatment with NaOH (FIG. 13F). Additionally, the observed changes in the α-carboxymethylation of PP2Ac positively correlated with DT-061 serum concentration (FIG. 13C), whose half-life obtained from serum was determined to be 6.3 hours (FIG. 14J). The temporal increase in α-carboxymethylation of PP2Ac and subsequent return to baseline following DT-061 treatment was also confirmed in the immunoblotting analysis of tumor lysates (FIGS. 14A-G and 14I).

The potent oncoprotein c-MYC is one of the most well characterized substrates of AB56αC holoenzymes. Dephosphorylation of c-MYC at serine 62 by AB56αC leads to the polyubiquitination and degradation of c-MYC (Arnold and Sears, 2006). It was therefore checked if monitoring carboxymethylated PP2Ac levels with the 7C10-C5 antibody allows to assess the (de)phosphorylation of the PP2A substrate c-MYC. Immunohistochemical analysis from the single dose treated xenograft tumors showed a significant decrease of c-MYC at early time points (one, two, and three hours) following DT-061 exposure which then return to basal levels by 6 hours (FIG. 13D). The kinetic loss and regain of c-MYC following DT-061 treatment revealed an anti-parallel trend when compared to the α-carboxymethylation of PP2Ac (as detected with anti-methyl-PP2Ac antibody of the present invention) further supporting the use of PP2Ac α-carboxymethylation as a pharmacodynamic marker of DT-061 enhanced stabilization/increase of active AB56αC holoenzymes.

In other words, a truly anti-methyl-PP2Ac-specific antibody (here: monoclonal antibody 7C10-C5) allows to detect the increase of the α-carboxymethylation of PP2Ac caused by PP2A activators, i.e. a SMAP, which correlates with the stabilization of specific trimeric PP2A holoenzymes (including AB56αC holoenzymes) and the dephosphorylation of specific substrates of the drug-stabilized holoenzyme (for example, c-MYC). The α-carboxymethylation of PP2Ac detected by the specific anti-methyl-PP2Ac antibody of the invention (i.e. 7C10-C5) may thus be used for screening drugs which potentially target PP2A, and i.e. modulate PP2A activity. In particular, potential PP2A activators may be tested if they enhance PP2A activity and hence may improve therapeutic efficacy and target engagement of PP2A for the treatment of certain diseases, such as inter alia cancer, wherein increased α-carboxymethylated PP2Ac levels correspond to an increased PP2A activity. In other words, the α-carboxymethylation level of PP2Ac as detected the truly anti-methyl-PP2Ac specific antibody of this invention (i.e. monoclonal antibody 7C10-C5) is a marker (in particular a pharmacodynamic) marker for PP2A activation including, but not limited to, SMAP enhanced stabilization of active AB56αC holoenzymes.

### Example 6. The α-Carboxymethylation Level of PP2Ac as Detected by an Antibody Specific for Methyl-PP2Ac (Anti-Methyl-PP2Ac-Specific Antibody of this Invention, in Particular Monoclonal Antibody 7C10-C5) is a Prognostic Biomarker in Cancer and Other Diseases

The assembly of trimeric tumor-suppressive PP2A holoenzymes is promoted/facilitated by PP2Ac α-carboxymethylation. The PP2Ac α-carboxymethylation levels correlate positively with the levels of methylation-sensitive trimeric PP2A holoenzymes. A truly specific anti-methyl-PP2Ac antibody (7C10-C5) can be used to determine the α-carboxymethylated PP2Ac levels in normal and disease tissue by IHC analyses of tissue microarrays of cancer, normal tissues and tissues obtained from other disease states including but not limited to heart disease, Alzheimers disease, and diabetes. The PP2A methylation status in disease tissue can be graded using the Allred scoring system that scores the proportion of cells that stain by IHC as well as the intensity of the IHC staining. Specifically, the Allied scoring system is based on the percentage of cells that stain by immunohistochemistry for carboxymethylated PP2Ac (on a scale of 0 to 5) and the intensity of that staining (on a scale of 0 to 3), for a possible total score of 8. Allied scoring is calculated the following way:

Proportion Score:

0—No cells, or essentially no cells (e.g. ≤0.01%) stain positively

1—≤1% (e.g. 0.5-1%) of cells stain positively

2—1-10% (i.e. >1% and ≤10%; e.g. 2-10%) of cells stain positively

3—11-33% of cells stain positively

4—34-66% of cells stain positively

5—67-100% of cells stain positively;

Intensity Score:

0—Negative.

1—Weak.

2—Intermediate.

3—Strong.

For example, if 67-100% of cells stain positively and the staining intensity in the sample is at maximum, the Allred score is 8 (5+3).

For example, Allied scores of 0-3 may indicate low PP2Ac α-carboxymethylation levels and low/very low amounts of tumor-suppressive PP2A holoenzymes and Allred scores of at least 6-8 may indicate intermediate to high α-carboxymethylation levels and high/very high amounts of tumor-suppressive PP2A holoenzymes. However, the thresholding of Allred scores may vary and/or be adjusted according to the sample or disease. For example, it is also possible that an Allred score below 2 (i.e. 0 or 1) indicates very low PP2Ac α-carboxymethylation levels, essentially no carboxymethylated PP2Ac, or no carboxymethylated PP2Ac, and an Allied score of at least 2 (i.e. 2, 3, 4, 5, 6, 7, or 8) indicates low/intermediate to high α-carboxymethylation levels.

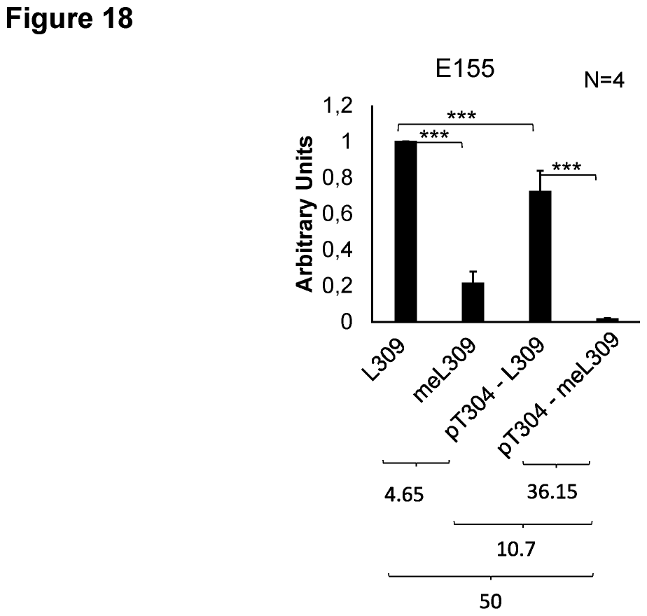

A truly specific anti-methyl-PP2Ac antibody (7C10-C5) is particularly useful for investigating cancers (lung adenocarcinoma, breast and prostate cancer) with oncogenic hyper-activated Akt, S6K and ERK/MAP kinase signaling pathways, because methylation-sensitive PP2A holoenzymes are known to negatively regulate these signaling pathways by dephosphorylating substrates downstream of the hyperactivated receptor tyrosine kinases (Jackson (2012), Neoplasia 14, 585-599). A critical step in tumor initiation and metastasis is the capacity of transformed cells to grow anchorage-independently, which is facilitated by resisting anoikis, a programmed cell death induced by the detachment from the extracellular matrix (ECM). Knockdown of the PP2A methyltransferase LCMT-1 or overexpression of the PP2A methylesterase PME1, which both lead to the reduction of the α-carboxymethylation levels of PP2Ac and as a consequence to the reduction of methylation-sensitive holoenzymes, enhance transformation/promote anchorage-independent growth by activating the AKT and p70/p85 S6K pathways (by preventing the inhibition of AKT and S6K) (Jackson (2012), Neoplasia 14, 585-599). Along these lines, inhibition/low activity of PP2A increases cancer cell survival under suspension conditions and also the tumor initiation capacities of cancer cells (Liu (2016), Nat Commun 7, 11798). Of note, although these authors claimed (by using the rabbit monoclonal antibody E155) that Tyr³⁰⁷ phosphorylation of PP2Ac is causing the inhibition of PP2Ac, re-evaluating the data based on the actual specificity of E155 (see FIGS. 18-20 and FIG. 22), rather suggests that the inhibition/low activity of PP2A might be based on increased non-methylated PP2Ac/reduced methylated PP2Ac levels. Thus, low levels of α-carboxymethylated PP2Ac might indicate reduced levels of tumor suppressive PP2A holoenzymes, which correlates with worse prognosis for the cancer patient, whereas high α-carboxymethylated PP2Ac levels might indicate high levels of tumor suppressive activity of PP2A and point to a more favorable outcome for the cancer patient. To test this hypothesis, the inventors have carried out IHC tissue microarray analyses of prostate cancer tissue with the 7C10-C5 antibody.

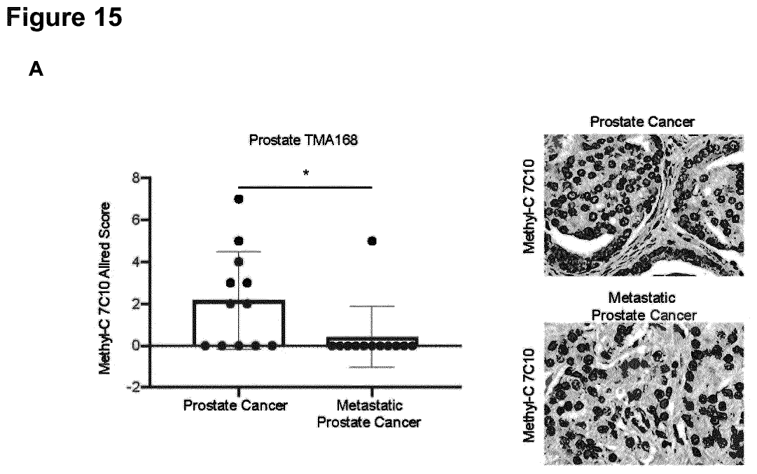

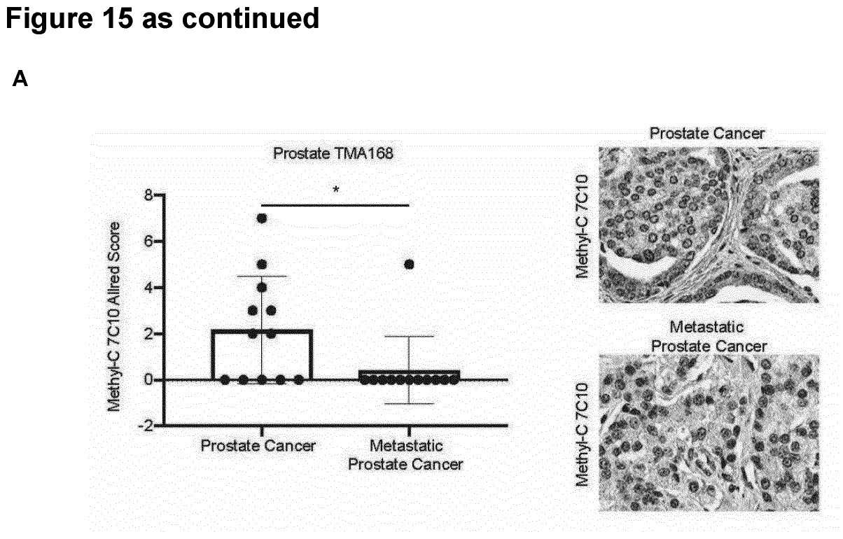

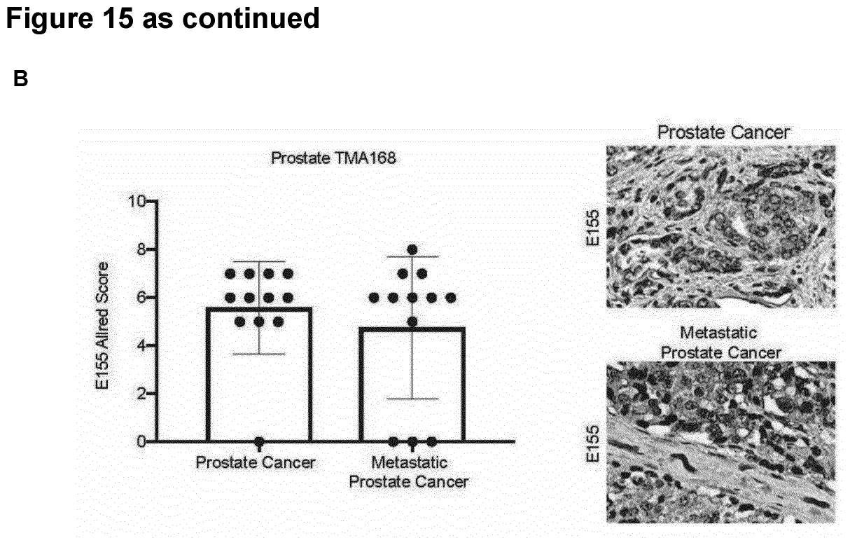

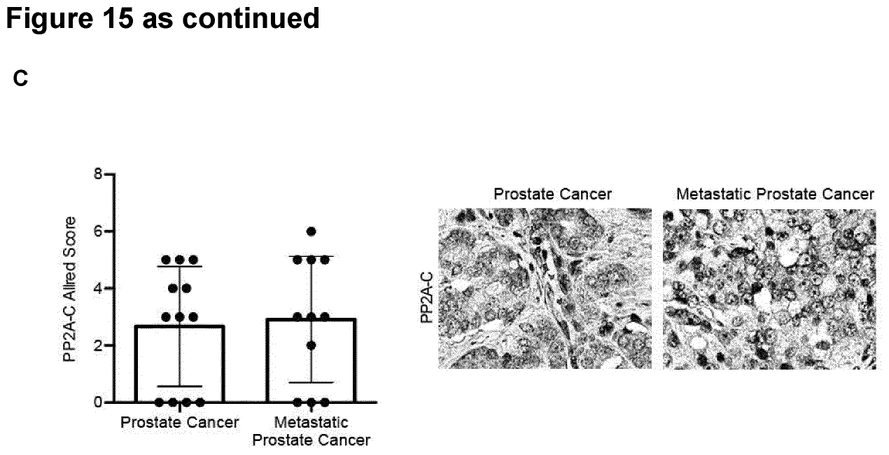

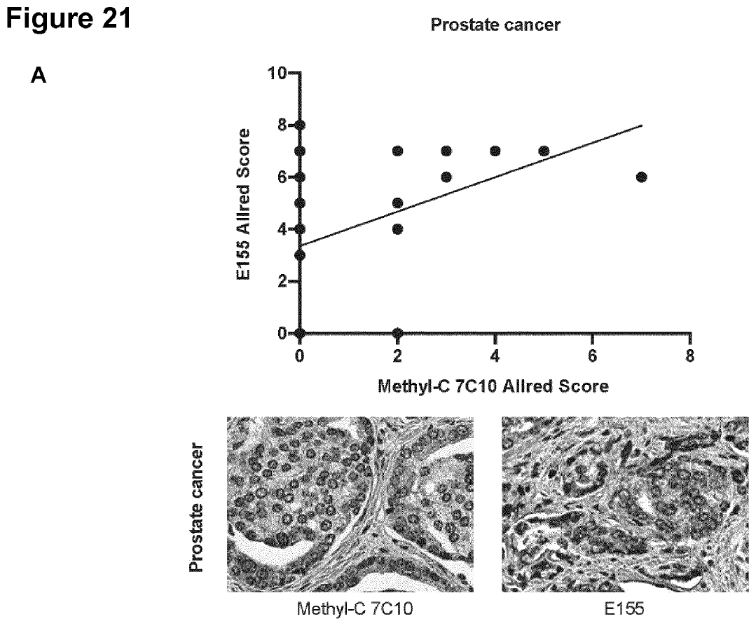

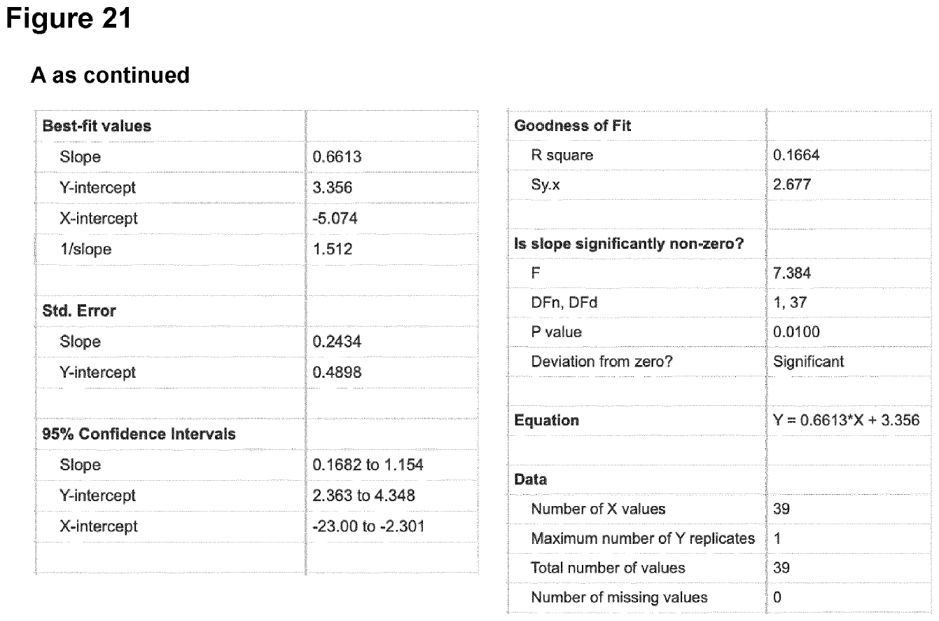

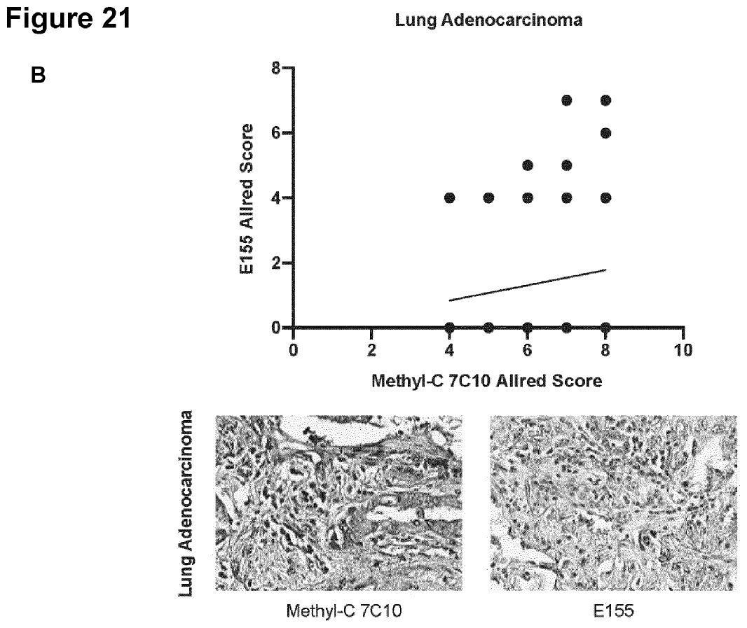

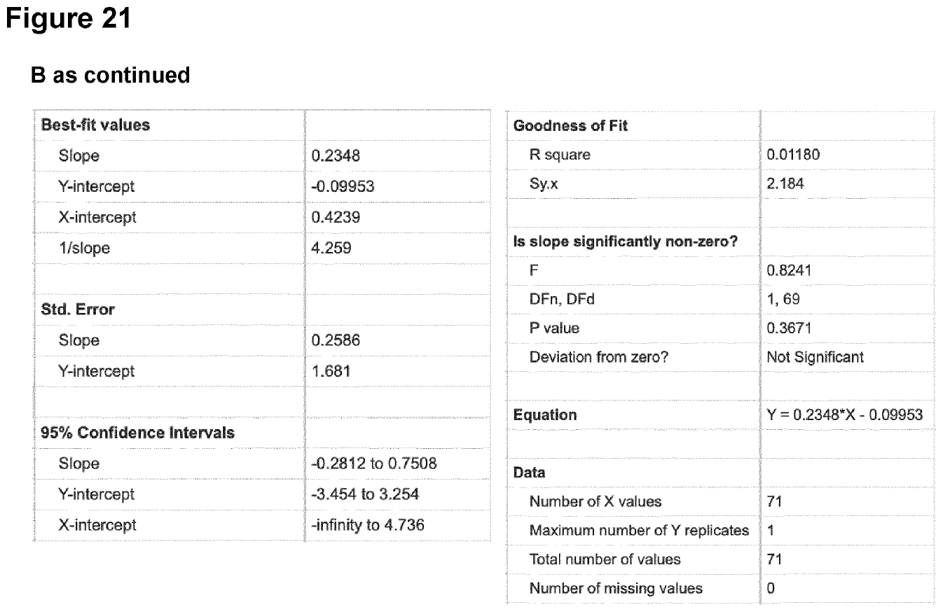

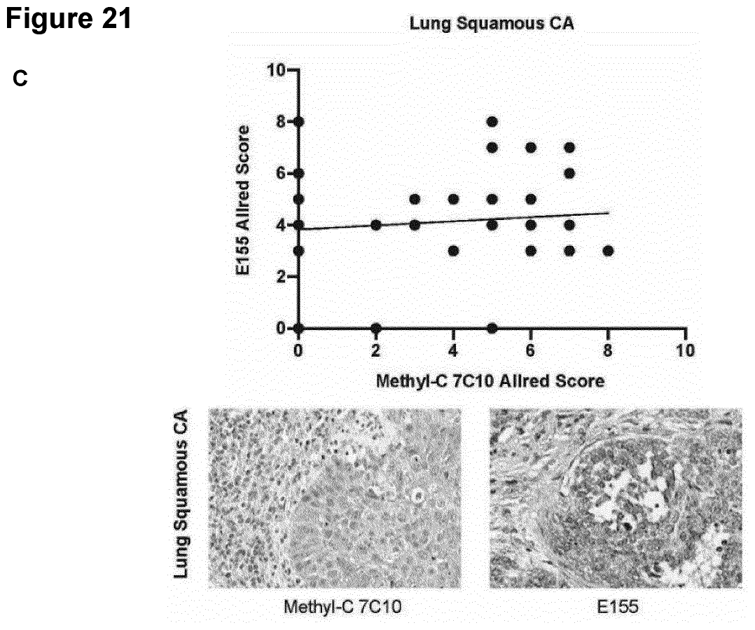

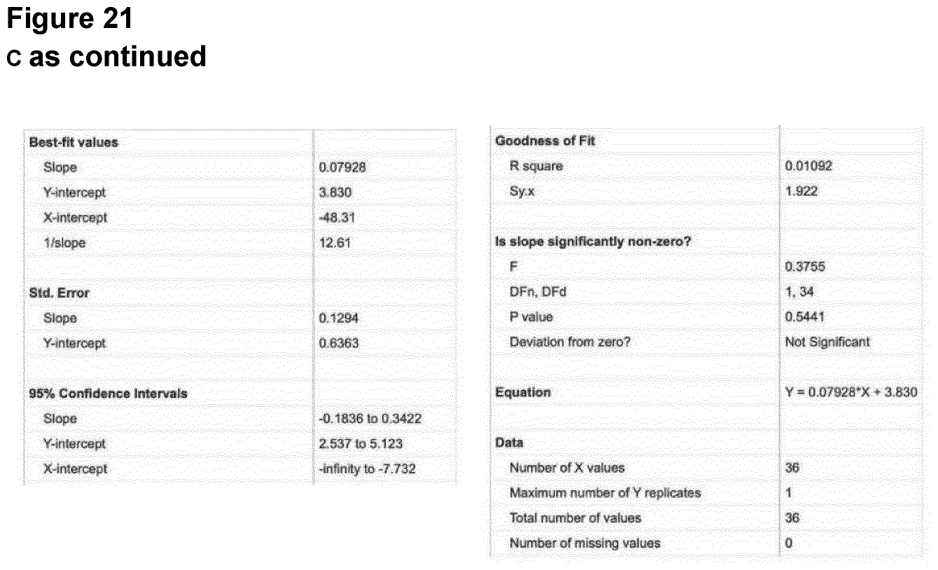

The experiments revealed that 7 out of 12 non-metastatic (localized) prostate cancer tissue samples had Allred scores between 2 and 7, whereas 11 out of 12 metastatic prostate cancer tissues (progressed tumor) had an Allied score of 0 suggesting a correlation between disease stage and the levels of α-carboxymethylated PP2Ac in prostate cancer tissue (FIG. 15). In particular, the localized prostate cancer tissues had an average Allred score of 2.2±2.3 (mean±standard deviation), whereas the metastatic prostate cancer tissues had an average Allred score of 0.4±1.4 (mean±standard deviation), as determined by IHC with the 7C10-C5 antibody. Thus, metastatic prostate cancer tissues had, in average, about 80%, e.g. 82%, lower α-carboxymethylated PP2Ac levels (according to the Allred Score) than localized prostate cancer tissue samples, as determined with the inventive anti-α-carboxymethylated PP2Ac antibody (e.g. 7C10-C5) provided herein. Thus, the data indicate that the lower the α-carboxymethylated PP2Ac levels and the lower the Allred score, the more progressed the tumor/cancer is and/or the faster the tumor/cancer will progress. In other words: low carboxymethylated PP2Ac levels (e.g. an Allied score <2) indicate low PP2A activity which indicates an unfavorable progression and/or a negative outcome, whereas high carboxymethylated PP2Ac levels (e.g. an Allred score of ≥2) indicate high PP2A activity which indicates a favorable progression and/or positive outcome of the tumor/cancer. This correlation, however, was not detected with the rabbit monoclonal E155 antibody that was used to detect phosphorylated Tyr³⁰⁷ as an indicator of PP2Ac inhibition, and which has been claimed to serve as a prognostic marker in certain cancer types Chen (2017), Hum Pathol 66, 93-100; Cristobal (2014), Br J Cancer 111, 756-762; Rincon (2015), Oncotarget 6, 4299-4314). In addition, the signals detected with E155 in the IHC analysis of 3 different cancer tissue types did not inversely correlate with the α-carboxymethyl PP2Ac signals detected with the monoclonal antibody 7C10-C5 (FIG. 21A-C) indicating that the specificity of E155 for PP2Ac is not reciprocal to the α-carboxymethylation specificity of 7C10-C5. Furthermore, evaluation of total PP2Ac with an anti-total PP2Ac antibody YE351 (ab32065, abcam) showed no significant differences between localized and metastatic prostate cancer samples (FIG. 15 C). This anti-total PP2Ac antibody is insensitive to methylation or phosphorylation of the carboxy terminus of the PP2A catalytic subunit because its antigen is located in in the N terminal region of PP2Ac according to the manufacturer's information (https://www.abcam.com/pp2a-alpha-beta-antibody-ye351-ab32065.html). Thus, these data suggest that the level of methylation-dependent PP2A holoenzymes is reduced in metastatic prostate cancer but not in localized prostate cancer. Furthermore, these data show that anti-PP2Ac antibodies that are not truly specific for carboxymethylation may be generally not suitable for distinguishing localized and metastatic cancers. Thus, the inventive anti-carboxymethylated PP2Ac-specific antibody provided herein is very advantageous for use in cancer diagnostics, e.g. in the diagnostic/prognostic methods described herein.

### Example 7. The α-Carboxymethylation Level of PP2Ac Detected by a Truly Anti-Methyl-PP2Ac Specific Antibody (Like 7C10-C5) Allows the Prediction of Responsiveness to Cancer and Other Disease Therapy

The α-carboxymethylated PP2Ac levels may be used for predicting, e.g. based on the Allied scores of the IHC analyses, the likelihood of a therapeutic response to drug-induced inhibition of signaling pathways that are known to be suppressed/negatively regulated by carboxymethylation-regulated, tumor-suppressive PP2A holoenzymes, for example the epidermal growth factor receptor (EGFR), the AKT, c-MYC, ERK, and β-catenin pathways because α-carboxymethylated PP2Ac levels are indicative of the levels of trimeric tumor-suppressive PP2A holoenzymes present in cancer/disease cells. The α-carboxymethylated PP2Ac levels determined by an anti-methyl-PP2Ac specific antibodies of this invention (like, e.g. 7C10-C5) may allow to predict the efficacy of kinase inhibitors in turning off the hyperactivated proliferation pathways in cells. In order to reset an hyperactivated kinase pathway to ground-state, it may not be sufficient to only inhibit a certain kinase because an active phosphatase, i.e. PP2A, may be needed to further dephosphorylate the still (hyper)phosphorylated substrate(s). Disease tissue with a high Allred score of 6-8 (with high α-carboxymethylated PP2Ac levels) may predict a high likelihood of response to cancer therapy, because high levels of α-carboxymethylated PP2Ac are indicative of high levels of active, tumor-suppressive PP2A holoenzymes that efficiently dephosphorylate the hyperphosphorylated substrates, thereby shutting down the hyperactivated pathways. In turn, low Allred scores between 0-3, i.e. 0 or 1, indicate low/very low levels of α-carboxymethylated PP2Ac or no carboxymethylated PP2Ac correlating with low PP2A activity and thus may predict a low likelihood of response to cancer therapy.

When SMAPs (e.g. DT-061) or other PP2A activating/modulating drugs such as forskolin are used in therapies, the α-carboxymethylation level of PP2Ac of tumor tissue as determined by the 7C10-C5 antibody might serve as a decision criterion for identifying treatment responsive patient populations (predictive biomarker). For example, when the carboxymethylation level of PP2Ac is low, the patient may be responsive to activation of PP2A. In other words, determining the α-carboxymethylation level of PP2Ac of tumor tissues of patients with the 7C10-C5 antibody may allow stratifying the patients into “likely drug (e.g. DT-061) responders” having low carboxymethylated PP2Ac levels and “unlikely drug (e.g. DT-061) responders” already having high carboxymethylated PP2Ac levels. Moreover, a drug induced increase of α-carboxymethylation PP2Ac levels might indicate (re-) activation of PP2A tumor suppressive functions.

### Example 8. Prior Art Anti-PP2Ac Antibodies do not Specifically Bind Carboxymethylated PP2Ac

Phosphorylation of PP2Ac subunit either on Tyr³⁰⁷ or an undefined threonine residue has been primarily associated with the inhibition of its catalytic activity (Chen et al., 1992; Chen et al., 1994; Damuni et al., 1994; Guo and Damuni, 1993). The most frequently cited antibody (112 publications) for the detection of pTyr³⁰⁷ is a rabbit monoclonal antibody clone E155 generated by Epitomics (catalog #1155-1, Abeam). High levels of Tyr³⁰⁷ phosphorylation detected by E155 were interpreted as evidence for PP2A inhibition and were claimed to correlate with poor outcome/overall survival in different human cancer types (Chen et al., 2017; Cristobal et al., 2014; Rincon et al., 2015). Later-on, the antibody has been re-examined, and it was clarified that E155 is not specific for pTyr³⁰⁷. Furthermore, it was found by the inventors that commercial pTyr³⁰⁷ antibodies including E155 and mouse monoclonal F-8 from Santa Cruz Biotechnology (SCBT) are not specific for pTyr³⁰⁷ but recognize the non-methylated C-terminus of PP2Ac and are impaired in recognition of PP2Ac by α-carboxymethylation of PP2Ac at Leu³⁰⁹ and to a lesser extent also by phosphorylation of Thr³⁰⁴.

Prior Art Antibodies Described as “Anti-Carboxymethylated PP2Ac Antibodies”

The only commercially available antibody which binds α-carboxymethylated PP2Ac specificity but not non-carboxymethylated PP2Ac is 2A10 that is sold by several companies including Upstate Biotechnology (now Merck-Millipore), Abcam, Biolegend/Covance, ImmuQuest and Santa Cruz Biotechnology. This clone, however, possesses cross-reactivity with the methylated PP4c and weakly also with methylated PP6c (FIG. 8B+C).

Later, a monoclonal antibody, termed 4D9 was reported to have specificity for the methylated PP2Ac (Tolstykh (2000), Embo J 19, 5682-5691). In contrast to 7C10-C5 (and also 2A10) this clone was raised against an amidated and not α-carboxymethylated C-terminal PP2Ac peptide (299-309). Amidation neutralizes the charge of the α-carboxyl-group and is thought to mimic the functional consequences of the in vivo occurring methylation. Thus, the 4D9 antibody may be specific for the uncharged, amidated C-terminus but not the α-carboxymethylated C-terminus of PP2Ac subunit per se. Its cross-reactivity with the PP2A-like phosphatases PP4c and PP6c that share the terminal 3 amino acids (FIG. 8A) with PP2Ac is unknown. It is also unknown to what extent its binding to carboxymethyl PP2Ac is influenced by the phosphorylation of Tyr³⁰⁷ and Thr³⁰⁴.

Anti-Non-Carboxymethylated PP2Ac Antibodies

Examples of antibodies with a preference for non-methylated PP2Ac are mouse monoclonal antibody 4B7 (Yu (2001), Mol Biol Cell 12, 185-199), mouse monoclonal antibody 1D7 generated by the Ogris lab but also the rabbit monoclonal E155 antibody from Abeam and the mouse monoclonal F-8 from SCBT that were thought to be specific for pTyr³⁰⁷. The inventors have analyzed the detection properties of 5 commercial antibodies that were raised against the unmodified C-terminus of PP2Ac and found that all of them displayed various degrees of being impaired by α-carboxymethylation of PP2Ac and cross-reactivities with PP4, and in addition their PP2Ac recognition was hampered by the phosphorylation of Tyr³⁰⁷ and Thr³⁰⁴ as summarized in Table 4.

Characterization of Prior Art Antibodies

E155 ELISAs

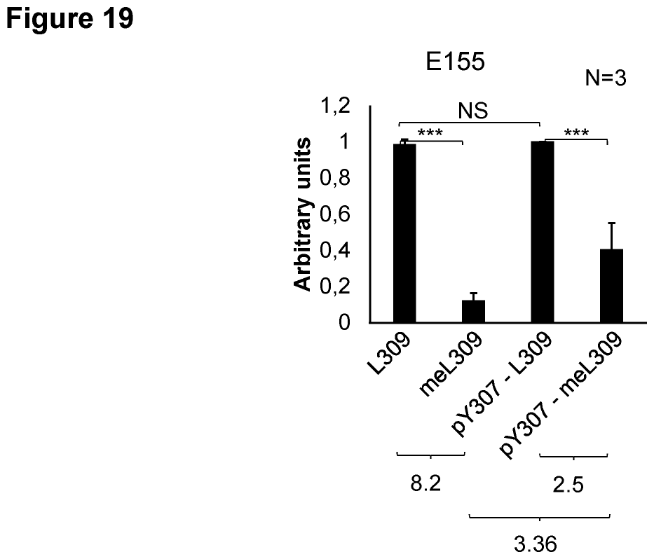

E155 was tested for its specificity to phosphorylation and methylation on the carboxy-terminus of PP2A on 6 different undecapeptides by ELISA (FIGS. 18 and 19). Flat-bottom Nunc-Immuno Medisorp 96-well ELISA plates (Thermo; 467320) were coated o/n at 4° C. with 50 μl of either a peptide of the sequence ac-HVTRRTPDYFL-OH (L309) (SEQ ID NO:18) at a concentration of 2 μg/ml, a peptide of the sequence ac-HVTRRTPDYFL-CH₃ (meL309) (SEQ ID NO:18) at a concentration of 2 μg/ml in TBS (137 mM NaCl (AppliChem, #131659, 2.7 mM KCl (AppliChem, #131494.), 24.8 mM Tris (AppliChem, #A1086), pH 7.4 with HCl), with a peptide of the sequence ac-HVTRRpTPDYFL-OH (pT304-L309) (SEQ ID NO:116) at a concentration of 2 μg/ml in TBS, or with a peptide of the sequence ac-HVTRRpTPDYFL-CH₃ (pT304-meL309) (SEQ ID NO:116) at a concentration of 2 μg/ml in TBS (FIG. 18). The wells were washed once with TBS, blocked with 200 μl of 2% bovine serum albumin (BSA) (Sigma, A9647) in TBS for 1 hour at RT and washed again once with TBS. The wells were incubated with 50 μl of E155 (Abeam, #ab32104 lot GR17965-24, 1 μg/ml) for 1 hour at RT. washed 3 times with TBS, incubated for 1 hour at RT with 50 μl peroxidase-conjugated AffiniPure goat anti-rabbit IgG Fcγ fragment specific (Jackson ImmunoResearch, 111 008) secondary antibody diluted 1:10,000 in TBS and washed 3 times with TBS. Bound antibodies were detected colorimetrically by addition of 50 μl of 33 μg/ml 3′,5′,5′,5′-tetramethylbenzidine (TMB) (Sigma, T2885) in 0.1 M sodium acetate pH 6.0 and 0.01% H₂O₂ (Sigma, H1009). The colorimetric reaction was stopped after 10 min by addition of 50 μl 0.5 M H₂SO₄, and absorbance was measured at 450 nm and 560 nm with a Perkin Elmer VICTOR® Nivo™ microplate reader. The results from 560 nm read were subtracted from the 450 nm read.

In a further experiment, flat-bottom Nunc-Immuno Medisorp 96-well ELISA plates (Thermo; 467320) were coated o/n at 4° C. with 50 μl of either a peptide of the sequence ac-HVTRRTPDYFL-OH (L309) (SEQ ID NO:18) at a concentration of 2 μg/ml in TBS (137 mM NaCl (AppliChem, #131659, 2.7 mM KCl (AppliChem, #131494.), 24.8 mM Tris (AppliChem, #A1086), pH 7.4 with HCl), a peptide of the sequence ac-HVTRRTPDYFL-CH₃ (meL309) (SEQ ID NO:18) at a concentration of 2 μg/ml in TBS (137 mM NaCl (AppliChem, #131659.1214, 2.7 mM KCl (AppliChem, #131494.1211) pH 7.4 with HCl), with a peptide of the sequence ac-HVTRRTPDpYFL-OH (pY307-L309) (SEQ ID NO:117) at a concentration of 2 μg/ml in TBS or with a peptide of the sequence ac-HVTRRTPDpYFL-CH₃ (pY307-meL309) (SEQ ID NO:117) at a concentration of 2 μg/ml in TBS (FIG. 19). The wells were washed once with TBS, blocked with 200 μl of 2% bovine serum albumin (BSA) (Sigma, A9647) in TBS for 1 hour at RT and washed again once with TBS. The wells were incubated with 50 μl of E155 (Abeam, #ab32104 lot GR17965-24), 1 μg/ml) for 1 hour at RT, washed 3 times with TBS, incubated for 1 hour at RT with 50 μl peroxidase-conjugated AffiniPure goat anti-rabbit IgG Fcγ fragment specific (Jackson ImmunoResearch, 111-035-008) secondary antibody diluted 1:10,000 in TBS and washed 3 times with TBS. Bound antibodies were detected colorimetrically by addition of 50 μl of 33 μg/ml 3′,5′,5′,5′-tetramethylbenzidine (TMB) (Sigma, T2885) in 0.1 M sodium acetate pH 6.0 and 0.01% H₂O₂ (Sigma, H1009). The colorimetric reaction was stopped after 10 min by addition of 50 μl 0.5 M H2SO4, and absorbance was measured at 450 nm and 570 nm with a Biotek Synergy™ H1 microplate reader. The results from 570 nm read were subtracted from the 450 nm read.

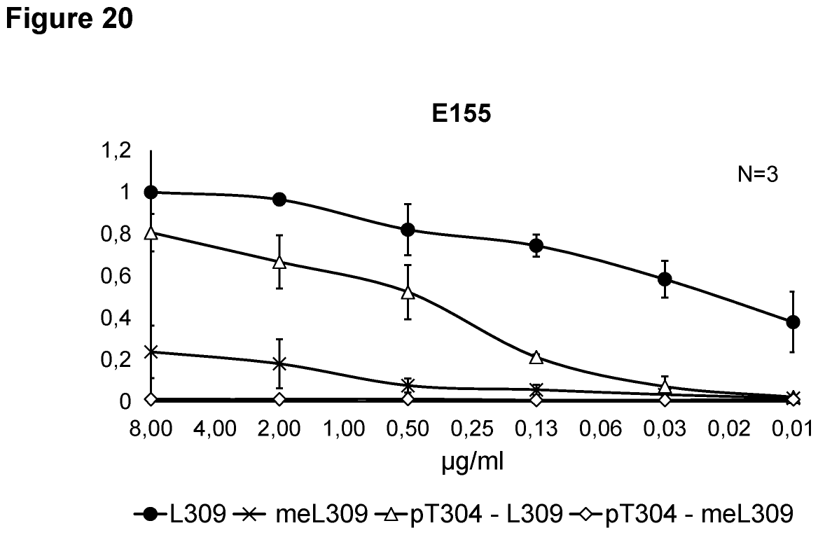

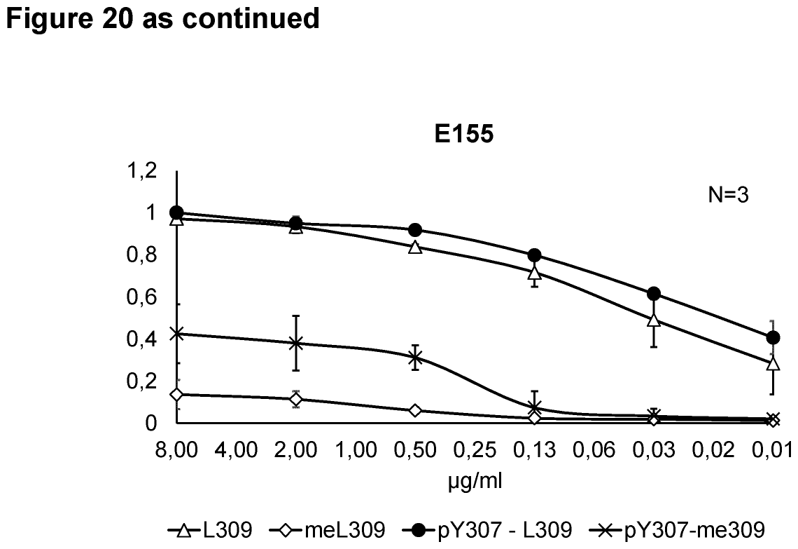

Furthermore, E155 was tested in a peptide dilution series for its affinity to 4 different undecapeptides (FIG. 20 top panel). Specifically, flat-bottom Nunc-Immuno Medisorp 96-well ELISA plates (Thermo; 467320) were coated o/n at 4° C. with 50 μl of either a peptide of the sequence ac-HVTRRTPDYFL-OH (L309) (SEQ ID NO:18) at a concentration of 8, 2, 0.5, 0.125, 0.03125 or 0.0078125 μg/ml in TBS (137 mM NaCl (AppliChem, #131659, 2.7 mM KCl (AppliChem, #131494.), 24.8 mM Tris (AppliChem, #A1086), pH 7.4 with HCl), a peptide of the sequence ac-HVTRRTPDYFL-CH₃ (meL309) (SEQ ID NO:18) at a concentration of 8, 2, 0.5, 0.125, 0.03125 or 0.0078125 μg/ml in TBS, with 50 μl of either a peptide of the sequence ac-HVTRRpTPDYFL-OH (pT304-L309) (SEQ ID NO:116) at a concentration of 8, 2, 0.5, 0.125, 0.03125 or 0.0078125 μg/ml or with a peptide of the sequence ac-HVTRRpTPDYFL-CH3 (pT304-meL309) (SEQ ID NO:116) at a concentration of 8, 2, 0.5, 0.125, 0.03125 or 0.0078125 μg/ml in TBS. The wells were washed once with TBS, blocked with 200 μl of 2% bovine serum albumin (BSA) (Sigma, A9647) in TBS for 1 hour at RT and washed again once with TBS. The wells were incubated with 50 μl of E155 (Abcam, #ab32104 lot GR17965-24, 1 μg/ml) for 1 hour at RT, washed 3 times with TBS, incubated for 1 hour at RT with 50 μl peroxidase-conjugated AffiniPure goat anti-rabbit IgG Fcγ fragment specific (Jackson ImmunoResearch, 111-035-008) secondary antibody diluted 1:10,000 in TBS and washed 3 times with TBS. Bound antibodies were detected colorimetrically by addition of 50 μl of 33 μg/ml 3′,5′,5′,5′-tetramethylbenzidine (TMB) (Sigma, T2885) in 0.1 M sodium acetate pH 6.0 and 0.01% H₂O₂ (Sigma, H1009). The colorimetric reaction was stopped after 10 min by addition of 50 μl 0.5 M H₂SO₄, and absorbance was measured at 450 nm and 560 nm with a Perkin Elmer VICTOR® Nivo™ microplate reader. The results from 560 nm read were subtracted from the 450 nm read.

E155 was tested in a further experiment (FIG. 20 bottom panel). Specifically, flat-bottom Nunc-Immuno Medisorp 96-well ELISA plates (Thermo; 467320) were coated o/n at 4° C. with 50 μl of either a peptide of the sequence ac-HVTRRTPDYFL-OH (L309) (SEQ ID NO:18) at a concentration of 8, 2, 0.5, 0.125, 0.03125 or 0.0078125 μg/ml in TBS (137 mM NaCl (AppliChem, #131659, 2.7 mM KCl (AppliChem, #131494.), 24.8 mM Tris (AppliChem, #A1086), pH 7.4 with HCl), a peptide of the sequence ac-HVTRRTPDYFL-CH₃ (meL309) (SEQ ID NO:18) at a concentration of 8, 2, 0.5, 0.125, 0.03125 or 0.0078125 μg/ml in TBS, with 50 μl of either a peptide of the sequence ac-HVTRRTPDpYFL-OH (pY307-L309) (SEQ ID NO:117) at a concentration of 8, 2, 0.5, 0.125, 0.03125 or 0.0078125 μg/ml, or with a peptide of the sequence ac-HVTRRTPDpYFL-CH₃ (pY307-meL309) (SEQ ID NO:117) at a concentration of 8, 2, 0.5, 0.125, 0.03125 or 0.0078125 μg/ml in TBS. The wells were washed once with TBS, blocked with 200 μl of 2% bovine serum albumin (BSA) (Sigma, A9647) in TBS for 1 hour at RT and washed again once with TBS. The wells were incubated with 50 μl of E155 (Abcam, #ab32104 lot GR17965-24, 1 μg/ml, 200 ng/ml or 50 ng/ml) for 1 hour at RT, washed 3 times with TBS, incubated for 1 hour at RT with 50 μl peroxidase-conjugated AffiniPure goat anti-rabbit IgG Fcγ fragment specific (Jackson ImmunoResearch, 111-035-008) secondary antibody diluted 1:10,000 in TBS and washed 3 times with TBS. Bound antibodies were detected colorimetrically by addition of 50 μl of 33 μg/ml 3′,5′,5′,5′-tetramethylbenzidine (TMB) (Sigma, T2885) in 0.1 M sodium acetate pH 6.0 and 0.01% 11202 (Sigma, H1009). The colorimetric reaction was stopped after 10 min by addition of 50 μl 0.5 M H₂SO₄, and absorbance was measured at 450 nm and 560 nm with a Perkin Elmer VICTOR® Nivo™ microplate reader. The results from 560 nm read were subtracted from the 450 nm read.

4B7 and 1D7 ELISAs

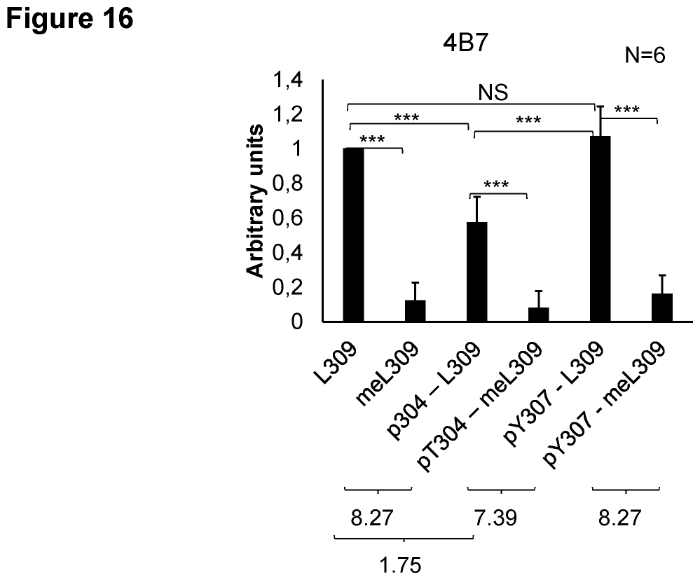

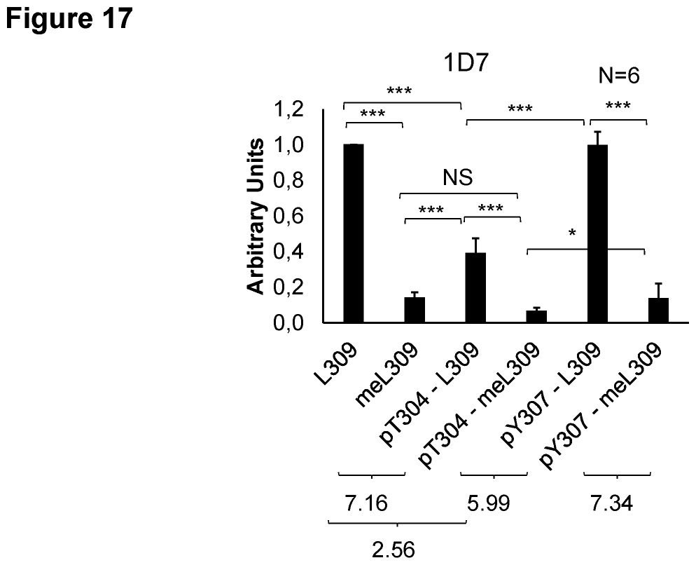

ELISAs have been performed as described in Example 2, “No impairment of 7C10-C5 binding specificity by co-cocurrent phosphorylation of tyrosine 307 or threonine 304 of PP2Ac”,but instead of the 7C10-C5 antibody with either 50 μl of monoclonal antibody 4B7 (SCBT, sc-13601, lot 0716) 1 μg/ml in 1% BSA in TBS for 1 hour at RT (FIG. 16) or hybridoma supernatant 1D7, 1:100 dilution in 1% BSA in TBS for 1 hour at RT (FIG. 17).

Results of ELISAs

The characterization of the antibodies with a preference for non-methylated PP2Ac, such as 4B7 and 1D7, but also the former “pTyr³⁰⁷” antibody E155 by ELISA revealed that none of them possesses an exclusive specificity for the non-methylated PP2Ac (FIGS. 16-20). In the ELISA, 4B7 detected the corresponding non-methylated PP2Ac peptide with 8-fold higher signal intensity than its α-carboxymethylated counterpart (FIG. 16), 1D7 with a 7-fold (FIG. 17) and E155 with a 4.7 to 8.2-fold higher signal intensity (FIGS. 18+19). These antibodies, however, are not only impaired in the recognition of PP2Ac by PP2Ac α-carboxymethylation but also by phosphorylation at position Thr³⁰⁴, although to a lesser extent than by α-carboxymethylation. At lower peptide concentrations, detection of PP2Ac by E155 was impaired by Thr³⁰⁴ phosphorylation to similar extents as by α-carboxymethylation (FIG. 20). Concomitant occurrence of α-carboxymethylation and phosphorylation at Tyr³⁰⁷ makes detection by E155 at high peptide concentrations less impaired by α-carboxymethylation than without concomitant Tyr³⁰⁷ phosphorylation.

Western Blotting

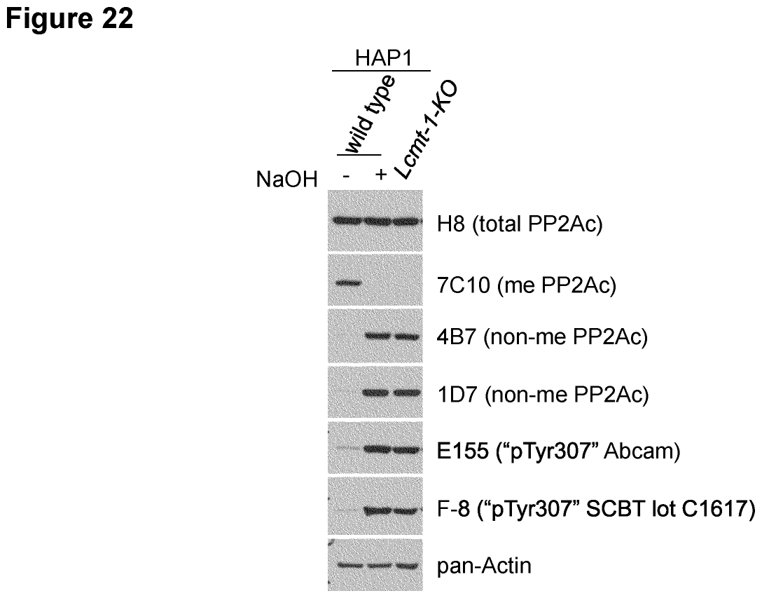

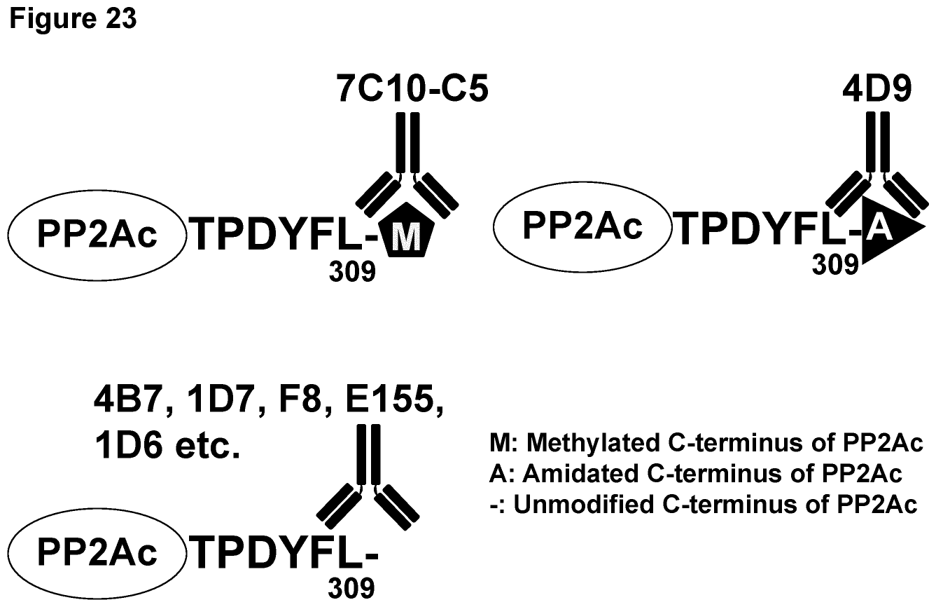

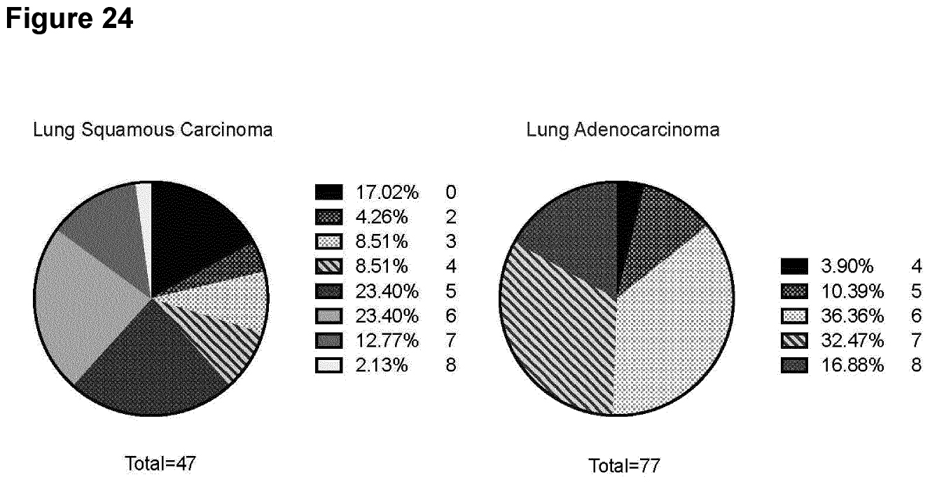

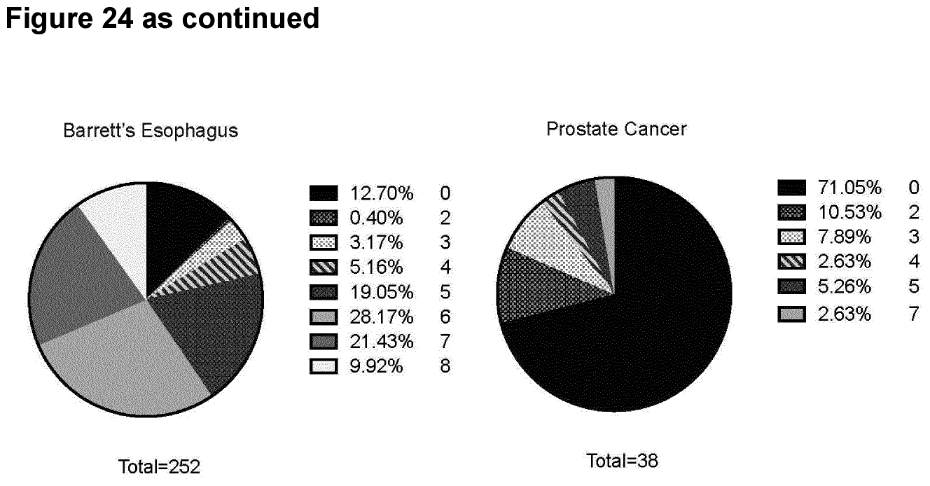

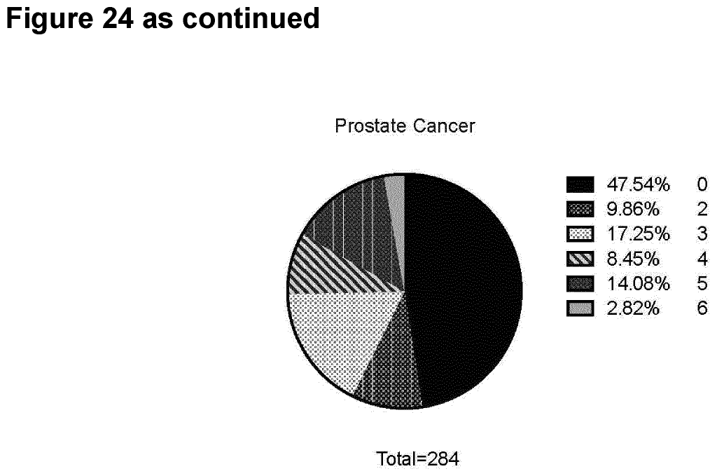

To test these antibodies in the western blot analysis with methylated and non-methylated full-length PP2Ac the methyl group was chemically removed from cellular PP2Ac by treating lysates of the human cell line, HAP1, with NaOH, as described in Example 2. The antibodies 4B7, 1D7, E155 and F-8 detected low levels of PP2Ac in the untreated cell lysates and after removal of PP2Ac methylation with NaOH high levels of PP2Ac, whereas actin and total PP2Ac levels remained unchanged by the NaOH treatment (FIG. 22). In agreement with the results of the NaOH experiments, high levels of PP2Ac were detected in HAP1 cells that lack the PP2A methyltransferase LCMT-1 indicating the preference of these antibodies for non-methylated PP2Ac (FIG. 22).

TABLE 4
Summary of detection properties and cross-reactivities of C-terminal PP2Ac antibodies.
This Table shows an overview of the properties of the
tested c-terminal PP2A C antibodies.
Antibodies screened for binding methylated PP2Ac

| | | | pThr³⁰⁴ | pTyr³⁰⁷ | PP4 | PP6 |
|---|---|---|---|---|---|---|
| | unmodified | meLeu³⁰⁹ | meLeu³⁰⁹ | meLeu³⁰⁹ | meLeu³⁰⁷ | meLeu³⁰⁵ |
| 7C10-C5 | — | *** | **** | *** | — | — |
| 2A10 | — | *** | nd | nd | *** | ** |

| | | | | | | |
|---|---|---|---|---|---|---|
| clone | unmodified | meLeu³⁰⁹ | pThr³⁰⁴ | pTyr³⁰⁷ | PP4 | PP6 |

Antibodies screened for binding non-methylated PP2Ac

| | | | | | | |
|---|---|---|---|---|---|---|
| 4B7 | *** | — | ** | *** | — | — |
| 1D7 | *** | — | ** | *** | — | — |

Antibodies raised against pTyr³⁰⁷ PP2Ac

| | | | | | | |
|---|---|---|---|---|---|---|
| E155 | *** | * | ** | *** | — | — |
| F8 | *** | — | — | *** | — | — |

PP2A Antibodies raised against the unmodified C-terminus of PP2Ac

| | | | | | | |
|---|---|---|---|---|---|---|
| 1D6 | *** | * | ** | — | *** | — |
| 7A6 | *** | * | ** | — | * | — |
| G-4 | *** | * | ** | — | * | — |
| 52F8 | *** | ** | nd | nd | — | — |
| 2038 | *** | * | nd | nd | * | * |

Summary of results,
**** >100%,
*** 80-100%,
** 40-80%,
* 15-40%,
— <15%, and
“nd” not determined

### Example 9. Illustrative Prognostic Assays Employing a Truly Anti-Methyl-PP2Ac Specific Antibody (Like 7C10-C5 of the Present Invention)

Reduction of the α-carboxymethylation levels of PP2Ac leads to the reduction of methylation-sensitive holoenzymes and enhances transformation and promotes anchorage-independent growth by activating pro-survival and pro-growth signaling pathways. In addition, inhibition of PP2A increases anchorage independent growth and augments the tumor initiation capacity of cancer cells (Liu (2016), Nat Commun 7, 11798). These findings indicate that a decrease of PP2Ac methylation in cancer cells promotes both their ability to form tumors and to metastasize.

PP2A has been implicated in the pathogenesis of prostate cancer. PP2A directly binds to and dephosphorylates the androgen receptor (Yang (2007), Mol Cell Biol 27, 3390-3404) and its downstream targets including Akt, Erk, c-MYC and Bcl2 (Kiely (2015), Cancers (Basel) 7, 648-669). Small-molecule activators of PP2A (SMAPs) show profound anti-cancer efficacy in preclinical models of castration-resistant prostate cancer (CRPC) (McClinch (2018), Cancer Res 78, 2065-2080).

Immunohistochemical (IHC) tissue microarray analyses of prostate cancer tissues with a truly anti-methyl-PP2Ac specific antibody (i.e., 7C10-C5) revealed that 7 out of 12 non-metastatic prostate cancer tissue samples had Allied scores between 2 and 7, whereas 11 out of 12 metastatic prostate cancer tissues had an Allred score of 0 suggesting a correlation between disease stage and the levels of α-carboxymethylated PP2Ac in prostate cancer tissue and that the complete loss of carboxymethylation is a common event in metastatic prostate cancer (FIG. 15). As described in Example 6, anti-PP2Ac antibodies that are not truly specific for carboxymethylated PP2Ac, did not reveal such an association.

Prognostic IHC Analysis:

Step A: Prostate cancer patients, whose cancer has not yet spread outside the prostate (Stage Ti and T2a-c), has not spread to lymph nodes (N0) or elsewhere in the body [M0] (American Joint Committee on Cancer/AJCC TNM Staging) will be studied.

Step B: Immunohistochemical (IHC) analyses of formalin-fixed paraffin-embedded (FFPE) samples from tumor biopsies (10-12 needle core biopsies for systematic mapping of the prostate) or resected tumor material from at least 4 different tumor sites

Step C: 7C10-C5 IHC staining to determine the methyl-PP2Ac levels; General PP2Ac antibody IHC staining to determine the total-PP2Ac levels; Counterstain with hematoxylin;

Step D: IHC staining will be graded according to the Allred scoring system that scores the proportion of cells that are methyl-PP2Ac positive as well as the intensity of the methy-PP2Ac staining.

All red scoring stratifies the methyl-PP2Ac status of a prostate cancer into cancers with a high risk to metastasize to those with a low risk.

Proportion Score

0—No cells or essentially no cells (e.g. ≤0.01%) are methyl-PP2Ac+

1—≤1% (e.g. 0.5-1%) of cells are methyl-PP2Ac+

2—1-10% (i.e. >1% and ≤10%; e.g. 2-10%) of cells are methyl-PP2Ac+

3—11-33% of cells are methyl-PP2Ac+.

4—34-66% of cells are methyl-PP2Ac+.

5—67-100% of cells are methyl-PP2Ac+.

Intensity Score

0—Negative methyl-PP2Ac staining.

1—Weak methyl-PP2Ac staining.

2—Intermediate methyl-PP2Ac staining.

3—Strong methyl-PP2Ac staining.

Allred Score

0-1—High probability to spread/metastasize.

2-6—Intermediate risk to spread/metastasize.

7-8—Low probability to spread/metastasize.

In addition, a low Allred score may be predictive of PP2A reactivating therapy response for these specific prostate cancer patients. In other words, if the sample from these specific prostate cancer patients has a low Allred score (i.e. 0 or 1), said cancer patients are predicted to be responsive for a PP2A reactivating therapy, e.g. using SMAPs.

This example of the use of the methyl-PP2Ac levels determined by a truly specific anti-methyl-PP2Ac antibody (7C10-C5) to assess the metastatic risk (and thus the prognosis) may be extended to all other cancers in whose pathogenesis the tumor suppressor PP2A has been shown to play role.

Additionally, the predictive nature of IHC score to PP2A reactivator therapy response may be extended to all cancers and other diseases treated with these therapies.

### Example 10. Illustrative Screen for Compounds with PP2A Activating Potential Using a Truly Antimethyl-PP2Ac-Specific Antibody (Like 7C10-C5)

In context of the present invention, also screening methods for potentially useful pharmaceuticals are provided. Such pharmaceuticals and/or medicaments may, inter alia, comprise activators of protein phosphatase 2A (PP2A). Without being bound by theory, such medicaments (i.e. activators of PP2A) are in particular useful in context of the present invention, since the protein phosphatase 2A complex is capable of stabilizing and/or increasing the methylation status of PP2Ac, in particular the stabilization of alpha-carboxymethylation of PP2Ac as described herein. Such a “stabilization” of said alpha-carboxymethylation of PP2Ac may lead to advantageous effects in disorders wherein it is desired to de-phosphorylate a hyperactive/hyperphosphorylated component of a disorder-related signaling pathway, like a disordered and/or modified signaling pathway in cancer. Accordingly, binding molecules as described in context of this invention, in particular the antimethyl-PP2Ac-specific antibody are particularly useful in drug screenings. One illustrative example of such a drug screening is provided herein below. In this context, a known activator of protein phosphatase 2A (PP2A) is employed as standard control, i.e. as a drug that fulfils this desired function. Such an activator may be DT-061, an activator of protein phosphatase 2A (PP2A) and proposed in the therapy of KRAS-mutant and MYC-driven tumorigenesis; see inter alia, Kauko (2018) “PP2A inhibition is a druggable MEK inhibitor resistance mechanism in KRAS-mutant lung cancer cells”. Sci Transl Med. 18; 10(450); McClinch (2018) Cancer Res.;78(8):2065-2080. The “read-out” of such drug screening methods may be the increase of methyl PP2Ac levels in response to the test compound, inter alia, versus (a) control compound(s). Control compounds may comprise negative controls, like compounds that do not lead to an increase of methyl PP2Ac levels and positive controls, like compounds that are known to lead to an increase of methyl PP2Ac levels (like activator of protein phosphatase 2A (PP2A), like DT-061 (CAS NO 1809427-19-7)) Other “positive controls may comprise known drugs that lead to an increase and/or stabilization of carboymehtylated PP2Ac, like PP2A activators such as phenothiazine derived small molecule PP2A activators (SMAPs) (Allen-Petersen (2019), Cancer Res 79, 209-219; Sangodkar (2017), J Clin Invest. 127, 2081-2090; Gutierrez (2014), J Clin Invest. 124(2), 644-55; Kastrinsky (2015), Bioorg Med Chem. October 1; 23(19):6528-34), drugs that counteract the endogenous PP2A inhibitors SET (I2PP2A, UniProt: Q01105) and CIP2A (UniProt: Q8TCG1-1). In the description herein above, further potential drugs are described. In such drug screenings, the inventive antibodies being specific for methyl-PP2Ac, in particular specific alpha-carboxymethylated PP2Ac, are particularly useful.

Also an illustrative in vitro drug screening assay is provided herein below wherein the antibodies of the present invention are useful.

Screening In Vivo Using Tumor Xenografts in Mice:

Step A: 5×10⁶ H358 cells (Sangodkar (2017), The Journal of clinical investigation 127, 2081-2090) or LNCap cells (McClinch (2018), Cancer Res 78, 2065-2080) will be subcutaneously injected into 6-8 week old athymic nude mice.

Step B: For an DT-061 time-course analysis, tumors may be grown to 300 mm3, then randomized into a control (DMA (Dimethylacetamide) 24 hour treatment group, a novel compound (5 mg/kg BID), and a DT-061 (5 mg/kg BID) treatment group which includes a 1 hour, 2 hour, 3 hour, 6 hour, 12 hour, and 24 hour group (13 total groups, 10 for randomization). After the respective treatment incubation, mice will be sacrificed, serum and tumor tissue were will be harvested.

Step C: Tumor tissue may be both formalin-fixed for IHC and snap frozen, along with collected serum, in liquid nitrogen for immunoblotting or molecule exposure analysis. For in vivo treatment studies, compounds as well as DT-061 may be dissolved in 10% DMA, 10% solutol, and 80% water.

PP2A activators equivalent and/or even superior to the existing DT-061 may be selected by the following 2 selection criteria:

1. Faster (than with DT-061) kinetics of methyl PP2Ac increase in response to novel compound compared to DT-061 and vehicle control determined by IHC of formalin-fixed tumor tissue and by western blot analysis with 7C10-C5 and an antibody specific for total PP2Ac such as mouse monoclonal H-8, with non-methyl specific mouse monoclonal antibody 1D7 and a loading control antibody specific for actin or tubulin.

2. Greater (than with DT-061) increase of methyl PP2Ac levels in response to novel compound compared to DT-061 and vehicle control and determined by IHC of formalin-fixed tumor tissue and by western blot analysis with 7C10-C5 and an antibody specific for total PP2Ac such as mouse monoclonal H-8, with non-methyl specific mouse monoclonal antibody 1D7 and a loading control antibody specific for actin or tubulin.

In Vitro Drug Screening

The effect of novel compounds on PP2Ac carboxymethylation may be screened in a 96 well plate assay using LNCaP cells. Cells may be plated in 96-well plates at a density of 5000 cells per well. After 24 hours of plating, cells may be treated with increasing concentrations of novel compounds starting at doses with 1 μM up to 100 μM for 1 h-3 h (always compared to identical concentrations of DT-061 as well as to vehicle control),

Cells may be washed 2× with PBS and fixed in 3.7% formaldehyde/PBS for 15 min, quenched with 50 mM NH₄Cl/PBS for 10 min, permeabilized with 0.2% Triton X-100/PBS for 10 min, blocked with 0.2% gelatine/PBS for 1 hour and then incubated with the primary antibodies 7C10-C5, 118 (for total PP2Ac), Abcam antibody (Cat #ab74272) for androgen receptor and Abcam (Cat #ab32072) for c-MYC expression diluted in 0.2% gelatine/PBS for 2 hours. Incubation with secondary antibodies, Alexa Fluor 594 goat anti-mouse IgG (H+L) (1:500, Molecular Probes), Alexa Fluor 488 donkey anti-mouse (H+L) (1:500, Molecular Probes), or donkey anti-goat IgG (H+L) Texas Red (1:200, Jackson ImmunoResearch) may be done for 1 hour. The DNA may be counterstained with Hoechst 33342 and the cover-slips mounted with Vectashield (Vector Laboratories). All steps should be performed at RT. Cells may be analyzed using an imaging instrument. 7C10-C5 fluorescence intensity may be normalized to the 118 fluorescence intensity.

The PP2A activating potential of novel compounds may be determined based on the respective normalized 7C10-C5 fluorescence values that may be compared to the methyl-PP2Ac increase achieved with the lead compound DT-061 and only those compounds with similar or higher induced increase of methyl-PP2Ac may be further considered.

Furthermore, it has been shown that DT-061 induced PP2A activation leads to lower levels of the androgen receptor, which is a known substrate of PP2A (McClinch (2018) Cancer Res.; 78(8):2065-2080) and also the known PP2A substrate c-MYC (Leonard (2020) Cell 30; 181(3):688-701).

Thus, the levels of androgen receptor (as determined by androgen receptor antibody (Abcam Cat #ab74272)) and the levels of c-MYC (as determined by c-MYC antibody (Abcam Cat #ab32072)) in the compound treated cells may be compared to the DT-061 induced levels and only those compounds with similar or higher induced downregulation of androgen receptor may be further considered.

### Example 11. About PP2A Inhibitors

Current PP2A inhibitors are not holoenzyme specific but act on the catalytic subunit. Most of them are highly toxic to cells because PP2A is an essential phosphatase. A less toxic inhibitor has been developed termed LB-100, which has been used in combination with chemo- and radiotherapy enhancing their activity. LB-100 prevents DNA repair by PP2A allowing for malignant cells to progress through the cell cycle with damaged DNA, which leads to tumor cell apoptosis. More recently it has been shown that LB-100 is not specific for PP2Ac but also inhibits the catalytic subunit of PP5, a related phosphatase (D'Arcy (2019), Mol Cancer Ther 18, 556-566).

### Example 12. Carboxymethyl PP2Ac IHC Staining Using the 7C10-C5 Antibody in a Panel of Human Dysplastic and Cancer Tissues

Immunohistochemical staining was performed on the DAKO Autostainer (DAKO, Carpinteria, Calif.) using Envision+ and diaminobenzadine (DAB) as the chromogen. De-paraffinized sections were labeled with a mouse monoclonal antibody to methyl-PP2Ac (clone 7C10-C5, 1:200) overnight at 4 C. Microwave 10 mM Tris HCl, pH9 containing 1 mM EDTA epitope retrieval was used prior to staining. Appropriate negative (no primary antibody) and positive controls (pancreas) were stained in parallel with each set of slides studied. Stained slides were scanned using an APERIO AT2 (Leica) and the digital images imported into QuPath (Bankhead (2017), Scientific Reports 7: 16878). TMA cores were analyzed for percentage of cells staining and intensity.

### Example 13. Use of an Anti-Methyl-PP2Ac Specific Antibody of the Invention for Prognosing Survival of Prostate Cancer Patients

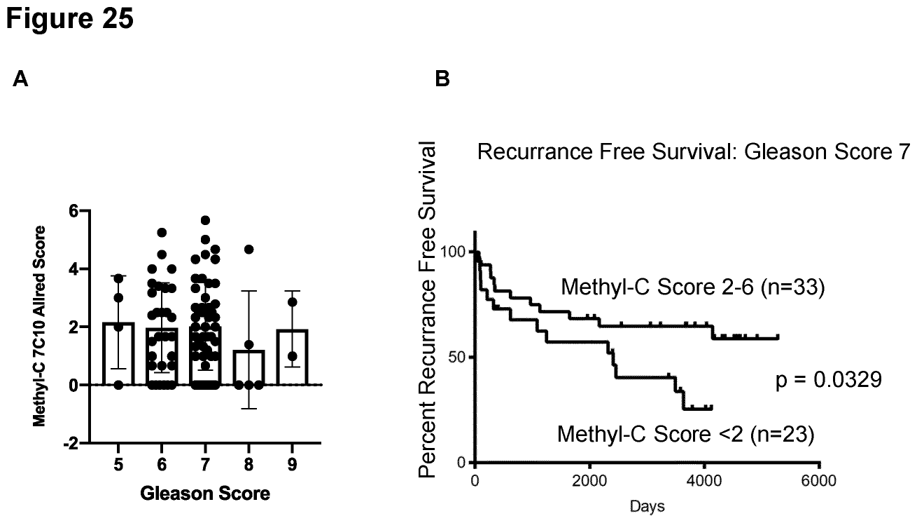

IHC analysis of 50 prostate cancer biopsies/resected tumor material with a Gleason score of 7 (Gleason (1974) J Urol. 111(1):58-64 and Gordetsky (2016) Diagn Pathol. 11:25) using a truly specific anti-methyl-PP2Ac antibody (7C10-C5) revealed a higher survival rate, i.e. recurrence free survival rate, for those patients whose tumor material stained with an Allred score of at least 2 (60% survival at 4000 days post biopsy), than for those patients whose tumor material stained with an Allied score of <2 (30% survival at 4000 days post biopsy) (FIG. 25). Those data demonstrate that carboxymethylated PP2Ac levels as determined, e.g., by immunostaining of patient prostate cancer samples with the 7C10-C5 antibody, allow to predict the survival, i.e. recurrence free survival, of patients. In particular, an Allred score of <2 predicts an unfavorable, i.e. negative, outcome (shorter survival) and an Allied score of at least 2 predicts a more favorable, i.e. positive, outcome (longer survival, i.e. recurrence free survival).

Moreover, since the prostate cancer samples were considered to be at the same stage by using the Gleason score, it is evident that assessing carboxymethylated PP2Ac levels in cancer samples, e.g. prostate cancer samples, using a truly specific anti-methyl-PP2Ac antibody (7C10-C5) allows to obtain a much more precise prognosis for the progression of the cancer (than by conventional staging alone or, by using other anti-PP2Ac antibodies that are not truly specific for carboxymethylated PP2Ac as suggested by the data shown, e.g., in Example 6 and FIG. 15). These data are fully in line with the findings described in Example 6 and FIG. 15 which demonstrate that carboxymethylated PP2Ac levels in metastatic prostate cancer tissues, using a truly specific anti-methyl-PP2Ac antibody of the invention (e.g. 7C10-C5), are much lower than in localized prostate cancer tissues (e.g. about 80% lower). It is thus plausible that low carboxymethylated PP2Ac levels (e.g. an Allred Score <2), as determined by the antibody of the invention, predict not only shorter survival of the patient but also the development, persistance and/or recurrence of metastases.

Thus, a further illustrative prognostic assay is contemplated:

Step A: Prostate cancer patients, whose cancer has not yet spread outside the prostate (Stage Ti and T2a-c) with a Gleason score 8 or less (Gleason (1974) J Urol. 111(1):58-64 and Gordetsky (2016) Diagn Pathol. 11:25) and which has not spread to lymph nodes (N0) or elsewhere in the body [M0] (American Joint Committee on Cancer/AJCC TNM Staging) may be studied.

Step B: Obtaining or providing formalin-fixed paraffin-embedded (FFPE) samples from tumor biopsies (10-12 needle core biopsies for systematic mapping of the prostate) or resected tumor material from at least 4 different tumor sites for immunohistochemical (IHC) analyses.

Step C: IHC staining with the 7C10-C5 antibody to determine the methyl-PP2Ac levels; General PP2Ac antibody IHC staining to determine the total-PP2Ac levels; Counterstain with hematoxylin;

Step D: IHC staining may be graded according to the Allied scoring system that scores the proportion of cells that are methyl-PP2Ac positive as well as the intensity of the methy-PP2Ac staining.

Allred scoring allows to categorize a cancer, i.e. a prostate cancer, based on its methyl-PP2Ac status into (1) the group of cancers with a high risk to metastasize and/or cancers associated with lower patient survival rate (prognosis of a negative outcome and/or unfavorable progression) and (2) the group of cancers with a low risk to metastasize and/or cancers associated with a higher patient survival rate (prognosis of a positive outcome and/or favorable progression).

Allred Scoring:

Proportion Score

0—No cells or essentially no cells (e.g. ≤0.01%) are methyl-PP2Ac+

1—≤1% (e.g. 0.5-1%) of cells are methyl-PP2Ac+

2—1-10% (i.e. >1% and ≤10%; e.g. 2-10%) of cells are methyl-PP2Ac+

3—11-33% of cells are methyl-PP2Ac+.

4—34-66% of cells are methyl-PP2Ac+.

5—67-100% of cells are methyl-PP2Ac+.

Intensity Score

0—Negative methyl-PP2Ac staining.

1—Weak methyl-PP2Ac staining.

2—Intermediate methyl-PP2Ac staining.

3—Strong methyl-PP2Ac staining.

Allred Score:

<2: High probability to spread/metastasize, lower patient survival rate (prognosis of a negative outcome and/or unfavorable progression);

≥2: Lower probability to spread/metastasize, higher survival rate (prognosis of a positive outcome and/or favorable progression).

In particular, if the Allred score is below 2, a 50% reduced survival rate 10 years post biopsy may be expected compared to samples with an Allied score of at least 2.

In addition, a low Allred score may be predictive of PP2A reactivating therapy response for these specific prostate cancer patients. In other words, if the sample from these specific prostate cancer patients has a low Allred score (i.e. 0 or 1), said cancer patients are predicted to be responsive for a PP2A reactivating therapy, e.g. using SMAPs.

The use of the methyl-PP2Ac levels determined by 7C10-C5 to assess the metastatic risk (and thus the prognosis) may be extended to all other cancers in whose pathogenesis the tumor suppressor PP2A has been shown to play role.

Additionally, the predictive nature of IHC score to PP2A reactivator therapy response may be extended to all cancers and other diseases treated with these therapies.

### Example 14. Use of an anti-methyl-PP2Ac specific antibody of the invention for Prognosing Responsiveness to Antiandrongen Therapy of Prostate Cancer

Profiling of Enzalutamide Resistant Prostate Cancer

The data shown in Examples 6 and 13 suggest that loss/reduction of PP2Ac carboxymethylation may be causally involved in the aggravation of the disease. The sole enzyme responsible for the carboxymethylation of PP2Ac (as well as for PP4c and PP6c) is the essential Leucine Carboxyl Methyltransferase 1 (LCMT-1) and for the demethylation the Phosphatase Methylesterase (PME-1). Loss/reduction of PP2Ac carboxymethylation could be due to inhibition and/or reduction of LCMT-1 or overexpression and/or hyperactivation of PME-1, which would lead to the loss/reduction of tumor-suppressive PP2A holoenzymes and therefore promote tumorigenesis (Pusey (2016), Tumour Biol 37, 11835-11842).

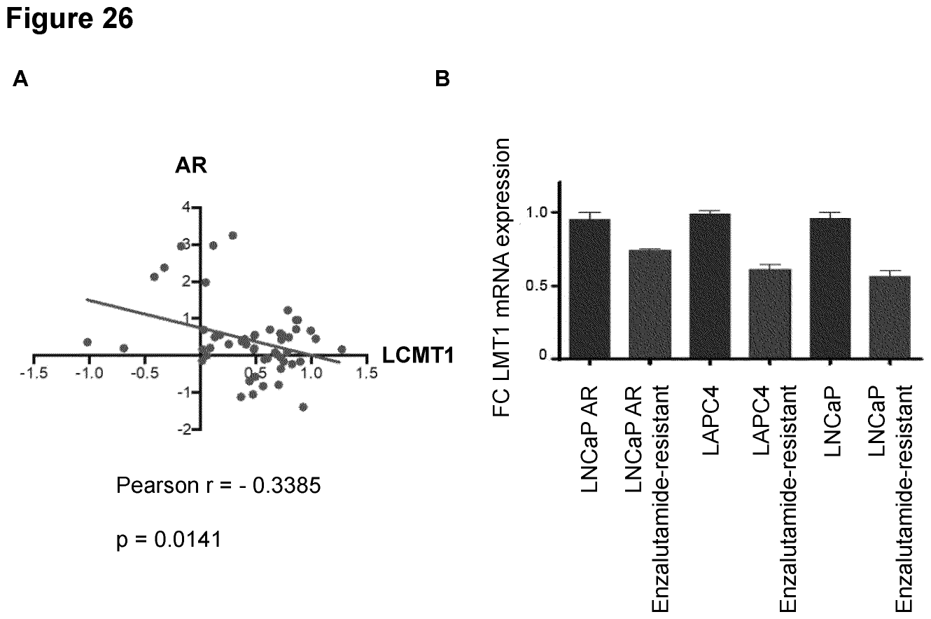

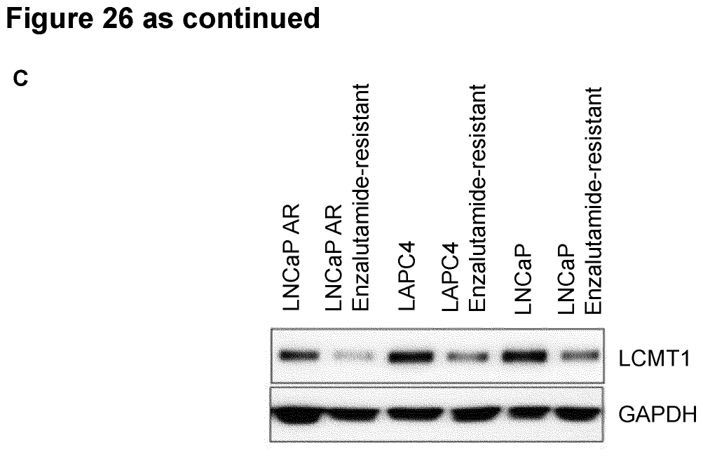

Iglesias-Gato et al. carried out a system wide quantitative proteomic analysis of metastatic prostate tumors, localized prostate tumors, and control adjacent prostate tissue (Iglesias-Gato (2018), Clin Cancer Res. 1; 24(21).) The inventors of the present invention assessed these quantitative proteomic data to evaluate changes in PP2A subunits, PP2A regulators, and PP2A substrates. The inventor's analysis surprisingly uncovered that there is a negative correlation between the expression of leucine carboxyl methyltransferase 1 (LCMT1) and the expression of androgen receptor indicating that reduction/loss of PP2A α-carboxymethylation may lead to increased AR expression levels (FIG. 26A). The inventors conclude that low levels of LCMT1 correlate with higher levels of AR in prostate cancer specimens.

To evaluate the role of LCMT1 in enzalutamide resistance, the inventors examined enzalutamide resistant cell lines derived by long-term exposure to enzalutamide. Previous studies have shown that these cell lines display increased PSA and androgen receptor (AR) levels as well as decreased PP2Ac levels (Rasool (2019), Cancer Discov. 9(11)).

In context of the present invention, examination of enzalutamide resistant derivatives of LNCaP, LNCaP-AR, LAPC4 prostate cancer cell lines by quantitative real-time PCR and western blotting surprisingly showed decreased LCMT1 mRNA expression and protein levels compared to non-enzalutamide resistant control cell lines (FIG. 26 B,C). Thus, the inventors surprisingly found that low levels of LCMT1 are not only associated with increased AR protein but also with resistance to androgen deprivation (antiandrogen) therapies. This further indicates that reduced LCTM1 expression may be also associated with increased AR signaling and unfavorable disease progression, e.g. of cancer such as prostate cancer, and or a negative outcome of the disease.

However, no anti-human LCMT antibody is currently available for immunohistochemistry (IHC). Thus, the inventors hypothesized whether carboxymethylation of PP2Ac determined by the inventive antibody provided herein (e.g. 7C10-C5) is associated with reduced LCMT expression in the context of prostate cancer. The inventors found that this was surprisingly the case, as detailed out in the following. Thus, the inventors surprisingly found that carboxymethylation of PP2Ac measured with the inventive antibody provided herein (e.g. 7C10-C5) may be used as a predictive marker for prognosing whether a cancer such as a prostate cancer is responsive to antiandrogen treatment, e.g. treatment with an androgen receptor antagonist such as enzalutamide.

LCMT1 Alters Sensitivity to Enzalutamide in Prostate Cancer Cells

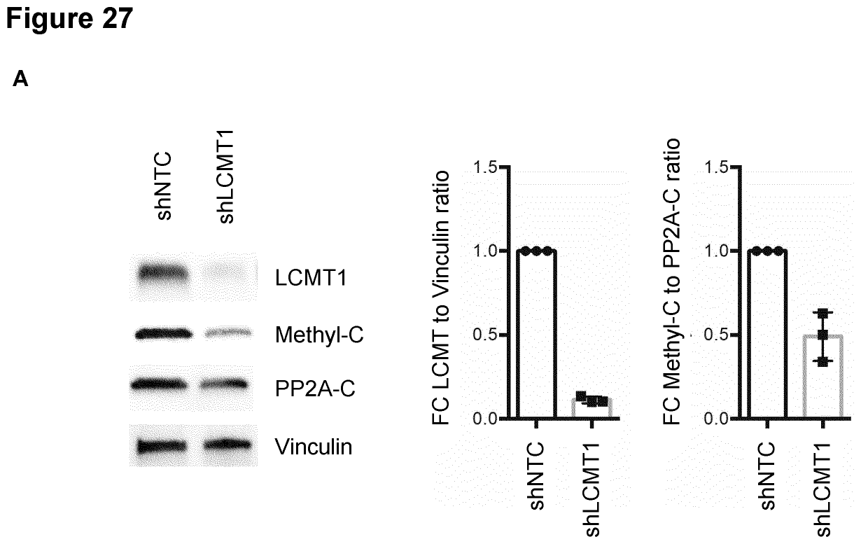

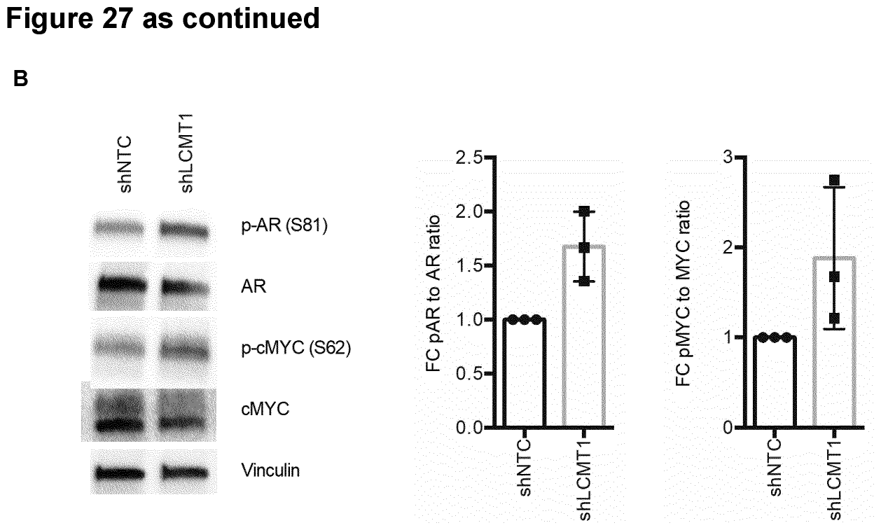

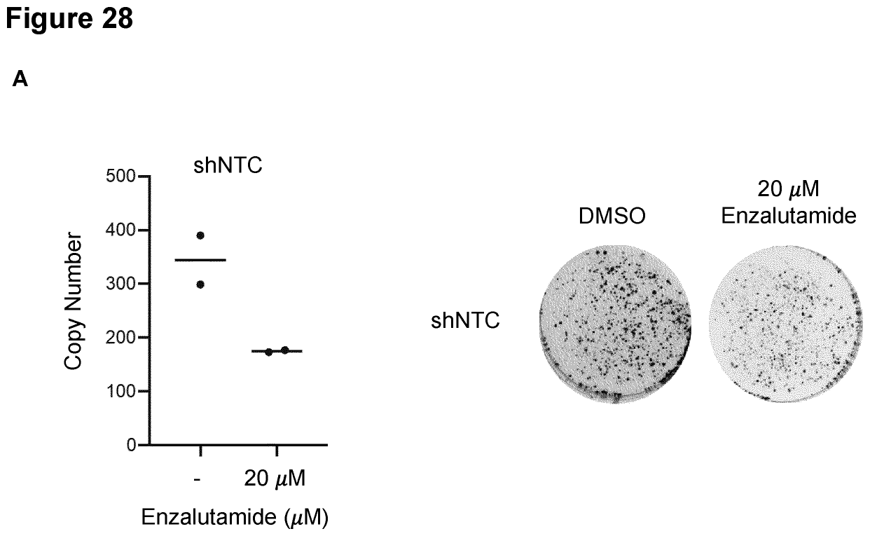

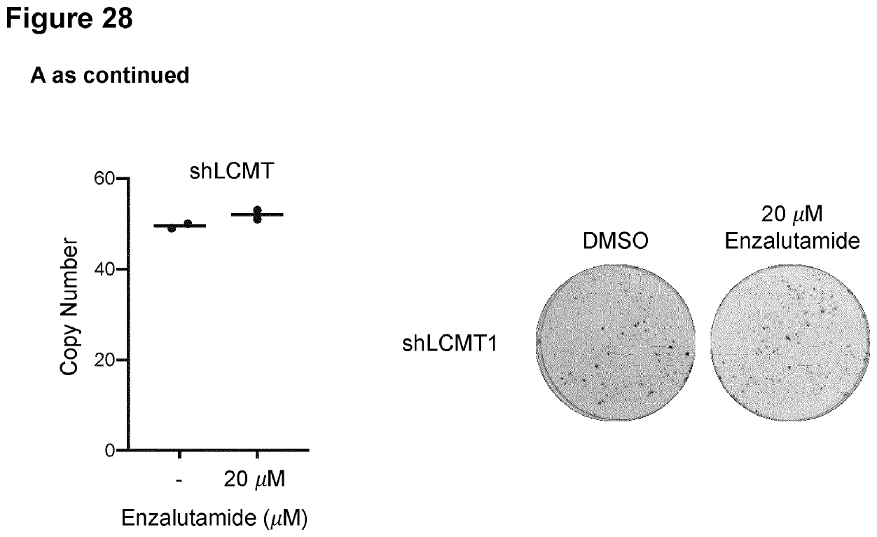

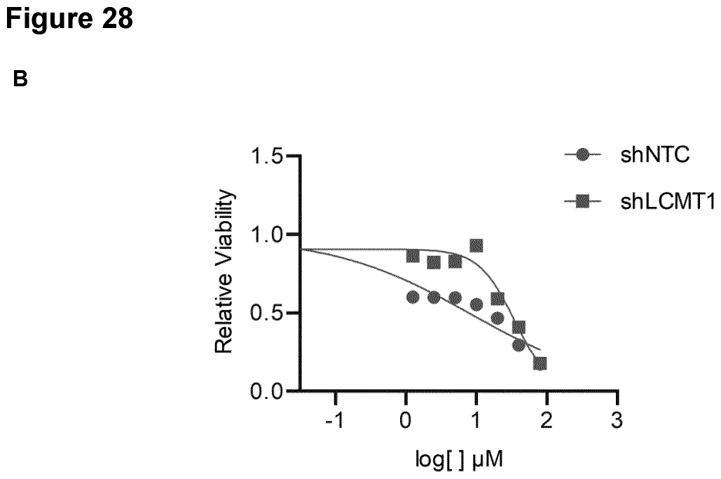

Given the decrease in carboxymethylated PP2Ac in metastatic prostate cancer (see, e.g. Example 6) and the decrease in LCMT1 in enzalutamide resistant prostate cancer cells, the inventors sought to understand whether decreased LCMT1 drives enzalutamide resistance in prostate cancer cells. Knockdown of LCMT1 resulted in decreased methylation of PP2Ac and increase in phosphorylation of androgen receptor (AR) and c-MYC as assessed by western blotting (FIG. 27). Furthermore, knockdown of LCMT1 decreased sensitivity to enzalutamide as assessed by colony formation and cell viability assays (FIG. 28).

Furthermore, without being bound by theory, reduced LCMT1 levels and correspondingly reduced carboxymethylated PP2Ac levels could lead to a PP2A bias towards methylation independent B subunits which could result in increased MYC stabilization and increased AR phosphorylation with or without changes in total AR levels, and/or loss of methylation dependent PP2A holoenzymes that have been shown to dephosphorylate c-MYC at serine 62 (Nat Cell Biol. 2004 April; 6(4):308-18. doi: 10.1038/ncb1110. Epub 2004 Mar. 14. and Mol Cell Biol. 2006 April; 26(7):2832-44. doi: 10.1128/MCB.26.7.2832-2844.2006.) and prime it for polyubiquitination and proteasomal degradation.

Altogether, this suggests that decreased levels of carboxymethylated PP2Ac measured using the inventive antibody (e.g. 7C10-C5) may be used as a predictive marker in human prostate cancer specimens to determine the responsiveness and/or response to antiandrogens, i.e. androgen receptor targeted drugs.

Materials and Methods

Cell Culture

Human cancer cell lines LNCaP, LNCAP-AR, and LAPC4 were purchased from ATCC cultured in RPMI, supplemented with 10% FBS (ThermoFisher, SH3007003) and 1% penicillin/streptomycin (GE Healthcare, SV30010). Lentiviral transduction with shLCMT1 or non-targeting control (shNTC) was done in 6 well plate for 24 hours. Cells were sorted for GFP expression. Knockdown was confirmed by western blotting. For western blotting, cells were washed 2× in PBS upon collection and then lysed in RIPA buffer (ThermoFisher Scientific, Waltham, Mass.) containing phosphatase and protease inhibitors (Roche, Basel, Switzerland). Proteins lysates were separated by SDS-PAGE 12% polyacrylamide gels (Bio-Rad, Hercules, Calif.) and transferred to nitrocellulose membranes (Bio-Rad, Hercules, Calif.). Membranes were probed with the inventive specific anti-carboxymethylated 7C10-C5 antibody, anti-total PP2Ac (Cell Signaling) antibody, anti-phospho AR s81 antibody (Abcam), anti-total androgen receptor (AR) (EMD Millipore) antibody, anti-phospho c-MYC s62 (Abcam) antibody, anti total c-MYC (Cell Signaling) antibody, or anti-vinculin antibody (Santa Cruz). Primary antibodies were probed with either goat anti-mouse (Abcam) or donkey anti-rabbit (GE Healthcare) conjugated to horseradish peroxidase and imaged and quantified using the Bio-Rad ChemiDoc XRS chemiluminescence imager and software. All values were normalized to vinculin, and expressed as fold change relative to control.

Cell Viability and Colony Formation

Cells were treated with increasing dose of Enzalutamide for 96 hours and cell viability was assessed through cell titer glo (Promega). For colony formation assays, cells were plated at a low density in 6-well plates. After 48 hours, cells were treated with DMSO (Sigma-Aldrich) or 20 uM Enzalutamide for 10-12 days. Drug medium was refreshed every 48 hours. Cells were fixed and stained with 1% crystal violet solution (Sigma-Aldrich). Quantification was performed through the cell counter function on ImageJ (imagej.nih.gov/ij/).

### Example 15. Binding Affinities of the 7C10-C5 Antibody to Carboxymethylated PP2Ac or Carboxymethylated PP4c as Determined by Surface Plasmon Resonance

The differential affinity of anti-carboxymethylated PP2Ac antibodies, 7C10-C5 and 2A10, to carboxymethylated PP2A catalytic subunit versus noncarboxymethylated PP2A catalytic subunit was determined by surface plasmon resonance (SPR) on a Biacore T200 (GE Healthcare) at the Core Facility Biomolecular & Cellular Analysis (University of Natural Resources and Life Sciences Vienna BOKU). The affinity of 7C10-C5 for carboxymethylated and noncarboxymethylated catalytic subunit of PP4 (PP4c), a PP2A-related phosphatase, was also determined. This analysis revealed a binding strength as follows: 7C10-C5 with carboxymethylated PP2Ac (K_{D} in low nM range, 11 nM)>7C10-C5 with carboxymethylated PP4c (K_{D} in higher nM range, 132 nM)>2A10 with carboxymethylated PP2Ac (K_{D} 448 nM)>2A10 with carboxymethylated PP4c (K_{D} 1130 nM) and a negligible/no binding of 7C10-C5 as well as of 2A10 to the nonmethylated PP2Ac and PP4c (Table 5). The K_{D} of the binding of 7C10-C5 (or 2A10) to non-carboxymethylated PP2Ac or PP4c could not be determined because at the highest analyte sample concentration (5000 nM) only very weak binding signals were detected indicating K_{D} values of at least >5000 nM or higher.

Thus, carboxymethylated PP2Ac was bound by 7C10-C5 41-fold, i.e. 40.7-fold, stronger than by 2A10. Furthermore, 7C10-C5 was binding to carboxymethylated PP2Ac 12-fold stronger than to carboxymethylated PP4c. In contrast, 2A10 was binding to carboxymethylated PP2Ac only 2.5-fold stronger than to carboxymethylated PP4c.

Furthermore, the data indicate that 7C10-C5 may bind to carboxymethylated PP2Ac at least about 400-fold stronger than to non-carboxymethylated PP2Ac.

Hence, these data further demonstrate that 7C10-C5 binds to carboxymethylated PP2Ac with much higher affinity than 2A10, that 7C10-C5 has a strongly reduced cross-reactivity with carboxymethylated PP4c compared to 2A10, and that 7C10-C5 does not cross-react with non-carboxymethylated PP2Ac or PP4c. In brief, the SPR analysis confirms that 7C10-C5 is highly and truly specific for carboxymethylated PP2Ac, whereas 2A10 is much less specific (i.e. due to considerable cross-reactivity with PP4c) and further has a lower affinity to carboxymethylated PP2Ac.

TABLE 5
7C10-C5 and 2A10 surface plasmon resonance (SPR) analysis
Steady state analysis

| | | | KD | Rmax |
|---|---|---|---|---|
| | | | (M) | (RU) |
| 50 μg/ml mAB | 7C10-C5 | PP2A Me 5-160 nM | 1.100E−08 | 25.14 |
| capturing | | PP4 Me 50-800 nM | 1.320E−07 | 20.17 |
| 150 sec | 2A10 | PP2A Me 30-480 nM | 4.480E−07 | 15.95 |
| | | PP4 Me 400-6400 nM | 1.130E−06 | 10.97 |

PP2A Me: 360 sec contact time, 40 μL/min
PP4 Me: 360 sec contact time, 40 μL/min

Material and Methods

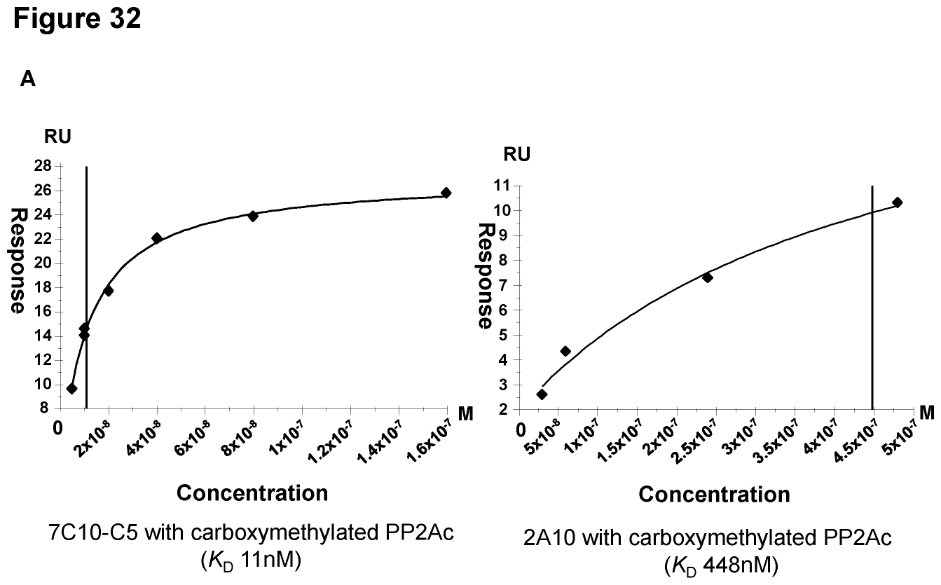

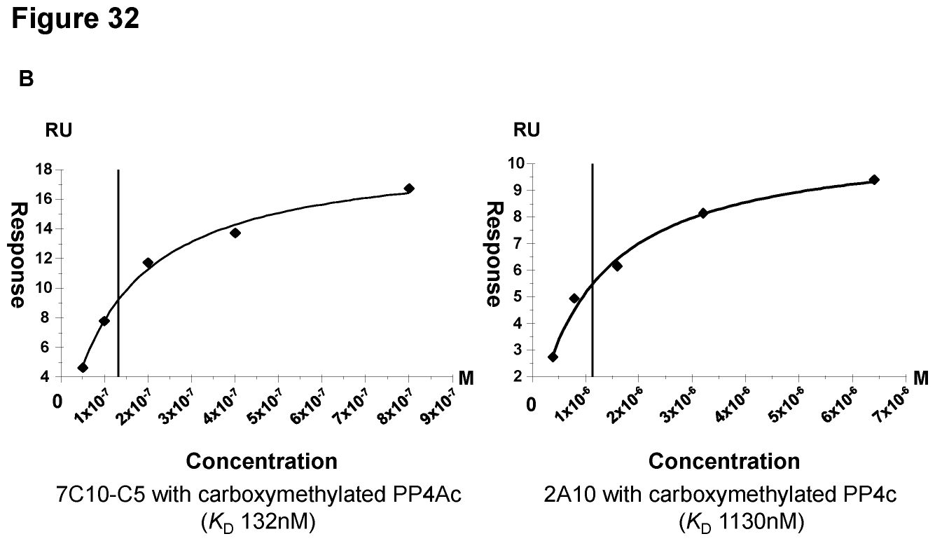

Series S Sensor Chip CM5 (GE Healthcare, 10296958) Flow cells (e.g. Flow cells 3 and 4 for the data shown in Table 5 and FIG. 32) were coated with 30 μg/mL anti-mouse IgG antibody (in immobilization buffer) using the mouse antibody capture kit (GE Healthcare, BR-1008-38, Lot 10294578) via amine coupling according to the manufacturer's protocol (i.e. https://www.cytivalifesciences. com/en/us/shop/protein-analysis/spr-lab el-free-analysis/capture-reagents/mouse-antibody-capture-kit-p-05986). The immobilisation levels obtained were 11620.2 RU (Flow cell 1), 10836.5 RU (Flow cell 2), 11605.9 RU (Flow cell 3) and 11333.9 RU (Flow cell 4), and thus were within the specifications. The antibodies (7C10-C5 or 2A10) were applied with a concentration of 50 μg/mL and reached a reproducible response level of approx. 1200 RU.

The following 11-mer peptides corresponding to the carboxymethylated or non-methylated carboxy-terminus of PP2A and PP4 catalytic subunits were used as antigens:

PP2Ac:
(SEQ ID NO: 18)
ac-HVTRRTPDYFL-OH
mePP2Ac:
(SEQ ID NO: 18)
ac-HVTRRTPDYFL-CH3
PP4c:
(SEQ ID NO: 20)
ac-PSKKPVADYFL-OH
mePP4c:
(SEQ ID NO: 20)
ac-PSKKPVADYFL-CH3

Analyte sample concentration (antigen) should be within a range of 0.1 to 10 times the K_{D} and equilibrium should be reached at all concentrations that are used for K_{D} calculation. Furthermore, to obtain a robust fit of a plot of Req against concentration for affinity determination, it is important that the range of analyte concentration is wide enough to reveal the full curvature of the plot (i.e. to reach saturation). Thus, the analytes (11-mer peptides) were used in different concentrations which is important to obtain accurate K_{D} values, wherein the optimal concentrations depend on the affinity of the binding partners.

The concentration ranges were:
mePP2Ac 5 nM, 10 nM, 20 nM, 40 nM, 80 nM and 160 nM for 7C10-C5
mePP2Ac 30 nM, 60 nM, 120 nM, 240 nM and 480 nM for 2A10
mePP4c 50 nM, 100 nM, 200 nM, 400 nM, 800 nM for 7C10-C5
mePP4c 400 nM, 800 nM, 1600 nM, 3200 nM and 6400 nM for 2A10

Antibody capturing time was adjusted to reach approx. 1200 RU, antigen association time was 360 sec, and dissociation time was 450 sec. The flow rate was 40 μL/min. Regeneration of the chip was performed by injecting 10 mM Glycin-HCl pH 1.7 for 180 sec with a flow rate of 20 μL/min. Flow cell 1 and 3, respectively, were used as reference cells. All experiments were performed in multi cycle kinetics mode.

Running buffer: PBS pH 7.4 (PAN-Biotech cat: P04-36500, lot: 5270820)+0.005% Tween (Roth cat: 9127)+0.1% BSA (Albumin, fraction V, protease free >98%, Roth, cat: T844.2, charge 299286373)

SPR experiments were performed with the BiacoreT200 instrument (Cytiva). All measurements were performed at 25° C. The sensorgrams obtained were analyzed with BiacoreT200 Evaluation Software (version 3.1). The value of the equilibrium dissociation constant (K_{D}) was obtained by fitting a plot of response at equilibrium (Req) against the respective concentration of the analyte. All binding curves reached equilibrium and all requirements for a steady state analysis were fulfilled (FIGS. 32A and B). Technical duplicates confirmed the results.

### Example 16. Recombinant 7C10-C5 Antibody Containing an IgG2 Portion

Cloning of Recombinant 7C10-C5 into pTrioz mIgG2a

pTrioz mIgG2a: see Invivogen, https://www.invivogen.com/ptrioz-migg2a

For cloning of the pTrioz 7C10 mIgG2a, the sequences of the first 20 base pairs (bp) of the heavy or light chain variable regions (SEQ ID NO:39 and SEQ ID NO:40, respectively) were taken as forward primers. As reverse primer for the light or heavy chain variable fragment a sequence in the joining region was used and cloned using the NEBuilder® HiFi DNA Assembly (NEB #E2621) into the pTrioz SP mIgG2a which already contained the sequence of signal peptide for the light chain (L1) and heavy chain (IL2) in the 5′ region of the IgCK and IgG2a sequences, respectively.

Additionally, the light chain forward primer contained a part of a signal peptide, which was cloned into the pTrioz. Of note, the signal peptide (L1) is thought to improve expression of the antibody (Haryadi (2015); PLoS One 10, e0116878). The sequence of the light chain forward primer was: 5′-GGCTCTCAGGTGCCAGATGTgatgttttgatgacccaaac-3′ (SEQ ID NO:127), with the signal peptide (overhang) in capital letters.

The light chain reverse primer additionally contained the overlapping region of the light chain constant domain IgCK present in the pTrioz. The sequence of the light chain reverse primer was: 5′-ACAGTTGGTGCAGCATCTGCtttgatttccagcttggtg-3′) (SEQ ID NO:128), with the IgCK sequence (overhang) in capital letters.

The heavy chain forward primer contained a part of a signal peptide (IL2), which was cloned into the pTrioz. Of note, the signal peptide sequence, IL2 was derived from the pFUSE-mIgG2a-Fc2 (InvivoGene) which is a cloning plasmid for the generation and secretion of a Fc-fusion protein expressing the Fc region (CH2 and CH3 domains) of the murine IgG2a heavy chain and the hinge region. The sequence of the heavy chain forward primer was: 5′-CTTGCACTTGTCACGAATTCGgatgtacagcttcaggagtc-3′ (SEQ ID NO:129), with the signal peptide sequence (overhang) in capital letters.

The heavy chain reverse primer additionally contained the overlapping region of the heavy chain constant domain IgG2a present in the pTrioz. The sequence of the heavy chain reverse primer was: 5′-AGACCGATGGGGCTGTTGTTTTAGCtgcagagacagtgaccag-3′ (SEQ ID NO:130), with the mIgG2a (overhang) in capital letters.

For the PCR, 100 ng of plasmid DNA from the Monovalent 7C10-C5 scFv (prepared as described above in Example 4) were mixed with 41 forward primer (10 μM) and 2 μl reverse primer (10 μM), 10 μl 5× Q5 Reaction Buffer (NEB #B9027), 0.41 dNTPs (10 μM), 33 μl nuclease free water (Sigma-Aldrich, #W4502) and 0.5 μl Q5® High-Fidelity DNA Polymerase (NEB, #M0491L). Reactions were performed in a Biometra TRIO thermocycler starting with a DNA denaturation step at 98° C. for 1 min followed by 31 cycles of denaturation of the DNA at 98° C. for 15 sec. annealing at 52° C. for 15 sec., and elongation at 72° C. for 30 sec. A 5 minutes 72° C. step completed the reaction. The PCR products were separated on a 1.5% TAE (for 1 Liter: 4.84 g Tris (AppliChem #A1086), 1.14 ml Glacial Acetic Acid, 2 ml 0.5M EDTA pH 8.0) agarose gel (Sigma, A9539) and stained with ethidium bromide. The bands corresponding to the light chain variable fragment and the heavy chain variable fragment were cut out and purified using a Wizard® DNA Clean Up Kit (Promega, #A9282), following the protocol as instructed, and eluted in 50 μl nuclease free water (Sigma, #W4502).

Next, the 7C10-C5 light and heavy chain variable fragments were cloned with the NEBuilder® HiFi DNA Assembly (NEB #E2621) into the pTrioz SP mIgG2a, which was cut with AscI, cutting between the light chain signal peptide (L1) and IgCK, and AgeI cutting between heavy chain signal peptide (IL2) and IgG2a sequence. 50 fmol of the respective light chain variable fragment with the overlapping L1 signal peptide on the 5′-end and the overlapping IgCK sequence at the 3 ‘-end and 50 fmol of the respective heavy variable fragment with the overlapping IL2 signal peptide sequence at the 5’-end and the overlapping IgG2a sequence at the 3′-end were mixed with 50 fmol of the purified fragments of the AscI and AgeI cut and gel purified fragments of the pTrioz SP mIgG2a vector and was incubated with the 2× HiFi DNA Assembly master mix in a final volume of 20 μl at 50° C. for 1 hour.

The NEBuilder mixture was used for bacterial transformation. 100 μl of heat shock competent E. coli HB101 were thawed on ice, the DNA was added and the cells were kept for another 20 minutes on ice. A heat shock for 1 minute at 42° C. was performed and after 10 minutes on ice the bacteria were resuspended in 500 μl LB medium without antibiotics for recovery. After 30 minutes at 30° C. bacteria were plated out on LB plates with Zeocin (50 μg/ml) and incubated over night at 30° C. Clones were subsequently inoculated in 5 ml LB with Zeocin, grown over night and DNA was eluted in 50 μl nuclease free water after purification with a Qiagen Miniprep Kit by following the manufacture's manual (Qiagen, #27106). The gained DNA was prepared for sequencing as demanded by the sequencing company (Microsynth). 10 μl with 100 ng/μl DNA was mixed with 4 μl primer (10 μM) using the EF1 alpha forward sequencing primer (5′-TAATACGACTCACTATAGGG-3′) (SEQ ID NO:111) to get sequences of the L1 signal peptide, light chain variable fragment and IgCK and the pTrioz H forward sequencing primer (5′-TTTGAGCGGAGCTAATTC-3′) (SEQ ID NO:126) to get sequences of the signal peptide, heavy variable fragments and beginning of the IgG2a region. The obtained sequences for the heavy and light variable fragments were checked with and matched the previously obtained sequences.

Expression of Recombinant 7C10-C5 Constructs.

For mammalian transfection, the clone with the correct expression constructs for the 7C10-C5 recombinant antibodies were subsequently inoculated in 100 ml LB with Ampicillin, grown overnight and DNA was eluted in 200 μl nuclease free water after purification with a Qiagen Midiprep Kit by following the manufacture's manual (Qiagen, #12943).

HEK293T cells were grown in DMEM+10% FCS+2 mM L-glutamine (Sigma, G2150)+100 units/ml Penicillin/0.1 mg/ml Streptomycin in a humidified incubator controlled at 37° C. with 7.5% CO2. For subculturing, cells were passaged every 2 to 3 days at a ratio of 1:3 to 1:8. 1.3×10⁶ HEK 293T cells were seeded in 6 cm dishes in 5 ml growth media the day before transfection. Cells were transfected by mixing 600 μl of OptiMEM (Gibco™ 31985047, #11524456) with 5 μg of DNA (7C10-C5 recombinant antibody constructs) 2 seconds of vortex-mixing, addition of 7.5 μl of TurboFectin 8.0 (Origin #TF81001) mixing by 5× inverting the tube, incubating the transfection mix for 15 min at room temperature and adding it to the cells. 72 hours later the cell supernatant was centrifuged at 1200 rpm in a Beckman SC6R table top centrifuge, and the supernatant was used for the ELISA (and Western blot) experiments.

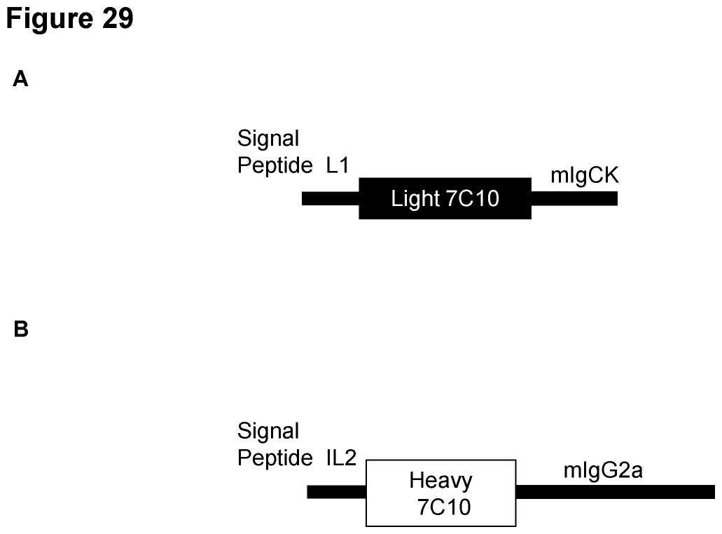

Sequences of the Recombinant Light Chain Variable Fragment and mIgCK of Antibody 7C10-C5 (See FIG. 29)

DNA sequence of the recombinant 7C10-C5
light chain
(SEQ ID NO: 122)
ATGGACATGAGGGTCCCTGCTCAGCTCCTGGGGCT
CCTGCTGCTCTGGCTCTCAGGTGCCAGATGTGATG
TTTTGATGACCCAAACTCCACTCTCCCTGCCTGTC
AGTCTTGGAGATCAAGCCTCCATCTCTTGCAGATC
TAGTCAGAGCATTGTACATAGTAATGGAAACACCT
ATTTAGAATGGTACCTGCAGAAACCAGGCCAGTCT
CCAAAGCTCCTGATCTACAAAGTTTCCAACCGATT
TTCTGGGGTCCCAGACAGGTTCAGTGGCAGTGGAT
CAGGGACAGATTTCACACTCAAGATCAACAGAGTG
GAGGCTGAGGATCTGGGAGTTTATTACTGCTTTCA
AGGTTCACATGTTCCGTGGACGTTCGGTGGAGGCA
CCAAGCTGGAAATCAAAGCAGATGCTGCACCAACT
GTATCCATCTTCCCACCATCCAGTGAGCAGTTAAC
ATCTGGAGGTGCCTCAGTCGTGTGCTTCTTGAACA
ACTTCTACCCCAAAGACATCAATGTCAAGTGGAAG
ATTGATGGCAGTGAACGACAAAATGGCGTCCTGAA
CAGTTGGACTGATCAGGACAGCAAAGACAGCACCT
ACAGCATGAGCAGCACCCTCACGTTGACCAAGGAC
GAGTATGAACGACATAACAGCTATACCTGTGAGGC
CACTCACAAGACATCAACTTCACCCATTGTCAAGA
GCTTCAACAGGAATGAGTGTTAG
Signal Peptide L1 is in italics; the
variable domain of the 7C10-C5 light
chain is in bold
Amino acid sequence of the recombinant
7C10-C5 light chain
(SEQ ID NO: 123)
MDMRVPAQLLGLLLLWLSGARCDVLMTQTPLSLPV
SLGDQASISCRSSQSIVHSNGNTYLEWYLQKPGQS
PKLLIYKVSNRFSGVPDRFSGSGSGTDFTLKINRV
EAEDLGVYYCFQGSHVPWTFGGGTKLEIKADAAPT
VSIFPPSSEQLTSGGASVVCFLNNFYPKDINVKWK
IDGSERQNGVLNSWTDQDSKDSTYSMSSTLTLTKD
EYERHNSYTCEATHKTSTSPIVKSFNRNEC
Signal Peptide L1 is in italics; the
variable domain of the 7C10-C5 light
chain is in bold

Sequence of the Recombinant Heavy Chain Variable Fragment and mIgG2a of Antibody 7C10-C5 (see FIG. 29).

DNA sequence of the recombinant 7C10-C5
heavy chain coding sequence
(SEQ ID NO: 124)
ATGTACAGGATGCAACTCCTGTCTTGCATTGCACT
AAGTCTTGCACTTGTCACGAATTCGGATGTACAGC
TTCAGGAGTCAGGACCTGGCCTCGTGAAACCTTCT
CAGTCTCTGTCTCTCACCTGCTCTGTCACTGGCTA
CTCCATCACCAGTGGTTATTACTGGAACTGGATCC
GGCAGTTTCCAGGAAACAAACTGGAATGGATGGGC
TACATAAGCTACGACGGTAGCAATAACTACAACCC
ATCTCTCAAAAATCGAATCTCCATCACTCGTGACA
CATCTAAGAACCAGTTTTTCCTGAAGTTGAATTCT
GTGACTACTGAGGACACAGCTACATATTACTGTGC
TGGACGGTTTGCTTACTGGGGCCAAGGGACTCTGG
TCACTGTCTCTGCAGCTAAAACAACAGCCCCATCG
GTCTATCCACTGGCCCCTGTGTGTGGAGATACAAC
TGGCTCCTCGGTGACTCTAGGATGCCTGGTCAAGG
GTTATTTCCCTGAGCCAGTGACCTTGACCTGGAAC
TCTGGATCCCTGTCCAGTGGTGTGCACACCTTCCC
AGCTGTCCTGCAGTCTGACCTCTACACCCTCAGCA
GCTCAGTGACTGTAACATCGAGCACCTGGCCCAGC
CAGTCCATCACCTGCAATGTGGCCCACCCGGCAAG
CAGCACCAAGGTGGACAAGAAAATTGAGCCCAGAG
GGCCCACAATCAAGCCCTGTCCTCCATGCAAATGC
CCAGCACCTAACCTCTTGGGTGGACCATCCGTCTT
CATCTTCCCTCCAAAGATCAAGGATGTACTCATGA
TCTCCCTGAGCCCCATAGTCACATGTGTGGTGGTG
GATGTGAGCGAGGATGACCCAGATGTCCAGATCAG
CTGGTTTGTGAACAACGTGGAAGTACACACAGCTC
AGACACAAACCCATAGAGAGGATTACAACAGTACT
CTCCGGGTGGTCAGTGCCCTCCCCATCCAGCACCA
GGACTGGATGAGTGGCAAGGAGTTCAAATGCAAGG
TCAACAACAAAGACCTCCCAGCGCCCATCGAGAGA
ACCATCTCAAAACCCAAAGGGTCAGTAAGAGCTCC
ACAGGTATATGTCTTGCCTCCACCAGAAGAAGAGA
TGACTAAGAAACAGGTCACTCTGACCTGCATGGTC
ACAGACTTCATGCCTGAAGACATTTACGTGGAGTG
GACCAACAACGGGAAAACAGAGCTAAACTACAAGA
ACACTGAACCAGTCCTGGACTCTGATGGTTCTTAC
TTCATGTACAGCAAGCTGAGAGTGGAAAAGAAGAA
CTGGGTGGAAAGAAATAGCTACTCCTGTTCAGTGG
TCCACGAGGGTCTGCACAATCACCACACGACTAAG
AGCTTCTCCCGGACTCCGGGTAAATAA
Signal Peptide IL2 is in italics; the
variable domain of the 7C10-C5 heavy
chain coding sequence is in bold
Amino acid sequence of the recombinant
7C10-C5 heavy chain
(SEQ ID NO: 125)
MYRMQLLSCIALSLALVTNSDVQLQESGPGLVKPS
QSLSLTCSVTGYSITSGYYWNWIRQFPGNKLEWMG
YISYDGSNNYNPSLKNRISITRDTSKNQFFLKLNS
VTTEDTATYYCAGRFAYWGQGTLVTVSAAKTTAPS
VYPLAPVCGDTTGSSVTLGCLVKGYFPE
PVTLTWNSGSLSSGVHTFPAVLQSDLYTLSSSVTV
TSSTWPSQSITCNVAHPASSTKVDKKIEPRGPTIK
PCPPCKCPAPNLLGGPSVFIFPPKIKDVLMISLSP
IVTCVVVDVSEDDPDVQISWFVNNVEVHTAQTQTH
REDYNSTLRVVSALPIQHQDWMSGKEFKCKVNNKD
LPAPIERTISKPKGSVRAPQVYVLPPPEEEMTKKQ
VTLTCMVTDFMPEDIYVEWTNNGKTELNYKNTEPV
LDSDGSYFMYSKLRVEKKNWVERNSYSCSVVHEGL
HNHHTTKSFSRTPGK
Signal Peptide IL2 is in italics; the
variable domain of the 7C10-C5 heavy
chain coding sequence is in bold

Recombinant 7C10-C5 Antibody is Specific for the α-Carboxymethylated PP2Ac as Determined by ELISA

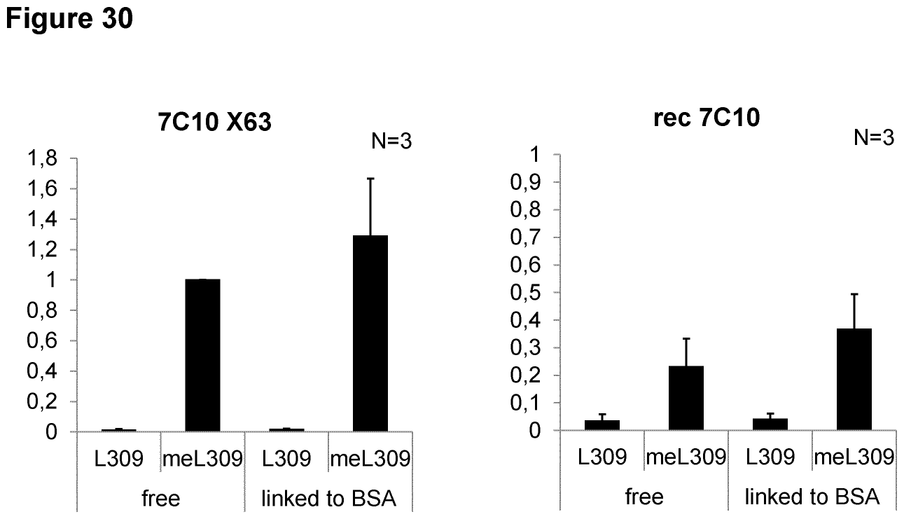

Nunc Medisorp 96-well ELISA flat-bottom plates were coated with peptides either free or linked to BSA, L309 (CGEPHVTRRTPDYFL) (SEQ ID NO:49) or meL309 (CGEPHVTRRTPDYFL-CH₃) (SEQ ID NO:49), on the ELISA plate at 0.5 μg/ml in TBS (free) or 1 μs/ml in TBS (linked to BSA) at 4° C. over night. After washing twice with TBS, the wells were incubated with 2% BSA in TBS for 1 h at RT. After washing once with TBS, the wells were incubated with single clone hybridoma cell culture supernatant, clone 7C10-C5 1:50 (7C10 X63), or recombinant antibody 7C10-C5 undiluted (rec 7C10). After washing 3× with TBS, incubation with anti-mouse-HRP coupled secondary antibody for 1 h at RT was performed. Bound antibodies were detected by colorimetric reaction with 3,3′,5,5′-Tetramethylbenzidine as substrate, and absorbance was measured at 450 nm. Values were normalized to meL309 incubated with 7C10 X63, which was arbitrarily set to 1. Average and standard deviation of N=3 experiments are shown (FIG. 30).

Recombinant 7C10-C5 Antibody is Specific for the α-Carboxymethylated PP2Ac as Determined by Western Blotting with Mammalian Cell Lysates

To confirm the methyl specificity of the recombinant 7C10-C5, the carboxy-terminal methyl group was chemically removed from cellular PP2Ac by treating lysates of HEK293Trex with NaOH as described in Favre (1994), J Biol Chem 269, 16311-16317. In brief, 100 μl of HEK293T cell lysate corresponding to 200 μg of protein was mixed with 1M NaOH to a final concentration of 0.2M and incubated for 10 min at RT. The reaction was neutralized by adding HCL to a final concentration of 0.2M and diluted to 200 μl with IP Lyse. The control reaction was treated with preneutralization solution (0.2M NaOH and 0.2M HCL) for 10 min at room temperature and diluted to 200 μl with IP Lyse. For immunoblot analysis, protein loading buffer was added to the protein samples and proteins were denatured by incubation at 95° C. for 5 min. 15 μg of untreated and NaOH treated protein samples were loaded on a 10% SDS-PAG.

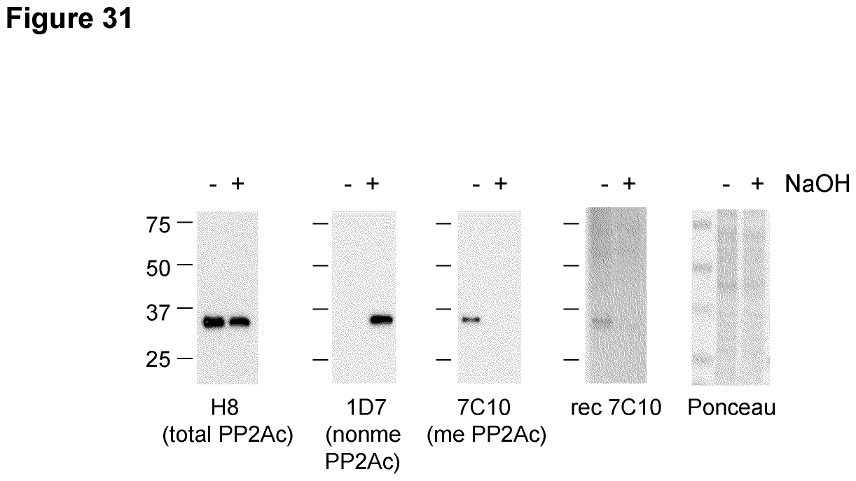

Like the X63 supernatant, the recombinant methyl-PP2Ac specific antibody 7C10-C5 only detected methylated PP2Ac in the untreated cell lysates but not in the NaOH treated lysates, the non-methyl specific antibody 1D7 detected the NaOH treated samples, whereas an anti-total PP2Ac antibody (H8) detected PP2A in treated and untreated samples (FIG. 31).

1. An antibody specifically binding the carboxymethylated catalytic subunit of protein phosphatase 2A (PP2Ac).

2. The antibody of claim 1 specifically binding the methylated carboxyl group of the C-terminal leucine, i.e. Leu309, of PP2Ac.

3. The antibody of claim 1 or 2 specifically binding an epitope comprised in the carboxymethylated C-terminal region of PP2Ac, wherein said C-terminal region has the sequence TPDYFL (SEQ ID NO:1).

4. The antibody of claim 3, wherein the epitope comprises (i) the methylated carboxyl group of the C-terminal leucine and (ii) the threonine and/or the proline within SEQ ID NO:1.

5. The antibody of any one of the preceding claims, wherein said antibody binds said carboxymethylated PP2Ac or, preferably, a peptide consisting of the last 11 amino acids of the carboxymethylated C-terminal region of PP2Ac (HVTRRTPDYFL; SEQ ID NO:18), with a dissociation constant (K_{D}) of 200 nM, 100 nM, 80 nM, 60 nM, 40 nM, 20 nM, 15 nM or less, preferably 40 nM or less, more preferably 20 nM or less, e.g. about 11 nM.

6. The antibody of claim 5, wherein said dissociation constant (K_{D}) is determined by surface plasmon resonance, preferably Biacore, more preferably a BiacoreT200 instrument, a Series S Sensor Chip CMS and a BiacoreT200 Evaluation Software, i.e. version 3.1.

7. The antibody of claim 5 or 6, wherein the conditions for determining said dissociation constant comprise:
(i) a temperature of 25° C.,
(ii) said antibody at a concentration of 50 μg/mL,
(iii) a peptide consisting of the last 11 amino acids of the carboxymethylated C-terminal region of PP2Ac (HVTRRTPDYFL-CH₃; SEQ ID NO:18), preferably at least 4, preferably at least 6 different concentrations, wherein the lowest concentration is about 5 nM and the highest concentration is about 160 nM, and the other concentrations are preferably between said lowest and highest concentrations; and/or
(iv) PBS with pH 7.4+0.005% Tween+0.1% BSA as a buffer;
in particular such that all binding curves reach equilibrium and all requirements for a steady state analysis are fulfilled.

8. The antibody of any one of the preceding claims comprising a heavy chain variable region and a light chain variable region, wherein
(a) the heavy chain variable region comprises at least one complementary determining region selected from a CDR-113, a CDR-112 and a CDR-H1, wherein
(i) the CDR-H3 sequence is RFAY (SEQ ID NO:2),
(ii) the CDR-H2 sequence is YISYDGSNNYNPSLKN (SEQ ID NO:3),
(iii) the CDR-H1 sequence is SGYYWN (SEQ ID NO:4),
and/or
(b) the light chain variable region comprises at least one complementary determining region selected from a CDR-L3, a CDR-L2 and a CDR-L1, wherein the
(iv) the CDR-L3 sequence is FQGSHVPWT (SEQ ID NO:5),
(v) the CDR-L2 sequence is KVSNRFS (SEQ ID NO:6), and
(vi) the CDR-L1 sequence is RSSQSIVHSNGNTYLE (SEQ ID NO:7).

9. The antibody of claim 8, wherein
(a) the heavy chain variable region further comprises at least one framework region selected from a H-FR1, a H-FR2, a H-FR3 and a H-FR4, wherein the framework regions are directly adjacent to the CDRs according to the formula (H-FR1)-(CDR-H1)-(H-FR2)-(CDR-H2)-(H-FR3)-(CDR-H3)-(H-FR4), and/or
(b) the light chain variable region further comprises at least one framework region selected from a L-FR1, a L-FR2, a L-FR3 and a L-FR4, wherein the framework regions are directly adjacent to the CDRs according to the formula (L-FR1)-(CDR-L1)-(L-FR2)-(CDR-L2)-(L-FR3)-(CDR-L3)-(L-FR4).

10. The antibody of claim 9, wherein
the H-FR1 sequence is DVQLQESGPGLVKPSQSLSLTCSVTGYSIT (SEQ ID NO:8),
the H-FR2 sequence is WIRQFPGNKLEWMG (SEQ ID NO:9),
the H-FR3 sequence is RISITRDTSKNQFFLKLNSVTTEDTATYYCAG (SEQ ID NO:10),
the H-FR4 sequence is WGQGTLVTVSA (SEQ ID NO:11),
the L-FR1 sequence is DVLMTQTPLSLPVSLGDQASISC (SEQ ID NO:12),
the L-FR2 sequence is WYLQKPGQSPKLLIY (SEQ ID NO:13),
the L-FR3 sequence is GVPDRFSGSGSGTDFTLKINRVEAEDLGVYYC (SEQ ID NO:14), and
the L-FR4 sequence is FGGGTKLEIK (SEQ ID NO:15)

11. The antibody of any one of the preceding claims, wherein the sequence of the CDR-H1, CDR-H2, CDR-H3, CDR-L1, CDR-L2, CDR-L3, H-FR1, H-FR2, H-FR3, H-FR4, L-FR1, L-FR2, L-FR3, L-FR4, heavy chain variable region and/or light chain variable region of said antibody is identical to the respective sequence of the monoclonal antibody produced by the single clone hybridoma cell line “7C10-C5” deposited under the accession number “DSM ACC3350” at “The Leipniz Institute DSMZ-German Collection of Microorganisms and Cell Cultures”.

12. The antibody of any one of the preceding claims, wherein said antibody is a monoclonal antibody.

13. The antibody of any one of claims 1 to 12, wherein said antibody is an IgG1 or IgG2 such as IgG2a, preferably IgG1.

14. The antibody of any one of claims 1 to 12, wherein said antibody is a single-chain variable fragment (scFv) comprising at least one heavy chain variable region and at least one light chain variable region.

15. The antibody of claim 14, wherein said antibody is a bivalent scFv.

16. The antibody of any one of the preceding claims, wherein the heavy chain variable region has the amino acid sequence DVQLQESGPGLVKPSQSLSLTCSVTGYSITSGYYWNWIRQFPGNKLEWMGYIS YDGSNNYNPSLKNRISITRDTSKNQFFLKLNSVTTEDTATYYCAGRFAYWGQ GTLVTVSA (SEQ ID NO:16) and/or the light chain variable region has the amino acid sequence
(SEQ ID NO: 17)
DVLMTQTPLSLPVSLGDQASISCRSSQSIVHSNGNT
YLEWYLQKPGQSPKLLIYKVSNRFSGVPDRFSGSGS
GTDFTLKINRVEAEDLGVYYCFQGSHVPWTFGGG
TKLEIK.

17. The antibody of any one of the preceding claims, wherein said antibody does not specifically bind the non-methylated catalytic subunit of PP2A (PP2Ac), for example, as determined by Biacore according to claim 6 and under the conditions according to claim 7, except that said peptide is not methylated (HVTRRTPDYFL-OH; SEQ ID NO:18).

18. The antibody of any one of the preceding claims, wherein said antibody binds carboxymethylated PP2Ac preferably at least 4, 8, 16, 24, 26, 32, 40 or 48-fold, preferably at least 26-fold stronger than non-carboxymethylated PP2Ac, for example, as determined by an ELISA.

19. The antibody of any one of the preceding claims, wherein said antibody binds a peptide comprising the carboxymethylated C-terminal region of PP2Ac preferably at least 4, 8, 16, 24, 26, 32, 40 or 48-fold, preferably at least 26-fold, stronger than a corresponding peptide comprising the non-methylated C-terminal region of PP2Ac, wherein said C-terminal region of PP2Ac has the sequence TPDYFL (SEQ ID NO:1), for example, as determined by an ELISA, and preferably wherein both peptides have the same length.

20. The antibody of claim 19, wherein the C-terminal region of PP2Ac comprised in the peptide has the sequence HVTRRTPDYFL (SEQ ID NO:18).

21. The antibody of any one of the preceding claims, wherein said antibody binds a peptide consisting of the last 11 amino acids of the carboxymethylated C-terminal region of PP2Ac (HVTRRTPDYFL-CH₃; SEQ ID NO:18) at least 4-, 6-, 8-, 10- or 12-fold, preferably at least 10-fold, e.g. about 12-fold, stronger than a peptide consisting of the last 11 amino acids of the carboxymethylated C-terminal region of PP4c
(PSKKPVADYFL-CH₃; SEQ ID NO: 20).

22. The antibody of claim 21, wherein the binding is determined by Biacore according to claim 6 under the conditions according to claim 7; preferably wherein each peptide is employed at different concentrations, e.g at least 4 different concentrations, wherein for the carboxymethylated peptide having the SEQ ID NO:18 the lowest concentration is about 5 nM and the highest concentration is about 160 nM, and wherein for the carboxymethylated peptide having the SEQ ID NO:20 the lowest concentration is about 50 nM and the highest concentration is about 800 nM.

23. The antibody of any one of the preceding claims, wherein said antibody binds a peptide comprising the C-terminal region of the carboxymethylated PP2Ac (SEQ ID NO:1) preferably at least 4, 8, 10, 16, 24, 32, 40 or 48-fold, preferably at least 10-fold, stronger than a peptide comprising the C-terminal region of the catalytic subunit of protein phosphatase 4 (PP4c), wherein the C-terminal region of PP4c has the sequence VADYFL (SEQ ID NO:19), and preferably wherein both peptides have the same length, for example, as determined by an ELISA.

24. The antibody of claim 23, wherein the C-terminal region of PP2Ac comprised in the peptide has the sequence HVTRRTPDYFL (SEQ ID NO:18) and the C-terminal region of PP4c comprised in the peptide has the sequence PSKKPVADYFL (SEQ ID NO:20).

25. The antibody of claim 23 or 24, wherein the carboxyl group of the C-terminal leucine of both peptides comprising either the C-terminal region of PP2Ac or PP4c is methylated.

26. The antibody of any one of the preceding claims, wherein said antibody binds a peptide comprising the C-terminal region of the carboxymethylated PP2Ac (SEQ ID NO:1) preferably at least 4, 8, 10, 16, 24, 32, 40 or 48-fold, preferably at least 10-fold, stronger than a peptide comprising the C-terminal region of the catalytic subunit of protein phosphatase 6 (PP6c), wherein the C-terminal region of PP6c has the sequence TTPYFL (SEQ ID NO:21), for example, as determined by an ELISA, and preferably wherein both peptides have the same length.

27. The antibody of claim 26, wherein the C-terminal region of PP2Ac comprised in the peptide has the sequence HVTRRTPDYFL (SEQ ID NO:18) and the C-terminal region of PP6c comprised in the peptide has the sequence IPPRTTTPYFL (SEQ ID NO:22).

28. The antibody of claim 26 or 27, wherein the carboxyl group of the C-terminal leucine of both peptides comprising either the C-terminal region of PP2Ac or PP6c is methylated.

29. The antibody of any one of the preceding claims, wherein said antibody binds a peptide comprising the carboxymethylated C-terminal region of PP2Ac preferably at least 4, 6, 8, 16, 24, 32, 40 or 48-fold, preferably at least 6-fold, stronger than a corresponding peptide comprising the amidated C-terminal region of PP2Ac, wherein said C-terminal region of PP2Ac has the sequence TPDYFL (SEQ ID NO:1) for example, as determined by an ELISA, and preferably wherein both peptides have the same length.

30. The antibody of any one of the preceding claims, wherein the binding strength of said antibody to a peptide comprising the carboxymethylated C-terminal region of PP2Ac (SEQ ID NO:1 or SEQ: ID NO:18) is not affected (+/−30%) when the tyrosine in said C-terminal region of PP2Ac is phosphorylated, for example, as determined by an ELISA.

31. The antibody of any one of the preceding claims, wherein the binding strength of said antibody to a peptide comprising the carboxymethylated C-terminal region of PP2Ac (SEQ ID NO:1 or SEQ: ID NO:18) is not affected (+/−30%), or is at most 4-fold, preferably at most 3-fold or 2-fold higher, when the most C-terminal threonine in said C-terminal region of PP2Ac is phosphorylated, for example, as determined by ELISA.

32. The antibody of any one of claims 7 to 31, wherein the peptide(s) is/are acetylated at the N-terminus.

33. The antibody of any one of claims 19, 23, 25, 26, 28, or 29 to 32, wherein the peptide comprising either the C-terminal region of PP2Ac, PP4c or PP6c consists of 6 to 16, preferably 9 to 13, preferably 11 amino acids.

34. The antibody of any one of claims 18 to 21 or 23 to 33, wherein the binding strength of said antibody to a certain peptide is determined by an ELISA, and preferably wherein said binding strength corresponds to the signal intensity determined by said ELISA.

35. The antibody of any one of the preceding claims, wherein said antibody is suitable for quantifying the amount of heterotrimeric PP2A holoenzyme and/or active PP2A in a biological sample.

36. The antibody of any one of the preceding claims, wherein said antibody has been raised against
(i) a peptide comprising the sequence of the 5, preferably 6, C-terminal amino acids of the carboxymethylated PP2Ac or
(ii) a peptide of the sequence TPDYFL (SEQ ID NO:1), PDYFL (SEQ ID NO:23) or RTPDYFL (SEQ ID NO:24), preferably SEQ ID NO:1, wherein the carboxyl group of the C-terminal leucine of said peptide is methylated.

37. The antibody of claim 36, wherein said peptide further includes at the N-terminus a cysteine followed by a β-alanine, and preferably wherein said peptide is coupled via the cysteine to a carrier protein, preferably to keyhole limpet hemocyanin (KLH).

38. A method for producing (i) an antibody specifically binding the carboxymethylated catalytic subunit of protein phosphatase 2A (PP2Ac), and/or (ii) the antibody of any one of claims 1 to 37, wherein said method comprises the steps of
(a) immunizing a non-human mammal, preferably a mouse, with a peptide comprising the sequence of the 5, preferably 6, C-terminal amino acids of the carboxymethylated PP2Ac,
(b) generating hybridoma clones from immune cells of said non-human animal,
(c) selecting a hybridoma clone whereof the supernatant binds preferably at least 4, 8, 16, 20, 24, 26, 32, 40, 50, 100, 200 or 500-fold, preferably at least 100-fold, stronger to the PP2Ac of a cell which contains a PP2A specific methyltransferase (PPM1/LCMT-1) than to the PP2Ac of a cell which lacks said PP2A specific methyltransferase, and
(d) obtaining said antibody from said selected hybridoma clone.

39. The method of claim 38, wherein the immunization peptide in (a) has the sequence TPDYFL (SEQ ID NO:1), PDYFL (SEQ ID NO:23) or RTPDYFL (SEQ ID NO:24), preferably SEQ ID NO:1, wherein the carboxyl group of the C-terminal leucine of said peptide is methylated.

40. The method of claim 38 or 39, wherein the immunization peptide in (a) further includes at the N-terminus a cysteine followed by a β-alanine, and preferably is coupled via the cysteine to a carrier protein, preferably to keyhole limpet hemocyanin (KLH).

41. The method of any one of claims 38 to 40, wherein the cell containing said PP2A specific methyltransferase lacks the PP2A specific demethylase (PPE-1/PME1).

42. The method of any one of claims 38 to 41, wherein the cells for assaying the binding of the hybridoma supernatant are yeast cells.

43. The method of any one of claims 38 to 42, wherein the PP2Ac of a cell is contained in the lysate of said cell, and preferably wherein the binding of the hybridoma supernatant to said PP2Ac is determined by western blotting.

44. An antibody obtainable by the method of any one of claims 38 to 42.

45. Use of an anti-carboxymethylated PP2Ac-specific antibody of any one of claims 1 to 37 for specifically detecting the carboxymethylated catalytic subunit of protein phosphatase 2A (PP2Ac) in a biological sample.

46. A method for specifically detecting the carboxymethylated catalytic subunit of protein phosphatase 2A (PP2Ac) in a biological sample, wherein said method comprises a step of contacting said biological sample with an anti-carboxymethylated PP2Ac-specific antibody of any one of claims 1 to 37.

47. The method of claim 47, wherein the biological sample does not have a pH greater than 8.0, preferably 8.5, and/or said sample is not treated with a liquid having a pH greater than 8.0, preferably 8.5, preferably wherein said sample is not treated with said liquid before and/or during contacting with said antibody.

48. An in vitro method for prognosing the outcome of a cancer in a patient, wherein said method comprises the steps of
(a) determining the level of carboxymethylated PP2Ac in a sample from said patient by contacting said sample with an anti-carboxymethylated PP2Ac-specific antibody according to any one of claims 1 to 37;
(b) comparing the level of carboxymethylated PP2Ac to a threshold value and/or reference level; and
(c) prognosing the outcome of said cancer, wherein
(i) a positive outcome is prognosed when the level of carboxymethylated PP2Ac is equal to or greater than said threshold value and/or reference level,
and/or
(ii) a negative outcome is prognosed when the level of carboxymethylated PP2Ac is lower than said threshold value and/or reference level.

49. The method of claim 48, wherein the positive outcome is survival of the patient, preferably recurrence free survival, for at least about 0.5, 1, 2, 3, 4, 5, 6, 7, 8, 9, 10, 11, 12, 13, 14, 15 or 20 years, preferably at least 5 or 6 years, more preferably at least 10 years; and/or wherein the negative outcome is death and/or recurrence of the cancer in less than about 0.5, 1, 2, 3, 4, 5, 6, 7, 8, 9, 10, 11, 12, 13, 14, 15 or 20 years, e.g. less than about 10 years, or less than about 5 or about 6 years.

50. An in vitro method for diagnosing whether a cancer is metastatic or prone to metastasize, wherein said method comprises the steps of
(a) determining the level of carboxymethylated PP2Ac in a sample from said patient by contacting said sample with an anti-carboxymethylated PP2Ac-specific antibody according to any one of claims 1 to 37;
(b) comparing the level of carboxymethylated PP2Ac to a threshold value and/or reference level; and
(c) diagnosing whether the cancer is metastatic or prone to metastasize, wherein
(i) it is diagnosed that said cancer is not metastatic or prone to metastasize when the level of carboxymethylated PP2Ac is equal to or greater than said threshold value and/or reference level,
and/or
(ii) it is diagnosed that said cancer is metastatic or prone to metastasize when the level of carboxymethylated PP2Ac is lower than said threshold value and/or reference level.

51. The method of any one of claims 48 to 50, wherein said threshold value refers to an Allred Score of 2 determined by immunohistochemistry staining with said anti-carboxymethylated PP2Ac-specific antibody, in particular wherein
(i) an Allred Score of equal to or higher than 2 means that some cells in the sample, e.g. at least 0.1%, 0.5% or 1%, preferably at least 0.5%, of the cells show some staining, e.g. a weak, intermediate or high staining, and/or
(ii) an Allied Score of lower than 2 means that essentially no cells in the sample, e.g. less than 0.1%, preferably less than 0.01%, more preferably no cells (0%), show at most a weak staining, preferably no staining.

52. The method of any one of claims 48 to 51, wherein said reference level is determined by analyzing the level of carboxymethylated PP2Ac in samples from a plurality of reference patients diagnosed with a cancer by contacting said sample with an anti-carboxymethylated PP2Ac-specific antibody according to any one of claims 1 to 37, wherein it is known whether the outcome of the cancer of said reference patients has been positive or negative,
in particular wherein the reference level allows to separate the reference patients with a positive outcome from the reference patients with a negative outcome in an optimal way, and/or
in particular wherein the level of carboxymethylated PP2Ac in the samples from said reference patients is determined by the same measurement method that is employed in said step (a).

53. A method of detecting an abnormal level of carboxymethylated PP2Ac in a sample from a patient, wherein said method comprises
(a) measuring the level of carboxymethylated PP2Ac in a sample from said patient by contacting said sample with an anti-carboxymethylated PP2Ac-specific antibody according to any one of claims 1 to 37; and
(b) determining whether the sample is abnormal, wherein the sample is determined to be abnormal if the level of carboxymethylated PP2Ac is at least about 20% lower than the amount determined for a reference sample.

54. The method of claim 53, wherein the sample is determined to be abnormal if the level of carboxymethylated PP2Ac is at least about 30%, about 40%, about 50%, about 60%, about 70%, or about 80%, preferably at least about 60%, more preferably at least about 80%, e.g. about 80%, lower than the amount determined for the reference sample.

55. The method of claim 53 or 54, wherein the method further comprises:
(c) reporting to said patient whether said sample is determined to be abnormal or normal.

56. The method of any one of claims 53 to 55, wherein said patient is suffering from a cancer, preferably a prostate cancer.

57. The method of any one of claims 53 to 56, wherein the reference sample is derived from at least one patient not suffering from a disease selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A).

58. The method of any one of claims 53 to 57, wherein the reference sample is derived from at least one patient not suffering from a cancer.

59. The method of any one of claims 53 to 56, wherein the reference sample is derived from at least one patient not suffering from a metastatic cancer, preferably a metastatic prostate cancer.

60. The method of any one of claims 55 to 59, wherein said patient having reported an abnormal level of carboxymethylated PP2Ac is also reported to expect death and/or recurrence of the cancer in less than about 0.5, 1, 2, 3, 4, 5, 6, 7, 8, 9, 10, 11, 12, 13, 14, 15 or 20 years, e.g. in less than about 10 years, or less than about 5 or about 6 years.

61. The method of any one of claims 55 to 59, wherein said patient having reported a normal level of carboxymethylated PP2Ac is also reported to expect survival, preferably recurrence free survival, for at least 0.5, 1, 2, 3, 4, 5, 6, 7, 8, 9, 10, 11, 12, 13, 14, 15 or 20 years, preferably at least 5 or 6 years, more preferably at least 10 years.

62. The method of any of claims 48 to 61, wherein said patient is suspected of having a metastatic cancer or developing a metastatic cancer.

63. The method of any one of claims 48 to 62, wherein said sample is a cancer sample.

64. The method of any one of claims 48 to 52 or 56 to 63, wherein said cancer is associated with and/or caused by hyperphosphorylation of androgen receptor, c-MYC, ERK, AKT, S6K, β-catenin, and/or Bcl2, preferably androgen receptor.

65. An in vitro method for prognosing the responsiveness of a cancer in a patient to treatment with an antiandrogen, wherein said method comprises the steps of
(a) determining the level of carboxymethylated PP2Ac in a sample from said patient by contacting said sample with an anti-carboxymethylated PP2Ac-specific antibody according to any one of claims 1 to 37;
(b) comparing the level of carboxymethylated PP2Ac to a reference level; and
(c) prognosing whether the cancer is responsive, wherein
(i) it is prognosed that said cancer is responsive when the level of carboxymethylated PP2Ac is equal to or greater than said reference level,
and/or
(ii) it is prognosed that said cancer is not responsive when the level of carboxymethylated PP2Ac is lower than said reference level;
in particular wherein a response corresponds to
(i) elimination of the cancer,
(ii) prevention and/or elimination of metastases,
(iii) a reduction of the cancer volume by at least 30%
(iv) survival of the cancer patient for at least about 0.5, 1, 2, 3, 4, 5, 6, 7, 8, 9, 10, 11, 12, 13, 14, 15 or 20 years, preferably at least 5 years; and/or
(v) a decline of at least one cancer tumor marker, e.g. prostate-specific antigen (PSA), by at least 50%.

66. The method of claim 65, wherein said reference level is determined by analyzing the level of carboxymethylated PP2Ac in samples from a plurality of reference patients diagnosed with a cancer by contacting said sample with an anti-carboxymethylated PP2Ac-specific antibody according to any one of claims 1 to 37, wherein it is known whether the cancer of said reference patients has been responsive to an antiandrogen treatment or not,
in particular wherein the reference level allows to separate the reference patients with a responsive cancer from the reference patients with an unresponsive cancer in an optimal way, and/or
in particular wherein the level of carboxymethylated PP2Ac in the samples from said reference patients is determined by the same measurement method that is employed in said step (a).

67. The method of claim 65 or 66, wherein the antiandrogen is an antagonist of androgen receptor signaling, preferably an androgen receptor antagonist, more preferably enzalutamide.

68. A method for prognosing the progression of a disease in a subject, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with an anti-carboxymethylated PP2Ac-specific antibody of any one of claims 1 to 37, and
(b) evaluating whether the disease progression is favorable or unfavorable, and indicating a favorable progression if
said disease is associated with hyperphosphorylation of said signaling pathway component and the level of said carboxymethylated PP2Ac is elevated, or
said disease is associated with hypophosphorylation of said signaling pathway component and the level of said carboxymethylated PP2Ac is reduced, and/or
indicating an unfavorable progression if
said disease is associated with hyperphosphorylation of said signaling pathway component and the level of said carboxymethylated PP2Ac is reduced, or
said disease is associated with hypophosphorylation of said signaling pathway component and the level of said carboxymethylated PP2Ac is elevated.

69. The method of claim 68, wherein an elevated level of said carboxymethylated PP2Ac corresponds to an Allied Score of 2 or greater, and/or wherein a reduced level of said carboxymethylated PP2Ac corresponds to an Allred Score lower than 2, preferably as defined in claim 51.

70. A method for predicting the responsiveness of a subject suffering from a disease to the treatment with a PP2A modulator, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in a sample by contacting said sample with an anti-carboxymethylated PP2Ac-specific antibody of any one of claims 1 to 37, and
(b) evaluating whether said subject will be responsive to the treatment with said PP2A modulator, and
indicating that said subject will be responsive if
said disease is associated with hyperphosphorylation of said signaling pathway component, said PP2A modulator activates PP2A, and the level of said carboxymethylated PP2Ac is reduced, or
said disease is associated with hypophosphorylation of said signaling pathway component, said PP2A modulator inhibits PP2A, and the level of said carboxymethylated PP2Ac is elevated, and/or
indicating that said subject will not be responsive if
said disease is associated with hyperphosphorylation of said signaling pathway component, said PP2A modulator activates PP2A, and the level of said carboxymethylated PP2Ac is elevated, or
said disease is associated with hypophosphorylation of said signaling pathway component, said PP2A modulator inhibits PP2A, and the level of said carboxymethylated PP2Ac is reduced.

71. A method for determining the responsiveness of a subject suffering from a disease to the treatment with a PP2A modulator, wherein said disease is selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation or hypophosphorylation of at least one component of said signaling pathway, and wherein said component can be dephosphorylated by protein phosphatase 2A (PP2A),
wherein said method comprises the steps of
(a) determining in vitro the level of carboxymethylated PP2Ac in sample by contacting said sample with an anti-carboxymethylated PP2Ac-specific antibody of any one of claims 1 to 37, wherein said biological sample is derived from a tissue that has been contacted in said subject with said PP2A modulator,
(b) determining that said subject responds to the treatment with said PP2A modulator, if the level of carboxymethylated PP2Ac in said sample is altered.

72. The method of any one of claims 68 to 71, wherein the sample is a sample from the subject to be assessed.

73. The method of any one of claims 48 to 72, wherein the patient or subject is a human.

74. The method of any one of claims 68 to 73, wherein the carboxymethylated PP2Ac level in said sample is compared to the carboxymethylated PP2Ac level in a reference/control sample, thereby determining if the carboxymethylated PP2Ac level is reduced or increased.

75. The method of any one of claims 70 to 74, wherein the PP2A modulator is a PP2A activator.

76. The method of claim 75, wherein the PP2A activator is a small molecule, preferably a modified phenothiazine and/or a small molecule derived from phenothiazine.

77. The method of claim 75 or 76, wherein the PP2A activator is DT-061 (CAS No.: 1809427-19-7 or CAS No.: 1809427-18-6) or forskolin (Colforsin; (3R,4aR,5S,6S,6aS,10S,10aR,10bS)-dodecahydro-5,6,10,10b-tetrahydroxy-3,4a,7,7,10a-pentamethyl-1-oxo-3-vinyl-1H-naphtho[2,1-b]pyran-5-yl acetat), preferably DT-061.

78. The method of any one claims 68 to 77, wherein said deregulation comprises the hyperphosphorylation of said component of said signaling pathway.

79. The method of any one claims 68 to 78, wherein said signaling pathway comprises a tyrosine kinase receptor, androgen receptor, Bcl2, PI3K, AKT, S6K, MAP, ERK, β-catenin and/or c-MYC, preferably wherein the tyrosine kinase receptor is EGFR.

80. The method of any one of claims 68 to 79, wherein the component that can be dephosphorylated by PP2A is selected from, c-MYC, ERK, AKT, S6K, β-catenin, androgen receptor and Bcl2.

81. The method of any one of claims 68 to 80, wherein the disease is a cancer, a neurodegenerative disorder, diabetes and/or a heart disease, preferably cancer.

82. The method of any one of claims 68 to 81, wherein the disease is a cancer that is associated with and/or caused by a hyperactive Akt, S6K and/or ERK/MAP signaling pathway(s) and/or the hyperphosphorylation of at least one component of said signaling pathways(s), wherein said component can be dephosphorylated by PP2A.

83. The method of any one of claims 68 to 82, wherein the disease is a cancer that is associated with and/or caused by abnormal androgen receptor signaling and/or hyperphosphorylation of androgen receptor.

84. The method of any one of claims 48 to 83, wherein the biological sample is derived from a diseased and/or pathogenic tissue from said subject.

85. The method of any one of claims 48 to 84, wherein the cancer is prostate cancer, lung adenocarcinoma, or breast cancer, preferably prostate cancer.

86. The method of any one of claims 68 to 84, wherein disease, in particular the neurodegenerative disorder, is Alzheimer's disease.

87. A method for evaluating whether a test agent modulates PP2Ac carboxymethylation, wherein said method comprises the steps of
(a) assessing effects of said test agent on PP2Ac carboxymethylation status in a PP2Ac methylation assay, wherein said assay contains PP2Ac and
(i) a PP2Ac methylase enzyme and/or a PP2Ac demethylase enzyme,
and/or
(ii) an A and B subunit of PP2A, and
wherein the PP2Ac carboxymethylation status is determined by an anti-carboxymethylated PP2Ac-specific antibody of any one of claims 1 to 37; and
(b) determining based on the assessed effects that said test agent modulates PP2Ac carboxymethylation or that said test agent does not modulate PP2Ac carboxymethylation.

88. A method for identifying agents that modulate PP2Ac carboxymethylation, wherein said method comprises the steps of
(a) providing a plurality of candidate test agents;
(b) assessing effects of an individual candidate test agent on PP2Ac carboxymethylation status in a PP2Ac methylation assay, wherein said assay contains PP2Ac and
(i) a PP2Ac methylase enzyme and/or a PP2Ac demethylase enzyme; and/or
(ii) an A and B subunit of PP2A, and
wherein the PP2Ac carboxymethylation status is determined by an anti-carboxymethylated PP2Ac-specific antibody of any one of claims 1 to 37; and
(c) identifying, based on the assessed effects, at least one agent that modulates PP2Ac methylation.

89. The method of claim 87 or 88, wherein said effects on PP2Ac carboxymethylation status comprise an increase or reduction of PP2Ac carboxymethylation compared to a reference and/or control.

90. The method of any one of claims 87 to 89, wherein the PP2Ac methylation assay comprises a cell and/or cell lysate and/or is performed in a cell and/or cell lysate.

91. The method of any one of claims 87 to 90, wherein assessing the effects of a test agent on the PP2Ac carboxymethylation status comprises the steps of
(b′) contacting the PP2Ac methylation assay with said test agent, and subsequently
(b″) contacting said PP2Ac methylation assay with an anti-carboxymethylated PP2Ac-specific antibody of any one of claims 1 to 37, thereby determining the PP2Ac carboxymethylation status.

92. A method for evaluating whether a test agent modulates the activity of PP2A, wherein said method comprises the steps of
(a) evaluating whether said test agent modulates PP2Ac carboxymethylation according to the evaluation method of any one claims 87 or 89 to 91, and
(b) determining that said test agent modulates the activity of PP2A, if said test agent modulates PP2Ac carboxymethylation.

93. A method for identifying an agent that modulates the activity of PP2A, wherein said method comprises the steps of
(a) identifying an agent that modulates PP2Ac carboxymethylation according to the screening method of any one claims 88 to 92, and
(b) selecting said identified agent.

94. The method of claim 92 or 93, wherein the agent or test agent activates PP2A and/or enhances the activity of PP2A, if said agent or test agent increases PP2Ac carboxymethylation.

95. The method of claim 92 or 93, wherein the agent or test agent inhibits the activity of PP2A and/or inhibits the activation of PP2A, if said agent or test agent reduces PP2Ac carboxymethylation.

96. The method of claim 93 for identifying an agent that activates PP2A and/or enhances the activity of PP2A, wherein said method comprises the steps of
(a) identifying an agent that increases PP2Ac carboxymethylation according to the screening method of any one claims 88 to 92, and
(b) selecting said identified agent.

97. The method of claim 93 for identifying an agent that inhibits the activity of PP2A and/or inhibits the activation of PP2A, wherein said method comprises the steps of
(a) identifying an agent that reduces PP2Ac carboxymethylation according to the screening method of any one claims 88 to 92, and
(b) selecting said identified agent.

98. The method of any one of claims 87 to 97, wherein the agent or test agent is a PP2A activator, if said agent/or est agent
(i) activates the PP2Ac methylase enzyme and/or enhances the activity of the PP2Ac methylase enzyme; and/or
(ii) inhibits the activity of the PP2Ac demethylase enzyme and/or inhibits the activation of the PP2Ac demethylase enzyme.

99. The method of any one of claims 87 to 98, wherein the agent or test agent is a PP2A activator, if said agent or test agent increases the amount of the trimeric PP2A holoenzyme.

100. The method of any one of claims 87 to 99, wherein the agent or test agent is a PP2A inhibitor, if said agent or test agent
(i) activates the PP2Ac demethylase enzyme and/or enhances the activity of the PP2Ac demethylase enzyme; and/or
(ii) inhibits the activity of the PP2Ac methylase enzyme and/or inhibits the activation of the PP2Ac methylase enzyme.

101. The method of any one of claims 87 to 100, wherein the agent or test agent is a PP2A inhibitor, if said agent or test agent reduces the amount of the trimeric PP2A holoenzyme.

102. The method of any one of claims 87 to 101, wherein, if said agent or test agent is a PP2A activator, said agent or test agent is DT-061 (CAS No.: 1809427-19-7 or CAS No.:
1809427-18-6) or forskolin (Colforsin; (3R,4aR,5S,6S,6aS,10S,10aR,10bS)-dodecahydro-5,6,10,10b-tetrahydroxy-3,4a,7,7,10a-pentamethyl-1-oxo-3-vinyl-1H-naphtho[2,1-b]pyran-5-yl acetat), preferably DT-061.

103. The screening method of any one claims 88 to 102 further comprising a step of obtaining the identified agent.

104. The method of any one of claims 87 to 102 or the screening method of claim 103, wherein said agent or test agent is a medicament and/or a pharmaceutical.

105. An PP2A activator obtained according to claim 103 for use in treating a disease selected from the group consisting of the diseases of the following (i) and/or (ii):
(i) cancer, a neurodegenerative disorder, diabetes and/or a heart disease;
(ii) a disease that is associated with and/or is caused by a deregulated signaling pathway, wherein said deregulation comprises the hyperphosphorylation of at least one component of said signaling pathway, and wherein said component can be dephosphorylated by protein phosphatase 2A (PP2A).

106. An PP2A inhibitor obtained according to claim 103 for use in chemotherapy and/or radiotherapy.

107. A method for identifying a PP2A modulator, wherein said method comprises the steps of
(a) contacting a cell or cell lysate containing an A, B and C subunit of PP2A with a candidate PP2A modulator,
(b) contacting said cell or cell lysate with an anti-carboxymethylated PP2Ac-specific antibody of any one of claims 1 to 37, thereby determining the level of carboxymethylated PP2Ac in said cell or cell lysate, and
(c) selecting said candidate PP2A modulator if the level of carboxymethylated PP2Ac in said cell or cell lysate is altered.

108. A screening method for evaluating whether a molecule modulates the activity of PP2A, wherein said method comprises the steps of
(a) contacting a cell or cell lysate containing an A, B and C subunit of PP2A with said molecule,
(b) contacting said cell or cell lysate with an anti-carboxymethylated PP2Ac-specific antibody of any one of claims 1 to 37, thereby determining the level of carboxymethylated PP2Ac in said cell or cell lysate, and
(c) indicating that said molecule modulates the activity of PP2A if the level of carboxymethylated PP2Ac in said cell or cell lysate is altered.

109. The method of claim 107 or 108, wherein said PP2A modulator activates PP2A and/or enhances the activity of PP2A, said modulation is the activation of PP2A and/or the enhancement of the PP2A activity, and said alteration is an elevated carboxymethylated PP2Ac level.

110. The method of any one of claims 107 to 109, wherein said PP2A modulator or activator is DT-061 (CAS No.: 1809427-19-7 or CAS No.: 1809427-18-6) or forskolin (Colforsin; (3R,4aR,5S,6S,6aS,10S,10aR,10bS)-dodecahydro-5,6,10,10b-tetrahydroxy-3,4a,7,7,10a-pentamethyl-1-oxo-3-vinyl-1H-naphtho[2,1-b]pyran-5-yl acetat), preferably DT-061.

111. A method for screening for (a) medicament(s) and/or drug(s), said method comprising the step of evaluating the capacity of said medicament or drug to stabilize and/or increase the methylation status of PP2Ac and/or increase methylated PP2Ac levels, wherein said stabilization, increase and/or methylation level is to be detected with an anti-carboxymethylated PP2Ac-specific antibody of any one of claims 1 to 37.

112. The method of claim 111, wherein said stabilization, said methylation status and/or said methylation level of PP2Ac is to be determined in a cell or tissue or in a cell lysate.

113. The method of claim 111 or 112, wherein said stabilization, said methylation status and/or said methylation level of PP2Ac is to be determined in or on an in vitro cell, in or on an in vitro tissue or in a cell or tissue of a test animal or in or on an isolated human cell and/or tissue sample.

114. The method of any one of claims 111 to 113, wherein said medicament and/or drug to be screened is an activator of protein phosphatase 2A.
